(19) United States
(12) Patent Application Publication
(10) Pub. No.: US 2026/0193170 A1
(43) Pub. Date: Jul. 9, 2026

(54) Noncovalent Keap1-Nrf2 Small-Molecule Inhibitors

(71) Applicant: University of Copenhagen, Copenhagen K (DK)

(72) Inventors: Chunyu Lin, Copenhagen K (DK); Jie Zang, Copenhagen K (DK); Dilip Narayanan, Copenhagen K (DK); Anders Bach, Copenhagen K (DK)

(21) Appl. No.: 19/133,416

(22) PCT Filed: Nov. 30, 2023
(86) PCT No.: PCT/EP2023/083694

§ 371 Date: May 28, 2025

(30) Foreign Application Priority Data
Nov. 30, 2022 (EP) 22210485.3

(51) Int. Cl. C07C 235/22; A61K 31/196; A61K 31/343; A61K 31/351; A61K 31/357; A61K 31/36; A61K 31/4192; A61K 31/427; A61K 31/437; A61K 31/4525; A61K 31/4709; A61K 31/5375; C07C 235/24; C07D 249/18; C07D 295/32; C07D 307/79; C07D 309/14; C07D 317/60; C07D 319/18; C07D 405/06; C07D 405/12; C07D 417/06; C07D 471/04

(52) U.S. Cl. CPC C07C 235/22; A61K 31/196; A61K 31/343; A61K 31/351; A61K 31/357; A61K 31/36; A61K 31/4192; A61K 31/427; A61K 31/437; A61K 31/4525; A61K 31/4709; A61K 31/5375; C07C 235/24; C07D 249/18; C07D 295/32; C07D 307/79; C07D 309/14; C07D 317/60; C07D 319/18; C07D 405/06; C07D 405/12; C07D 417/06; C07D 471/04; C07C 2601/14; C07C 2601/16; C07C 2601/18

(57) ABSTRACT

The present invention relates to a series of noncovalent Keap1-Nrf2 small-molecule inhibitors and their use as medicaments. The series comprises novel small-molecule analogues with strong affinity for the Keap1 Kelch domain and ability to inhibit the Keap1-Nrf2 interaction.

### TECHNICAL FIELD

The present invention relates to noncovalent Keap1-Nrf2 small-molecule inhibitors and their use as medicaments.

### BACKGROUND

Reactive oxygen species (ROS) generated in low levels during cellular homeostasis are used for redox signalling events and controlled by endogenous antioxidants and neutralizing enzymes. Several disease conditions, however, lead to ROS amounts exceeding the capacity of these endogenous defence molecules, which can result in oxidative stress and inflammation.^{1, 2}

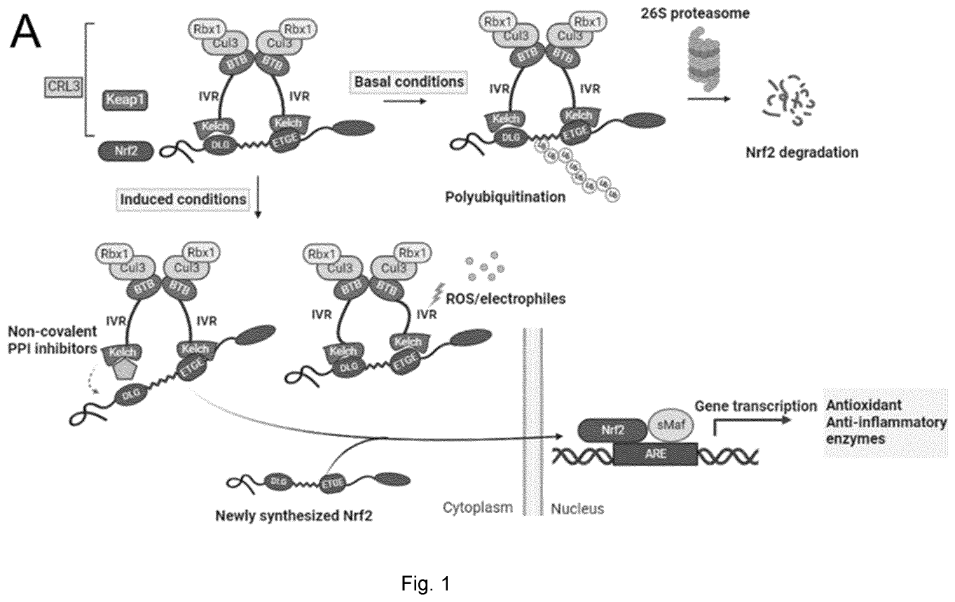

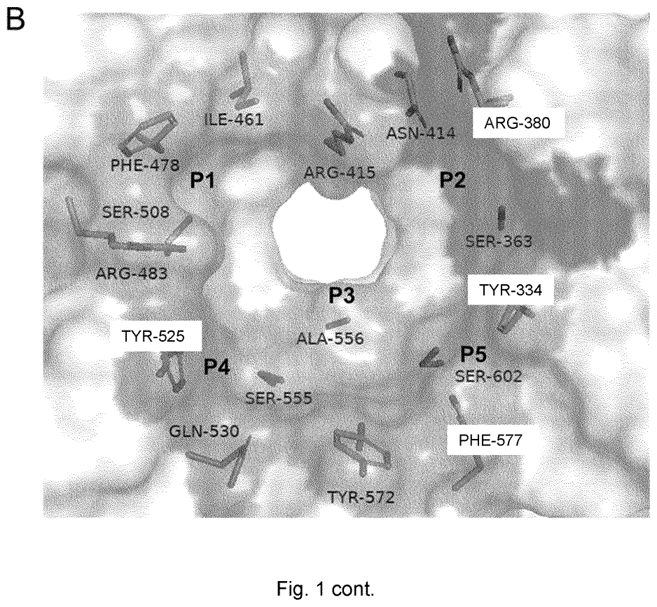

The protein-protein interaction (PPI) between the transcription factor nuclear erythroid-related factor 2 (Nrf2) and the Kelch domain of Kelch-like ECH-associated protein 1 (Keap1) is central in the cellular adaptive response to fluctuating levels of ROS and exogenous electrophiles. Keap1 is a substrate adaptor protein and component of the cullin 3 E3 ubiquitin ligase complex that recognizes and ubiquitinates Nrf2, marking it for proteasomal degradation.³ However, when ROS levels increase, key sensor cysteine residues on the broad complex, tramtrack, and bric-à-brac (BTB) domain and intervening region (IVR) of Keap1 are modified, which leads to a conformational change in Keap1 that prevents Cullin 3-mediated ubiquitination.^{4,5} This results in cytosolic accumulation of Nrf2 followed by translocation into the nucleus, where Nrf2 forms a transcription factor complex that induces expression of detoxifying antioxidant enzymes, such as NAD (P) H: quinone oxidoreductase 1 (NQO1), heme oxygenase 1 (HO-1), and glutathione reductase, and suppresses pro-inflammatory genes (FIG. 1A).⁶

Pharmacological inhibition of the Keap1-Nrf2 PPI has emerged as a promising therapeutic strategy to alleviate oxidative stress and reduce inflammation in many diseases,⁶⁻⁹ for example CNS injuries (cerebral ischemia,¹⁰ intracerebral haemorrhage,¹¹ ischemic stroke,¹²⁻¹⁴ and traumatic brain injury¹⁵), retinal ischemia-reperfusion (I/R) injury,¹⁶ neurodegenerative disorders (e.g. Alzheimer's disease,^{17, 18} Parkinson's disease, and multiple sclerosis),^{6, 19} lung diseases (e.g. chronic obstructive pulmonary disease,²⁰ pulmonary hypertension,⁶ and acute lung inflammation^{21, 22}), metabolic and and/or inflammatory liver conditions (such as non-alcoholic steatohepatitis, NASH)^{23, 24}, chronic kidney disease (CKD;²⁵ including Alport syndrome,^{6, 26}) skin diseases (e.g. atopic dermatitis and psoriasis²⁷), rheumatoid arthritis,²⁸ ulcerative colitis,²⁹ acetaminophen-induced hepatotoxicity,³⁰ and some cancer types.⁶ Thus, Keap1 is a promising drug target for a diverse set of pathologies.

Electrophilic Keap1-Nrf2 inhibitors, which covalently bind the sensor cysteines of Keap1, have been thoroughly explored and are often effective in enhancing the antioxidant defence response.⁶ A prominent example is dimethyl fumarate,³¹ which is clinically used against multiple sclerosis and psoriasis. However, unspecific reactivity of covalent binders can lead to toxicity and uncertainties about the mode of action.^{32, 33} Thus, noncovalent reversible Keap1-Nrf2 PPI inhibitors comprise an alternative and more attractive strategy. These bind the Keap1 Kelch domain and displace Nrf2 either partly or fully from Keap1, hence allowing Nrf2 to escape proteasomal degradation and initiate the expression Nrf2-controlled genes.^{4,5} Such inhibitors could provide specific, less toxic, and potent chemical probes and drug leads.^{4, 6, 7, 34}

Today, there exist several small-molecule noncovalent reversible Keap1-Nrf2 inhibitors. However, they often have suboptimal properties, such as low specificity, low affinity, low solubility, mutagenic activity, low metabolic stability, low membrane permeability, or low bioavailability, and/or high clearance.³⁵⁻³⁷ Also, their size (molecular weight and total polar surface area), number of hydrogen bond donors, and inclusion of carboxylic acids prevent brain permeability and thus limit their use as chemical probes for investigating CNS diseases.⁴ These properties are likely a result of the nature of the Keap1 Kelch binding pocket, as, to obtain high affinity, it appears that both the polar arginine-containing subpockets (P1 and/or P2) and the hydrophobic subpockets (P4 and/or P5) need to be occupied (FIG. 1B), generally leading to large and charged compounds.^{4, 6, 38}

Fragment-based drug discovery (FBDD) has in recent years become a state-of-the-art strategy for the development of high-quality chemical probes and clinical candidates.^{39, 40} FBDD has been used to make three series of noncovalent Keap1-Nrf2 inhibitors.^{20, 36, 41} The principle of FBDD is to screen for small substructures (fragments) of drug-like compounds with molecular weights of 100-300 Daltons and few functional and hydrogen-bonding groups and use these as starting points for further optimization.⁴² This contrasts with conventional high-throughput screening (HTS) where larger molecules of 250-600 Daltons are tested. The advantage of FBDD is that small fragments can fit easier into protein binding pockets, whereby they contain higher binding energies relative to their size, compared to a typical and larger HTS hit that often form low-quality interactions and therefore is difficult to optimize. Chemical growing or linking of the fragment hits, preferably guided by X-ray crystallographic data, can convert the fragments into highly optimized and potent molecules. By making sure that the molecule does not grow excessively large or lipophilic during the fragment-to-lead optimization process, molecules with more drug-like properties (e.g. good solubility, metabolic stability, membrane permeability, absorption, pharmacokinetic properties etc.) are achieved.

### SUMMARY

Keap1 is a promising drug target for a diverse set of pathologies, where oxidative stress and inflammation play key roles. The present inventors have developed a series of novel noncovalent small-molecule inhibitors of the Keap1-Nrf2 interaction by FBDD. A crystallographic screening against the Keap1 Kelch domain followed by optimization of one of the fragment hits by structure-based drug design, led to an up to 171,000-fold increase in affinity and thereby a series of analogues with strong affinity for the Keap 1 Kelch domain and ability to inhibit the Keap1-Nrf2 interaction.

In one aspect, the present invention relates to a compound of Formula (XI):

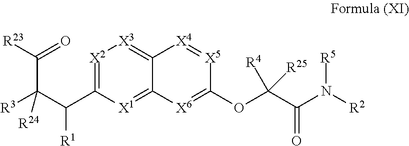

wherein
X¹ is CH or N;
X² is CH or N;
X³ is CH or N;
X⁴ is CH or N;
X⁵ is CH or N;
X⁶ is CH or N;
R¹ is phenyl, pyridinyl or H, wherein the phenyl or pyridinyl is optionally substituted with one or more, identical or different, substituents R⁶, wherein two of the substituents R⁶ optionally are linked together to form a ring;
R² is selected from the group consisting of C₅₋₇ cycloalkyl, C₅₋₇ cycloalkenyl, C₅₋₇ heterocycloalkyl, phenyl, C₅₋₈ bicycloalkyl, C₃₋₆ alkyl, and C₉₋₁₂ tricycloalkyl, wherein said C₅₋₇ cycloalkyl, C₅₋₇ cycloalkenyl, C₅₋₇ heterocycloalkyl, phenyl, C₅₋₈ bicycloalkyl, C₃₋₆ alkyl, and C₉₋₁₂ tricycloalkyl is optionally substituted with one or more, identical of different substituents R²⁸;
R⁵ is H or CH₃;
or R² is C₃₋₆ alkanediyl optionally substituted with R²⁸, R⁵ is a bond, and R² and R⁵ are linked together to form a ring;
R³ is H or CH₃;
R⁴ is H or CH₃;
R⁶ is selected from the group consisting of C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group of the C₁₋₅ alkyl, —OC₁₋₅ alkyl, or C₂₋₅ alkenyl is optionally replaced with O or N(R⁷); and/or
   wherein one or more methine group of the C₂₋₅ alkenyl is optionally replaced with N;
R⁷ is H or C₁₋₃ alkyl;
R²³ is selected from the group consisting of —OH;

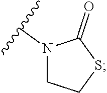 [SMILES: *N1CCSC1=O]

—OC₁₋₅ alkyl optionally substituted with one or more, identical or different, substituents R²⁶; —OC₂₋₅ alkenyl optionally substituted with one or more, identical or different, substituents R²⁶; —OC₂₋₅ alkynyl optionally substituted with one or more, identical or different, substituents R²⁶, —OC₃₋₆ cycloalkyl optionally substituted with one or more, identical or different, substituents R²⁶; —O-phenyl optionally substituted with one or more, identical or different, substituents R²⁷; and —O-benzyl optionally substituted with one or more, identical or different, substituents R²⁷; and —NH(CH₃);
R²⁴ is H or CH₃;
R²⁵ is H or CH₃;
R²⁶ is independently selected from the group consisting of H, deuterium, F, Cl and —OC₁₋₃ alkyl;
R²⁷ is independently selected from the group consisting of deuterium, methoxy, nitro, cyano, Cl, Br, I, and F; and
R²⁸ is individually selected from C₁₋₃ alkyl, —OC₁₋₃ alkyl, halogen, and deuterium, wherein said C₁₋₃ alkyl is optionally substituted with one or more halogens; or a pharmaceutically acceptable salt, hydrate, polymorph, tautomer, or solvate thereof.

### DESCRIPTION OF DRAWINGS

FIG. 1: The Keap1-Nrf2 PPI.
A) Molecular mechanisms of the Keap1-Nrf2 pathway showing the effect of ROS, electrophiles and non-covalent PPI inhibitors.
B) X-ray crystal structure of the Neh2-binding pocket (P1-P5) of the Keap1 Kelch domain (modified from PDB ID 6ZF8). The five subpockets are labelled P1-5.

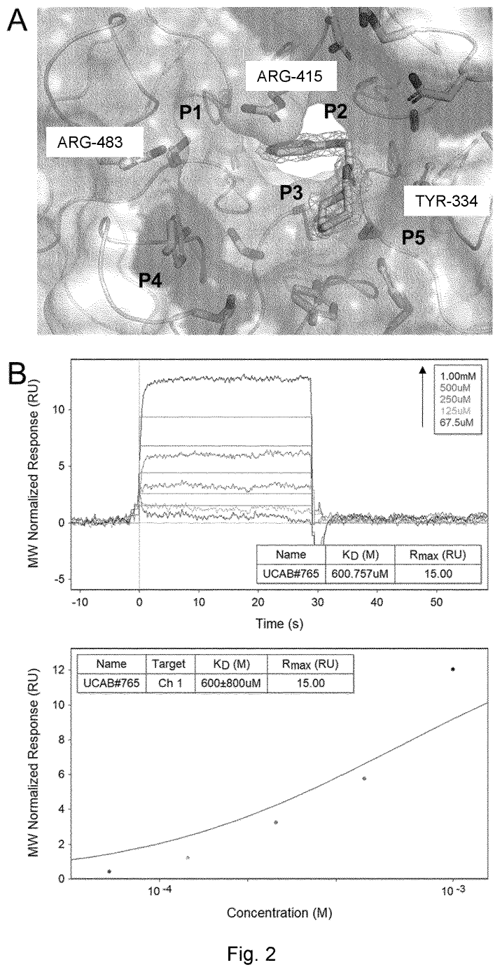

FIG. 2: Fragment hit XCF #0252 binding to the Keap1 Kelch domain.
A) X-ray crystal structures of XCF #0252 in complex with the Keap1 Kelch domain with indication of the P1-P5 subpockets. Standard 2Fo-Fc electron density map carved around the fragments at 1.6 Å contoured at 10 are shown.
B) SPR sensorgrams of XCF #0252 (=UCAB #765) injected in 2-fold serial dilutions over immobilized Keap1 Kelch (up), and plots of equilibrium binding responses against the injected concentrations (down). K_{d} was determined to 0.6 mM when constraining Rₘₐₓ to 15 RU (theoretical max).

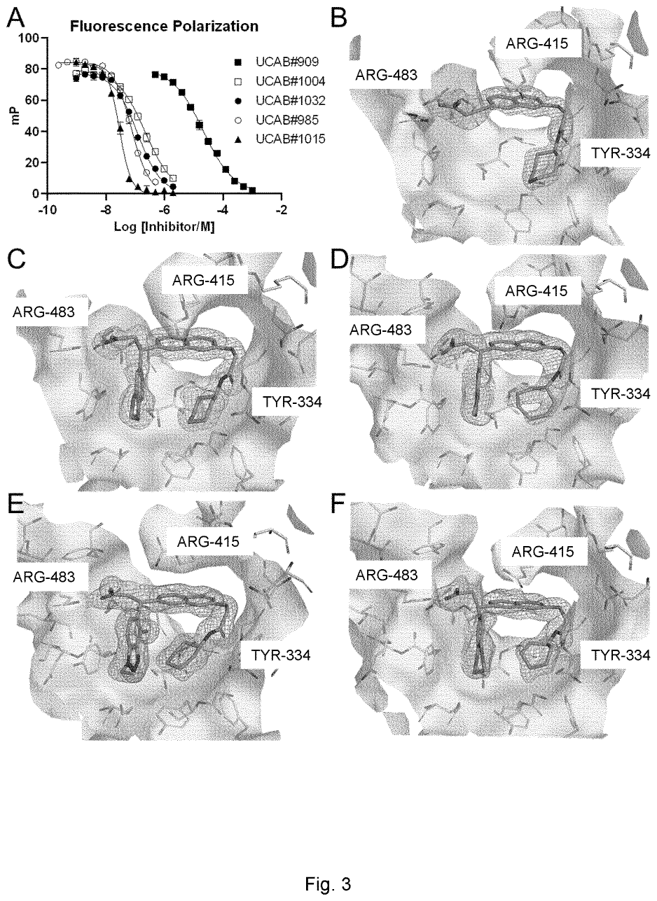

FIG. 3: Binding to Keap1 Kelch.
A) FP data of selected compounds.
B-F) X-ray structures of UCAB #909, UCAB #1004, UCAB #985, UCAB #1015, and UCAB #1032, respectively, binding in the Keap1 Kelch binding pocket.

### DETAILED DESCRIPTION

### Definitions

The terms “C₁₋₃ alkyl” and “C₁₋₅ alkyl” refers to a branched or unbranched alkyl group having from one to three or one to five carbon atoms respectively, including but not limited to methyl, ethyl, prop-1-yl, prop-2-yl, 2-methyl-prop-1-yl, 2-methyl-prop-2-yl, 2,2-dimethyl-prop-1-yl, but-1-yl, but-2-yl, 3-methyl-but-1-yl, 3-methyl-but-2-yl, pent-1-yl, pent-2-yl and pent-3-yl.

The term “C₂₋₅ alkenyl” refers to a branched or unbranched alkenyl group having from two to three or two to five carbon atoms respectively, two of which are connected by a double bond, including but not limited to ethenyl, propenyl, isopropenyl, butenyl, isobutenyl, pentenyl and isopentenyl.

The term “C₅₋₇ cycloalkyl” refers to a group having five to seven carbon atoms respectively including a monocyclic or bicyclic carbocycle, including but not limited to cyclopentyl, cyclohexyl and cycloheptyl.

The term “C₅₋₇ heterocycloalkyl” as used herein refers to a saturated carbocyclic molecule wherein the cyclic framework has 5 to 7 carbons and wherein one or more carbon atoms are substituted with heteroatom(s) selected from N, O, and S. In one embodiment, a “C₅₋₇ heterocycloalkyl” refers to a saturated carbocyclic molecule wherein the cyclic framework has 5 to 7 carbons and wherein one carbon atom is substituted with a heteroatom selected from N, O, and S. If the C₅₋₇ heterocycloalkyl group is a C₆ heterocycloalkyl, one or two carbon atoms are substituted with a heteroatom independently selected from N, O, and S. In one embodiment, a “C₅₋₇ heterocycloalkyl” refers to a saturated carbocyclic molecule wherein the cyclic framework has 5 to 7 carbons and wherein one carbon atom is substituted with a heteroatom selected from N, O, and S. In one embodiment, a “C₅₋₇ heterocycloalkyl” refers to a saturated carbocyclic molecule wherein the cyclic framework has 5 to 7 carbons and wherein two carbon atoms are substituted with a heteroatom individually selected from N, O, and S. Representative examples of C₃₋₆ heterocycloalkyl include, but are not limited to piperidinyl, morpholinyl, tetrahydropyranyl, pyrrolidinyl, tetrahydrofuranyl and piperazinyl.

The term “C₅₋₈ bicycloalkyl” as used herein refers a bicyclic ring system, wherein both cycloalkyl rings share the same two ring atoms. The term “C₅₋₈ bicycloalkyl” includes bridged bicyclic compounds, i.e, wherein the two rings share three or more atoms, separating the two bridgehead atoms by a bridge containing at least one atom. For example, the term “C₅₋₈ bicycloalkyl” includes bicyclo[2.2.1]heptan-2-yl. For example, the term “C₅₋₈ bicycloalkyl” includes

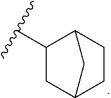 [SMILES: *C1CC2CCC1C2]

The term “C₉₋₁₂ tricycloalkyl” means three saturated hydrocarbon ring having a total of 9-12 carbon atoms, wherein said rings share at least one carbon atom. For example, the term “C₉₋₁₂ tricycloalkyl” includes adamantyl. For example, the term “C₉₋₁₂ tricycloalkyl” includes

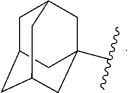 [SMILES: *C12CC3CC(CC(C3)C1)C2]

“Bicycloalkyl” and “tricycloalkyl” groups include non-aromatic saturated cyclic alkyl moieties consisting of two or three rings respectively, wherein said rings share at least one carbon atom.

### Compounds

In one aspect, the present invention relates to a compound of Formula (XI):

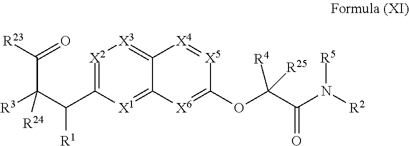

wherein
X¹ is CH or N;
X² is CH or N;
X³ is CH or N;
X⁴ is CH or N;
X⁵ is CH or N;
X⁶ is CH or N;
R¹ is phenyl, pyridinyl or H, wherein the phenyl or pyridinyl is optionally substituted with one or more, identical or different, substituents R⁶, wherein two of the substituents R⁶ optionally are linked together to form a ring;
R² is selected from the group consisting of C₅₋₇ cycloalkyl, C₅₋₇ cycloalkenyl, C₅₋₇ heterocycloalkyl, phenyl, C₅₋₈ bicycloalkyl, C₃₋₆ alkyl, and C₉₋₁₂ tricycloalkyl, wherein said C₅₋₇ cycloalkyl, C₅₋₇ cycloalkenyl, C₅₋₇ heterocycloalkyl, phenyl, C₅₋₈ bicycloalkyl, C₃₋₆ alkyl, and C₉₋₁₂ tricycloalkyl is optionally substituted with one or more, identical of different substituents R²⁸;
R⁵ is H or CH₃;
or R² is C₃₋₆ alkanediyl optionally substituted with R²⁸, R⁵ is a bond, and R² and R⁵ are linked together to form a ring;
R³ is H or CH₃;
R⁴ is H or CH₃;
R⁶ is selected from the group consisting of C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group of the C₁₋₅ alkyl, —OC₁₋₅ alkyl, or C₂₋₅ alkenyl is optionally replaced with O or N(R⁷); and/or
   wherein one or more methine group of the C₂₋₅ alkenyl is optionally replaced with N;
R⁷ is H or C₁₋₃ alkyl;
R²³ is selected from the group consisting of —OH;

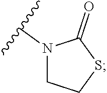 [SMILES: *N1CCSC1=O]

—OC₁₋₅ alkyl optionally substituted with one or more, identical or different, substituents R²⁶; —OC₂₋₅ alkenyl optionally substituted with one or more, identical or different, substituents R²⁶; —OC₂₋₅ alkynyl optionally substituted with one or more, identical or different, substituents R²⁶; —OC₃₋₆ cycloalkyl optionally substituted with one or more, identical or different, substituents R²⁶; —O-phenyl optionally substituted with one or more, identical or different, substituents R²⁷; and —O-benzyl optionally substituted with one or more, identical or different, substituents R²⁷; and —NH(CH₃);
R²⁴ is H or CH₃;
R²⁵ is H or CH₃;
R²⁶ is independently selected from the group consisting of H, deuterium, F, Cl and —OC₁₋₃ alkyl;
R²⁷ is independently selected from the group consisting of deuterium, methoxy, nitro, cyano, Cl, Br, I, and F; and
R²⁸ is individually selected from C₁₋₃ alkyl, —OC₁₋₃ alkyl, halogen, and deuterium, wherein said C₁₋₃ alkyl is optionally substituted with one or more halogens; or a pharmaceutically acceptable salt, hydrate, polymorph, tautomer, or solvate thereof.

In one embodiment, R²³ is —OH. In one embodiment, R²³ is —OC₁₋₅ alkyl. In one embodiment, R²³ is —OCH₃. In one embodiment, R²³ is —OCH₂CH₃. In one embodiment, R²³ is

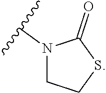 [SMILES: *N1CCSC1=O]

In one aspect, the present invention concerns a compound of Formula (XII):

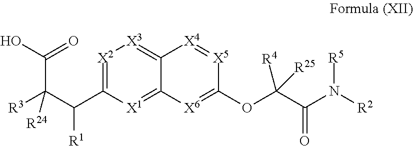

wherein
X¹ is CH or N;
X² is CH or N;
X³ is CH or N;
X⁴ is CH or N;
X⁵ is CH or N;
X⁶ is CH or N;
R¹ is phenyl or pyridinyl optionally substituted with one or more, identical or different, substituents R⁶, wherein two of the substituents R⁶ optionally are linked together to form a ring;
R² is selected from the group consisting of C₅₋₇ cycloalkyl, C₅₋₇ cycloalkenyl C₅₋₇ heterocycloalkyl and phenyl,
   wherein said C₅₋₇ cycloalkyl, C₅₋₇ cycloalkenyl C₅₋₇ heterocycloalkyl and phenyl is optionally substituted with one or more substituents individually selected from C₁₋₃ alkyl, —OC₁₋₃ alkyl, halogen and deuterium,
      wherein said C₁₋₃ alkyl is optionally substituted with one or more halogens;
R³ is H or CH₃;
R⁴ is H or CH₃;
R⁵ is H or CH₃;
R⁶ is selected from the group consisting of C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group of the C₁₋₅ alkyl, —OC₁₋₅ alkyl, or C₂₋₅ alkenyl is optionally replaced with O or N(R⁷); and/or
   wherein one or more methine group of the C₂₋₅ alkenyl is optionally replaced with N;
R⁷ is H or C₁₋₃ alkyl;
R²⁴ is H or CH₃; and
R²⁵ is H or CH₃;
or a pharmaceutically acceptable salt, hydrate, polymorph, tautomer, or solvate thereof.

In one embodiment, R²⁴ is H. In one embodiment, R²⁵ is H. In one embodiment, R²⁴ and R²⁵ are H.

In one aspect, the present invention relates to a compound of Formula (XI):

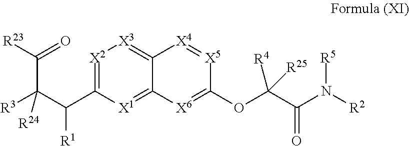

wherein
X¹ is CH or N;
X² is CH or N;
X³ is CH or N;
X⁴ is CH or N;
X⁵ is CH or N;
X⁶ is CH or N;
R²³ is selected from the group consisting of —OH;

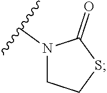 [SMILES: *N1CCSC1=O]

—OC₁₋₅ alkyl optionally substituted with one or more, identical or different, substituents R²⁶; —OC₂₋₅ alkenyl optionally substituted with one or more, identical or different, substituents R²⁶; —OC₂₋₅ alkynyl optionally substituted with one or more, identical or different, substituents R²⁶;—OC₃₋₆ cycloalkyl optionally substituted with one or more, identical or different, substituents R²⁶; —O-phenyl optionally substituted with one or more, identical or different, substituents R²⁷; and —O-benzyl optionally substituted with one or more, identical or different, substituents R²⁷; and —NH(CH₃);
R¹ is selected from the group consisting of Formula (III), Formula (V) and Formula (VI):

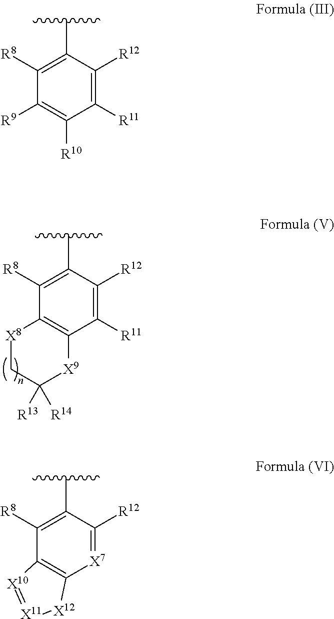

R² is selected from the group consisting of Formula (VIII), Formula (X), C₅₋₈ bicycloalkyl, C₃₋₆ alkyl, and C₉₋₁₂ tricycloalkyl, wherein said bicycloalkyl, C₃₋₆ alkyl, and C₉₋₁₂ tricycloalkyl is optionally substituted with one or more, identical of different substituents R²⁸:

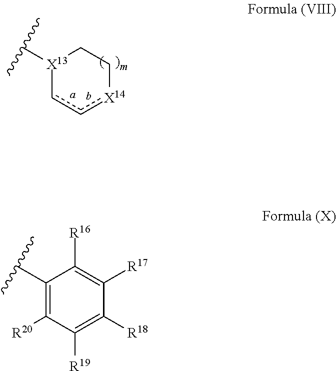

R³ is H or CH₃;
R²⁴ is H or CH₃;
R⁴ is H or CH₃;
R²⁵ is H or CH₃;
R⁵ is H or CH₃;
R⁸ is H or C₁₋₃ alkyl;
R⁹ is selected from the group consisting of H, C₁₋₃ alkyl, halogen, —OH and —OC₁₋₃ alkyl;
R¹⁰ is selected from the group consisting of H, halogen, —OH and —OC₁₋₃ alkyl;
R¹¹ is H;
R¹² is selected from the group consisting of H, C₁₋₃ alkyl and halogen;
R¹³ is selected from the group consisting of H, C₁₋₃ alkyl and halogen;
R¹⁴ is selected from the group consisting of H, C₁₋₃ alkyl and halogen;
R¹⁵ is H or C₁₋₅ alkyl;
R¹⁶ is H or C₁₋₃ alkyl;
R¹⁷ is H or C₁₋₃ alkyl;
R¹⁸ is H or C₁₋₃ alkyl;
R¹⁹ is H or C₁₋₃ alkyl; and
R²⁰ is H or C₁₋₃ alkyl.
R²¹ is selected from the group consisting of H, deuterium, halogen and C₁₋₃ alkyl, wherein said C₁₋₃ alkyl is optionally substituted with one or more halogens;
R²² is selected from the group consisting of H, deuterium, halogen and C₁₋₃ alkyl, wherein said C₁₋₃ alkyl is optionally substituted with one or more halogens;
R²⁶ is independently selected from the group consisting of H, deuterium, F, Cl and —OC₁₋₃ alkyl;
R²⁷ is independently selected from the group consisting of deuterium, methoxy, nitro, cyano, Cl, Br, I, and F; and
R²⁸ is individually selected from C₁₋₃ alkyl, —OC₁₋₃ alkyl, halogen, and deuterium, wherein said C₁₋₃ alkyl is optionally substituted with one or more halogens;
R²⁹ is H or C₁₋₃ alkyl;
X⁷ is C(R¹¹) or N;
X⁸ is O or CH₂;
X⁹ is O;
X¹⁰ is N or CH;
X¹¹ is N or CH;
X¹² is O or N(R¹⁵);
X¹³ is C(R²⁹) or N;
X¹⁴ is (1) C(H) and b is a double bond; (2) C(R²¹)(R²²) and b is a single bond; or (3) O and a is a single bond;
a is a double bond or a single bond;
b is a double bond or a single bond;
n is 0 or 1;
m is 0, 1 or 2;
with the proviso that no more than one of a and b is a double bond,
or a pharmaceutically acceptable salt, hydrate, polymorph, tautomer, or solvate thereof.

In one embodiment, R²³ is —OH, R²⁴ is H, and R²⁵ is H.

In one aspect, the present invention relates to a compound of Formula (I):

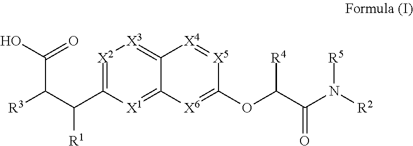

wherein
X¹ is CH or N;
X² is CH or N;
X³ is CH or N;
X⁴ is CH or N;
X⁵ is CH or N;
X⁶ is CH or N;
R¹ is phenyl, pyridinyl or H, wherein the phenyl or pyridinyl is optionally substituted with one or more, identical or different, substituents R⁶, wherein two of the substituents R⁶ optionally are linked together to form a ring;
R² is selected from the group consisting of C₅₋₇ cycloalkyl, C₅₋₇ cycloalkenyl C₅₋₇ heterocycloalkyl and phenyl,
   wherein said C₅₋₇ cycloalkyl, C₅₋₇ cycloalkenyl C₅₋₇ heterocycloalkyl and phenyl is optionally substituted with one or more substituents individually selected from C₁₋₃ alkyl, —OC₁₋₃ alkyl, halogen and deuterium,
      wherein said C₁₋₃ alkyl is optionally substituted with one or more halogens;
R³ is H or CH₃;
R⁴ is H or CH₃;
R⁵ is H or CH₃;
R⁶ is selected from the group consisting of C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group (—C(H)₂—) of the C₁₋₅ alkyl, —OC₁₋₅ alkyl, or C₂₋₅ alkenyl is optionally replaced with O or N(R⁷); and/or
   wherein one or more methine group (—C(H)═) of the C₂₋₅ alkenyl is optionally replaced with N; and
R⁷ is H or C₁₋₃ alkyl;
or a pharmaceutically acceptable salt, hydrate, polymorph, tautomer, or solvate thereof.

In one embodiment, the compound is of Formula (IA):

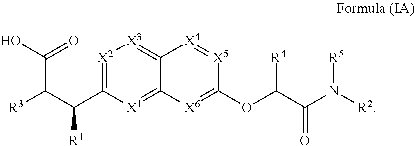

In one embodiment, the compound is of Formula (IB):

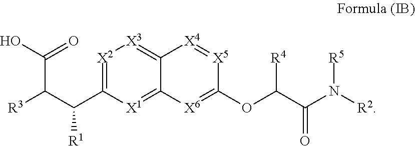

In one embodiment, X¹ is CH. In one embodiment, X¹ is N. In one embodiment, X² is CH. In one embodiment, X² is N. In one embodiment, X³ is CH. In one embodiment, X³ is N. In one embodiment, X⁴ is CH. In one embodiment, X⁴ is N. In one embodiment, X⁵ is CH. In one embodiment, X⁵ is N. In one embodiment, X⁶ is CH. In one embodiment, X⁶ is N. In one embodiment, X¹, X², X³, X⁴, X⁵ and X⁶ are CH. In one embodiment, no more than 2, such as no more than 1, of X¹, X², X³, X⁴, X⁵ and X⁶ are N. In one embodiment, X¹, X², X³, X⁴ and X⁵ are CH, and X⁶ is N. In one embodiment, X¹, X², X³, X⁵ and X⁶ are CH, and X⁴ is N. In one embodiment, X¹, X², X⁴, X⁵ and X⁶ are CH, and X³ is N.

In one embodiment, R³ is H. In one embodiment, R⁴ is H. In one embodiment, R⁵ is H. In one embodiment, R³, R⁴ and R⁵ are H. In one embodiment, X¹, X², X³, X⁴, X⁵ and X⁶ are CH, and R³, R⁴ and R⁵ are H.

In one embodiment, R⁴ is —CH₃. In one embodiment, R⁴ is —CH₃, and R³, R²⁴, R²⁵, and R⁵ are H. In one embodiment, R⁵ is —CH₃. In one embodiment, R⁵ is —CH₃, and R³, R²⁴, R²⁵, and R⁴ are H. In one embodiment, R³ is —CH₃. In one embodiment, R³ is —CH₃, and R⁴, R²⁴, R²⁵, and R⁵ are H. In one embodiment, R³ and R²⁴ are —CH₃. In one embodiment, R³ and R²⁴ are —CH₃, and R⁴, R²⁵, and R⁵ are H. In one embodiment, R³, R²⁴, R⁴, R²⁵, and R⁵ are H.

In one embodiment, R¹ is of Formula (II):

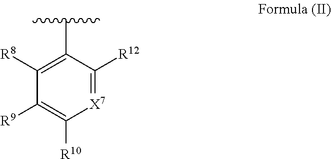

wherein
X⁷ is C(R¹¹) or N;
R⁷ is H or C₁₋₃ alkyl;
R⁸ is H or C₁₋₅ alkyl;
R⁹ is selected from the group consisting of H, C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group of the C₁₋₅ alkyl, —OC₁₋₅ alkyl, or C₂₋₅ alkenyl is optionally replaced with O or N(R⁷); and/or
   wherein one or more methine group of the C₂₋₅ alkenyl is optionally replaced with N;
R¹⁰ is selected from the group consisting of H, C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group of the C₁₋₅ alkyl, —OC₁₋₅ alkyl, or C₂₋₅ alkenyl is optionally replaced with O or N(R⁷); and/or
   wherein one or more methine group of the C₂₋₅ alkenyl is optionally replaced with N;
R¹¹ is H or —OC₁₋₅ alkyl;
R¹² is H, C₁₋₅ alkyl or —OC₁₋₅ alkyl; and
R⁹ and R¹⁰ are optionally linked together to form a ring.

In one embodiment, R¹ is of Formula (II), wherein
X⁷ is C(R¹¹) or N;
R⁸ is H or C₁₋₅ alkyl;
R⁹ is selected from the group consisting of H, C₁₋₅ alkyl, halogen, —OH and —OC₁₋₅ alkyl;
R¹⁰ is selected from the group consisting of H, C₁₋₅ alkyl, halogen, —OH and —OC₁₋₅ alkyl;
R¹¹ is H; and
R¹² is H or C₁₋₅ alkyl.

In one embodiment, X⁷ is C(R¹¹). In one embodiment, R¹ is of Formula (III):

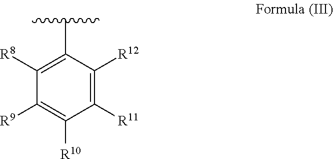

wherein
R⁸ is H or C₁₋₃ alkyl;
R⁹ is selected from the group consisting of H, C₁₋₃ alkyl, halogen, —OH and —OC₁₋₃ alkyl;
R¹⁰ is selected from the group consisting of H, halogen, —OH and —OC₁₋₃ alkyl;
R¹¹ is H; and
R¹² is H or C₁₋₃ alkyl.

In one embodiment, R¹ is of Formula (III), wherein
R⁸ is H or Me;
R⁹ is selected from the group consisting of H, Me, Cl, —OH and —OMe;
R¹⁰ is selected from the group consisting of H, Cl, —OH, OMe, OEt and OPr;
R¹¹ is H; and
R¹² is H or Me.

In one embodiment, R¹ is H.

In one embodiment, R¹ is phenyl. In one embodiment, R¹ is

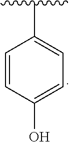 [SMILES: *c1ccc(O)cc1]

In one embodiment, R¹ is

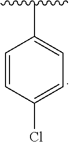 [SMILES: *c1ccc(Cl)cc1]

In one embodiment, R¹ is

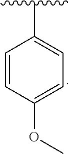 [SMILES: *c1ccc(OC)cc1]

In one embodiment, R¹ is

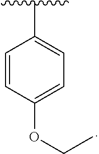 [SMILES: *c1ccc(OCC)cc1]

In one embodiment, R¹ is

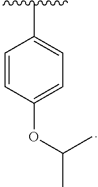 [SMILES: *c1ccc(OC(C)C)cc1]

In one embodiment, R¹ is

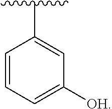 [SMILES: *c1cccc(O)c1]

In one embodiment, R¹ is

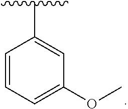 [SMILES: *c1cccc(OC)c1]

In one embodiment, R¹ is

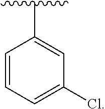 [SMILES: *c1cccc(Cl)c1]

In one embodiment, R¹ is

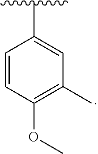 [SMILES: *c1ccc(OC)c(C)c1]

In one embodiment, R¹ is

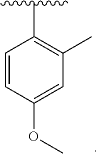 [SMILES: *c1ccc(OC)cc1C]

In one embodiment, R¹ is

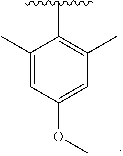

In one embodiment, R¹ is

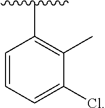 [SMILES: *c1cccc(Cl)c1C]

In one embodiment, R¹ is

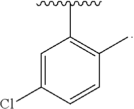 [SMILES: *c1cc(Cl)ccc1C]

In one embodiment, R¹ is

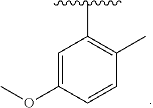

In some embodiments, R¹ is phenyl or pyridinyl substituted with one or more, identical or different, substituents R⁶, wherein two of the substituents R⁶ are linked together to form a ring. In one embodiment, R⁶ is C₁₋₃ alkyl or —OC₁₋₃ alkyl, wherein said C₁₋₃ alkyl and —OC₁₋₃ alkyl optionally are substituted with one or more substituents individually selected from C₁₋₃ alkyl and halogen. In one embodiment, R¹ is of Formula (IV):

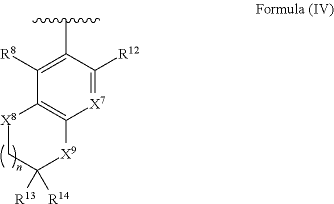

wherein
X⁷ is C(R¹¹) or N;
X⁸ is O or CH₂;
X⁹ is O;
R⁸ is H or C₁₋₅ alkyl;
R¹³ is selected from the group consisting of H, C₁₋₃ alkyl and halogen;
R¹⁴ is selected from the group consisting of H, C₁₋₃ alkyl and halogen;
R¹¹ is H;
R¹² is selected from the group consisting of H, C₁₋₅ alkyl and halogen; and
n is 0 or 1.

In one embodiment, R¹ is of Formula (IV):

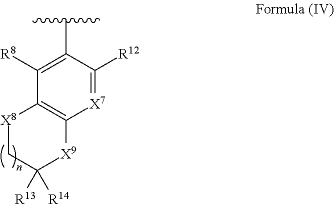

wherein
X⁷ is C(R¹¹) or N;
X⁸ is O or CH₂;
X⁹ is O;
R⁸ is H or C₁₋₅ alkyl;
R¹³ is selected from the group consisting of H, C₁₋₃ alkyl and halogen;
R¹⁴ is selected from the group consisting of H, C₁₋₃ alkyl and halogen;
R¹¹ is H;
R¹² is H or C₁₋₅ alkyl; and
n is 0 or 1.

In one embodiment, R¹ is of Formula (V):

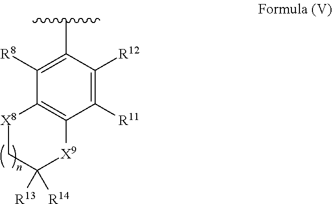

X⁸ is O or CH₂;
X⁹ is O;
R⁸ is H or C₁₋₃ alkyl;
R¹³ is selected from the group consisting of H, C₁₋₃ alkyl and halogen;
R¹⁴ is selected from the group consisting of H, C₁₋₃ alkyl and halogen;
R¹¹ is H;
R¹² is selected from the group consisting of H, C₁₋₃ alkyl and halogen; and
n is 0 or 1.

In one embodiment, X⁷ is C(R¹¹). In one embodiment, R¹ is of Formula (V):

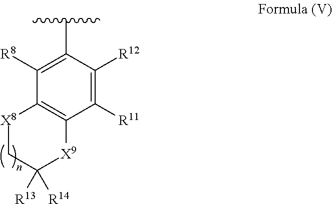

wherein
X⁸ is O or CH₂;
X⁹ is O;
R⁸ is H or C₁₋₃ alkyl;
R¹³ is selected from the group consisting of H, C₁₋₃ alkyl and halogen;
R¹⁴ is selected from the group consisting of H, C₁₋₃ alkyl and halogen;
R¹¹ is H;
R¹² is H or C₁₋₃ alkyl; and
n is 0 or 1.

In one embodiment, R¹ is of Formula (V), wherein
X⁸ is O or CH₂;
X⁹ is O;
R⁸ is H or Me;
R¹³ is selected from the group consisting of H, Me and F;
R¹⁴ is selected from the group consisting of H, Me and F;
R¹¹ is H;
R¹² is H or Me; and
n is 0 or 1.

In one embodiment, R¹ is

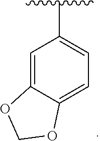 [SMILES: *c1ccc2c(c1)OCO2]

In one embodiment, R¹ is

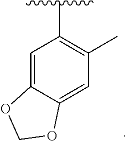 [SMILES: *c1cc2c(cc1C)OCO2]

In one embodiment, R¹ is

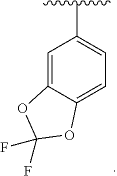 [SMILES: *c1ccc2c(c1)OC(F)(F)O2]

In one embodiment, R¹ is

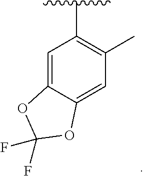 [SMILES: *c1cc2c(cc1C)OC(F)(F)O2]

In one embodiment, R¹ is

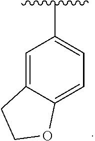 [SMILES: *c1ccc2c(c1)CCO2]

In one embodiment, R¹ is

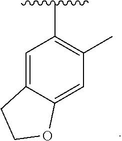 [SMILES: *c1cc2c(cc1C)OCC2]

In one embodiment, R¹ is

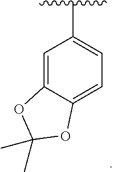 [SMILES: *c1ccc2c(c1)OC(C)(C)O2]

In one embodiment, R¹ is

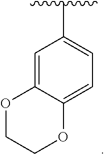 [SMILES: *c1ccc2c(c1)OCCO2 ; "."]

In one embodiment, R¹ is

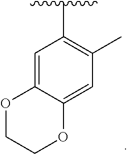 [SMILES: *c1cc2c(cc1C)OCCO2]

In one embodiment, R¹ is

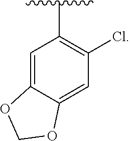 [SMILES: *c1cc2c(cc1Cl)OCO2]

In one embodiment, R¹ is phenyl or pyridinyl substituted with one or more, identical or different, substituents R⁶, wherein two of the substituents R⁶ are linked together to form a ring. In one embodiment, R⁶ is individually selected from C₁₋₅ alkyl, —OC₁₋₅ alkyl and C₂₋₅ alkenyl, wherein said C₁₋₅ alkyl, —OC₁₋₅ alkyl and C₂₋₅ alkenyl are optionally substituted with one or more substituents individually selected from C₁₋₃ alkyl and halogen; and wherein one or more methylene group of the C₁₋₅ alkyl, —OC₁₋₅ alkyl, or C₂₋₅ alkenyl is optionally replaced with O or N(R⁷); and/or wherein one or more methine group of the C₂₋₅ alkenyl is optionally replaced with N. In one embodiment, R¹ is of Formula (VI):

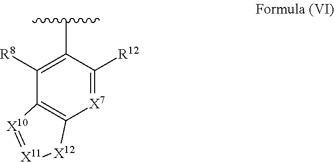

wherein
X⁷ is C(R¹¹) or N;
X¹⁰ is N or CH;
X¹¹ is N or CH;
X¹² is O or N(R¹⁵);
R⁸ is H or C₁₋₅ alkyl;
R¹¹ is H or C₁₋₅ alkyl;
R¹² is H or C₁₋₅ alkyl; and
R¹⁵ is H or C₁₋₅ alkyl.

In one embodiment, X⁷ is C(R¹¹). In one embodiment, R¹ is of Formula (VII):

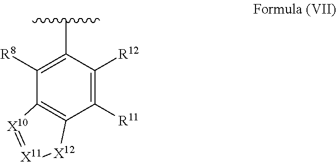

wherein
X¹⁰ is N or CH;
X¹¹ is N or CH;
X¹² is O or N(R¹⁵);
R⁸ is H or C₁₋₃ alkyl;
R¹¹ is H or C₁₋₃ alkyl;
R¹² is H or C₁₋₃ alkyl; and
R¹⁵ is H or C₁₋₃ alkyl.

In one embodiment, R¹ is of Formula (VII), wherein
X¹⁰ is N or CH;
X¹¹ is N or CH;
X¹² is O or N(R¹⁵);
R⁸ is H;
R¹¹ is H or Me;
R¹² is H or Me; and
R¹⁵ is H, Me or Et.

In one embodiment, R¹ is

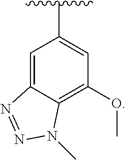 [SMILES: *c1cc(OC)c2c(c1)nnn2C]

In one embodiment, R¹ is

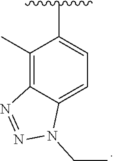 [SMILES: *c1ccc2c(nnn2CC)c1C]

In one embodiment, R¹ is

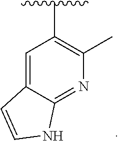 [SMILES: *c1cc2cc[nH]c2nc1C]

In one embodiment, R¹ is

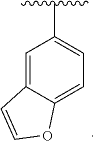 [SMILES: *c1ccc2occc2c1]

In one embodiment, R¹ is

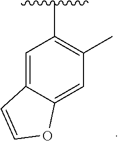 [SMILES: *c1cc2ccoc2cc1C]

In one embodiment, R¹ is phenyl optionally substituted with one or more, identical or different, substituents R⁶, wherein two of the substituents R⁶ optionally are linked together to form a ring; and
R⁶ is selected from the group consisting of C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group of the C₁₋₅ alkyl, —OC₁₋₅ alkyl, or C₂₋₅ alkenyl is optionally replaced with O or N(R⁷); and/or
   wherein one or more methine group of the C₂₋₅ alkenyl is optionally replaced with N; and
R⁷ is H or C₁₋₃ alkyl.

In one embodiment, X⁷ is C(R¹¹). In one embodiment, X⁷ is N.

In one embodiment, X⁸ is O. In one embodiment, X⁸ is CH₂.

In one embodiment, X⁹ is O.

In one embodiment, X¹⁰ is N. In one embodiment, X¹⁰ is CH.

In one embodiment, X¹¹ is N. In one embodiment, X¹¹ is CH.

In one embodiment, X¹² is O. In one embodiment, X¹² is N(R¹⁵) and R¹⁵ is H or C₁₋₅ alkyl. In one embodiment, X¹² is N(H). In one embodiment, X¹² is N(CH₃). In one embodiment, X¹² is N(CH₂CH₃).

In one embodiment, R⁸ is H. In one embodiment, R⁸ is C₁₋₅ alkyl, such as C₁₋₃ alkyl. In one embodiment, R⁸ is Me.

In one embodiment, R⁹ is H. In one embodiment, R⁹ is C₁₋₅ alkyl, such as C₁₋₃ alkyl. In one embodiment, R⁹ is Me. In one embodiment, R⁹ is C₁₋₅ alkyl substituted with one or more halogens. In one embodiment, R⁹ is halogen. In one embodiment, R⁹ is Cl. In one embodiment, R⁹ is —OH. In one embodiment, R⁹ is —OC₁₋₅ alkyl, such as is —OC₁₋₃ alkyl. In one embodiment, R⁹ is —OMe.

In one embodiment, R¹⁰ is H. In one embodiment, R¹⁰ is C₁₋₅ alkyl, such as C₁₋₃ alkyl. In one embodiment, wherein R¹⁰ is Me. In one embodiment, R¹⁰ is C₁₋₅ alkyl substituted with one or more halogens. In one embodiment, R¹⁰ is halogen. In one embodiment, R¹⁰ is Cl. In one embodiment, R¹⁰ is —OH. In one embodiment, R¹⁰ is —OC₁₋₅ alkyl, such as is —OC₁₋₃ alkyl. In one embodiment, R¹⁰ is —OMe.

In one embodiment, R¹¹ is H. In one embodiment, R¹¹ is —OC₁₋₅ alkyl, such as-OC₁₋₃ alkyl. In one embodiment, R¹¹ is —OMe.

In one embodiment, R¹² is H. In one embodiment, R¹² is C₁₋₅ alkyl, such as C₁₋₃ alkyl. In one embodiment, R¹² is Me. In one embodiment, R¹² is halogen. In one embodiment, R¹² is Cl.

In one embodiment, R¹³ is H. In one embodiment, R¹³ is C₁₋₃ alkyl. In one embodiment, R¹³ is Me. In one embodiment, R¹³ is halogen. In one embodiment, R¹³ is F.

In one embodiment, R¹⁴ is H. In one embodiment, R¹⁴ is C₁₋₃ alkyl. In one embodiment, R¹⁴ is Me. In one embodiment, R¹⁴ is halogen. In one embodiment, R¹⁴ is F. In one embodiment, R¹³ and R¹⁴ are identical.

In one embodiment, R¹⁵ is H. In one embodiment, R¹⁵ is C₁₋₅ alkyl, such as C₁₋₃ alkyl. In one embodiment, R¹⁵ is Me. In one embodiment, R¹⁵ is Et.

In one embodiment, n is 0. In one embodiment, n is 1.

In one embodiment, R² is of Formula (VIII):

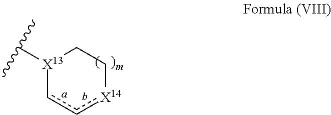

wherein
X¹³ is C(R²⁹) or N;
X¹⁴ is (1) C(H) and b is a double bond; (2) C(R²¹)(R²²) and b is a single bond; or (3) O and a is a single bond;
R²¹ is selected from the group consisting of H, deuterium, halogen and C₁₋₃ alkyl, wherein said C₁₋₃ alkyl is optionally substituted with one or more halogens;
R²² is selected from the group consisting of H, deuterium, halogen and C₁₋₃ alkyl,
wherein said C₁₋₃ alkyl is optionally substituted with one or more halogens; a is a double bond or a single bond;
b is a double bond or a single bond;
R²⁹ is H or C₁₋₃ alkyl; and
m is 0, 1 or 2,
with the proviso that no more than one of a and b is a double bond.

In one embodiment, R² is of Formula (VIII), wherein
X¹³ is C(H) or N;
X¹⁴ is (1) C(H) and b is a double bond; (2) C(R²¹)(R²²) and b is a single bond; or (3) O and a is a single bond;
R²¹ is selected from the group consisting of H, deuterium, halogen and C₁₋₃ alkyl, wherein said C₁₋₃ alkyl is optionally substituted with one or more halogens;
R²² is selected from the group consisting of H, deuterium, halogen and C₁₋₃ alkyl, wherein said C₁₋₃ alkyl is optionally substituted with one or more halogens;
a is a double bond or a single bond;
b is a double bond or a single bond; and
m is 1 or 2,
with the proviso that no more than one of a and b is a double bond.

In one embodiment, R² is of Formula (IX):

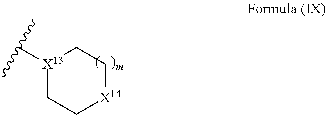

wherein
X¹³ is C(R²⁹) or N;
X¹⁴ is C(R²¹)(R²²) or O;
R²¹ is selected from the group consisting of H, deuterium, halogen and C₁₋₃ alkyl, wherein said C₁₋₃ alkyl is optionally substituted with one or more halogens;
R²² is selected from the group consisting of H, deuterium, halogen and C₁₋₃ alkyl, wherein said C₁₋₃ alkyl is optionally substituted with one or more halogens;
R²⁹ is H or C₁₋₃ alkyl; and
m is 0, 1 or 2.

In one embodiment, R² is of Formula (IX), wherein
X¹³ is C(H) or N;
X¹⁴ is C(R²¹)(R²²) or O;
R²¹ is selected from the group consisting of H, deuterium, halogen and C₁₋₃ alkyl, wherein said C₁₋₃ alkyl is optionally substituted with one or more halogens;
R²² is selected from the group consisting of H, deuterium, halogen and C₁₋₃ alkyl, wherein said C₁₋₃ alkyl is optionally substituted with one or more halogens; and
m is 1 or 2.

In one embodiment, m is 1. In one embodiment, m is 2.

In one embodiment, X¹³ is C(H). In one embodiment, X¹³ is N. In one embodiment, X¹³ is C(R²⁹). In one embodiment, R²⁹ is H. In one embodiment, R²⁹ is C₁₋₃ alkyl. In one embodiment, R²⁹ is —CH₃. In one embodiment, X¹³ is C(CH₃).

In one embodiment, X¹⁴ is C(R²¹)(R²²). In one embodiment, R²¹ is H. In one embodiment, R²² is H. In one embodiment, X¹⁴ is C(H)₂. In one embodiment, R²¹ is deuterium (D). In one embodiment, X¹⁴ is C(H) (D). In one embodiment, R²¹ is halogen, such as F. In one embodiment, R²² is halogen, such as F. In one embodiment, X¹⁴ is C(F)₂. In one embodiment, R²¹ is C₁₋₃ alkyl, such as Me. In one embodiment, R²² is C₁₋₃ alkyl, such as Me. In one embodiment, X¹⁴ is C(CH₃)₂. In one embodiment, R²¹ is C₁₋₃ alkyl, such as Me, substituted with one or more halogens, such as F. In one embodiment, R²¹ is —CF₃. In one embodiment, X¹⁴ is C(H)(CF₃). In one embodiment, X¹⁴ is O. In one embodiment, R²¹ is —CH₃. In one embodiment, R²² is —CH₃

In one embodiment, R² is of Formula (IX) and X¹³ is C(H), X¹⁴ is C(H)₂ and m is 1. In one embodiment, R² is cyclohexyl. In one embodiment, R² is of Formula (IX) and X¹³ is C(H), X¹⁴ is C(H)₂ and m is 2. In one embodiment, R² is cycloheptyl. In one embodiment, R² is of Formula (IX) and X¹³ is C(H), X¹⁴ is O and m is 1. In one embodiment, R² is tetrahydro-2H-pyran-4-yl. In one embodiment, R² is of Formula (IX) and X¹³ is N, X¹⁴ is O and m is 1. In one embodiment, R² is N-morpholinyl. In one embodiment, R² is of Formula (IX) and X¹³ is C(H), X¹⁴ is C(H) (D) and m is 1. In one embodiment, R² is of Formula (IX) and X¹³ is C(H), X¹⁴ is C(F)₂ and m is 1. In one embodiment, R² is of Formula (IX) and X¹³ is C(H), X¹⁴ is C(CH₃)₂ and m is 1. In one embodiment, R² is of Formula (IX) and X¹³ is C(H), X¹⁴ is C(H)(CF₃) and m is 1. In one embodiment, R² is of Formula (VIII) and X¹³ is C(H), X¹⁴ is C(H)₂, m is 1, a is a double bond and b is a single bond. In one embodiment, R² is cyclohex-2-enyl. In one embodiment, R² is of Formula (VIII) and X¹³ is C(H), X¹⁴ is C(H)₂, m is 1, a is a single bond and b is a double bond. In one embodiment, R² is cyclohex-3-enyl. In one embodiment, R² is of Formula (IX) and X¹³ is C(H), X¹⁴ is C(H)₂ and m is 0. In one embodiment, R² is cyclopentyl. In one embodiment, R² is of Formula (IX) and X¹³ is C(CH₃), X¹⁴ is C(H)₂ and m is 1.

In one embodiment, R² is

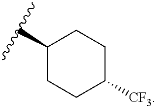 [SMILES: *[C@H]1CC[C@H](C(F)(F)F)CC1]

In one embodiment, R² is

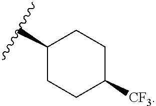 [SMILES: *[C@H]1CC[C@@H](C(F)(F)F)CC1]

In one embodiment, R² is of Formula (X), R¹⁶ is H, R¹⁷ is H, R¹⁸ is H, R¹⁹ is H and R²⁰ is H. In one embodiment, R² is phenyl. In one embodiment, R² is C₅₋₈ bicycloalkyl. In one embodiment, R² is bicyclo[2.2.1]heptan-2-yl.

In one embodiment, R² is

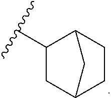 [SMILES: *C1CC2CCC1C2]

In one embodiment, R² is

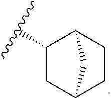 [SMILES: *[C@H]1C[C@@H]2CC[C@H]1C2]

In one embodiment, R² is C₉₋₁₂ tricycloalkyl. In one embodiment, R² is adamantyl. In one embodiment, R² is adamant-1-yl. In one embodiment, R² is

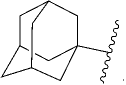 [SMILES: *C12CC3CC(CC(C3)C1)C2]

In one embodiment, R² is C₃₋₆ alkyl. In one embodiment, R² is C₄ alkyl. In one embodiment, R² is iso-butyl.

In one embodiment, R² is C₃₋₆ alkanediyl optionally substituted with R²⁸, R⁵ is a bond, and R² and R⁵ are linked together to form a ring. In one embodiment, R² is C₄ alkanediyl optionally substituted with R²⁸, R⁵ is a bond, and R² and R⁵ are linked together to form a ring. In one embodiment, R² is C₄ alkanediyl substituted with propyl, R⁵ is a bond, and R² and R⁵ are linked together to form a ring.

In one embodiment,

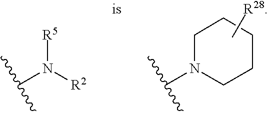

In one embodiment,

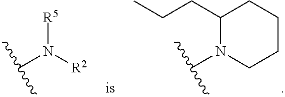

In one embodiment, the compound is of Formula (XIII):

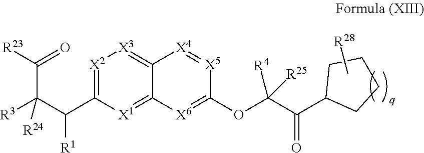

wherein q is 0, 1, 2 or 3, and R²³, R³, R²⁴, R¹, X¹, X², X³, X⁴, X⁵, X⁶, R⁴, R²⁵, and R²⁸ are as defined herein.

In one embodiment, R² is of Formula (X):

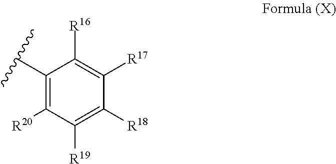

wherein
R¹⁶ is H or C₁₋₃ alkyl;
R¹⁷ is H or C₁₋₃ alkyl;
R¹⁸ is H or C₁₋₃ alkyl;
R¹⁹ is H or C₁₋₃ alkyl; and
R²⁰ is H or C₁₋₃ alkyl.

In one embodiment, R¹⁶ is H. In one embodiment, R¹⁶ is C₁₋₃ alkyl. In one embodiment, R¹⁶ is Me.

In one embodiment, R¹⁷ is H. In one embodiment, R¹⁷ is C₁₋₃ alkyl. In one embodiment, R¹⁷ is Me.

In one embodiment, R¹⁸ is H. In one embodiment, R¹⁸ is C₁₋₃ alkyl. In one embodiment, R¹⁸ is Me.

In one embodiment, R¹⁹ is H. In one embodiment, R¹⁹ is C₁₋₃ alkyl. In one embodiment, R¹⁹ is Me.

In one embodiment, R²⁰ is H. In one embodiment, R²⁰ is C₁₋₃ alkyl. In one embodiment, R²⁰ is Me.

In one embodiment, R² is of Formula (X), R¹⁶ is Me, R¹⁷ is H, R¹⁸ is Me, R¹⁹ is H and R²⁰ is Me. In one embodiment, R² is of Formula (X), R¹⁶ is Me, R¹⁷ is Me, R¹⁸ is H, R¹⁹ is Me and R²⁰ is Me.

In one embodiment, R²⁴ is H. In one embodiment, R²⁵ is H. In one embodiment, R²⁴ and R²⁵ are H. In one embodiment, R²⁴ is CH₃. In one embodiment, R²⁵ is CH₃. In one embodiment, R²⁴ and R²⁵ are CH₃.

In one embodiment, the C₁₋₅ alkyl is C₁₋₃ alkyl. In one embodiment, the C₁₋₃ alkyl is Me, Et, ⁿPr or ⁱPr. In one embodiment, wherein the halogen is Cl. In one embodiment, the halogen is F.

In one aspect, the present invention relates to a compound of Formula (I):

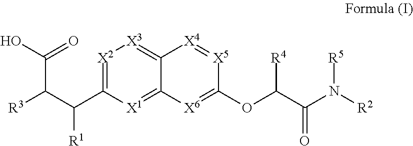

wherein
X¹ is CH;
X² is CH;
X³ is CH;
X⁴ is CH;
X⁵ is CH;
X⁶ is CH;
R¹ is selected from the group consisting of Formula (III), Formula (V) and Formula (VI):

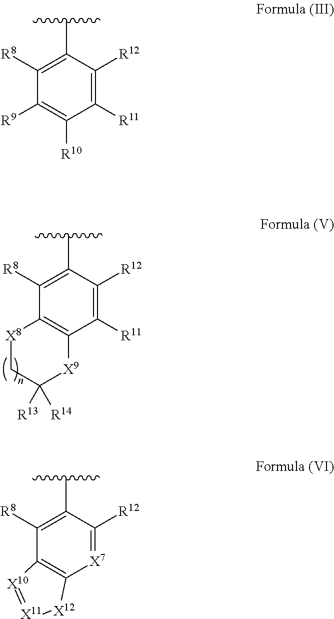

R² is of Formula (VIII) or Formula (X):

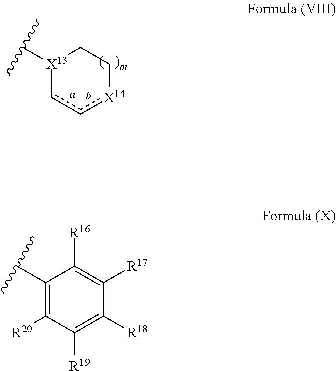

R³ is H or CH₃;
R⁴ is H or CH₃;
R⁵ is H or CH₃;
R⁸ is H or C₁₋₃ alkyl;
R⁹ is selected from the group consisting of H, C₁₋₃ alkyl, halogen, —OH and —OC₁₋₃ alkyl;
R¹⁰ is selected from the group consisting of H, halogen, —OH and —OC₁₋₃ alkyl;
R¹¹ is H;
R¹² is H or C₁₋₃ alkyl;
R¹³ is selected from the group consisting of H, C₁₋₃ alkyl and halogen;
R¹⁴ is selected from the group consisting of H, C₁₋₃ alkyl and halogen;
R¹⁵ is H or C₁₋₅ alkyl;
R¹⁶ is H or C₁₋₃ alkyl;
R¹⁷ is H or C₁₋₃ alkyl;
R¹⁸ is H or C₁₋₃ alkyl;
R¹⁹ is H or C₁₋₃ alkyl; and
R²⁰ is H or C₁₋₃ alkyl.
R²¹ is selected from the group consisting of H, deuterium, halogen and C₁₋₃ alkyl, wherein said C₁₋₃ alkyl is optionally substituted with one or more halogens;
R²² is selected from the group consisting of H, deuterium, halogen and C₁₋₃ alkyl, wherein said C₁₋₃ alkyl is optionally substituted with one or more halogens;
X⁷ is C(R¹¹) or N;
X⁸ is O or CH₂;
X⁹ is O;
X¹⁰ is N or CH;
X¹¹ is N or CH;
X¹² is O or N(R¹⁵);
X¹³ is C(H) or N;
X¹⁴ is (1) C(H) and b is a double bond; (2) C(R²¹)(R²²) and b is a single bond; or (3) O and a is a single bond;
a is a double bond or a single bond;
b is a double bond or a single bond;
n is 0 or 1;
m is 1 or 2;
with the proviso that no more than one of a and b is a double bond.
or a pharmaceutically acceptable salt, hydrate, polymorph, tautomer, or solvate thereof.

In one embodiment, the compound is selected from the group consisting of:

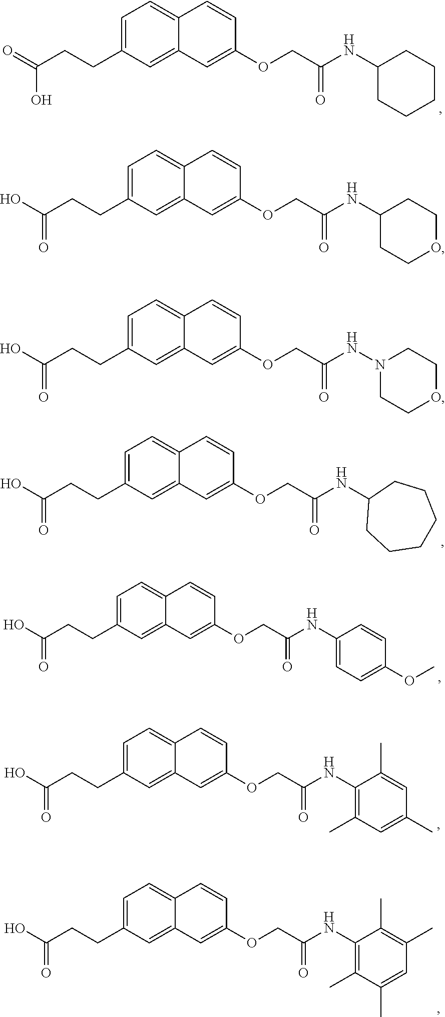

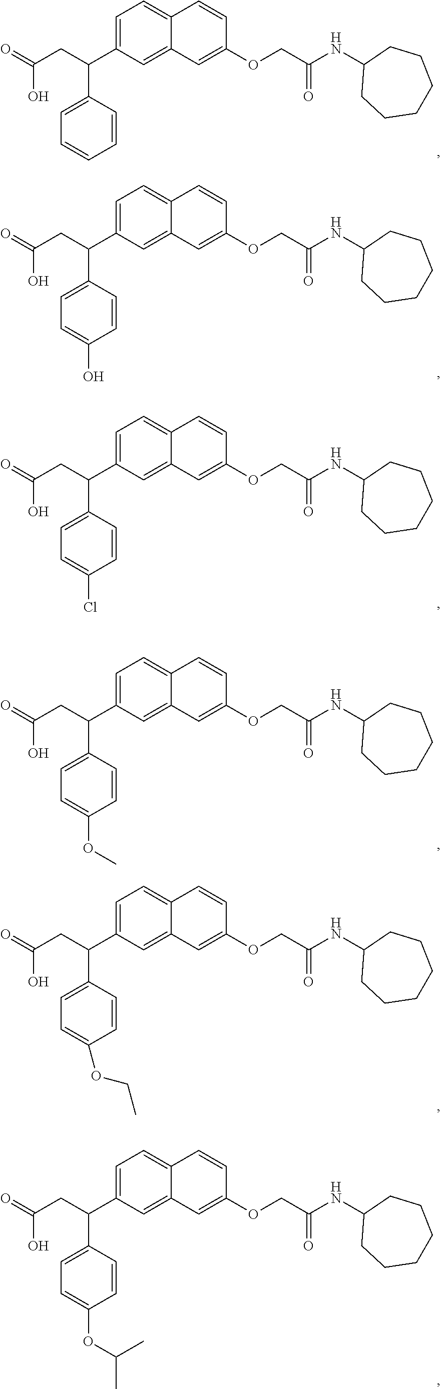

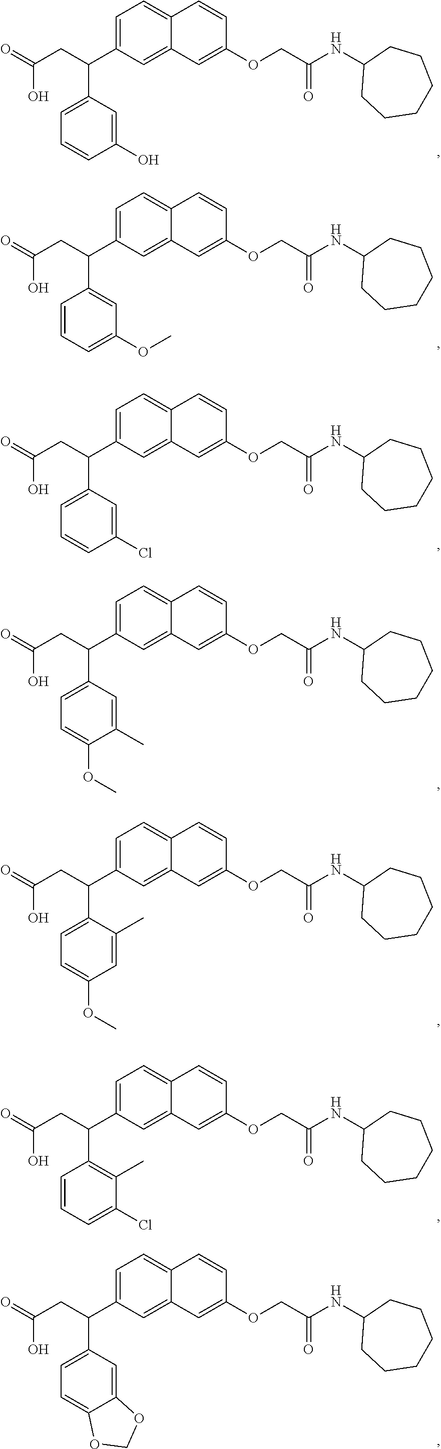

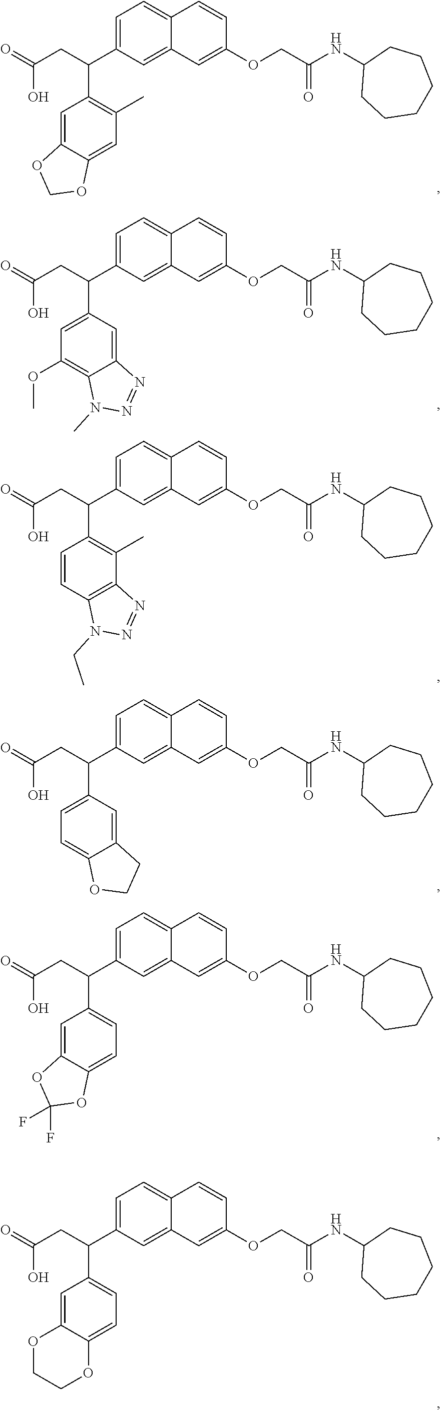

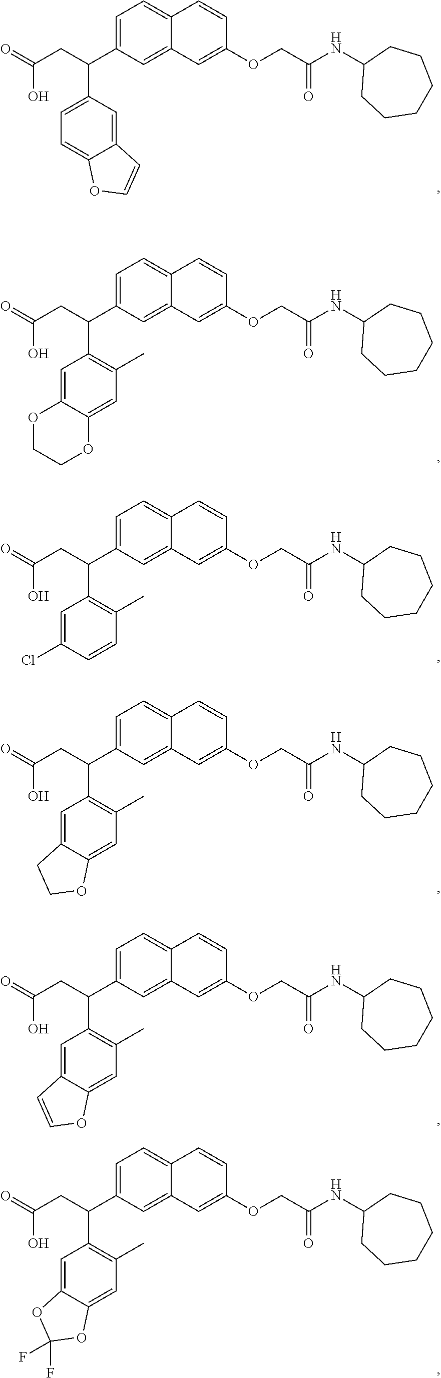 [SMILES: O=C(O)CC(c1ccc2ccc(OCC(=O)NC3CCCCCC3)cc2c1)c1ccc2occc2c1 ; Cc1cc2c(cc1C(CC(=O)O)c1ccc3ccc(OCC(=O)NC4CCCCCC4)cc3c1)OCCO2 ; Cc1ccc(Cl)cc1C(CC(=O)O)c1ccc2ccc(OCC(=O)NC3CCCCCC3)cc2c1 ; Cc1cc2c(cc1C(CC(=O)O)c1ccc3ccc(OCC(=O)NC4CCCCCC4)cc3c1)CCO2 ; Cc1cc2occc2cc1C(CC(=O)O)c1ccc2ccc(OCC(=O)NC3CCCCCC3)cc2c1 ; Cc1cc2c(cc1C(CC(=O)O)c1ccc3ccc(OCC(=O)NC4CCCCCC4)cc3c1)OC(F)(F)O2]

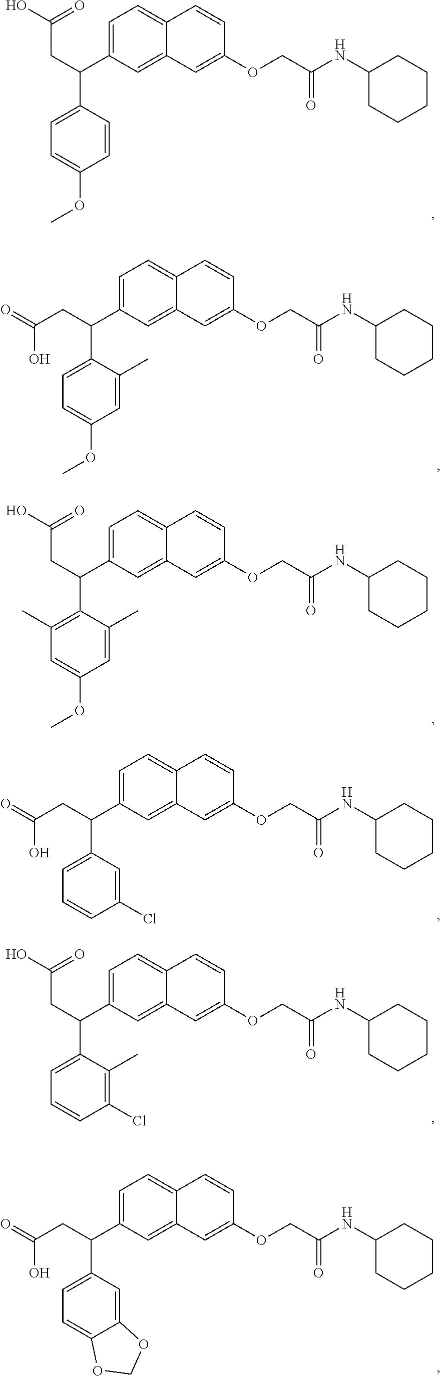

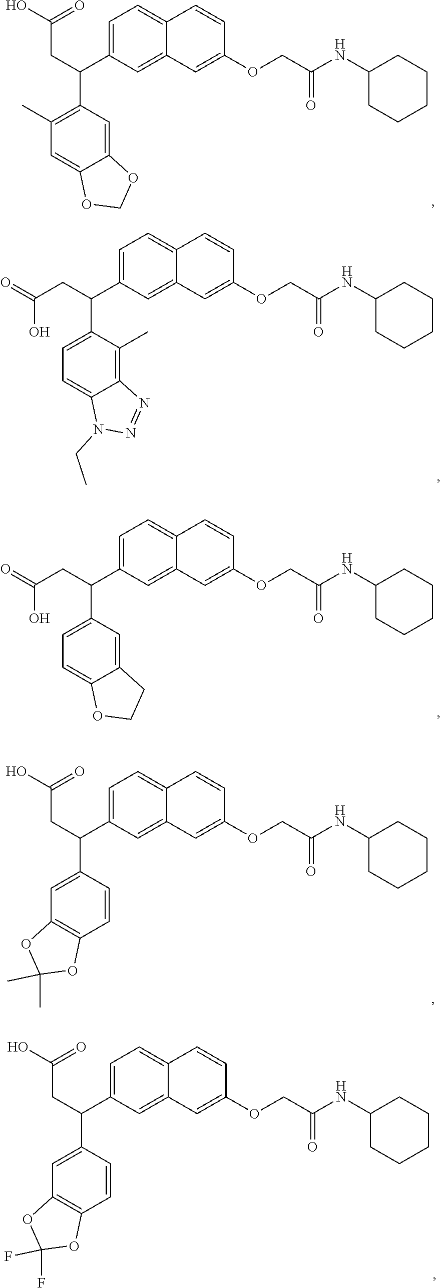

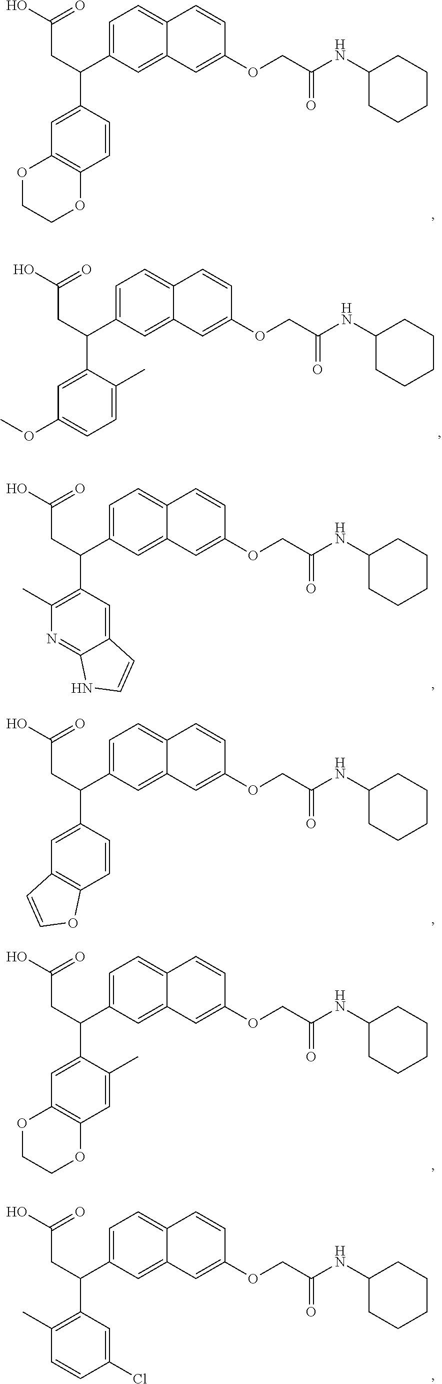

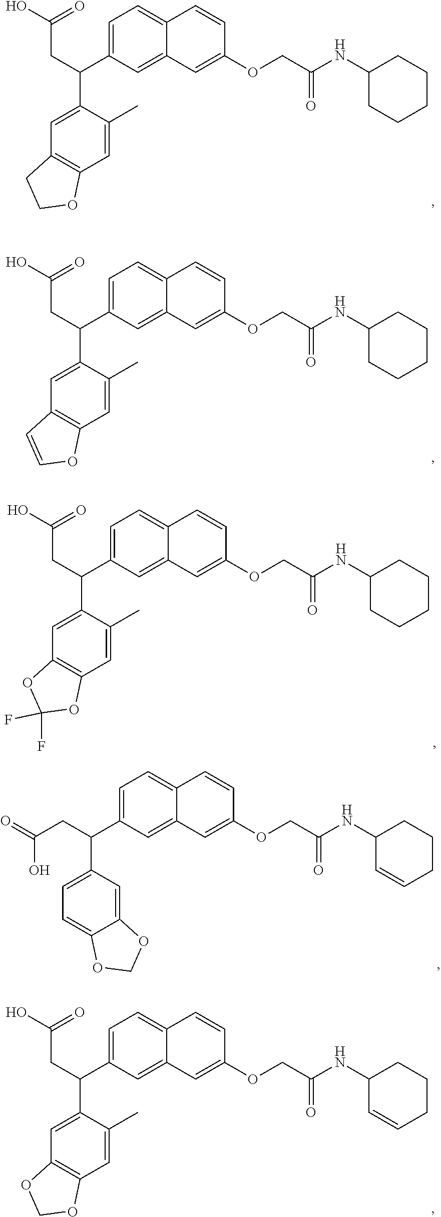

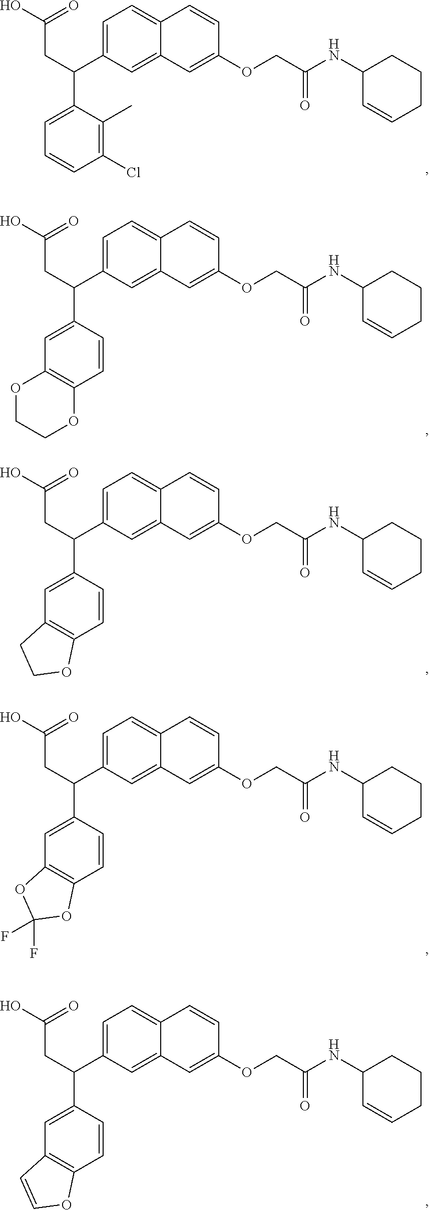

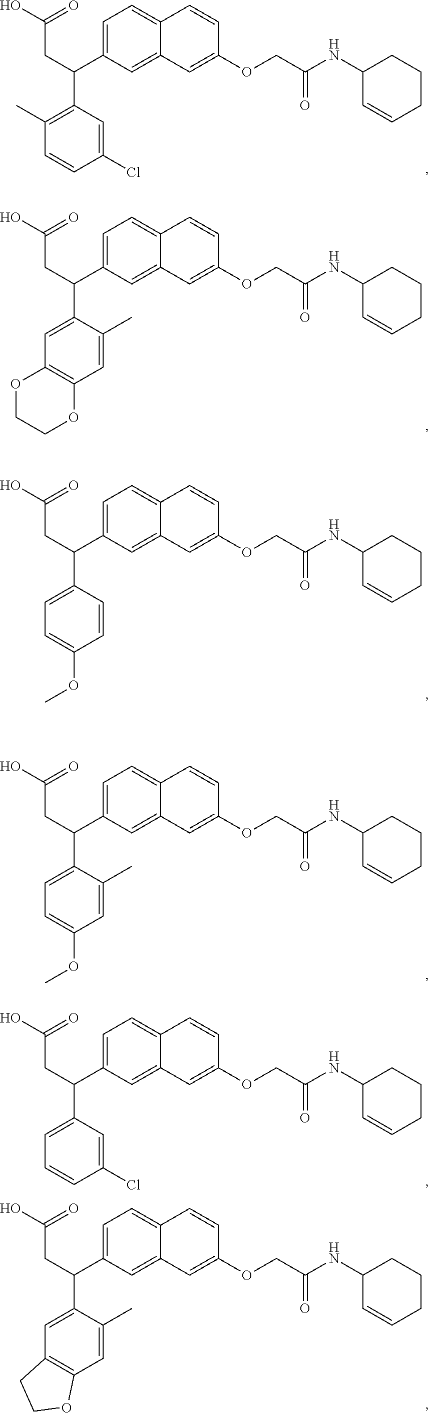

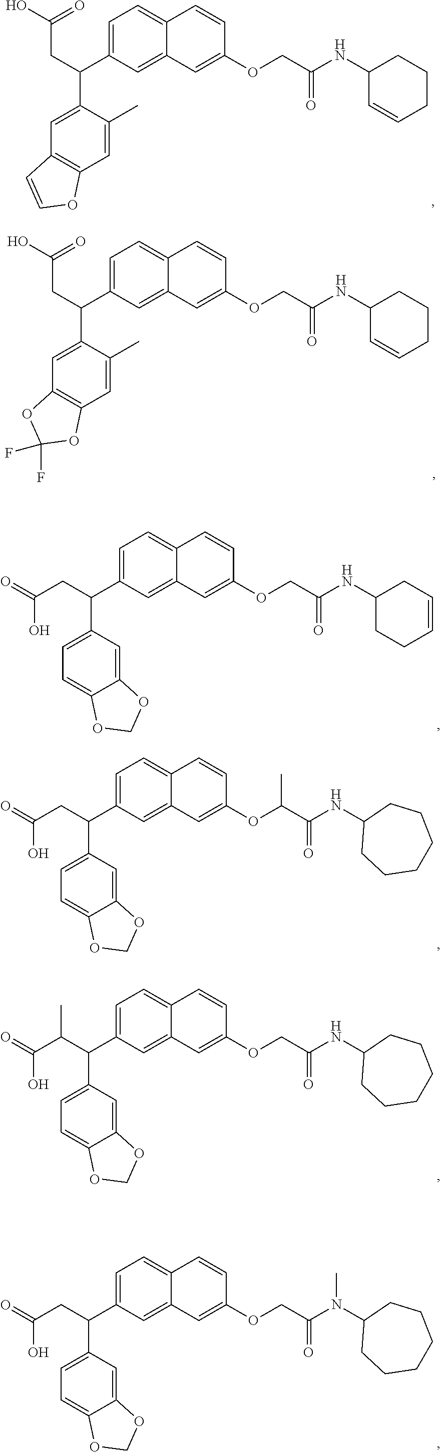

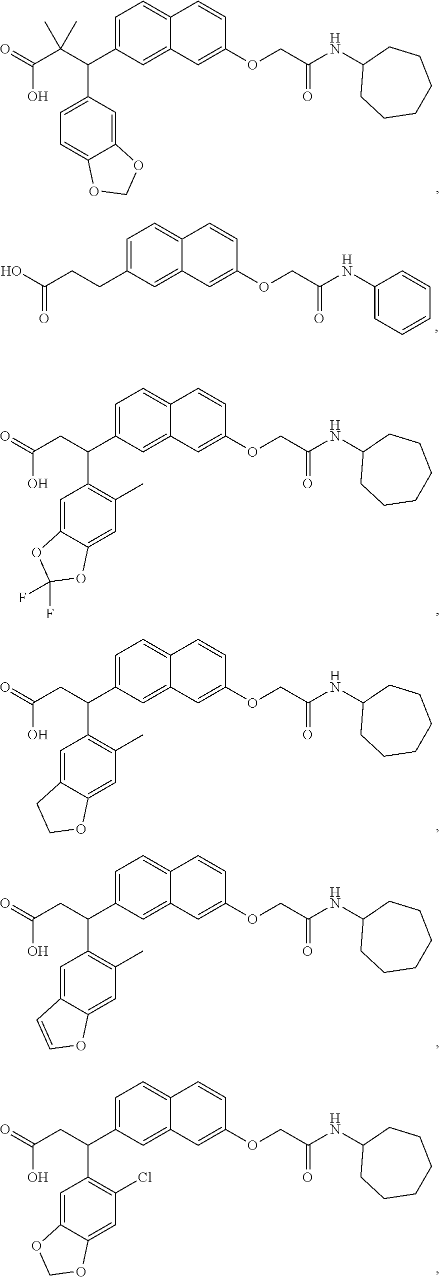

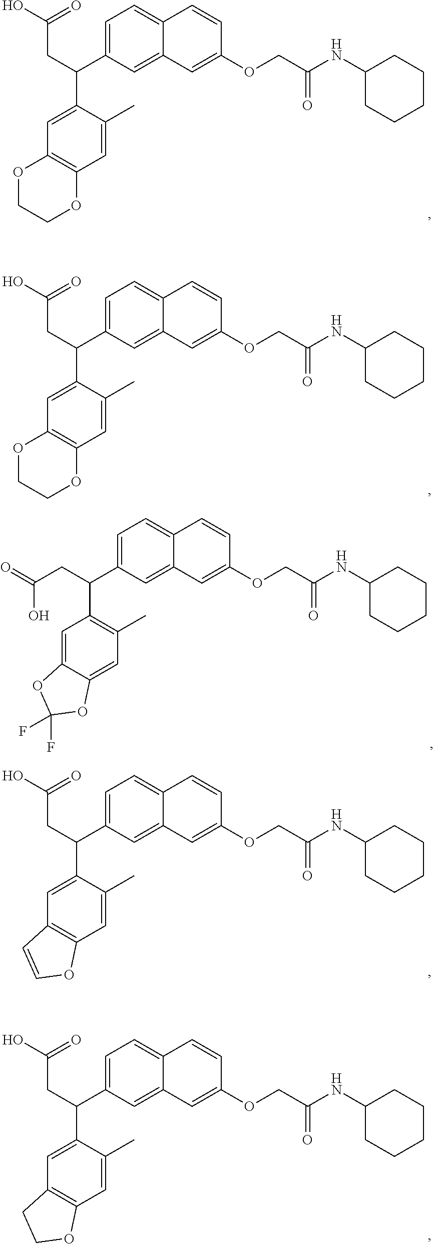

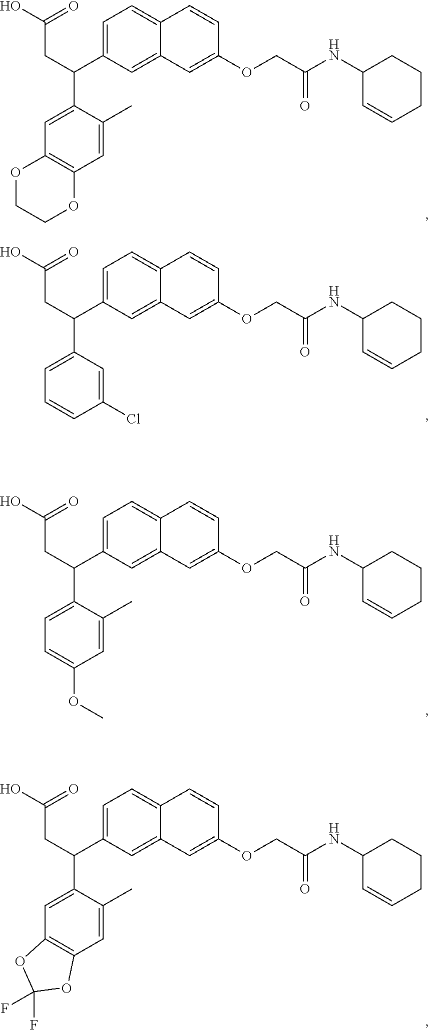

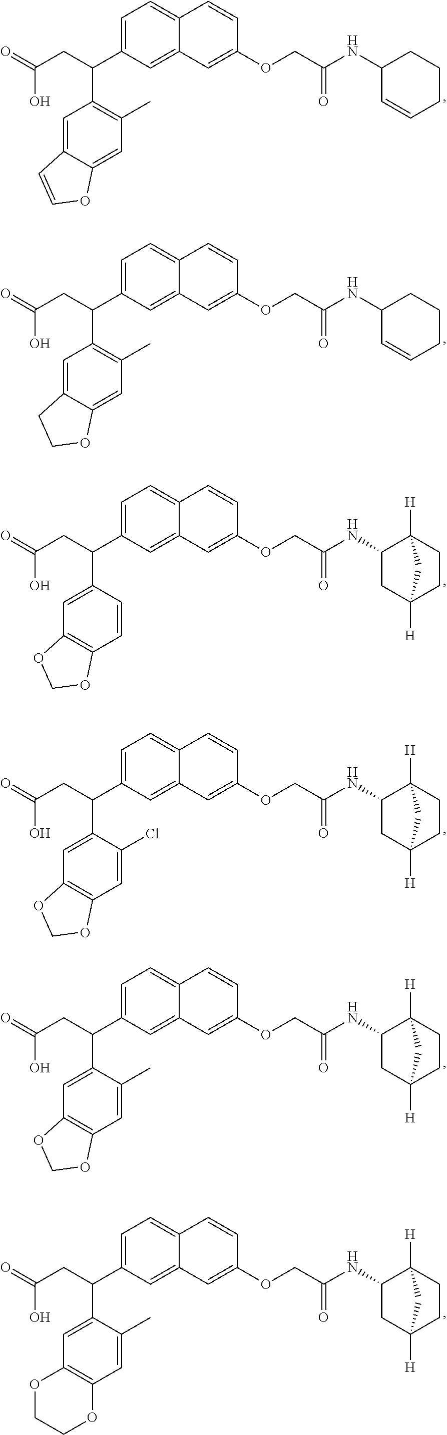

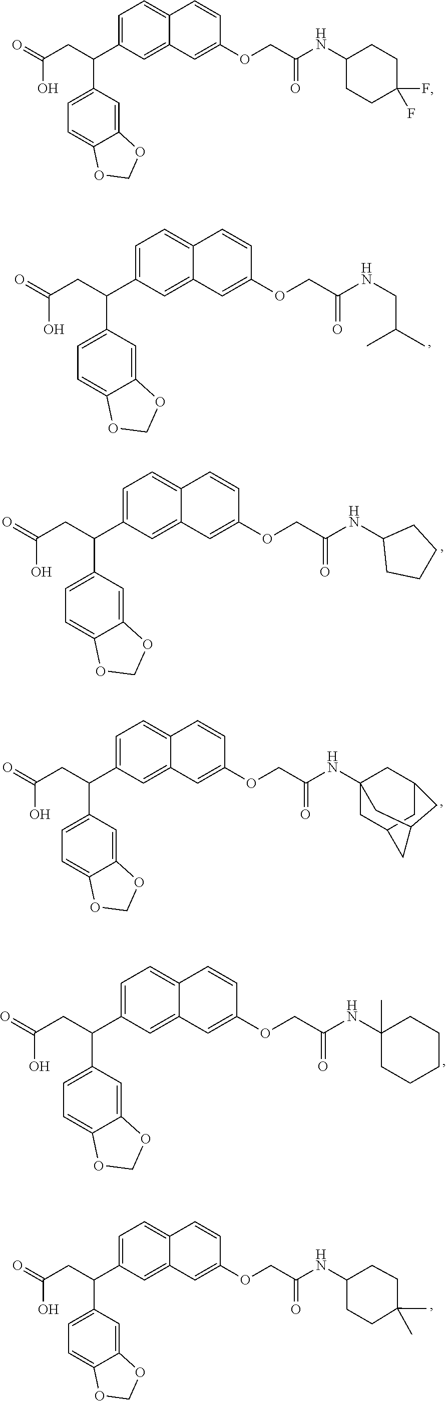

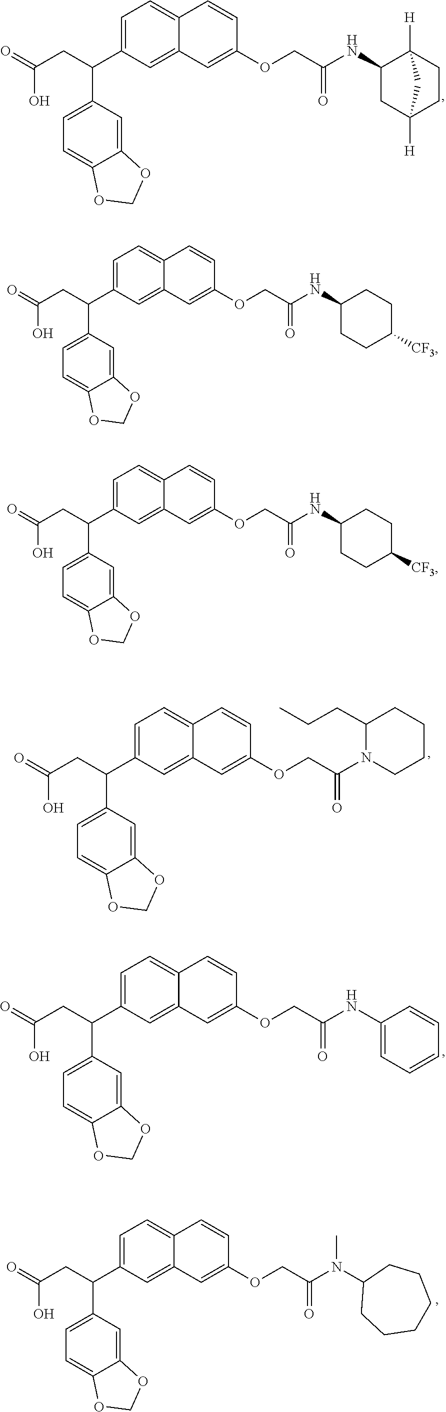

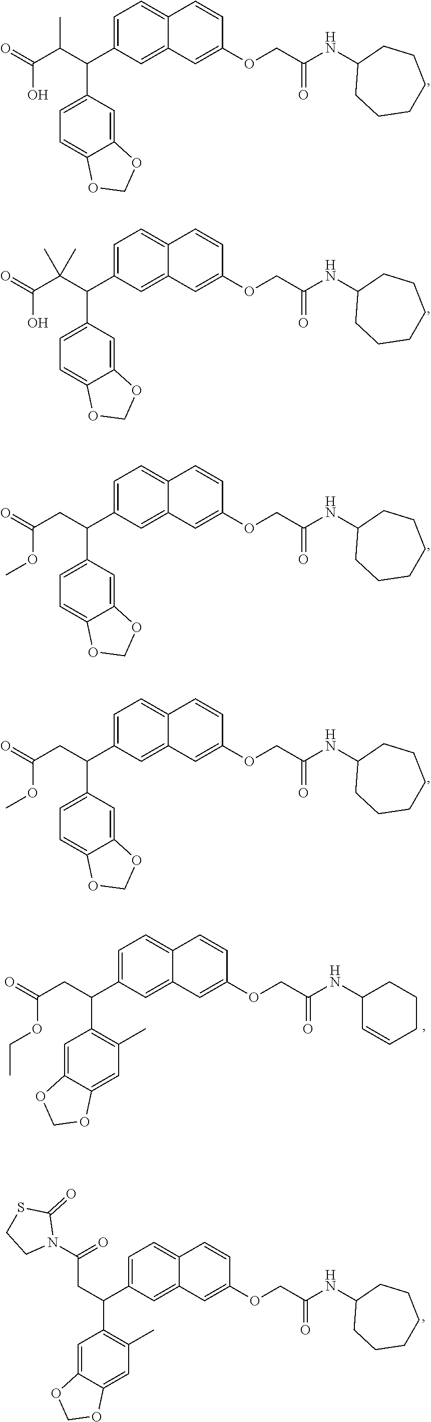

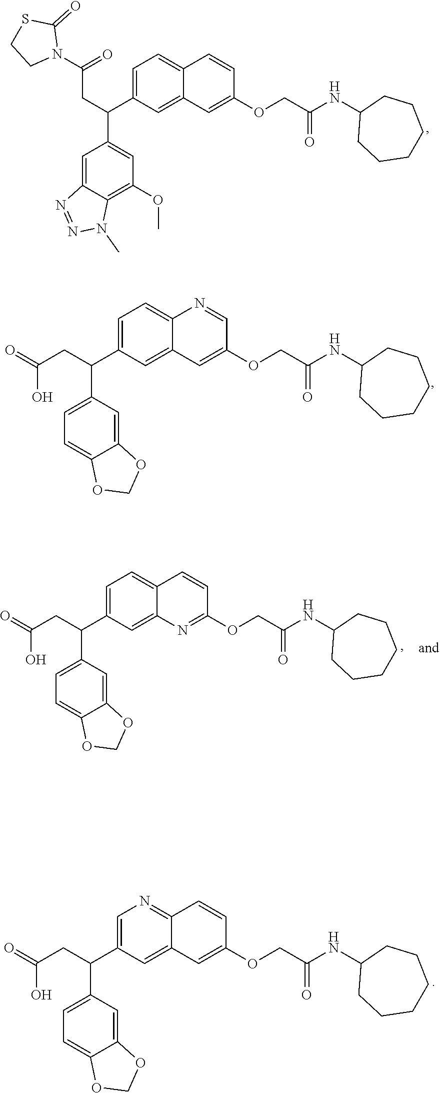 [SMILES: COc1cc(C(CC(=O)N2CCSC2=O)c2ccc3ccc(OCC(=O)NC4CCCCCC4)cc3c2)cc2nnn(C)c12 ; O=C(O)CC(c1ccc2c(c1)OCO2)c1ccc2ncc(OCC(=O)NC3CCCCCC3)cc2c1 ; O=C(O)CC(c1ccc2c(c1)OCO2)c1ccc2ccc(OCC(=O)NC3CCCCCC3)nc2c1 ; O=C(O)CC(c1ccc2c(c1)OCO2)c1cnc2ccc(OCC(=O)NC3CCCCCC3)cc2c1]

In one embodiment, the compound is 3-(7-(2-(Cyclohexylamino)-2-oxoethoxy)naphthalen-2-yl)propanoic acid, or a pharmaceutically acceptable salt thereof. In one embodiment, the compound is 3-(7-(2-Oxo-2-((tetrahydro-2H-pyran-4-yl)amino)ethoxy)naphthalen-2-yl)propanoic acid, or a pharmaceutically acceptable salt thereof. In one embodiment, the compound is 3-(7-(2-Morpholinoamino-2-oxoethoxy)naphthalen-2-yl)propanoic acid, or a pharmaceutically acceptable salt thereof. In one embodiment, the compound is 3-(7-(2-(Cycloheptylamino)-2-oxoethoxy)naphthalen-2-yl)propanoic acid, or a pharmaceutically acceptable salt thereof. In one embodiment, the compound is 3-(7-(2-((4-Methoxyphenyl)amino)-2-oxoethoxy)naphthalen-2-yl)propanoic acid, or a pharmaceutically acceptable salt thereof. In one embodiment, the compound is 3-(7-(2-(Mesitylamino)-2-oxoethoxy)naphthalen-2-yl)propanoic acid, or a pharmaceutically acceptable salt thereof. In one embodiment, the compound is 3-(7-(2-Oxo-2-((2,3,5,6-tetramethylphenyl)amino)ethoxy)naphthalen-2-yl)propanoic acid, or a pharmaceutically acceptable salt thereof. In one embodiment, the compound is 3-(7-(2-oxo-2-(phenylamino)ethoxy)naphthalen-2-yl)propanoic acid, or a pharmaceutically acceptable salt thereof. In one embodiment, the compound is 3-(7-(2-(Cycloheptylamino)-2-oxoethoxy)naphthalen-2-yl)-3-phenylpropanoic acid, or a pharmaceutically acceptable salt thereof. In one embodiment, the compound is 3-(7-(2-(Cycloheptylamino)-2-oxoethoxy)naphthalen-2-yl)-3-(4-hydroxyphenyl)propanoic acid, or a pharmaceutically acceptable salt thereof. In one embodiment, the compound is 3-(4-Chlorophenyl)-3-(7-(2-(cycloheptylamino)-2-oxoethoxy)naphthalen-2-yl)propanoic acid, or a pharmaceutically acceptable salt thereof. In one embodiment, the compound is 3-(7-(2-(Cycloheptylamino)-2-oxoethoxy)naphthalen-2-yl)-3-(4-methoxyphenyl)propanoic acid, or a pharmaceutically acceptable salt thereof. In one embodiment, the compound is 3-(7-(2-(Cycloheptylamino)-2-oxoethoxy)naphthalen-2-yl)-3-(4-ethoxyphenyl)propanoic acid, or a pharmaceutically acceptable salt thereof. In one embodiment, the compound is 3-(7-(2-(Cycloheptylamino)-2-oxoethoxy)naphthalen-2-yl)-3-(4-isopropoxyphenyl)propanoic acid, or a pharmaceutically acceptable salt thereof. In one embodiment, the compound is 3-(7-(2-(Cycloheptylamino)-2-oxoethoxy)naphthalen-2-yl)-3-(3-hydroxyphenyl)propanoic acid, or a pharmaceutically acceptable salt thereof. In one embodiment, the compound is 3-(7-(2-(Cycloheptylamino)-2-oxoethoxy)naphthalen-2-yl)-3-(3-methoxyphenyl)propanoic acid, or a pharmaceutically acceptable salt thereof. In one embodiment, the compound is 3-(3-Chlorophenyl)-3-(7-(2-(cycloheptylamino)-2-oxoethoxy)naphthalen-2-yl)propanoic acid, or a pharmaceutically acceptable salt thereof. In one embodiment, the compound is 3-(7-(2-(Cycloheptylamino)-2-oxoethoxy)naphthalen-2-yl)-3-(4-methoxy-3-methylphenyl)propanoic acid, or a pharmaceutically acceptable salt thereof. In one embodiment, the compound is 3-(7-(2-(Cycloheptylamino)-2-oxoethoxy)naphthalen-2-yl)-3-(4-methoxy-2-methylphenyl)propanoic acid, or a pharmaceutically acceptable salt thereof. In one embodiment, the compound is 3-(3-chloro-2-methylphenyl)-3-(7-(2-(cycloheptylamino)-2-oxoethoxy)naphthalen-2-yl)propanoic acid, or a pharmaceutically acceptable salt thereof. In one embodiment, the compound is 3-(benzo[d][1,3]dioxol-5-yl)-3-(7-(2-(cycloheptylamino)-2-oxoethoxy)naphthalen-2-yl)propanoic acid, or a pharmaceutically acceptable salt thereof. In one embodiment, the compound is 3-(7-(2-(cycloheptylamino)-2-oxoethoxy)naphthalen-2-yl)-3-(6-methylbenzo[d][1,3]dioxol-5-yl)propanoic acid, or a pharmaceutically acceptable salt thereof. In one embodiment, the compound is 3-(7-(2-(cycloheptylamino)-2-oxoethoxy)naphthalen-2-yl)-3-(7-methoxy-1-methyl-1H-benzo[d][1,2,3]triazol-5-yl)propanoic acid, or a pharmaceutically acceptable salt thereof. In one embodiment, the compound is 3-(7-(2-(cycloheptylamino)-2-oxoethoxy)naphthalen-2-yl)-3-(1-ethyl-4-methyl-1H-benzo[d][1,2,3]triazol-5-yl)propanoic acid, or a pharmaceutically acceptable salt thereof. In one embodiment, the compound is 3-(7-(2-(cycloheptylamino)-2-oxoethoxy) naphthalen-2-yl)-3-(2,3-dihydrobenzofuran-5-yl)propanoic acid, or a pharmaceutically acceptable salt thereof. In one embodiment, the compound is 3-(7-(2-(cycloheptylamino)-2-oxoethoxy)naphthalen-2-yl)-3-(2,2-difluorobenzo[d][1,3]dioxol-5-yl)propanoic acid, or a pharmaceutically acceptable salt thereof. In one embodiment, the compound is 3-(7-(2-(cycloheptylamino)-2-oxoethoxy)naphthalen-2-yl)-3-(2,3-dihydrobenzo[b][1,4]dioxin-6-yl)propanoic acid, or a pharmaceutically acceptable salt thereof. In one embodiment, the compound is 3-(Benzofuran-5-yl)-3-(7-(2-(cycloheptylamino)-2-oxoethoxy)naphthalen-2-yl)propanoic acid, or a pharmaceutically acceptable salt thereof. In one embodiment, the compound is 3-(7-(2-(Cycloheptylamino)-2-oxoethoxy)naphthalen-2-yl)-3-(7-methyl-2,3-dihydrobenzo[b][1,4]dioxin-6-yl)propanoic acid, or a pharmaceutically acceptable salt thereof. In one embodiment, the compound is 3-(5-chloro-2-methylphenyl)-3-(7-(2-(cycloheptylamino)-2-oxoethoxy)naphthalen-2-yl)propanoic acid, or a pharmaceutically acceptable salt thereof. In one embodiment, the compound is 3-(7-(2-(cycloheptylamino)-2-oxoethoxy)naphthalen-2-yl)-3-(6-methyl-2,3-dihydrobenzofuran-5-yl)propanoic acid, or a pharmaceutically acceptable salt thereof. In one embodiment, the compound is 3-(7-(2-(cycloheptylamino)-2-oxoethoxy)naphthalen-2-yl)-3-(6-methylbenzo[d][1,3]dioxol-5-yl)propanoic acid, or a pharmaceutically acceptable salt thereof. In one embodiment, the compound is 3-(6-chlorobenzo[d][1,3]dioxol-5-yl)-3-(7-(2-(cycloheptylamino)-2-oxoethoxy)naphthalen-2-yl)propanoic acid, or a pharmaceutically acceptable salt thereof. In one embodiment, the compound is 3-(7-(2-(cyclohexylamino)-2-oxoethoxy)naphthalen-2-yl)-3-(4-methoxyphenyl)propanoic acid, or a pharmaceutically acceptable salt thereof. In one embodiment, the compound is 3-(7-(2-(cyclohexylamino)-2-oxoethoxy)naphthalen-2-yl)-3-(4-methoxy-2-methylphenyl)propanoic acid 3-(7-(2-(cyclohexylamino)-2-oxoethoxy)naphthalen-2-yl)-3-(4-methoxy-2,6-dimethylphenyl)propanoic acid 3-(3-chlorophenyl)-3-(7-(2-(cyclohexylamino)-2-oxoethoxy)naphthalen-2-yl)propanoic acid, or a pharmaceutically acceptable salt thereof. In one embodiment, the compound is 3-(3-chloro-2-methylphenyl)-3-(7-(2-(cyclohexylamino)-2-oxoethoxy)naphthalen-2-yl)propanoic acid, or a pharmaceutically acceptable salt thereof. In one embodiment, the compound is 3-(benzo[d][1,3]dioxol-5-yl)-3-(7-(2-(cyclohexylamino)-2-oxoethoxy)naphthalen-2-yl)propanoic acid, or a pharmaceutically acceptable salt thereof. In one embodiment, the compound is 3-(7-(2-(cyclohexylamino)-2-oxoethoxy)naphthalen-2-yl)-3-(6-methylbenzo[d][1,3]dioxol-5-yl)propanoic acid, or a pharmaceutically acceptable salt thereof. In one embodiment, the compound is 3-(7-(2-(cyclohexylamino)-2-oxoethoxy)naphthalen-2-yl)-3-(1-ethyl-4-methyl-1H-benzo[d][1,2,3]triazol-5-yl)propanoic acid 3-(7-(2-(cyclohexylamino)-2-oxoethoxy)naphthalen-2-yl)-3-(2,3-dihydrobenzofuran-5-yl)propanoic acid, or a pharmaceutically acceptable salt thereof. In one embodiment, the compound is 3-(7-(2-(cyclohexylamino)-2-oxoethoxy)naphthalen-2-yl)-3-(2,2-dimethylbenzo[d][1,3]dioxol-5-yl)propanoic acid, or a pharmaceutically acceptable salt thereof. In one embodiment, the compound is 3-(7-(2-(cyclohexylamino)-2-oxoethoxy)naphthalen-2-yl)-3-(2,2-difluorobenzo[d][1,3]dioxol-5-yl)propanoic acid, or a pharmaceutically acceptable salt thereof. In one embodiment, the compound is 3-(7-(2-(cyclohexylamino)-2-oxoethoxy)naphthalen-2-yl)-3-(2,3-dihydrobenzo[b][1,4]dioxin-6-yl)propanoic acid, or a pharmaceutically acceptable salt thereof. In one embodiment, the compound is 3-(7-(2-(cyclohexylamino)-2-oxoethoxy)naphthalen-2-yl)-3-(5-methoxy-2-methylphenyl)propanoic acid, or a pharmaceutically acceptable salt thereof. In one embodiment, the compound is 3-(7-(2-(cyclohexylamino)-2-oxoethoxy)naphthalen-2-yl)-3-(6-methyl-1H-pyrrolo[2,3-b]pyridin-5-yl)propanoic acid, or a pharmaceutically acceptable salt thereof. In one embodiment, the compound is 3-(Benzofuran-5-yl)-3-(7-(2-(cyclohexylamino)-2-oxoethoxy)naphthalen-2-yl)propanoic acid, or a pharmaceutically acceptable salt thereof. In one embodiment, the compound is 3-(5-Chloro-2-methylphenyl)-3-(7-(2-(cyclohexylamino)-2-oxoethoxy)naphthalen-2-yl)propanoic acid, or a pharmaceutically acceptable salt thereof. In one embodiment, the compound is 3-(7-(2-(cyclohexylamino)-2-oxoethoxy)naphthalen-2-yl)-3-(7-methyl-2,3-dihydrobenzo[b][1,4]dioxin-6-yl)propanoic acid, or a pharmaceutically acceptable salt thereof. In one embodiment, the compound is 3-(7-(2-(cycloheptylamino)-2-oxoethoxy)naphthalen-2-yl)-3-(2,2-difluoro-6-methylbenzo[d][1,3]dioxol-5-yl)propanoic acid, or a pharmaceutically acceptable salt thereof. In one embodiment, the compound is 3-(7-(2-(cyclohexylamino)-2-oxoethoxy)naphthalen-2-yl)-3-(6-methylbenzofuran-5-yl)propanoic acid, or a pharmaceutically acceptable salt thereof. In one embodiment, the compound is 3-(7-(2-(cyclohexylamino)-2-oxoethoxy)naphthalen-2-yl)-3-(6-methyl-2,3-dihydrobenzofuran-5-yl)propanoic acid, or a pharmaceutically acceptable salt thereof. In one embodiment, the compound is 3-(Benzo[d][1,3]dioxol-5-yl)-3-(7-(2-(cyclohex-2-en-1-ylamino)-2-oxoethoxy)naphthalen-2-yl)propanoic acid, or a pharmaceutically acceptable salt thereof. In one embodiment, the compound is 3-(7-(2-(Cyclohex-2-en-1-ylamino)-2-oxoethoxy)naphthalen-2-yl)-3-(6-methylbenzo[d][1,3]dioxol-5-yl)propanoic acid, or a pharmaceutically acceptable salt thereof. In one embodiment, the compound is 3-(3-Chloro-2-methylphenyl)-3-(7-(2-(cyclohex-2-en-1-ylamino)-2-oxoethoxy)naphthalen-2-yl)propanoic acid, or a pharmaceutically acceptable salt thereof. In one embodiment, the compound is 3-(7-(2-(cyclohex-2-en-1-ylamino)-2-oxoethoxy)naphthalen-2-yl)-3-(2,3-dihydrobenzo[b][1,4]dioxin-6-yl)propanoic acid, or a pharmaceutically acceptable salt thereof. In one embodiment, the compound is 3-(7-(2-(Cyclohex-2-en-1-ylamino)-2-oxoethoxy)naphthalen-2-yl)-3-(2,3-dihydrobenzofuran-5-yl)propanoic acid, or a pharmaceutically acceptable salt thereof. In one embodiment, the compound is 3-(7-(2-(Cyclohex-2-en-1-ylamino)-2-oxoethoxy)naphthalen-2-yl)-3-(2,2-difluorobenzo[d][1,3]dioxol-5-yl)propanoic acid, or a pharmaceutically acceptable salt thereof. In one embodiment, the compound is 3-(Benzofuran-5-yl)-3-(7-(2-(cyclohex-2-en-1-ylamino)-2-oxoethoxy)naphthalen-2-yl)propanoic acid, or a pharmaceutically acceptable salt thereof. In one embodiment, the compound is 3-(5-Chloro-2-methylphenyl)-3-(7-(2-(cyclohex-2-en-1-ylamino)-2-oxoethoxy)naphthalen-2-yl)propanoic acid, or a pharmaceutically acceptable salt thereof. In one embodiment, the compound is 3-(7-(2-(cyclohex-2-en-1-ylamino)-2-oxoethoxy)naphthalen-2-yl)-3-(7-methyl-2,3-dihydrobenzo[b][1,4]dioxin-6-yl)propanoic acid, or a pharmaceutically acceptable salt thereof. In one embodiment, the compound is 3-(3-chlorophenyl)-3-(7-(2-(cyclohex-2-en-1-ylamino)-2-oxoethoxy)naphthalen-2-yl)propanoic acid, or a pharmaceutically acceptable salt thereof. In one embodiment, the compound is 3-(7-(2-(cyclohex-2-en-1-ylamino)-2-oxoethoxy)naphthalen-2-yl)-3-(4-methoxy-2-methylphenyl)propanoic acid, or a pharmaceutically acceptable salt thereof. In one embodiment, the compound is 3-(7-(2-(cyclohex-2-en-1-ylamino)-2-oxoethoxy)naphthalen-2-yl)-3-(2,2-difluoro-6-methylbenzo[d][1,3]dioxol-5-yl)propanoic acid, or a pharmaceutically acceptable salt thereof. In one embodiment, the compound is 3-(7-(2-(cycloheptylamino)-2-oxoethoxy)naphthalen-2-yl)-3-(6-methylbenzo[d][1,3]dioxol-5-yl)propanoic acid, or a pharmaceutically acceptable salt thereof. In one embodiment, the compound is 3-(7-(2-(cyclohex-2-en-1-ylamino)-2-oxoethoxy)naphthalen-2-yl)-3-(6-methyl-2,3-dihydrobenzofuran-5-yl)propanoic acid, or a pharmaceutically acceptable salt thereof. In one embodiment, the compound is3-(benzo[d][1,3]dioxol-5-yl)-3-(7-(2-(((1S,2S,4R)-bicyclo[2.2.1]heptan-2-yl)amino)-2-oxoethoxy)naphthalen-2-yl)propanoic acid, or a pharmaceutically acceptable salt thereof. In one embodiment, the compound is 3-(7-(2-(((1S,2S,4R)-bicyclo[2.2.1]heptan-2-yl)amino)-2-oxoethoxy)naphthalen-2-yl)-3-(6-chlorobenzo[d][1,3]dioxol-5-yl)propanoic acid, or a pharmaceutically acceptable salt thereof. In one embodiment, the compound is 3-(7-(2-(((1S,2S,4R)-bicyclo[2.2.1]heptan-2-yl)amino)-2-oxoethoxy)naphthalen-2-yl)-3-(6-methylbenzo[d][1,3]dioxol-5-yl)propanoic acid, or a pharmaceutically acceptable salt thereof. In one embodiment, the compound is 3-(7-(2-(((1S,2S,4R)-bicyclo[2.2.1]heptan-2-yl)amino)-2-oxoethoxy)naphthalen-2-yl)-3-(7-methyl-2,3-dihydrobenzo[b][1,4]dioxin-6-yl)propanoic acid, or a pharmaceutically acceptable salt thereof. In one embodiment, the compound is 3-(Benzo[d][1,3]dioxol-5-yl)-3-(7-(2-(cyclohex-3-en-1-ylamino)-2-oxoethoxy)naphthalen-2-yl)propanoic acid, or a pharmaceutically acceptable salt thereof. In one embodiment, the compound is 3-(benzo[d][1,3]dioxol-5-yl)-3-(7-(2-((4,4-difluorocyclohexyl)amino)-2-oxoethoxy)naphthalen-2-yl)propanoic acid, or a pharmaceutically acceptable salt thereof. In one embodiment, the compound is 3-(benzo[d][1,3]dioxol-5-yl)-3-(7-(2-(isobutylamino)-2-oxoethoxy)naphthalen-2-yl)propanoic acid, or a pharmaceutically acceptable salt thereof. In one embodiment, the compound is 3-(benzo[d][1,3]dioxol-5-yl)-3-(7-(2-(cyclopentylamino)-2-oxoethoxy)naphthalen-2-yl)propanoic acid, or a pharmaceutically acceptable salt thereof. In one embodiment, the compound is 3-(7-(2-(((1S,3s)-adamantan-1-yl)amino)-2-oxoethoxy)naphthalen-2-yl)-3-(benzo[d][1,3]dioxol-5-yl)propanoic acid, or a pharmaceutically acceptable salt thereof. In one embodiment, the compound is 3-(benzo[d][1,3]dioxol-5-yl)-3-(7-(2-((1-methylcyclohexyl)amino)-2-oxoethoxy)naphthalen-2-yl)propanoic acid, or a pharmaceutically acceptable salt thereof. In one embodiment, the compound is 3-(benzo[d][1,3]dioxol-5-yl)-3-(7-(2-((4,4-dimethylcyclohexyl)amino)-2-oxoethoxy)naphthalen-2-yl)propanoic acid, or a pharmaceutically acceptable salt thereof. In one embodiment, the compound is 3-(benzo[d][1,3]dioxol-5-yl)-3-(7-(2-(((1S,2R,4R)-bicyclo[2.2.1]heptan-2-yl)amino)-2-oxoethoxy)naphthalen-2-yl)propanoic acid, or a pharmaceutically acceptable salt thereof. In one embodiment, the compound is 3-(benzo[d][1,3]dioxol-5-yl)-3-(7-(2-oxo-2-((trans-4-(trifluoromethyl)cyclohexyl)amino)ethoxy)naphthalen-2-yl)propanoic acid, or a pharmaceutically acceptable salt thereof. In one embodiment, the compound is 3-(benzo[d][1,3]dioxol-5-yl)-3-(7-(2-oxo-2-(2-propylpiperidin-1-yl)ethoxy)naphthalen-2-yl)propanoic acid, or a pharmaceutically acceptable salt thereof. In one embodiment, the compound is 3-(benzo[d][1,3]dioxol-5-yl)-3-(7-(2-oxo-2-(phenylamino)ethoxy)naphthalen-2-yl)propanoic acid, or a pharmaceutically acceptable salt thereof. In one embodiment, the compound is 3-(Benzo[d][1,3]dioxol-5-yl)-3-(7-((1-(cycloheptylamino)-1-oxopropan-2-yl)oxy)naphthalen-2-yl)propanoic acid, or a pharmaceutically acceptable salt thereof. In one embodiment, the compound is 3-(benzo[d][1,3]dioxol-5-yl)-3-(7-(2-(cycloheptyl(methyl)amino)-2-oxoethoxy)naphthalen-2-yl)propanoic acid, or a pharmaceutically acceptable salt thereof. In one embodiment, the compound is 3-(benzo[d][1,3]dioxol-5-yl)-3-(7-(2-(cycloheptylamino)-2-oxoethoxy)naphthalen-2-yl)-2-methylpropanoic acid, or a pharmaceutically acceptable salt thereof. In one embodiment, the compound is 3-(benzo[d][1,3]dioxol-5-yl)-3-(7-(2-(cycloheptylamino)-2-oxoethoxy)naphthalen-2-yl)-2,2-dimethylpropanoic acid, or a pharmaceutically acceptable salt thereof. In one embodiment, the compound is Methyl 3-(benzo[d][1,3]dioxol-5-yl)-3-(7-(2-(cycloheptylamino)-2-oxoethoxy)naphthalen-2-yl)propanoate, or a pharmaceutically acceptable salt thereof. In one embodiment, the compound is Ethyl 3-(7-(2-(cyclohex-2-en-1-ylamino)-2-oxoethoxy)naphthalen-2-yl)-3-(6-methylbenzo[d][1,3]dioxol-5-yl)propanoate, or a pharmaceutically acceptable salt thereof. In one embodiment, the compound is N-cycloheptyl-2-((7-(1-(6-methylbenzo[d][1,3]dioxol-5-yl)-3-oxo-3-(2-oxothiazolidin-3-yl)propyl)naphthalen-2-yl)oxy) acetamide, or a pharmaceutically acceptable salt thereof. In one embodiment, the compound is N-cycloheptyl-2-((7-(1-7-methoxy-1-methyl-1H-benzo[d][1,2,3]triazol-5-yl)-3-oxo-3-(2-oxothiazolidin-3-yl)propyl)naphthalen-2-yl)oxy) acetamide, or a pharmaceutically acceptable salt thereof. In one embodiment, the compound is 3-(benzo[d][1,3]dioxol-5-yl)-3-(3-(2-(cycloheptylamino)-2-oxoethoxy) quinolin-6-yl)propanoic acid, or a pharmaceutically acceptable salt thereof. In one embodiment, the compound is 3-(benzo[d][1,3]dioxol-5-yl)-3-(2-(2-(cycloheptylamino)-2-oxoethoxy) quinolin-7-yl)propanoic acid, or a pharmaceutically acceptable salt thereof. In one embodiment, the compound is 3-(benzo[d][1,3]dioxol-5-yl)-3-(6-(2-(cycloheptylamino)-2-oxoethoxy) quinolin-3-yl)propanoic acid, or a pharmaceutically acceptable salt thereof.

### Pharmaceutically Acceptable Salts

The compound of the invention may be provided in any form suitable for the intended administration, including pharmaceutically (i.e. physiologically) acceptable salts. Such salts may be formed by procedures well known and described in the art.

### Prodrugs

In one aspect, the present invention relates to a prodrug of a compound of Formula (I). As used herein “prodrug” refers to a compound which is converted to a therapeutically active compound after administration, and the term should be interpreted as broadly herein as is generally understood in the art. In one embodiment, the conversion occurs by hydrolysis of an ester group or some other biologically labile group. Generally, but not necessarily, a prodrug is inactive or less active than the therapeutically active compound to which it is converted. Ester prodrugs of the compounds disclosed herein are contemplated. While not intending to be limiting, an ester may be an alkyl ester, an aryl ester, or a heteroaryl ester.

In one embodiment, the prodrug is based on masking the carboxylic acid of the compound of Formula (I), for example with an ester, a methyl amide or thiazolidinones. Thus, in one aspect, the present invention relates to a compound of Formula (XI):

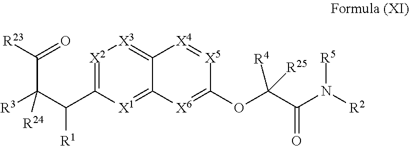

wherein
X¹ is CH or N;
X² is CH or N;
X³ is CH or N;
X⁴ is CH or N;
X⁵ is CH or N;
X⁶ is CH or N;
R¹ is phenyl, pyridinyl or H, wherein the phenyl or pyridinyl is optionally substituted with one or more, identical or different, substituents R⁶, wherein two of the substituents R⁶ optionally are linked together to form a ring;
R² is selected from the group consisting of C₅₋₇ cycloalkyl, C₅₋₇ cycloalkenyl, C₅₋₇ heterocycloalkyl, phenyl, C₅₋₈ bicycloalkyl, C₃₋₆ alkyl, and C₉₋₁₂ tricycloalkyl, wherein said C₅₋₇ cycloalkyl, C₅₋₇ cycloalkenyl, C₅₋₇ heterocycloalkyl, phenyl, C₅₋₈ bicycloalkyl, C₃₋₆ alkyl, and C₉₋₁₂ tricycloalkyl is optionally substituted with one or more, identical of different substituents R²⁸;
R⁵ is H or CH₃;
or R² is C₃₋₆ alkanediyl optionally substituted with R²⁸, R⁵ is a bond, and R² and R⁵ are linked together to form a ring;
R³ is H or CH₃;
R⁴ is H or CH₃;
R⁶ is selected from the group consisting of C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group of the C₁₋₅ alkyl, —OC₁₋₅ alkyl, or C₂₋₅ alkenyl is optionally replaced with O or N(R⁷); and/or
   wherein one or more methine group of the C₂₋₅ alkenyl is optionally replaced with N;
R⁷ is H or C₁₋₃ alkyl;
R²³ is selected from the group consisting of —OH;

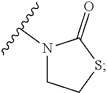 [SMILES: *N1CCSC1=O]

—OC₁₋₅ alkyl optionally substituted with one or more, identical or different, substituents R²⁶; —OC₂₋₅ alkenyl optionally substituted with one or more, identical or different, substituents R²⁶; —OC₂₋₅ alkynyl optionally substituted with one or more, identical or different, substituents R²⁶; —OC₃₋₆ cycloalkyl optionally substituted with one or more, identical or different, substituents R²⁶; —O-phenyl optionally substituted with one or more, identical or different, substituents R²⁷; and —O-benzyl optionally substituted with one or more, identical or different, substituents R²⁷; and —NH(CH₃);
R²⁴ is H or CH₃;
R²⁵ is H or CH₃;
R²⁶ is independently selected from the group consisting of H, deuterium, F, Cl and —OC₁₋₃ alkyl;
R²⁷ is independently selected from the group consisting of deuterium, methoxy, nitro, cyano, Cl, Br, I, and F; and
R²⁸ is individually selected from C₁₋₃ alkyl, —OC₁₋₃ alkyl, halogen, and deuterium, wherein said C₁₋₃ alkyl is optionally substituted with one or more halogens;
or a pharmaceutically acceptable salt, hydrate, polymorph, tautomer, or solvate thereof.

### Pharmaceutical Formulations

In one aspect, the present invention relates to a composition comprising the compound as disclosed herein. The composition according to the present disclosure may be used for treating, ameliorating and/or preventing a disease caused by formation of reactive oxygen species (ROS). Thus, preferably, the compositions and compounds are pharmaceutically acceptable. In one embodiment the composition as described herein is in the form of a pharmaceutical formulation. In one embodiment, the composition as described herein further comprises a pharmaceutically acceptable carrier. In one aspect, the present invention concerns a composition comprising the compound as defined herein and a pharmaceutically acceptable carrier.

### Method of Manufacturing

In one aspect, the present disclosure relates to methods of manufacturing compounds according to formula (XI).

Compounds according to the present invention may be prepared according to any conventional methods of chemical synthesis known by the skilled person, e.g. those described in the working examples. The starting materials for the processes described in the present application are known or may readily be prepared by conventional methods known by the skilled artisan from commercially available chemicals.

The end products of the reactions described herein may be isolated by conventional technique such as extraction, crystallisation, distillation, chromatography etc.

The compounds of this invention may exist in unsolvated as well as in solvated forms with pharmaceutically acceptable solvents such as water, ethanol and the like. In general, the solvated forms are considered equivalent to the unsolvated forms for the purposes of this invention.

### Biological Activity

The compounds of the present invention are capable of binding to the Keap1 Kelch domain and inhibiting the Keap1-Nrf2 interaction. In one embodiment, the compound is capable of reducing binding of Keap1 to Nrf2. In one aspect, the present invention relates to a method for reducing binding of Keap1 to Nrf2, said method comprising administering a compound or a composition as defined herein. In one embodiment, the compound has a Kᵢ value for inhibiting binding of Keap1 to Nrf2 of less than 500 μM, such as less than 100 μM, such as less than 50 μM, such as less than 25 μM, such as less than 10 μM, such as less than 5 μM, such as less than 1 μM, such as less than 0.5 μM, such as less than 0.1 μM, such as less than 0.01 μM. Said Kᵢ value may be determined using a fluorescence polarization (FP) competition assay.

By inhibiting the Keap1-Nrf2 interaction, the cellular concentration of detoxifying and antioxidant enzymes are increased, and level of reactive oxygen species (ROS) is reduced. Thus, in one embodiment, the compound is capable of reducing the cellular concentration of ROS. In one aspect, the present invention relates to a method for reducing the cellular concentration of ROS, said method comprising administering a compound or composition as defined herein. In one aspect, the present invention relates to an in vitro method for reducing the cellular concentration of ROS, said method comprising contacting cells expressing Keap1 with a compound or composition as defined herein.

ROS are known to contribute to oxidative stress. Thus, by inhibiting the Keap 1-Nrf2 interaction and thereby reducing the cellular concentration of ROS, the oxidative stress is reduced. In one embodiment, the compound is capable of reducing oxidative stress. In one aspect, the present invention relates to a method for reducing oxidative stress in a subject, said method comprising administering a compound or composition as defined herein to a subject in need thereof. In one aspect, the present invention relates to an in vitro method for reducing oxidative stress, said method comprising contacting cells expressing Keap1 with a compound or composition as defined herein.

Oxidative stress may trigger inflammation. In one aspect, the present invention relates to a compound or composition as defined herein for use in treating, ameliorating and/or preventing inflammation. In one aspect, the present invention relates to a method of treating, ameliorating and/or preventing inflammation, said method comprising administering a therapeutically effective dose of a compound or composition as defined herein to said subject in need thereof. In one aspect, the present invention relates to use of a compound or composition as defined herein in the manufacture of a medicament for treating, ameliorating and/or preventing inflammation.

In one aspect, the present invention relates to a method for inhibiting Keap1, said method comprising contacting cells expressing Keap1 with a compound or composition as defined herein.

### Medical Use

In one aspect, the present invention relates to a compound or composition as defined herein for use as a medicament.

In one aspect, the present invention relates to a compound or composition as defined herein for use in treating, ameliorating and/or preventing a disease where reactive oxygen species (ROS) and/or inflammation is involved. For example, the disease may be caused by excessive ROS formation and/or by misplaced ROS. In one embodiment, the disease caused by formation of ROS is a disease in which inflammation plays a role in pathogenesis. In one embodiment, the disease caused by formation of ROS involves inflammation. In one embodiment, the disease caused by ROS is selected from the group consisting of Central Nervous System (CNS) diseases; lung diseases; autoimmune disorders; cancer; metabolic and/or inflammatory liver conditions; CKD, incl. Alport syndrome; skin diseases; retinal ischemia-reperfusion (I/R) injury; ulcerative colitis; and acetaminophen-induced hepatotoxicity.

In one aspect, the present invention relates to a compound or composition as defined herein for use in treating, ameliorating and/or preventing a disease selected from the group consisting of a Central Nervous System (CNS) disease; a lung disease; an autoimmune disorder; cancer; a metabolic and/or inflammatory liver condition; CKD, incl. Alport syndrome; a skin disease; retinal ischemia-reperfusion (I/R) injury; ulcerative colitis; and acetaminophen-induced hepatotoxicity, in a subject.

In one embodiment, the disease is a CNS disease. In one embodiment, the CNS disease is selected from the group consisting of ischemic stroke, traumatic brain injury, cerebral ischemia, intracerebral haemorrhage, Alzheimer's disease, Parkinson's disease, and multiple sclerosis. In one embodiment, the CNS disorder is a CNS injury, such as selected from the group consisting of cerebral ischemia, intracerebral haemorrhage, ischemic stroke, and traumatic brain injury. In one embodiment, the CNS disorder is a neurodegenerative disorder, such as selected from the group consisting of Alzheimer's disease, Parkinson's disease, and multiple sclerosis.

In one embodiment, the disease is retinal ischemia-reperfusion (I/R) injury.

In one embodiment, the disease is a lung disease. In one embodiment, the lung disease is selected from the group consisting of acute lung inflammation, for example mediated by viral infections; chronic obstructive pulmonary disease; and pulmonary hypertension.

In one embodiment, the disease is a metabolic and/or inflammatory liver condition. In one embodiment, the disease is non-alcoholic steatohepatitis (NASH).

In one embodiment, the disease is cancer.

In one embodiment, the disease is a chronic kidney disease (CKD). In one embodiment, the disease is Alport syndrome.

In one embodiment, the disease is ulcerative colitis. In one embodiment, the disease is acetaminophen-induced hepatotoxicity.

In one embodiment, the disease is an autoimmune disorder. In one embodiment, the autoimmune disorder is rheumatoid arthritis.

In one embodiment, the disease is a skin disease. In one embodiment, the skin disease is atopic dermatitis or psoriasis.

The term “treatment” refers to the combating of a disease or disorder. “Treatment” or “treating,” as used herein, includes any desirable effect on the symptoms or pathology of a disease or condition as described herein, and may include even minimal changes or improvements in one or more measurable markers of the disease or condition being treated. “Treatment” or “treating” does not necessarily indicate complete eradication or cure of the disease or condition, or associated symptoms thereof. In some embodiments, the term “treatment” encompasses amelioration and prevention.

The term “amelioration” refers to moderation in the severity of the symptoms of a disease or condition. Improvement in a patient's condition, or the activity of making an effort to correct, or at least make more acceptable, conditions that are difficult to endure related to patient's conditions is considered “ameliorative” treatment.

The term “prevent” or “preventing” refers to precluding, averting, obviating, forestalling, stopping, or hindering something from happening, especially by advance action.

In one embodiment, the compound is administered at a therapeutically effective dose. A “therapeutically effective dose” refers to that amount of active ingredient, which ameliorates the symptoms or condition. In one embodiment, the compound or composition as defined herein is administered at a daily dosage of between 0.1 μg compound per kg and 1000 mg compound per kg bodyweight.

In one aspect, the present invention relates to a method of treating a disease caused by ROS in a subject, said method comprising administering a therapeutically effective dose of a compound or composition as defined herein to said subject in need thereof.

In one aspect, the present invention relates to use of a compound or composition as defined herein in the manufacture of a medicament for treatment of a disease caused by ROS.

In one embodiment, the subject is a mammal. In one embodiment, the subject is a human.

In one aspect, the present invention relates to a method of reducing inflammation, said method comprising administering a therapeutically effective amount of the compound or composition as defined herein to a subject in need thereof.

In one embodiment, the subject is a mammal. In one embodiment, the subject is a human. In one embodiment, the subject is suffering from a condition selected from the group consisting of a Central Nervous System (CNS) disease; a lung disease; an autoimmune disorder; cancer; a metabolic and fibrotic kidney and liver condition; Alport syndrome; and a skin disease.

### Items

1. A compound of Formula (XI):

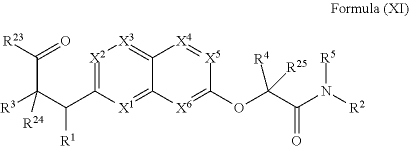

wherein
X¹ is CH or N;
X² is CH or N;
X³ is CH or N;
X⁴ is CH or N;
X⁵ is CH or N;
X⁶ is CH or N;
R¹ is phenyl, pyridinyl or H, wherein the phenyl or pyridinyl is optionally substituted with one or more, identical or different, substituents R⁶, wherein two of the substituents R⁶ optionally are linked together to form a ring;
R² is selected from the group consisting of C₅₋₇ cycloalkyl, C₅₋₇ cycloalkenyl, C₅₋₇ heterocycloalkyl, phenyl, C₅₋₈ bicycloalkyl, C₃₋₆ alkyl, and C₉₋₁₂ tricycloalkyl, wherein said C₅₋₇ cycloalkyl, C₅₋₇ cycloalkenyl, C₅₋₇ heterocycloalkyl, phenyl, C₅₋₈ bicycloalkyl, C₃₋₆ alkyl, and C₉₋₁₂ tricycloalkyl is optionally substituted with one or more, identical of different substituents R²⁸;
R⁵ is H or CH₃;
or R² is C₃₋₆ alkanediyl optionally substituted with R²⁸, R⁵ is a bond, and R² and R⁵ are linked together to form a ring;
R³ is H or CH₃;
R⁴ is H or CH₃;
R⁶ is selected from the group consisting of C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group of the C₁₋₅ alkyl, —OC₁₋₅ alkyl, or C₂₋₅ alkenyl is optionally replaced with O or N(R⁷); and/or
   wherein one or more methine group of the C₂₋₅ alkenyl is optionally replaced with N;
R⁷ is H or C₁₋₃ alkyl;
R²³ is selected from the group consisting of —OH;

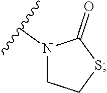 [SMILES: *N1CCSC1=O]

—OC₁₋₅ alkyl optionally substituted with one or more, identical or different, substituents R²⁶, —OC₂₋₅ alkenyl optionally substituted with one or more, identical or different, substituents R²⁶; —OC₂₋₅ alkynyl optionally substituted with one or more, identical or different, substituents R²⁶; —OC₃₋₆ cycloalkyl optionally substituted with one or more, identical or different, substituents R²⁶; —O-phenyl optionally substituted with one or more, identical or different, substituents R²⁷; and —O-benzyl optionally substituted with one or more, identical or different, substituents R²⁷; and —NH(CH₃);
R²⁴ is H or CH₃;
R²⁵ is H or CH₃;
R²⁶ is independently selected from the group consisting of H, deuterium, F, Cl and —OC₁₋₃ alkyl;
R²⁷ is independently selected from the group consisting of deuterium, methoxy, nitro, cyano, Cl, Br, I, and F; and
R²⁸ is individually selected from C₁₋₃ alkyl, —OC₁₋₃ alkyl, halogen, and deuterium, wherein said C₁₋₃ alkyl is optionally substituted with one or more halogens; or a pharmaceutically acceptable salt, hydrate, polymorph, tautomer, or solvate thereof.

2. A compound of Formula (XI):

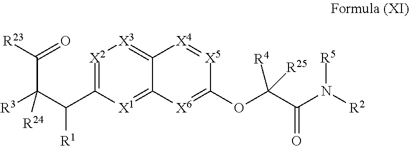

wherein
X¹ is CH or N;
X² is CH or N;
X³ is CH or N;
X⁴ is CH or N;
X⁵ is CH or N;
X⁶ is CH or N;
R¹ is phenyl, pyridinyl or H, wherein the phenyl or pyridinyl is optionally substituted with one or more, identical or different, substituents R⁶, wherein two of the substituents R⁶ optionally are linked together to form a ring;
R² is selected from the group consisting of C₅₋₇ cycloalkyl, C₅₋₇ cycloalkenyl C₅₋₇ heterocycloalkyl and phenyl,
   wherein said C₅₋₇ cycloalkyl, C₅₋₇ cycloalkenyl C₅₋₇ heterocycloalkyl and phenyl is optionally substituted with one or more substituents individually selected from C₁₋₃ alkyl, —OC₁₋₃ alkyl, halogen and deuterium,
      wherein said C₁₋₃ alkyl is optionally substituted with one or more halogens;
R³ is H or CH₃;
R⁴ is H or CH₃;
R⁵ is H or CH₃;
R⁶ is selected from the group consisting of C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group of the C₁₋₅ alkyl, —OC₁₋₅ alkyl, or C₂₋₅ alkenyl is optionally replaced with O or N(R⁷); and/or
   wherein one or more methine group of the C₂₋₅ alkenyl is optionally replaced with N;
R⁷ is H or C₁₋₃ alkyl;
R²³ is selected from the group consisting of —OH;

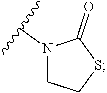 [SMILES: *N1CCSC1=O]

—OC₁₋₅ alkyl optionally substituted with one or more, identical or different, substituents R²⁶;—OC₂₋₅ alkenyl optionally substituted with one or more, identical or different, substituents R²⁶; —OC₂₋₅ alkynyl optionally substituted with one or more, identical or different, substituents R²⁶; —OC₃₋₆ cycloalkyl optionally substituted with one or more, identical or different, substituents R²⁶; —O-phenyl optionally substituted with one or more, identical or different, substituents R²⁷; and —O-benzyl optionally substituted with one or more, identical or different, substituents R²⁷; and —NH(CH₃);
R²⁴ is H or CH₃;
R²⁵ is H or CH₃;
R²⁶ is independently selected from the group consisting of H, deuterium, F, Cl and —OC₁₋₃ alkyl; and
R²⁷ is independently selected from the group consisting of deuterium, methoxy, nitro, cyano, Cl, Br, I, and F;
or a pharmaceutically acceptable salt, hydrate, polymorph, tautomer, or solvate thereof.

3. A compound of Formula (XI):

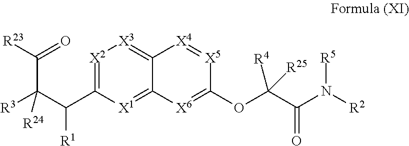

wherein
X¹ is CH or N;
X² is CH or N;
X³ is CH or N;
X⁴ is CH or N;
X⁵ is CH or N;
X⁶ is CH or N;
R²³ is selected from the group consisting of —OH;

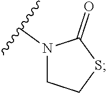 [SMILES: *N1CCSC1=O]

—OC₁₋₅ alkyl optionally substituted with one or more, identical or different, substituents R²⁶, —OC₂₋₅ alkenyl optionally substituted with one or more, identical or different, substituents R²⁶; —OC₂₋₅ alkynyl optionally substituted with one or more, identical or different, substituents R²⁶; —OC₃₋₆ cycloalkyl optionally substituted with one or more, identical or different, substituents R²⁶; —O-phenyl optionally substituted with one or more, identical or different, substituents R²⁷; and —O-benzyl optionally substituted with one or more, identical or different, substituents R²⁷; and —NH(CH₃);
R¹ is selected from the group consisting of Formula (III), Formula (V) and Formula (VI):

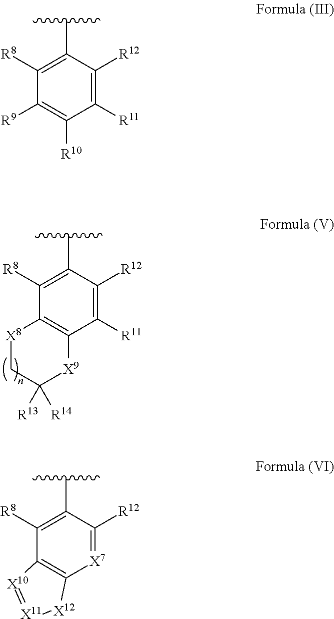

R² is selected from the group consisting of Formula (VIII), Formula (X), C₅₋₈ bicycloalkyl, C₃₋₆ alkyl, and C₉₋₁₂ tricycloalkyl, wherein said bicycloalkyl, C₃₋₆ alkyl, and C₉₋₁₂ tricycloalkyl is optionally substituted with one or more, identical of different substituents R²⁸:

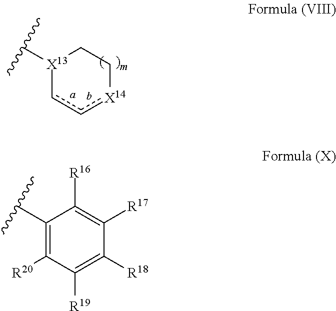

R³ is H or CH₃;
R²⁴ is H or CH₃;
R⁴ is H or CH₃;
R²⁵ is H or CH₃;
R⁵ is H or CH₃;
R⁸ is H or C₁₋₃ alkyl;
R⁹ is selected from the group consisting of H, C₁₋₃ alkyl, halogen, —OH and —OC₁₋₃ alkyl;
R¹⁰ is selected from the group consisting of H, halogen, —OH and —OC₁₋₃ alkyl;
R¹¹ is H;
R¹² is selected from the group consisting of H, C₁₋₃ alkyl and halogen;
R¹³ is selected from the group consisting of H, C₁₋₃ alkyl and halogen;
R¹⁴ is selected from the group consisting of H, C₁₋₃ alkyl and halogen;
R¹⁵ is H or C₁₋₅ alkyl;
R¹⁶ is H or C₁₋₃ alkyl;
R¹⁷ is H or C₁₋₃ alkyl;
R¹⁸ is H or C₁₋₃ alkyl;
R¹⁹ is H or C₁₋₃ alkyl; and
R²⁰ is H or C₁₋₃ alkyl.
R²¹ is selected from the group consisting of H, deuterium, halogen and C₁₋₃ alkyl, wherein said C₁₋₃ alkyl is optionally substituted with one or more halogens;
R²² is selected from the group consisting of H, deuterium, halogen and C₁₋₃ alkyl, wherein said C₁₋₃ alkyl is optionally substituted with one or more halogens;
R²⁶ is independently selected from the group consisting of H, deuterium, F, Cl and —OC₁₋₃ alkyl;
R²⁷ is independently selected from the group consisting of deuterium, methoxy, nitro, cyano, Cl, Br, I, and F; and
R²⁸ is individually selected from C₁₋₃ alkyl, —OC₁₋₃ alkyl, halogen, and deuterium, wherein said C₁₋₃ alkyl is optionally substituted with one or more halogens;
R²⁹ is H or C₁₋₃ alkyl;
X⁷ is C(R¹¹) or N;
X⁸ is O or CH₂;
X⁹ is O;
X¹⁰ is N or CH;
X¹¹ is N or CH;
X¹² is O or N(R¹⁵);
X¹³ is C(R²⁹) or N;
X¹⁴ is (1) C(H) and b is a double bond; (2) C(R²¹)(R²²) and b is a single bond; or (3) O and a is a single bond;
a is a double bond or a single bond;
b is a double bond or a single bond;
n is 0 or 1;
m is 0, 1 or 2;
with the proviso that no more than one of a and b is a double bond, or a pharmaceutically acceptable salt, hydrate, polymorph, tautomer, or solvate thereof.

4. The compound according to item 1, wherein the compound is of Formula (XIII):

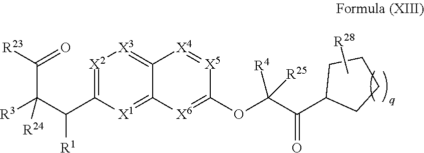

wherein q is 0, 1, 2 or 3.

5. The compound according to any one of items 1 to 4, wherein R²³ is —OH.

6. The compound according to any one of items 1 to 4, wherein R²³ is —OC₁₋₅ alkyl.

7. The compound according to any one of items 1 to 4, wherein R²³ is —OCH₃.

8. The compound according to any one of items 1 to 4, wherein R²³ is —OCH₂CH₃.

9. The compound according to any one of items 1 to 4, wherein R²³ is

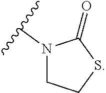 [SMILES: *N1CCSC1=O]

10. The compound according to any one of items 1 or 2, wherein the compound is of Formula (XII):

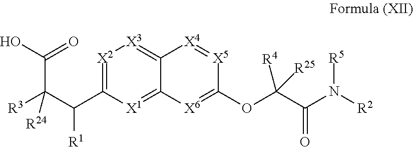

wherein
X¹ is CH or N;
X² is CH or N;
X³ is CH or N;
X⁴ is CH or N;
X⁵ is CH or N;
X⁶ is CH or N;
R¹ is phenyl, pyridinyl or H, wherein the phenyl or pyridinyl is optionally substituted with one or more, identical or different, substituents R⁶, wherein two of the substituents R⁶ optionally are linked together to form a ring;
R² is selected from the group consisting of C₅₋₇ cycloalkyl, C₅₋₇ cycloalkenyl C₅₋₇ heterocycloalkyl and phenyl,
   wherein said C₅₋₇ cycloalkyl, C₅₋₇ cycloalkenyl C₅₋₇ heterocycloalkyl and phenyl is optionally substituted with one or more substituents individually selected from C₁₋₃ alkyl, —OC₁₋₃ alkyl, halogen and deuterium,
      wherein said C₁₋₃ alkyl is optionally substituted with one or more halogens;
R³ is H or CH₃;
R⁴ is H or CH₃;
R⁵ is H or CH₃;
R⁶ is selected from the group consisting of C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group of the C₁₋₅ alkyl, —OC₁₋₅ alkyl, or C₂₋₅ alkenyl is optionally replaced with O or N(R⁷); and/or
   wherein one or more methine group of the C₂₋₅ alkenyl is optionally replaced with N;
R⁷ is H or C₁₋₃ alkyl;
R²⁴ is H or CH₃; and
R²⁵ is H or CH₃;
or a pharmaceutically acceptable salt, hydrate, polymorph, tautomer, or solvate thereof.

11. The compound according to any one of the preceding items, wherein R²⁴ is H.

12. The compound according to any one of the preceding items, wherein R²⁵ is H.

13. The compound according to any one of the preceding items, wherein R²⁴ and R²⁵ are H.

14. The compound according to any one of items 1 to 10 or 12, wherein R²⁴ is —CH₃.

15. The compound according to any one of items 1 to 11 or 14, wherein R²⁵ is —CH₃.

16. The compound according to any one of items 1 to 10, wherein R²⁴ and R²⁵ are —CH₃.

17. The compound according to any one of the preceding items, wherein R⁴ is —CH₃.

18. The compound according to any one of items 1 to 10, wherein R⁴ is —CH₃, and R³, R²⁴, R²⁵, and R⁵ are H.

19. The compound according to any one of items 1 to 17, wherein R⁵ is —CH₃.

20. The compound according to any one of items 1 to 10, wherein R⁵ is —CH₃, and R³, R²⁴, R²⁵, and R⁴ are H.

21. The compound according to any one of items 1 to 17 or 19, wherein R³ is —CH₃.

22. The compound according to any one of items 1 to 10, wherein R³ is —CH₃, and R⁴, R²⁴, R²⁵, and R⁵ are H.

23. The compound according to any one of items 1 to 17 or 19, wherein R³ and R²⁴ are —CH₃.

24. The compound according to any one of items 1 to 10, wherein R³ and R²⁴ are —CH₃, and R⁴, R²⁵, and R⁵ are H.

25. The compound according to any one of items 1 to 10, wherein R³, R²⁴, R⁴, R²⁵, and R⁵ are H.

26. The compound according to any one of items 1 to 10, wherein R²³ is —OH, R²⁴ is H, and R²⁵ is H.

27. The compound according to any one of items 1 to 5, wherein the compound is of Formula (I):

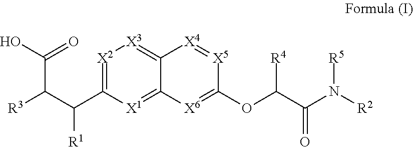

wherein
X¹ is CH or N;
X² is CH or N;
X³ is CH or N;
X⁴ is CH or N;
X⁵ is CH or N;
X⁶ is CH or N;
R¹ is phenyl, pyridinyl or H, wherein the phenyl or pyridinyl is optionally substituted with one or more, identical or different, substituents R⁶, wherein two of the substituents R⁶ optionally are linked together to form a ring;
R² is selected from the group consisting of C₅₋₇ cycloalkyl, C₅₋₇ cycloalkenyl C₅₋₇ heterocycloalkyl and phenyl,
   wherein said C₅₋₇ cycloalkyl, C₅₋₇ cycloalkenyl C₅₋₇ heterocycloalkyl and phenyl is optionally substituted with one or more substituents individually selected from C₁₋₃ alkyl, —OC₁₋₃ alkyl, halogen and deuterium,
      wherein said C₁₋₃ alkyl is optionally substituted with one or more halogens;
R³ is H or CH₃;
R⁴ is H or CH₃;
R⁵ is H or CH₃;
R⁶ is selected from the group consisting of C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group of the C₁₋₅ alkyl, —OC₁₋₅ alkyl, or C₂₋₅ alkenyl is optionally replaced with O or N(R⁷); and/or
   wherein one or more methine group of the C₂₋₅ alkenyl is optionally replaced with N; and
R⁷ is H or C₁₋₃ alkyl;
or a pharmaceutically acceptable salt, hydrate, polymorph, tautomer, or solvate thereof.

28. The compound according to any one of items 1 to 3, wherein the compound is of Formula (IA):

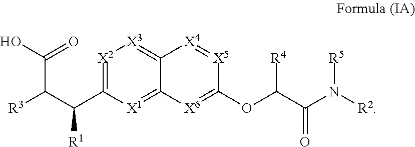

29. The compound according to any one of items 1 to 3, wherein the compound is of Formula (IB):

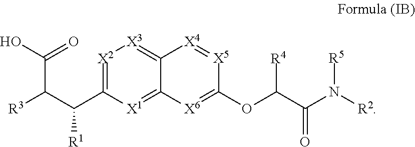

30. The compound according to any one of the preceding items, wherein X¹ is CH.

31. The compound according to any one of items 1 to 29, wherein X¹ is N.

32. The compound according to any one of the preceding items, wherein X² is CH.

33. The compound according to any one of items 1 to 31, wherein X² is N.

34. The compound according to any one of the preceding items, wherein X³ is CH.

35. The compound according to any one of items 1 to 33, wherein X³ is N.

36. The compound according to any one of the preceding items, wherein X⁴ is CH.

37. The compound according to any one of items 1 to 35, wherein X⁴ is N.

38. The compound according to any one of the preceding items, wherein X⁵ is CH.

39. The compound according to any one of items 1 to 37, wherein X⁵ is N.

40. The compound according to any one of the preceding items, wherein X⁶ is CH.

41. The compound according to any one of items 1 to 39, wherein X⁶ is N.

42. The compound according to any one of items 1 to 29, wherein X¹, X², X³, X⁴, X⁵ and X⁶ are CH.

43. The compound according to any one of items 1 to 41, wherein no more than 2, such as no more than 1, of X¹, X², X³, X⁴, X⁵ and X⁶ are N.

44. The compound according to any one of items 1 to 29, wherein X¹, X², X³, X⁴ and X⁵ are CH, and X⁶ is N.

45. The compound according to any one of items 1 to 29, wherein X¹, X², X³, X⁵ and X⁶ are CH, and X⁴ is N.

46. The compound according to any one of items 1 to 29, wherein X¹, X², X⁴, X⁵ and X⁶ are CH, and X₃ is N.

47. The compound according to any one of the preceding items, wherein R³ is H.

48. The compound according to any one of the preceding items, wherein R⁴ is H.

49. The compound according to any one of the preceding items, wherein R⁵ is H.

50. The compound according to any one of the preceding items, wherein R³, R⁴ and R⁵ are H.

51. The compound according to any one of items 1 to 16, 27 or 28, wherein X¹, X², X³, X⁴, X⁵ and X⁶ are CH, and R³, R⁴ and R⁵ are H.

52. The compound according to any one of the preceding items, wherein R¹ is of Formula (II):

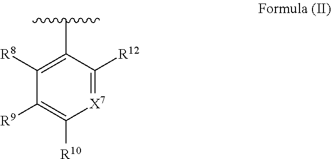

wherein
X⁷ is C(R¹¹) or N;
R⁷ is H or C₁₋₃ alkyl;
R⁸ is H or C₁₋₅ alkyl;
R⁹ is selected from the group consisting of H, C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group of the C₁₋₅ alkyl, —OC₁₋₅ alkyl, or C₂₋₅ alkenyl is optionally replaced with O or N(R⁷); and/or
   wherein one or more methine group of the C₂₋₅ alkenyl is optionally replaced with N;
R¹⁰ is selected from the group consisting of H, C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group of the C₁₋₅ alkyl, —OC₁₋₅ alkyl, or C₂₋₅ alkenyl is optionally replaced with O or N(R⁷); and/or
   wherein one or more methine group of the C₂₋₅ alkenyl is optionally replaced with N;
R¹¹ is H or —OC₁₋₅ alkyl;
R¹² is H, C₁₋₅ alkyl or —OC₁₋₅ alkyl; and
R⁹ and R¹⁰ are optionally linked together to form a ring.

53. The compound according to any one of the preceding items, wherein R¹ is of Formula (II):

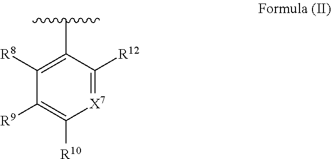

wherein
X⁷ is C(R¹¹) or N;
R⁸ is H or C₁₋₅ alkyl;
R⁹ is selected from the group consisting of H, C₁₋₅ alkyl, halogen, —OH and —OC₁₋₅ alkyl;
R¹⁰ is selected from the group consisting of H, C₁₋₅ alkyl, halogen, —OH and —OC₁₋₅ alkyl;
R¹¹ is H; and
R¹² is H or C₁₋₅ alkyl.

54. The compound according to any one of the preceding items, wherein R¹ is of Formula (III):

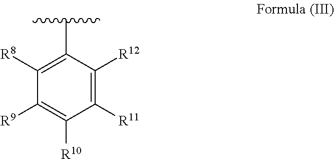

wherein
R⁸ is H or C₁₋₃ alkyl;
R⁹ is selected from the group consisting of H, C₁₋₃ alkyl, halogen, —OH and —OC₁₋₃ alkyl;
R¹⁰ is selected from the group consisting of H, halogen, —OH and —OC₁₋₃ alkyl;
R¹¹ is H; and
R¹² is H or C₁₋₃ alkyl.

55. The compound according to any one of the preceding items, wherein
R⁸ is H or Me;
R⁹ is selected from the group consisting of H, Me, Cl, —OH and —OMe;
R¹⁰ is selected from the group consisting of H, Cl, —OH, OMe, OEt and OPr;
R¹¹ is H; and
R¹² is H or Me.

56. The compound according to any one of items 1 to 53, wherein R¹ is phenyl.

57. The compound according to any one of items 1 to 53, wherein R¹ is

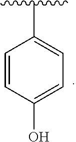 [SMILES: *c1ccc(O)cc1]

58. The compound according to any one of items 1 to 53, wherein R¹ is

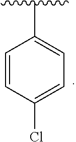 [SMILES: *c1ccc(Cl)cc1]

59. The compound according to any one of items 1 to 53, wherein R¹ is

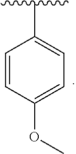 [SMILES: *c1ccc(OC)cc1]

60. The compound according to any one of items 1 to 53, wherein R¹ is

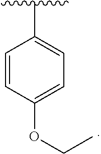 [SMILES: *c1ccc(OCC)cc1]

61. The compound according to any one of items 1 to 53, wherein R¹ is

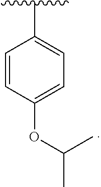 [SMILES: *c1ccc(OC(C)C)cc1]

62. The compound according to any one of items 1 to 53, wherein R¹ is

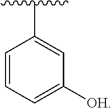 [SMILES: *c1cccc(O)c1]

63. The compound according to any one of items 1 to 53, wherein R¹ is

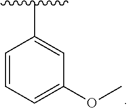 [SMILES: *c1cccc(OC)c1]

64. The compound according to any one of items 1 to 53, wherein R¹ is

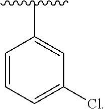 [SMILES: *c1cccc(Cl)c1]

65. The compound according to any one of items 1 to 53, wherein R¹ is

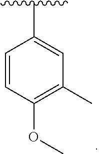 [SMILES: *c1ccc(OC)c(C)c1]

66. The compound according to any one of items 1 to 53, wherein R¹ is

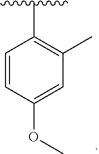 [SMILES: *c1ccc(OC)cc1C]

67. The compound according to any one of items 1 to 53, wherein R¹ is

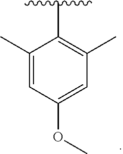

68. The compound according to any one of items 1 to 53, wherein R¹ is

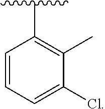 [SMILES: *c1cccc(Cl)c1C]

69. The compound according to any one of items 1 to 53, wherein R¹ is

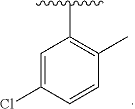 [SMILES: *c1cc(Cl)ccc1C]

70. The compound according to any one of items 1 to 53, wherein R¹ is

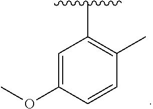

71. The compound according to any one of items 1 to 52, wherein R¹ is of Formula (IV):

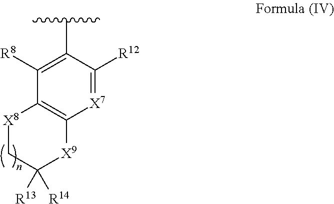

wherein
X⁷ is C(R¹¹) or N;
X⁸ is O or CH₂;
X⁹ is O;
R⁸ is H or C₁₋₅ alkyl;
R¹³ is selected from the group consisting of H, C₁₋₃ alkyl and halogen;
R¹⁴ is selected from the group consisting of H, C₁₋₃ alkyl and halogen;
R¹¹ is H;
R¹² is selected from the group consisting of H, C₁₋₅ alkyl and halogen; and
n is 0 or 1.

72. The compound according to any one of items 1 to 52, wherein R¹ is of Formula (IV):

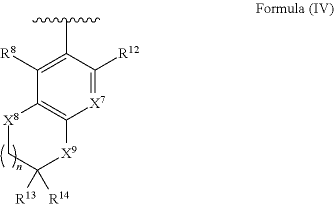

wherein
X⁷ is C(R¹¹) or N;
X⁸ is O or CH₂;
X⁹ is O;
R⁸ is H or C₁₋₅ alkyl;
R¹³ is selected from the group consisting of H, C₁₋₃ alkyl and halogen;
R¹⁴ is selected from the group consisting of H, C₁₋₃ alkyl and halogen;
R¹¹ is H;
R¹² is H or C₁₋₅ alkyl; and
n is 0 or 1.

73. The compound according to item 71, wherein R¹ is of Formula (V):

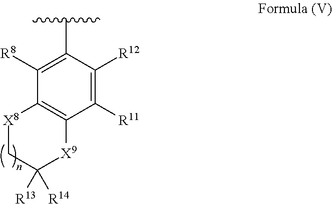

X⁸ is O or CH₂;
X⁹ is O;
R⁸ is H or C₁₋₃ alkyl;
R¹³ is selected from the group consisting of H, C₁₋₃ alkyl and halogen;
R¹⁴ is selected from the group consisting of H, C₁₋₃ alkyl and halogen;
R¹¹ is H;
R¹² is selected from the group consisting of H, C₁₋₃ alkyl and halogen; and
n is 0 or 1.

74. The compound according to any one of items 71 to 73, wherein R¹ is of Formula (V):

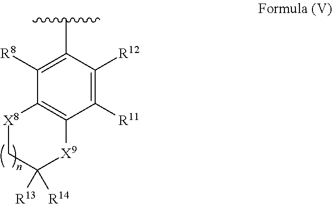

X⁸ is O or CH₂;
X⁹ is O;
R⁸ is H or C₁₋₃ alkyl;
R¹³ is selected from the group consisting of H, C₁₋₃ alkyl and halogen;
R¹⁴ is selected from the group consisting of H, C₁₋₃ alkyl and halogen;
R¹¹ is H;
R¹² is H or C₁₋₃ alkyl; and
n is 0 or 1.

75. The compound according to any one of items 71 to 74, wherein
X⁸ is O or CH₂;
X⁹ is O;
R⁸ is H or Me;
R¹³ is selected from the group consisting of H, Me and F;
R¹⁴ is selected from the group consisting of H, Me and F;
R¹¹ is H;
R¹² is H or Me; and
n is 0 or 1.

76. The compound according to any one of items 1 to 52 or 71 to 75, wherein R¹ is

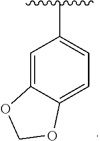 [SMILES: *c1ccc2c(c1)OCO2]

77. The compound according to any one of items 1 to 52 or 71 to 75, wherein R¹ is

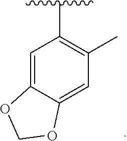 [SMILES: *c1cc2c(cc1C)OCO2]

78. The compound according to any one of items 1 to 5250 or 71 to 75, wherein R¹ is

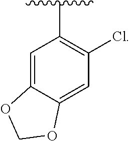 [SMILES: *c1cc2c(cc1Cl)OCO2]

79. The compound according to any one of items 1 to 52 or 71 to 75, wherein R¹ is

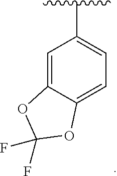 [SMILES: *c1ccc2c(c1)OC(F)(F)O2]

80. The compound according to any one of items 1 to 52 or 71 to 75, wherein R¹ is

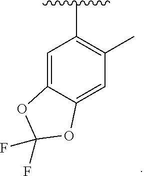 [SMILES: *c1cc2c(cc1C)OC(F)(F)O2]

81. The compound according to any one of items 1 to 52 or 71 to 75, wherein R¹ is

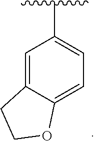 [SMILES: *c1ccc2c(c1)CCO2]

82. The compound according to any one of items 1 to 52 or 71 to 75, wherein R¹ is

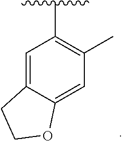 [SMILES: *c1cc2c(cc1C)OCC2]

83. The compound according to any one of items 1 to 52 or 71 to 75, wherein R¹ is

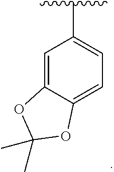 [SMILES: *c1ccc2c(c1)OC(C)(C)O2]

84. The compound according to any one of items 1 to 52 or 71 to 75, wherein R¹ is

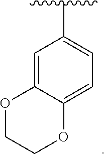 [SMILES: *c1ccc2c(c1)OCCO2 ; "."]

85. The compound according to any one of items 1 to 52 or 71 to 75, wherein R¹ is

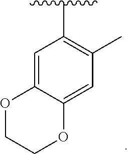 [SMILES: *c1cc2c(cc1C)OCCO2]

86. The compound according to any one of items 1 to 52, wherein R¹ is of Formula (VI):

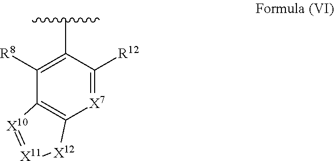

wherein
X⁷ is C(R¹¹) or N;
X¹⁰ is N or CH;
X¹¹ is N or CH;
X¹² is O or N(R¹⁵);
R⁸ is H or C₁₋₅ alkyl;
R¹¹ is H or C₁₋₅ alkyl;
R¹² is H or C₁₋₅ alkyl; and
R¹⁵ is H or C₁₋₅ alkyl.

87. The compound according to any one of items 1 to 52 or 86, wherein R¹ is of Formula (VII):

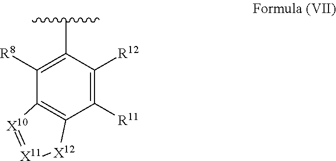

wherein
X¹⁰ is N or CH;
X¹¹ is N or CH;
X¹² is O or N(R¹⁵);
R⁸ is H or C₁₋₃ alkyl;
R¹¹ is H or C₁₋₃ alkyl;
R¹² is H or C₁₋₃ alkyl; and
R¹⁵ is H or C₁₋₃ alkyl.

88. The compound according to any one of items 86 or 87, wherein
X¹⁰ is N or CH;
X¹¹ is N or CH;
X¹² is O or N(R¹⁵);
R⁸ is H;
R¹¹ is H or Me;
R¹² is H or Me; and
R¹⁵ is H, Me or Et.

89. The compound according to any one of items 1 to 52 or 86 to 88, wherein R¹ is

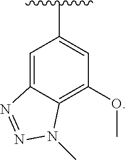 [SMILES: *c1cc(OC)c2c(c1)nnn2C]

90. The compound according to any one of items 1 to 52 or 86 to 88, wherein R¹ is

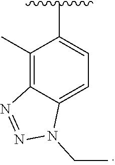 [SMILES: *c1ccc2c(nnn2CC)c1C]

91. The compound according to any one of items 1 to 52 or 86 to 88, wherein R¹ is

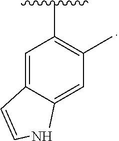 [SMILES: *c1cc2cc[nH]c2cc1C]

92. The compound according to any one of items 1 to 52 or 86 to 88, wherein R¹ is

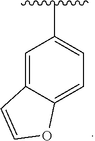 [SMILES: *c1ccc2occc2c1]

93. The compound according to any one of items 1 to 52 or 86 to 88, wherein R¹ is

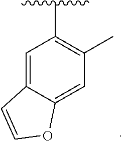 [SMILES: *c1cc2ccoc2cc1C]

94. The compound according to any one of items 1 to 51, wherein R¹ is phenyl substituted with one or more, identical or different, substituents R⁶, wherein two of the substituents R⁶ optionally are linked together to form a ring;
R⁶ is selected from the group consisting of C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group of the C₁₋₅ alkyl, —OC₁₋₅ alkyl, or C₂₋₅ alkenyl is optionally replaced with O or N(R⁷); and/or
   wherein one or more methine group of the C₂₋₅ alkenyl is optionally replaced with N; and
R⁷ is H or C₁₋₃ alkyl.

95. The compound according to any one of items 1 to 51, wherein R¹ is pyridinyl substituted with one or more, identical or different, substituents R⁶, wherein two of the substituents R⁶ optionally are linked together to form a ring;
R⁶ is selected from the group consisting of C₁₋₅ alkyl, halogen, —OH, —OC₁₋₅ alkyl and C₂₋₅ alkenyl,
   wherein said C₁₋₅ alkyl, —OC₁₋₅ alkyl and C₂₋₅ alkenyl are optionally substituted with one or more substituents individually selected from C₁₋₃ alkyl and halogen;
   wherein one or more methylene group of the C₁₋₅ alkyl, —OC₁₋₅ alkyl, or C₂₋₅ alkenyl is optionally replaced with O or N(R⁷); and/or
   wherein one or more methine group of the C₂₋₅ alkenyl is optionally replaced with N; and
R⁷ is H or C₁₋₃ alkyl.

96. The compound according to any one of items 1 to 51, wherein R¹ is pyridinyl.

97. The compound according to any one of items 1 to 51, wherein R¹ is H.

98. The compound according to any one of items 52 to 95, wherein X⁷ is C(R¹¹).

99. The compound according to any one of items 52 to 95, wherein X⁷ is N.

100. The compound according to any one of items 71 to 99, wherein X⁸ is O.

101. The compound according to any one of items 71 to 99, wherein X⁸ is CH₂.

102. The compound according to any one of items 71 to 101, wherein X⁹ is O.

103. The compound according to any one of items 86 to 99, wherein X¹⁰ is N.

104. The compound according to any one of items 86 to 99, wherein X¹⁰ is CH.

105. The compound according to any one of items 86 to 104, wherein X¹¹ is N.

106. The compound according to any one of items 86 to 88, 98, 99, 103 or 104, wherein X¹¹ is CH.

107. The compound according to any one of items 86 to 106, wherein X¹² is O

108. The compound according to any one of items 86 to 106, wherein X¹² is N(R¹⁵).

109. The compound according to any one of items 86 to 106, wherein X¹² is N(H).

110. The compound according to any one of items 86 to 106, wherein X¹² is N(CH₃).

111. The compound according to any one of items 86 to 106, wherein X¹² is N(CH₂CH₃).

112. The compound according to any one of items 52 to 111, wherein R⁸ is H.

113. The compound according to any one of 52 to 111, wherein R⁸ is C₁₋₅ alkyl, such as C₁₋₃ alkyl.

114. The compound according to any one of items 52 to 111, wherein R⁸ is Me.

115. The compound according to any one of items 52 to 114, wherein R⁹ is H.

116. The compound according to any one of items 52 to 114, wherein R⁹ is C₁₋₅ alkyl, such as C₁₋₃ alkyl.

117. The compound according to any one of items 52 to 114, wherein R⁹ is Me.

118. The compound according to any one of items 52 to 114, wherein R⁹ is C₁₋₅ alkyl substituted with one or more halogens.

119. The compound according to any one of items 52 to 114, wherein R⁹ is halogen.

120. The compound according to any one of items 52 to 114, wherein R⁹ is Cl.

121. The compound according to any one of items 52 to 114, wherein R⁹ is —OH.

122. The compound according to any one of items 52 to 114, wherein R⁹ is —OC₁₋₅ alkyl, such as is —OC₁₋₃ alkyl.

123. The compound according to any one of items 52 to 114, wherein R⁹ is —OMe.

124. The compound according to any one of items 52 to 123, wherein R¹⁰ is H. 125. The compound according to any one of items 52 to 123, wherein R¹⁰ is C₁₋₅ alkyl, such as C₁₋₃ alkyl.

126. The compound according to any one of items 52 to 123, wherein R¹⁰ is Me.

127. The compound according to any one of items 52 to 123, wherein R¹⁰ is C₁₋₅ alkyl substituted with one or more halogens.

128. The compound according to any one of items 52 to 123, wherein R¹⁰ is halogen.

129. The compound according to any one of items 52 to 123, wherein R¹⁰ is Cl.

130. The compound according to any one of items 52 to 123, wherein R¹⁰ is —OH.

131. The compound according to any one of items 52 to 123, wherein R¹⁰ is —OC₁₋₅ alkyl, such as is —OC₁₋₃ alkyl.

132. The compound according to any one of items 52 to 123, wherein R¹⁰ is —OMe.

133. The compound according to any one of items 52 to 132, wherein R¹¹ is H.

134. The compound according to any one of items 52 to 132, wherein R¹¹ is —OC₁₋₅ alkyl, such as-OC₁₋₃ alkyl.

135. The compound according to any one of items 52 to 132, wherein R¹¹ is —OMe.

136. The compound according to any one of items 52 to 135, wherein R¹² is H.

137. The compound according to any one of items 52 to 135, wherein R¹² is C₁₋₅ alkyl, such as C₁₋₃ alkyl.

138. The compound according to any one of items 52 to 135, wherein R¹² is Me.

139. The compound according to any one of items 52 to 135, wherein R¹² is halogen.

140. The compound according to any one of items 52 to 135, wherein R¹² is Cl.

141. The compound according to any one of items 71 to 140, wherein R¹³ is H.

142. The compound according to any one of items 71 to 140, wherein R¹³ is C₁₋₃ alkyl.

143. The compound according to any one of items 71 to 140, wherein R¹³ is Me.

144. The compound according to any one of items 71 to 140, wherein R¹³ is halogen.

145. The compound according to any one of items 71 to 140, wherein R¹³ is F.

146. The compound according to any one of items 71 to 145, wherein R¹⁴ is H.

147. The compound according to any one of items 71 to 145, wherein R¹⁴ is C₁₋₃ alkyl.

148. The compound according to any one of items 71 to 145, wherein R¹⁴ is Me.

149. The compound according to any one of items 71 to 145, wherein R¹⁴ is halogen.

150. The compound according to any one of items 71 to 145, wherein R¹⁴ is F.

151. The compound according to any one of items 71 to 145, wherein R¹³ and R¹⁴ are identical.

152. The compound according to any one of items 86 to 151, wherein R¹⁵ is H.

153. The compound according to any one of items 86 to 151, wherein R¹⁵ is C₁₋₅ alkyl, such as C₁₋₃ alkyl.

154. The compound according to any one of items 86 to 151, wherein R¹⁵ is Me.

155. The compound according to any one of items 86 to 151, wherein R¹⁵ is Et.

156. The compound according to any one of items 71 to 155, wherein n is 0.

157. The compound according to any one of items 71 to 155, wherein n is 1.

158. The compound according to any one of the preceding items, wherein R² is of Formula (VIII):

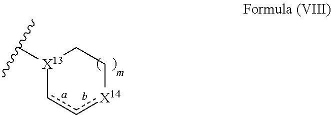

wherein
X¹³ is C(R²⁹) or N;
X¹⁴ is (1) C(H) and b is a double bond; (2) C(R²¹)(R²²) and b is a single bond; or (3) O and a is a single bond;
R²¹ is selected from the group consisting of H, deuterium, halogen and C₁₋₃ alkyl, wherein said C₁₋₃ alkyl is optionally substituted with one or more halogens;
R²² is selected from the group consisting of H, deuterium, halogen and C₁₋₃ alkyl, wherein said C₁₋₃ alkyl is optionally substituted with one or more halogens;
a is a double bond or a single bond;
b is a double bond or a single bond;
R²⁹ is H or C₁₋₃ alkyl; and
m is 0, 1 or 2,
with the proviso that no more than one of a and b is a double bond.

159. The compound according to any one of the preceding items, wherein R² is of Formula (VIII):

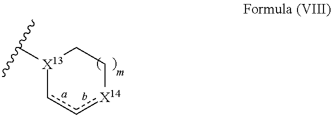

wherein
X¹³ is C(H) or N;
X¹⁴ is (1) C(H) and b is a double bond; (2) C(R²¹)(R²²) and b is a single bond; or (3) O and a is a single bond;
R²¹ is selected from the group consisting of H, deuterium, halogen and C₁₋₃ alkyl, wherein said C₁₋₃ alkyl is optionally substituted with one or more halogens;
R²² is selected from the group consisting of H, deuterium, halogen and C₁₋₃ alkyl, wherein said C₁₋₃ alkyl is optionally substituted with one or more halogens; a is a double bond or a single bond;
b is a double bond or a single bond; and
m is 1 or 2,
with the proviso that no more than one of a and b is a double bond.

160. The compound according to any one of the preceding items, wherein R² is of Formula (IX):

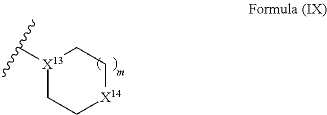

wherein
X¹³ is C(R²⁹) or N;
X¹⁴ is C(R²¹)(R²²) or O;
R²¹ is selected from the group consisting of H, deuterium, halogen and C₁₋₃ alkyl, wherein said C₁₋₃ alkyl is optionally substituted with one or more halogens;
R²² is selected from the group consisting of H, deuterium, halogen and C₁₋₃ alkyl, wherein said C₁₋₃ alkyl is optionally substituted with one or more halogens;
R²⁹ is H or C₁₋₃ alkyl; and
m is 0, 1 or 2.

161. The compound according to any one of the preceding items, wherein R² is of Formula (IX):

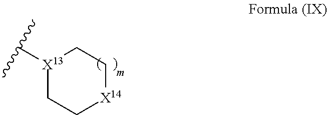

wherein
X¹³ is C(H) or N;
X¹⁴ is C(R²¹)(R²²) or O;
R²¹ is selected from the group consisting of H, deuterium, halogen and C₁₋₃ alkyl, wherein said C₁₋₃ alkyl is optionally substituted with one or more halogens;
R²² is selected from the group consisting of H, deuterium, halogen and C₁₋₃ alkyl, wherein said C₁₋₃ alkyl is optionally substituted with one or more halogens; and
m is 1 or 2.

162. The compound according to any one of items 158 to 161, wherein m is 1.

163. The compound according to any one of items 158 to 161, wherein m is 2.

164. The compound according to any one of items 158 to 163, wherein X¹³ is C(R²⁹).

165. The compound according to any one of items 158 to 164, wherein R²⁹ is H.

166. The compound according to any one of items 158 to 164, wherein R²⁹ is C₁₋₃ alkyl.

167. The compound according to any one of items 158 to 164, wherein R²⁹ is —CH₃.

168. The compound according to any one of items 158 to 164, wherein X¹³ is C(H).

169. The compound according to any one of items 158 to 164, wherein X¹³ is C(CH₃).

170. The compound according to any one of items 158 to 164, wherein X¹³ is N.

171. The compound according to any one of items 158 to 170, wherein X¹⁴ is C(R²¹)(R²²).

172. The compound according to any one of items 158 to 171, wherein R²¹ is H.

173. The compound according to any one of items 158 to 171, wherein R²¹ is deuterium (D).

174. The compound according to any one of items 158 to 171, wherein R²¹ is halogen, such as F.

175. The compound according to any one of items 158 to 171, wherein R²¹ is C₁₋₃ alkyl.

176. The compound according to any one of items 158 to 171, wherein R²¹ is —CH₃.

177. The compound according to any one of items 158 to 171, wherein R²¹ is C₁₋₃ alkyl, such as Me, substituted with one or more halogens, such as F.

178. The compound according to any one of items 158 to 171, wherein R²¹ is —CF₃.

179. The compound according to any one of items 158 to 178, wherein R²² is H.

180. The compound according to any one of items 158 to 178, wherein R²² is halogen, such as F.

181. The compound according to any one of items 158 to 178, wherein R²² is C₁₋₃ alkyl.

182. The compound according to any one of items 158 to 178, wherein R²² is —CH₃.

183. The compound according to any one of items 158 to 182, wherein X¹⁴ is C(H)₂.

184. The compound according to any one of items 158 to 182, wherein X¹⁴ is C(H) (D).

185. The compound according to any one of items 158 to 182, wherein X¹⁴ is C(F)₂.

186. The compound according to any one of items 158 to 182, wherein X¹⁴ is C(CH₃)₂.

187. The compound according to any one of items 158 to 182, wherein X¹⁴ is C(H)(CF₃).

188. The compound according to any one of items 158 to 182, wherein X¹⁴ is O.

189. The compound according to any one of items 160 or 161, wherein R² is of Formula (IX) and X¹³ is C(H), X¹⁴ is C(H)₂ and m is 0.

190. The compound according to any one of items 1 to 161, wherein R² is cyclopentyl.

191. The compound according to any one of items 160 or 161, wherein R² is of Formula (IX) and X¹³ is C(H), X¹⁴ is C(H)₂ and m is 1.

192. The compound according to any one of items 1 to 161, wherein R² is cyclohexyl.

193. The compound according to any one of items 160 or 161, wherein R² is of Formula (IX) and X¹³ is C(H), X¹⁴ is C(H)₂ and m is 2.

194. The compound according to any one of items 1 to 161, wherein R² is cycloheptyl.

195. The compound according to any one of items 160 or 161, wherein R² is of Formula (IX) and X¹³ is C(H), X¹⁴ is O and m is 1.

196. The compound according to any one of items 1 to 157, wherein R² is tetrahydro-2H-pyran-4-yl.

197. The compound according to any one of items 160 or 161, wherein R² is of Formula (IX) and X¹³ is N, X¹⁴ is O and m is 1.

198. The compound according to any one of items 1 to 157, wherein R² is N-morpholinyl.

199. The compound according to any one of items 160 or 161, wherein R² is of Formula (IX) and X¹³ is C(H), X¹⁴ is C(H) (D) and m is 1.

200. The compound according to any one of items 160 or 161 wherein R² is of Formula (IX) and X¹³ is C(H), X¹⁴ is C(F)₂ and m is 1.

201. The compound according to any one of items 160 or 161, wherein R² is of Formula (IX) and X¹³ is C(H), X¹⁴ is C(CH₃)₂ and m is 1.

202. The compound according to any one of items 160 or 161, wherein R² is of Formula (IX) and X¹³ is C(CH₃), X¹⁴ is C(H)₂ and m is 1.

203. The compound according to any one of items 160 or 161, wherein R² is of Formula (IX) and X¹³ is C(H), X¹⁴ is C(H)(CF₃) and m is 1.

204. The compound according to any one of items 1 to 157, wherein R² is

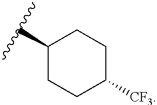 [SMILES: *[C@H]1CC[C@H](C(F)(F)F)CC1]

205. The compound according to any one of items 1 to 157, wherein R² is

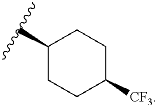 [SMILES: *[C@H]1CC[C@@H](C(F)(F)F)CC1]

206. The compound according to any one of items 158 or 159, wherein R² is of Formula (VIII) and X¹³ is C(H), X¹⁴ is C(H)₂, m is 1, a is a double bond and b is a single bond.

207. The compound according to any one of items 1 to 157, wherein R² is cyclohex-2-enyl.

208. The compound according to any one of items 158 or 159, wherein R² is of Formula (VIII) and X¹³ is C(H), X¹⁴ is C(H)₂, m is 1, a is a single bond and b is a double bond.

209. The compound according to any one of items 1 to 157, wherein R² is cyclohex-3-enyl.

210. The compound according to any one of items 1 to 157, wherein R² is of Formula (X):

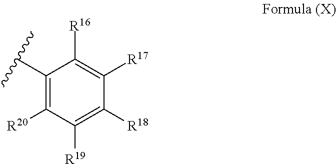

wherein
R¹⁶ is H or C₁₋₃ alkyl;
R¹⁷ is H or C₁₋₃ alkyl;
R¹⁸ is H or C₁₋₃ alkyl;
R¹⁹ is H or C₁₋₃ alkyl; and
R²⁰ is H or C₁₋₃ alkyl.

211. The compound according to item 210, wherein R¹⁶ is H.

212. The compound according to item 210, wherein R¹⁶ is C₁₋₃ alkyl.

213. The compound according to item 210, wherein R¹⁶ is Me.

214. The compound according to any one of items 210 to 213, wherein R¹⁷ is H.

215. The compound according to any one of items 210 to 213, wherein R¹⁷ is C₁₋₃ alkyl.

216. The compound according to any one of items 210 to 213, wherein R¹⁷ is Me.

217. The compound according to any one of items 210 to 216, wherein R¹⁸ is H.

218. The compound according to any one of items 210 to 216, wherein R¹⁸ is C₁₋₃ alkyl.

219. The compound according to any one of items 210 to 216, wherein R¹⁸ is Me.

220. The compound according to any one of items 210 to 219, wherein R¹⁹ is H.

221. The compound according to any one of items 210 to 219, wherein R¹⁹ is C₁₋₃ alkyl.

222. The compound according to any one of items 210 to 219, wherein R¹⁹ is Me.

223. The compound according to any one of items 210 to 222, wherein R²⁰ is H.

224. The compound according to any one of items 210 to 222, wherein R²⁰ is C₁₋₃ alkyl.

225. The compound according to any one of items 210 to 222, wherein R²⁰ is Me.

226. The compound according to item 210, wherein R² is of Formula (X), R¹⁶ is Me, R¹⁷ is H, R¹⁸ is Me, R¹⁹ is H and R²⁰ is Me.

227. The compound according to item 210, wherein R² is of Formula (X), R¹⁶ is Me, R¹⁷ is Me, R¹⁸ is H, R¹⁹ is Me and R²⁰ is Me.

228. The compound according to item 210, wherein R² is of Formula (X), R¹⁶ is H, R¹⁷ is H, R¹⁸ is H, R¹⁹ is H and R²⁰ is H.

229. The compound according to any one of items 1 to 157, wherein R² is phenyl.

230. The compound according to any one of items 1 to 157, wherein R² is C₅₋₈ bicycloalkyl.

231. The compound according to any one of items 1 to 157, wherein R² is bicyclo[2.2.1]heptan-2-yl.

232. The compound according to any one of items 1 to 157, wherein R² is

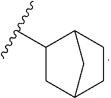 [SMILES: *C1CC2CCC1C2]

233. The compound according to any one of items 1 to 157, wherein R² is

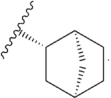 [SMILES: *[C@H]1C[C@@H]2CC[C@H]1C2]

234. The compound according to any one of items 1 to 157, wherein R² is C₉₋₁₂ tricycloalkyl.

235. The compound according to any one of items 1 to 157, wherein R² is adamantyl.

236. The compound according to any one of items 1 to 157, wherein R² is adamant-1-yl.

237. The compound according to any one of items 1 to 157, wherein R² is

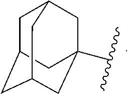 [SMILES: *C12CC3CC(CC(C3)C1)C2]

238. The compound according to any one of items 1 to 157, wherein R² is C₃₋₆ alkyl.

239. The compound according to any one of items 1 to 157, wherein R² is C₄ alkyl.

240. The compound according to any one of items 1 to 157, wherein R² is iso-butyl.

241. The compound according to any one of items 1 to 157, wherein R² is C₃₋₆ alkanediyl optionally substituted with R²⁸, R⁵ is a bond, and R² and R⁵ are linked together to form a ring.

242. The compound according to any one of items 1 to 157, wherein R² is C₄ alkanediyl optionally substituted with R²⁸, R⁵ is a bond, and R² and R⁵ are linked together to form a ring.

243. The compound according to any one of items 1 to 157, wherein R² is C₄ alkanediyl substituted with propyl, R⁵ is a bond, and R² and R⁵ are linked together to form a ring.

244. The compound according to any one of items 1 to 157, wherein

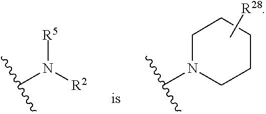

245. The compound according to any one of items 1 to 157, wherein

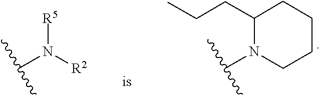

246. The compound according to any one of the preceding items wherein the C₁₋₅ alkyl is C₁₋₃ alkyl.

247. The compound according to any one of the preceding items wherein the C₁₋₃ alkyl is Me, Et, ⁿPr or ⁱPr.

248. The compound according to any one of the preceding items, wherein the halogen is Cl.

249. The compound according to any one of items 1 to 247, wherein the halogen is F.

250. The compound according to any one of the preceding items, wherein the compound is capable of binding to the Keap1 Kelch domain and inhibiting the Keap1-Nrf2 interaction.

251. The compound according to any one of the preceding items, wherein the compound has a Kᵢ of less than 500 μM, such as less than 100 μM, such as less than 50 μM, such as less than 25 μM, such as less than 10 μM, such as less than 5 M, such as less than 1 μM, such as less than 0.5 μM, such as less than 0.1 μM, such as less than 0.01 μM.

### EXAMPLES

### Example 1. Expression and Purification of Human and Mouse Keap1 Kelch Domains

The recombinant His-tagged human Keap1 Kelch domain (residue 321-609, UniProt Q14145) and the recombinant His-tagged mouse Keap1 Kelch domain (residue 322-624, UniProt Q9Z2X8; 97% identical to the human sequence) were cloned into a pRSET A vector and expressed in Escherichia coli BL21 (DE3) pLysS. Keap1 was grown in a preculture of 50 mL LB media supplemented with 1% glucose and 100 μg/mL ampicillin overnight (ON) at 37° C. to an OD600 of ~1.0. The preculture was transferred to 1 L of LB medium supplemented with 1% glucose and 100 μg/mL ampicillin and grown at 37° C./180 rpm to an OD600 of ~0.5, before induction with isopropyl β-D-1 thiogalactopyranoside (final concentration, 0.5-1 mM) ON at 15° C./180 rpm. Cells were harvested by centrifugation at 4000 g for 30 min. The cells were resuspended in lysis buffer (50 mM HEPES pH 7.5, complete Protease Inhibitor Cocktail (1 tablet/50 ml of buffer), 25 μg/mL DNase, 40 mM Mg₂SO₄, 150 mM NaCl, 5 mM imidazole, 5% glycerol, 0.5% Triton X-100, 3 mM DTT, 1 mg/mL lysozyme) and lysed using a cell disruptor at 26 KPsi in 4° C. The cell lysate was spun down at 35 000 g for 1 h at 4° C. The supernatant was filtered on a 0.45 μm filter and loaded onto a 5 mL HisTrap HP column (GE Healthcare). The column was washed with five column volumes of His Trap binding buffer (50 mM HEPES pH 7.5, 150 mM NaCl, 10 mM imidazole, 3 mM DTT) followed by eluting the protein using a gradient of HisTrap elution buffer (50 mM HEPES pH 7.5, 150 mM NaCl, 1 M imidazole, 3 mM DTT). The human Keap1 Kelch was eluted between 5 and 10% elution buffer and concentrated to 5 mL. The protein was then loaded onto a Superdex 75 16/600 column (GE Healthcare), equilibrated with SEC buffer (25 mM HEPES pH 7.5, 150mMNaCl, 1 mM TCEP) with a flow rate at 1 mL/min, and was eluted at 65 mL. The pure human Keap1 Kelch protein was concentrated to 12 mg/ml and stored at −80° C.

The mouse Keap1 Kelch domain was eluted between 10 and 15% elution buffer from the HisTrap column. The His-tag of mouse Keap1 Kelch domain was cleaved by adding 500 μL of 1 mg/mL His-tagged human rhinovirus-3C protease to approx. 50 mg of protein and incubated overnight at 4° C. in HisTrap binding buffer. After 16 h, the cleaved mouse Keap1 Kelch domain was purified using “reverse purification” on the HisTrap HP column and concentrated to 5 mL. The cleaved protein was loaded onto a Superdex 75 16/600 column (GE Healthcare), equilibrated with SEC buffer (20 mM Tris-HCl PH 8.3, 20 mM DTT and 10 mM benzamidine) with a flow rate of 1 mL/min, and was eluted at 65 mL. Protein was concentrated to 18 mg/mL for crystallography analysis and stored at −80° C.

The human and mouse Keap1 Kelch domains were analyzed using sodium dodecyl sulfate polyacrylamide gel electrophoresis for purity, and the concentrations were measured by absorbance (Nanodrop) using molar extinction coefficients calculated based on the amino acid sequence. The exact molecular weights of purified human and mouse Keap1 Kelch were confirmed by ESI-LC-MS.

### Example 2. Method for Determining Activity of Keap1-Nrf2 Inhibition by the Fluorescence Polarization (FP) Assay

The binding affinities between the fluorescent peptide probe Cy5-Nrf2 (SEQ: Cy5-LDEETGEFL-NH₂) and the human Keap1 Kelch domain were determined as K_{d} values by saturation binding experiments, where increasing concentrations of Keap1 (0-300 nM) were added to a fixed concentration of peptide probe (3 nM). The assay was performed in a 1× HBSTET buffer (10 mM HEPES, 150 mM NaCl, 0.005% Tween20, 3 mM ethylenediaminetetraacetic acid (EDTA), 1 mM TCEP, pH=7.4) using black flat-bottom 384-well plates (Corning Life Sciences, NY), a volume of 30 μL/well, and a final DMSO concentration of 4% or 8%. The assay plate was spun down to ascertain proper mixing and removal of potential air bubbles and incubated for 10-15 min at room temperature before measuring the FP levels on a Safire2 plate reader (Tecan, Mannedorf, Switzerland). The g-factor was adjusted at each experiment so that a series of three blank wells containing probe but no Keap1 Kelch defined the baseline FP value. The Cy5-Nrf2 probe was measured at excitation/emission values of 635:670 nm. The FP values were fitted to the one-site specific binding equation: Y=Bₘₐₓ×X/(K_{d}+X), with Bₘₐₓ being the maximal FP value, X the Keap1 Kelch concentration, and Y the variable FP values. The K_{d} values were derived from the resulting binding saturation curve as being equal to the Keap1 Kelch concentration, where the curve is half-saturated.

The affinities between nonfluorescent small molecule Keap1 inhibitors and Keap1 Kelch were determined as Kᵢ values in a heterologous competition FP binding assay. This was done by adding increasing concentration of test compound (2-fold serial dilutions, 12 concentrations tested per inhibition curve as duplicates) to a fixed concentration of Keap1 Kelch (14 nM) and the Cy5-Nrf2 peptide probe (3 nM) in the same HBSTET buffer and conditions as described above (final DMSO concentrations were 4%). FP values were fitted to the equation Y=Bottom+(Top−Bottom)/[1+(10^{HillSlope×(log IC50-X)})], where X is the logarithmic value of compound concentration. Hereby, the IC₅₀ value was obtained, which together with the Ka value and probe and Keap1 Kelch concentrations was used to calculate the theoretical competitive inhibition constant, the Kᵢ value. 43 Kᵢ values are shown as mean±SEM (standard error of mean) if based on two or more individual measurements.

### Example 3. Method for Determining the Binding Mode of Fragments and Compounds to the Keap1 Kelch Domain by X-Ray Crystallography

Crystals of the apo mouse Keap1 Kelch domain were obtained within 4 days through crystallization trials in a sitting drop with 100 nL Crystal Screen HT condition—F3 (Hampton Research, USA) and 100 nL protein solution (12.93 mg/mL) using Crystal Gryphon Robot (ARI, USA) in a Swissci 3 Well 96-Midi Crystallization Plate (Hampton Research, USA) at 293 K. Best apo crystals were grown within 2 days by vapor-diffusion with 1 μL of screening solution (modified Crystal Screen HT condition—F3: 0.1 M sodium citrate pH 5.6, 0.5 M lithium sulfate, and 0.7-0.9 M ammonium sulfate) and 1 μL of protein solution (12.93 mg/mL) using the hanging drop method at 293 K. For obtaining protein-fragment/compound complexes, crystals of mouse Keap1 Kelch domain were soaked for 2-5 hours (50-100 mM; 10-20% DMSO) or 2-16 hours (0.5-10 mM; 10% DMSO) with fragments or compounds, respectively, in a solution containing 0.1 M Na citrate pH 5.6, 0.5-0.6 M ammonium sulphate, 0.9-1.1 M lithium sulphate, and then harvested in liquid nitrogen for X-ray diffraction. Data for mouse Keap1 Kelch domain in complex with fragments and larger compounds were collected from synchrotron beamline facilities (Diamond Light Source, Oxfordshire and BioMAX at MAXIV, Lund, respectively). Diffraction images were integrated, scaled, and merged using autoprocessed beamline tools, while in some cases the data were reprocessed and scaled using XDS. The structures were solved using PHASER with PDB ID 6ZF4 as the search model for molecular replacement. Restraints for the fragments/compounds were prepared using the AceDRG followed by Model building and refinement using COOT and Phenix.refine.³⁷ Figures were prepared using PyMOL (The PyMOL Molecular Graphics System, Version 2.3.4 Schrödinger, LLC).

### Example 4. Fragment Screening by Crystallization and Affinity Measurement of Hit by Surface Plasmon Resonance (SPR)

768 fragments from the commercial available (Enamine) DSI-poised library were screened against the human Keap1 Kelch domain using X-ray crystallography. After data collection and processing (example 3), several crystal structures with high-resolution (1.0-2.1 Å) were identified showing fragments bound to the Keap1 binding pocket. Fragment XCF #0252 (also named UCAB #765) was found to occupy the P3 and P5 subpockets of the Keap1 Kelch domain (FIG. 2A). To confirm XCF #0252 as a hit and determine its affinity to human Keap1 Kelch, this fragment was resynthesized (Scheme 1) and tested by surface plasmon resonance (SPR). In SPR, a dose-dependent activity was observed and an affinity of K_{d}=0.6 mM was measured (FIG. 2B).

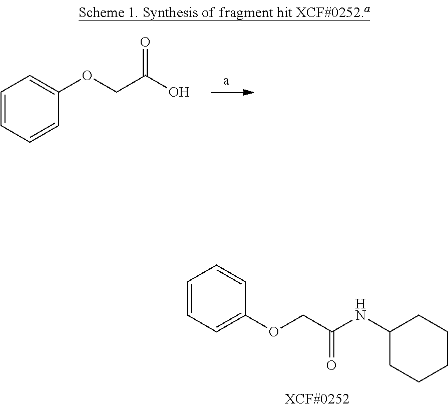 [SMILES: O=C(O)COc1ccccc1 ; O=C(COc1ccccc1)NC1CCCCC1]

^{a}Reagents and conditions: (a) Cyclohexanamine, EDC-HCl, HOBt, DIPEA, DMF, 0° C. to RT, 12 h (57%).

### Synthesis Protocols for Making XCF #0252:

N-Cyclohexyl-2-phenoxyacetamide (XCF #0252; UCAB #765). To a solution of 2-phenoxyacetic acid (0.20 g, 1.32 mmol) in DMF (4.0 mL) cooled to 0° C. was added EDC·HCl (0.38 g, 1.97 mmol), HOBt (0.27 g, 1.97 mmol), and DIPEA (0.46 mL, 2.63 mmol). The mixture was stirred at 0° C. for 30 min. After pre-activation, a solution of cyclohexanamine (0.13 g, 1.32 mmol) in DMF (2.6 mL) was added by syringe and the mixture stirred under nitrogen at room temperature overnight. Water (15 mL) was added and the mixture extracted with EtOAc (3×15 mL). The combined organic layers were washed with 1 M HCl (15 mL), sat. Na₂CO₃ (15 mL) and brine (15 mL), dried over Na₂SO₄, filtered and evaporated to dryness in vacuo. The crude was purified by silica gel column chromatography to afford the desired product. White solid (0.175 g, 57%). ¹H NMR (400 MHz, DMSO) δ 7.83 (d, J=8.1 Hz, 1H), 7.35-7.25 (m, 2H), 7.00-6.91 (m, 3H), 4.44 (s, 2H), 3.62 (s, 1H), 1.76-1.61 (m, 4H), 1.56 (d, J=12.5 Hz, 1H), 1.26 (qd, J=10.5, 2.7 Hz, 4H), 1.11 (d, J=10.7 Hz, 1H). ¹³C NMR (101 MHz, DMSO) δ 166.65, 158.00, 129.61 (2C), 121.25, 114.87 (2C), 67.13, 47.59, 32.42 (2C), 25.33, 24.83 (2C). LC-MS (ESI): m/z=234.1, t_{R}=4.35 min.

SPR measurements were performed at 25° C. using a Pioneer FE FE instrument (Sartorius). The Keap1 Kelch domain was covalently immobilized on the biosensor chip surface by amine coupling to a level of 2150 RU, using a 10 mM NaOAc pH 5 immobilization buffer. The 1×HBSTET buffer supplemented with 4% DMSO was used as running buffer for the experiments. Microcalibration was performed for all SPR experiments to adjust for DMSO bulk effects (low limit of 3% and a high limit of 4%). The compound was injected in a concentration series of twofold serial dilutions (67.5-1000 μM) using standard injections at a 30 μL/min flow rate. The data were analyzed using Qdat Data Analysis Tool version 2.6.3.0 (Sartorius). The sensorgrams were corrected for buffer effects and unspecific binding to the chip matrix by subtraction of blank and reference surfaces (a blank flow cell channel activated by the injection of EDC/NHS and inactivated by the injection of ethanolamine). The dissociation constant (Kd) was estimated by plotting MW normalized response levels (RUnorm) at equilibrium (Req) against the injected concentration and curve-fitted to a Langmuir (1:1) binding isotherm or by kinetic global fit of the SPR sensorgram to a simple 1:1 interaction model, while R_{norm,max} was fixed to the theoretical possible maximum (15 RU) normalized Rₘₐₓ.

### Example 5. Design and Synthesis of a Novel Series of Noncovalent Keap1-Nrf2 Inhibitors by Optimizing Fragment Hit XCF #0252

We designed and synthesized several analogues (1a-h, 2a-z, 3a-t, 4a-h, 5a-d, 6a-I, 7a-d, 8a-d and 9a-c; Scheme 2-10; Table 1) of XCF #0252 in order to improve and optimize the affinity. The analogues were designed by applying a fragment growing strategy guided by the X-ray structure of XCF #0252.

In the first stage of the optimization process, compound 1a-h were synthesized (Scheme 2). Treatment of commercially available 7-bromonaphthalen-2-ol with ethyl bromoacetate by Williamson ether synthesis affording I-1, followed by ester hydrolysis under basic condition, yielded the acid building block I-2. Then, treating I-2 with a variety of amines via amide coupling gave a series of amide intermediates (I-3a-h). A subsequent Heck reaction with methyl acrylate yielded I-4a-h, which were then hydrogenated to give I-5a-h followed by a base-catalyzed hydrolysis reaction to produce the final analogues 1a-h (Scheme 2).

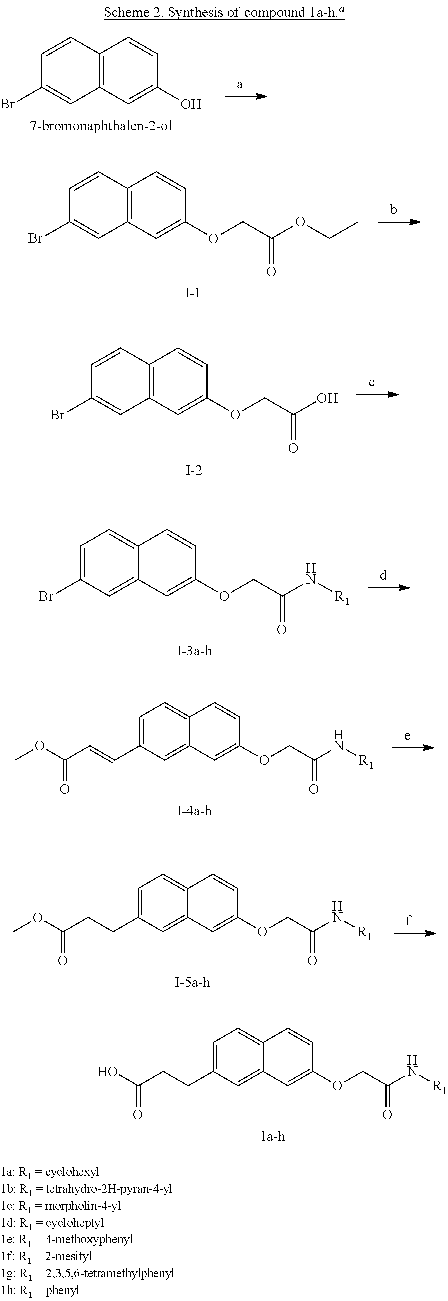

^{a}Reagents and conditions: (a) Ethyl bromoacetate, K₂CO₃, MeCN, 60° C., 18 h (78%); (b) 1M NaOH, MeOH, RT, 12 h (80%); (c) various amine, HATU, HOBt, DIPEA, DMF, 0° C. to RT, 12 h (32-95%); (d) Methyl acrylate, Pd(OAc)₂, P(o-tol)₃, Et₃N, MeCN, N₂, reflux, 1.5 h (22-97%); (e) 5% Pd/C, H₂ (balloon) MeOH, RT, 22 h (62-98%); (f) 1 M NaOH, MeOH, RT, 12 h (4-96%).

In the following round of design and synthesis, the cycloheptyl-containing analogues (2a-z) were generated (Scheme 3). The synthesis of these began by converting the intermediate I-3d (Scheme 2) to the key boronate ester intermediate I-6 by a palladium-catalyzed Miyaura borylation reaction (Scheme 3A). To synthesize the ‘left’ side of the analogues (later found to bind the P4 subpocket of Keap1 Kelch), different substituted bromobenzene building blocks (I-8a-z), which are commercially available except for I-8p and I-8d, were reacted with methyl acrylate via the Heck reaction to produce various methyl cinnamate products (I-9a-z) (Scheme 3B). The final analogues (2a-z) were then produced by a rhodium-catalyzed conjugation addition of I-6 to the corresponding methyl cinnamate intermediates (I-9a-z) to get I-7a-z, followed by saponification (Scheme 3A). The building block I-8p needed for making I-9p and then 2p followed the synthesis route showed in Scheme 3C. Here, the starting material 1-fluoro-3-methyl-2-nitrobenzene was converted to I-10 by a S_{N}Ar reaction, then treated with N-bromosuccinimide to produce intermediate I-11, which was then converted to the diamine compound I-12 by reduction of the nitro group. Finally, I-12 was cyclized to I-8p by acid-mediated elimination (Scheme 3C). The building block I-8u for making I-9u and then 2u were made by treating 6-methyl-2,3-dihydrobenzo[b][1,4]dioxine with N-bromosuccinimide (Scheme 3D).

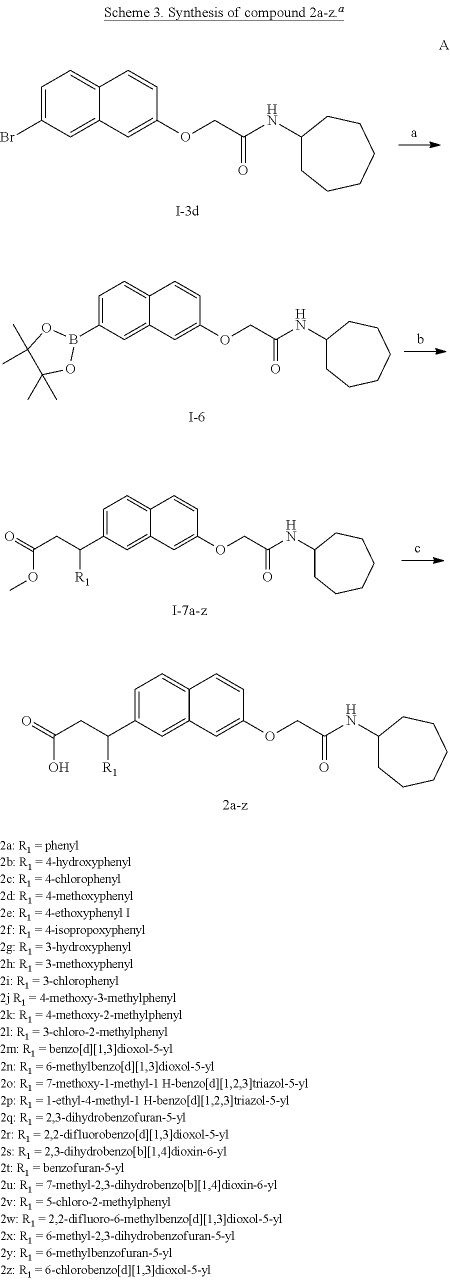

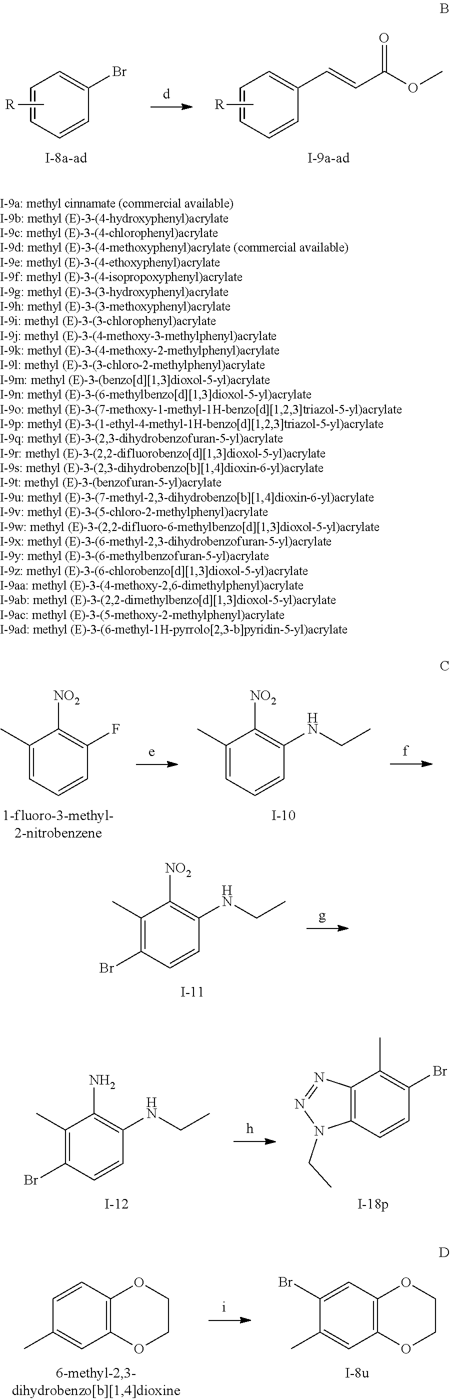

^{a}Reagents and conditions: (a) PinBBPin, Pd(dppf)Cl₂ KOAc, Dioxane, N₂ 100° C., 4 h (90%); (b) Methyl cinnamate products (1-9a-z), [Rh(COD)Cl]₂, TEA, dioxane/H₂O, N₂, 150° C. Microwave heating, 50 min (15-92%); (c) 2M NaOH, MeOH, 100° C. Microwave heating, 30 min (4-71%); (d) Methyl acrylate, Pd(OAc)₂, P(o-tol)₃, Et₃N, MeCN, N₂, reflux, 1.5 h (12-99.6%); (e) Ethanamine hydrochloride, TEA, K₂CO₃, EtOH, 90° C., 12 h (69%); (f) NBS, DMF, 0° C.-RT 12 h (76%); (g) Zn, NH₄Cl, EtOH/H₂O, 80° C., 1 h (52%); (h) NaNO₂, H₂SO₄, H₂O, 0° C., 2 h (57%). (i) NBS, DMF, 0° C.-RT 12 h (54%).

In a parallel analogue series, the cyclohexyl-containing analogues (compound 3a-t) were made (Scheme 4). The synthetic route was similar to the cycloheptyl series (Scheme 3), as the cyclohexyl intermediate I-3a (Scheme 2) was subjected to a palladium-catalyzed Miyaura borylation reaction to obtain the key boronate ester intermediate I-13. Then, I-13 reacted with the corresponding methyl cinnamate ester reagents (Scheme 3B) via a rhodium-catalyzed conjugation addition followed by saponification to yield final analogues 3a-t.

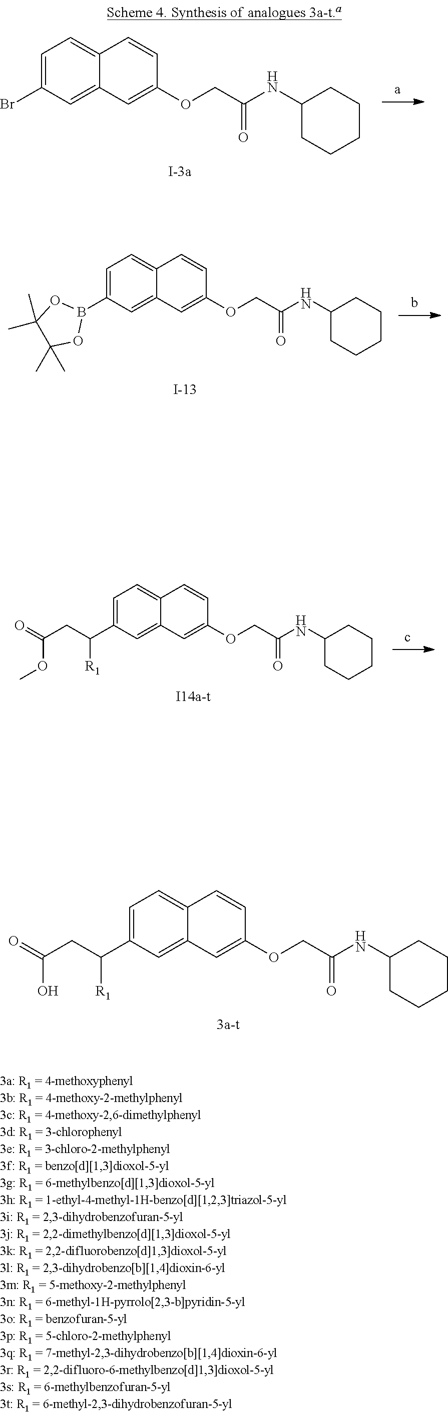

^{a}Reagents and conditions: (a) PinBBPin, Pd(dppf)Cl₂ KOAc, Dioxane, N₂, 100° C., 4 h (89%); (b) Methyl cinnamate products (I-9d, k, aa, i, l, m, n, p, q, ab, r, s, ac, ad, t, v, u, w, y, x, respectively), [Rh(COD)Cl]₂, TEA, dioxane/H₂O, N₂, 150° C. Microwave heating, 50 min (21-70%); (c) 2M NaOH, MeOH, 100° C. Microwave heating, 30 min (5.2-90%)

In another series of analogues, the cyclohex-2-en-yl-containing analogues (compound 4a-n) were made (Scheme 5) using the same procedure as for the cycloheptyl and cyclohexyl series (Scheme 3-4). Here, I-2 was treated with cyclohex-2-en-1-amine to provide amide I-15, which was subjected to the palladium-catalyzed Miyaura borylation reaction to obtain the key boronate ester intermediate I-16. Then, I-16 reacted with the corresponding methyl cinnamate ester reagents (Scheme 3B) via the rhodium-catalyzed conjugation addition followed by saponification to yield final analogues 4a-n.

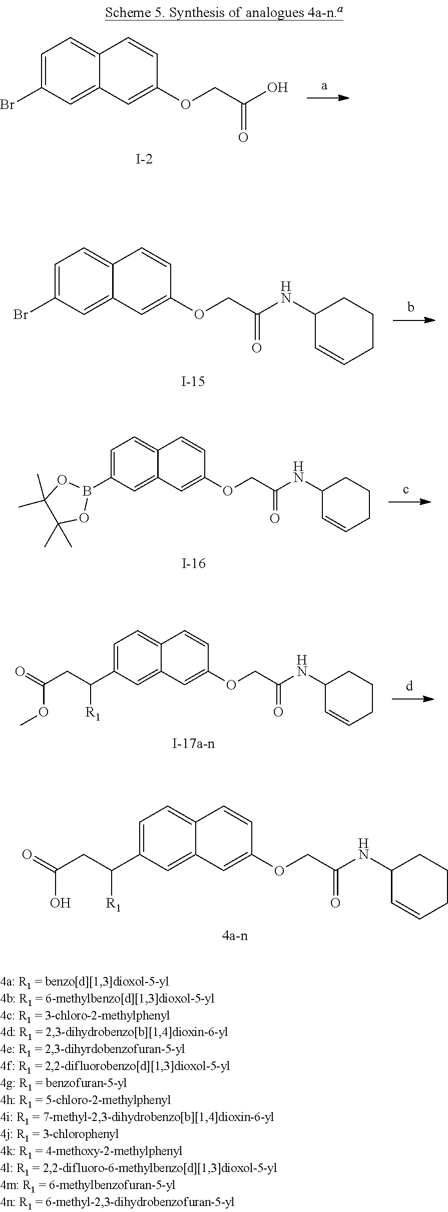

^{a}Reagents and conditions: (a) HATU, DIPEA, cyclohex-2-en-1-amine hydrogenchoride, DMF, 0° C. to RT, 12 h (98%); (b) PinBBPin, Pd(dppf)Cl₂ KOAc, Dioxane, N₂, 100° C., 4 h (89%); (c) Methyl cinnamate products (I-9m, n, l, s, q, r, t, v, u, i, k, w, y, x, respectively), [Rh(COD)Cl]₂, TEA, dioxane/H₂O, N₂, 150° C. Microwave heating, 50 min (21-48%); (d) 2M NaOH, MeOH, 100° C. Microwave heating, 30 min (17-78%)

Also, the bicyclo[2.2.1]heptan-2-yl-containing analogues (compound 5a-d) were made (Scheme 6) using the same procedure as for the cycloheptyl, cyclohexyl and cyclohex-2-en-yl series (Scheme 3-5). Here, I-2 was treated with (1S,2S,4R)-bicyclo[2.2.1]heptan-2-amine to provide amide I-18, which was subjected to the palladium-catalyzed Miyaura borylation reaction to obtain the key boronate ester intermediate I-19. Then, I-19 reacted with the corresponding methyl cinnamate ester reagents (Scheme 3B) via the rhodium-catalyzed conjugation addition followed by saponification to yield final analogues 5a-d.

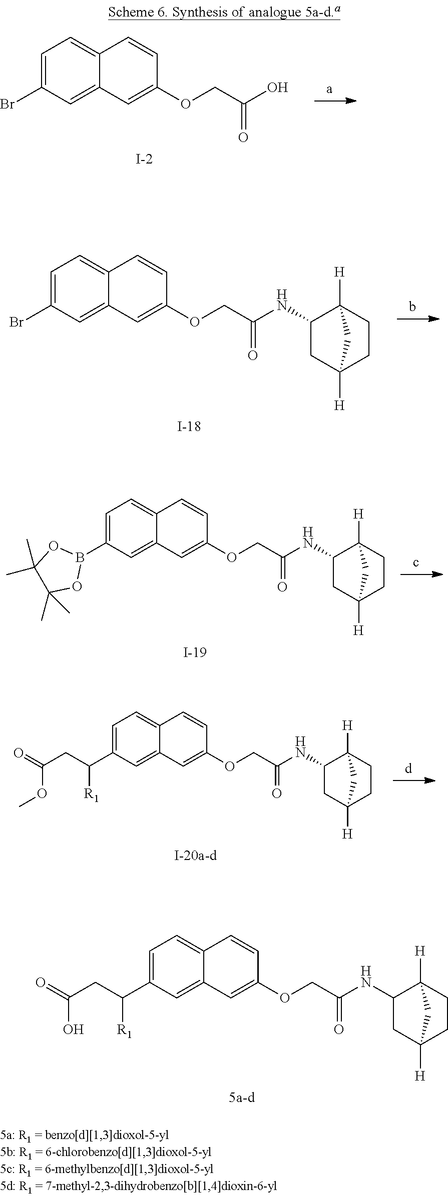

^{a}Reagents and conditions: (a) HATU, DIPEA, (1S,2S,4R)-bicyclo[2.2.1]heptan-2-amine, DMF, 0° C. to RT, 12 h (80%); (b) PinBBPin, Pd(dppf)Cl₂ KOAc, Dioxane, N₂, 100° C., 4 h (86%); (c) Methyl cinnamate products (I-9m, z, n, u, respectively), [Rh(COD)Cl]₂, TEA, dioxane/H₂O, N₂, 150° C. Microwave heating, 50 min (34-68%); (d) 2M NaOH, MeOH, 100° C. Microwave heating, 30 min (18-64%)

To explore the SAR of P5, an additional series of benzodioxol-5-yl-containing compound 6a-I were generated (Scheme 7). The synthesis of these began by treating I-2 with a variety of amines via amide coupling giving a series of amide intermediates (I-21a-I), which were subjected to the palladium-catalyzed Miyaura borylation reaction to obtain the corresponding boronate ester intermediate I-22a-I. Then, I-22a-I reacted with the methyl (E)-3-(benzo[d][1,3]dioxol-5-yl) acrylate (I-9m, Scheme 3B) via the rhodium-catalyzed conjugation addition followed by saponification to yield the final analogues 6a-I.

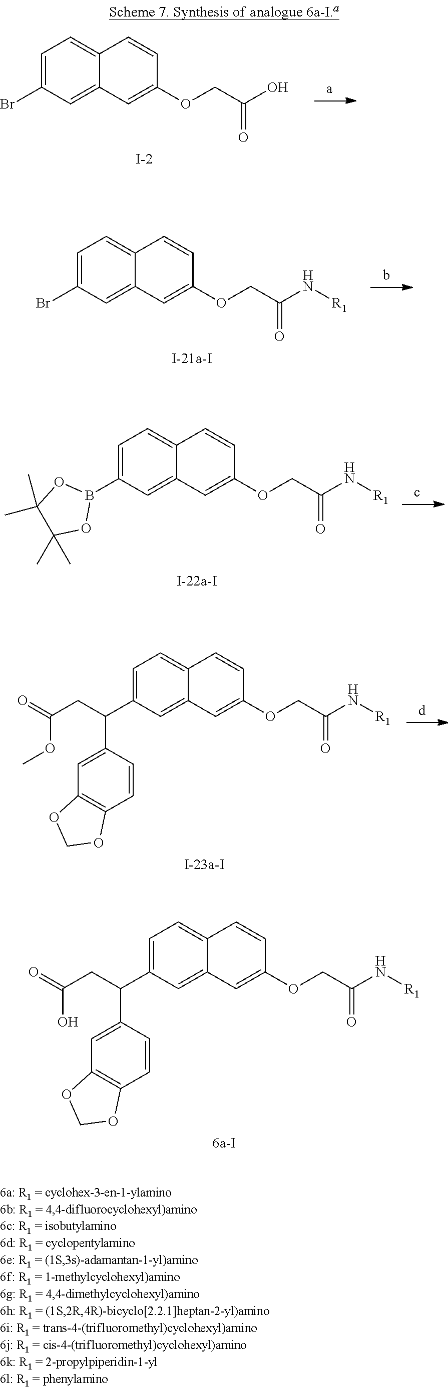

^{a}Reagents and conditions: (a) HATU, DIPEA, various amines, DMF, 0° C. to RT, 12 h (75-95%); (b) PinBBPin, Pd(dppf)Cl₂ KOAc, Dioxane, N₂, 100° C., 4 h (34-97%); (c) I-9m, [Rh(COD)Cl]₂, TEA, dioxane/H₂O, N₂, 150° C. Microwave heating, 50 min (13-68%); (d) 2M NaOH, MeOH, 100° C. Microwave heating, 30 min (17-52%).

In an additional series, we designed and synthesized analogues where methyl groups were added to the scaffold, as exemplified by compound 7a-d (Scheme 8). Here, 7-bromonaphthalen-2-ol was treated with commercially available ethyl 2-bromopropanoate in a Williamson ether synthesis way to yield I-24. This ester intermediate was hydrolysed with base followed byamide coupling with cycloheptanamine to yield I-26. From I-26, the palladium-catalyzed Miyaura borylation reaction provided the boronate ester intermediate I-27, which was reacted with the methyl cinnamate intermediate I-9m (Scheme 3B) by the rhodium-catalyzed conjugation addition to provide I-28. Basic hydrolysis of the ester, then gave the final product 7a (Scheme 8A); Treating I-7m (Scheme 3A) with NaH to deprotonate amide, which was then substituted by nucleophile Mel, yielded the final analogue 7b (Scheme 8B); For the synthesis of 7c and 7d, 7-bromonaphthalen-2-ol underwent a palladium-catalyzed Miyaura borylation reaction to produce the boronate ester intermediate I-30. Then, I-30 was reacted with the methyl (E)-3-(benzo[d][1,3]dioxol-5-yl) acrylate (I-9m, Scheme 3B) via the rhodium-catalyzed conjugation addition followed by TBS protection of the hydroxyl group to create the key intermediate I-32. By controlling the equivalent amount of LDA, treating I-32 with Mel formed either a mono-methyl (1-33a) or a di-methyl (I-33b) substituted product through S_{N}2 reaction. Treating I-33a-b with TBAF in THF afforded the deprotected intermediates I-34a-b. Commercially available 2-bromoacetyl chloride was treated with cycloheptanamine to form the amide bond building block I-36 (Scheme 8D). I-34a and I-34b were then reacted with the budling block I-36 via Williamson ether synthesis, followed by ester hydrolysis under basic conditions, affording the final analogues 7c and 7d, respectively (Scheme 8C).

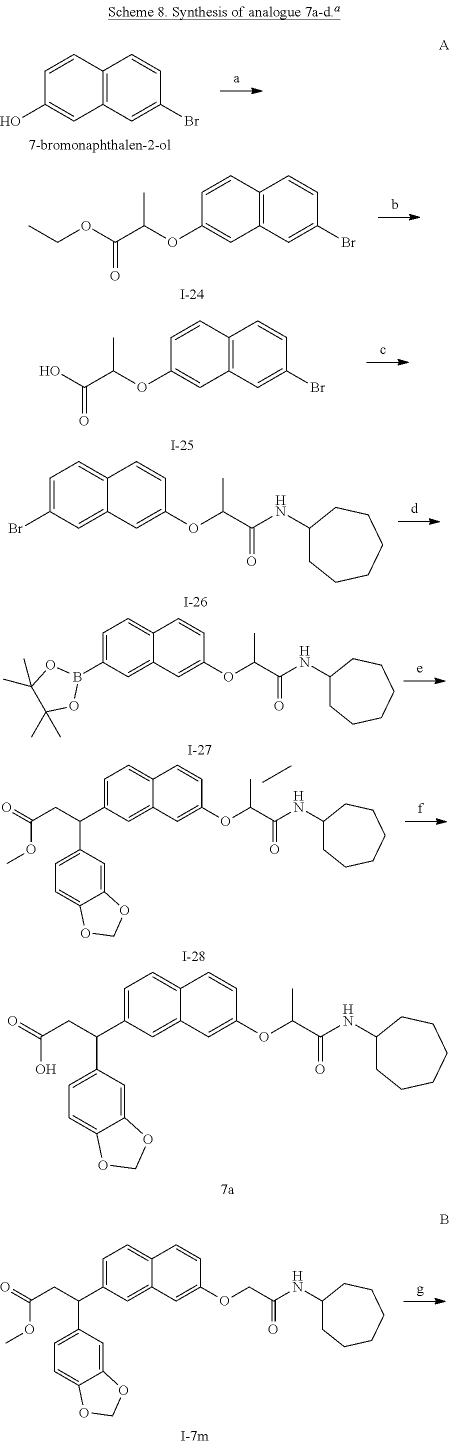

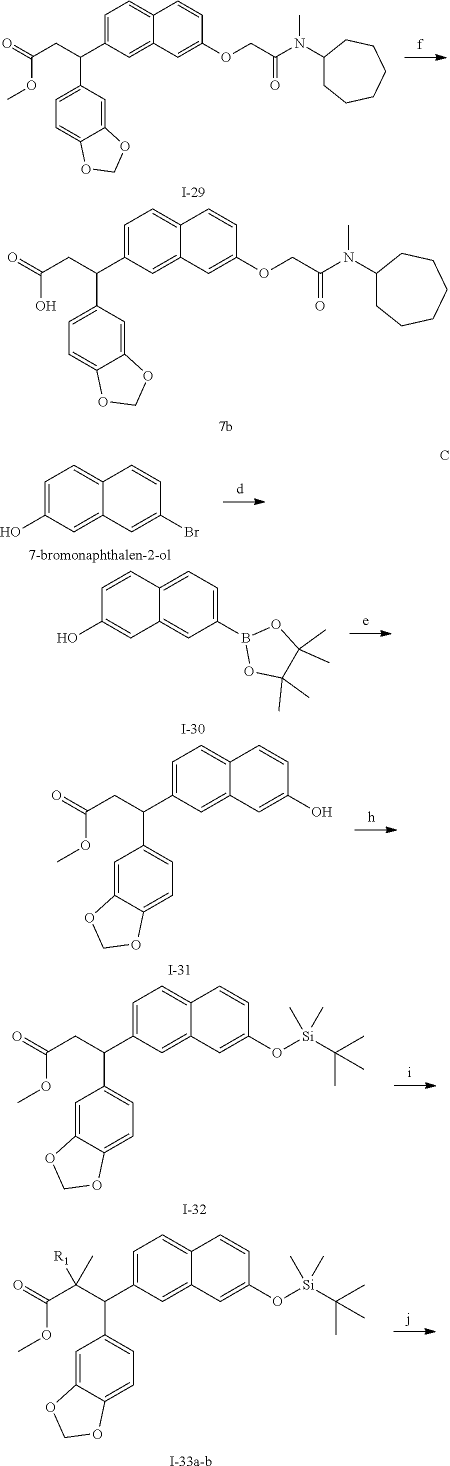

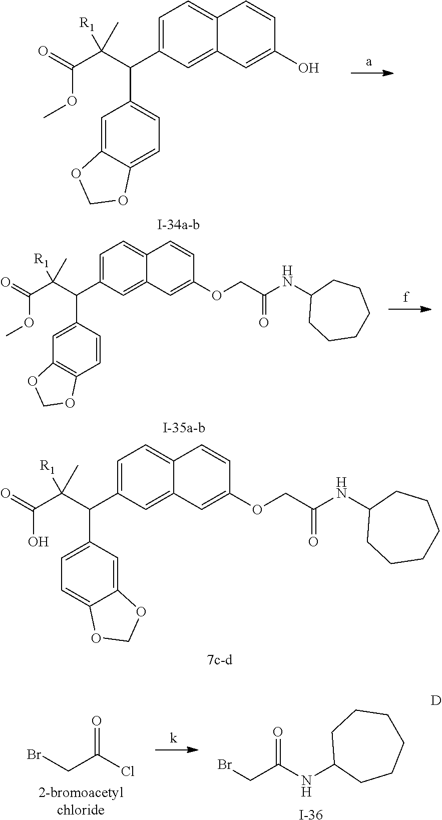

^{a}Reagents and conditions: (a) Ethyl 2-bromopropanoate, K₂CO₃, MeCN, 60° C., 18 h (31-96%); (b) 1M NaOH, MeOH, RT, 12 h (99%); (c) HATU, DIPEA, cycloheptanamine, DMF, 0° C. to RT, 12 h (20%); (d) PinBBPin, Pd(dppf)Cl₂ KOAc, Dioxane, N₂, 100° C., 4 h (69-83%); (e) I-9m, [Rh(COD)Cl]₂, TEA, dioxane/H₂O, N₂, 150° C. Microwave heating, 50 min (20-35%); (f) 2M NaOH, MeOH, 100° C. Microwave heating, 30 min (30-60%); (g) NaH, Mel, DMF, 0° C. to RT, 2 h (60%); (h) tert-butyldimethylsilyl chloride, imidazole, N, N-dimethyl-4-aminopyridine, DMF, RT, 30 min (76%); (i) lithium diisopropylamide, Mel, THR, −78° C. to RT 1.5 h (9-28%); (j) TBAF, THF, RT (100%); (k) cycloheptanamine, DIPEA, DCM, −10° C. to RT, N₂, 30 min (95%).

To explore various prodrug strategies in order to mask the carboxylic acid, we designed and synthesized prodrug analogues where ester prodrugs and thiazolidinone as exemplified by compound 8a-d (Scheme 9). The intermediate I-7m (Scheme 3A) was served as an example of a methyl prodrug; The synthesis of ethyl prodrug 8b began with the conversion of commercially available 5-bromo-6-methylbenzo[d][1,3]dioxole into ethyl cinnamate product I-37 via the Heck reaction (Scheme 9A). Then, I-16 (Scheme 5) reacted with the intermediate I-37 via the rhodium-catalyzed conjugation addition to yield the final analogue 8b (Scheme 9B); For the synthesis of thiazolidinone prodrugs, thiazolidin-2-one was treated with the previous final carboxylic acid analogues via an amide coupling to yield the corresponding final thiazolidinone prodrugs 8c and 8d (Scheme 9C).

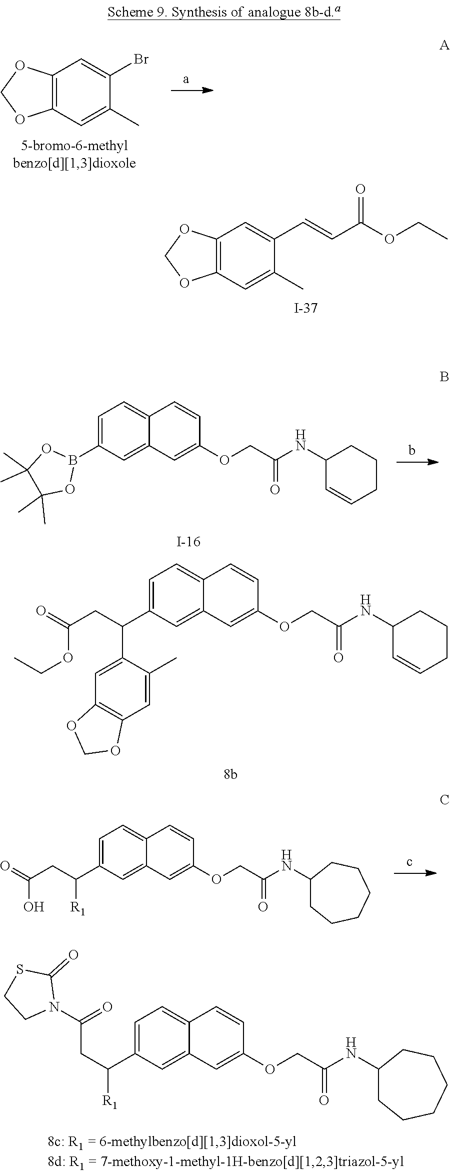

^{a}Reagents and conditions: (a) Ethyl acrylate, Pd(OAc)₂, P(o-tol)₃, Et₃N, MeCN, N₂, reflux, 1.5 h (64%); (b) 1-37, [Rh(COD)Cl]₂, TEA, dioxane/H₂O, N₂, 150° C., Microwave heating, 50 min (45%); (c) DCC, DMAP, thiazolidin-2-one, RT, overnight (6-14%); In an additional series, we designed and synthesized analogues by introducing an N-scan to the scaffold, as exemplified by compound 9a-c (Scheme 10). Here, 6-bromoquinolin-3-ol was treated with ethyl bromoacetate in a Williamson ether synthesis way to yield intermediate I-38. This ester intermediate was hydrolysed with base and coupled with cycloheptanamine to give I-40. The palladium-catalyzed Miyaura borylation reaction applied to I-40 provided the boronate ester intermediate I-41, which was then reacted with the methyl cinnamate intermediate I-9m (Scheme 3B) through rhodium-catalyzed conjugation addition to create I-42. Basic hydrolysis of the ester yielded final the product 9a (Scheme 10A); Treatment of commercially available 7-bromoquinolin-2 (1H)-one with the amide building block 1-36 (Scheme 8D) obtained I-43, which underwent a palladium-catalyzed Miyaura borylation reaction to produce the boronate ester intermediate I-44. Then, I-44 was reacted with the methyl (E)-3-(benzo[d][1,3]dioxol-5-yl) acrylate (I-9m, Scheme 3B) via the rhodium-catalyzed conjugation addition followed by the based-mediated ester hydrolysis to create the final analogue 9b (Scheme 10b); For the synthesis of compound 9c, commercially available 3-bromoquinolin-6-ol was treated with the amide building block 1-36 (Scheme 8D) to obtain 1-46, which was reacted with methyl acrylate via the Heck reaction to produce the methyl cinnamate intermediate I-47. The palladium-catalyzed Miyaura borylation reaction applied to 5-bromobenzo[d][1,3]dioxole provided the boronate ester intermediate I-49, which was then reacted with the methyl cinnamate intermediate I-47 through rhodium-catalyzed conjugation addition to create I-48. Basic hydrolysis of the ester yielded the final compound 9c (Scheme 10C);

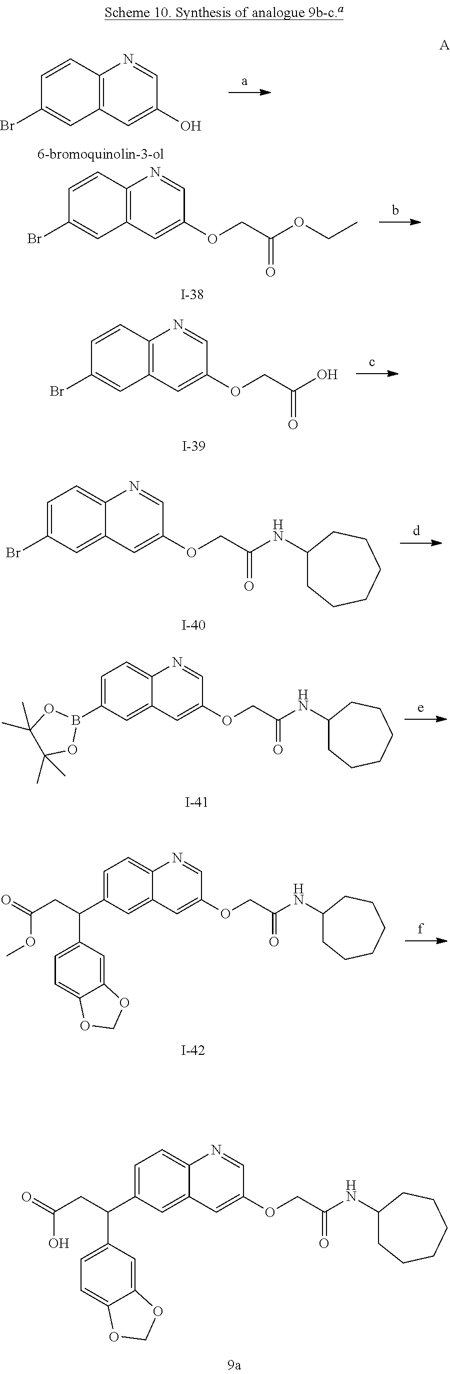

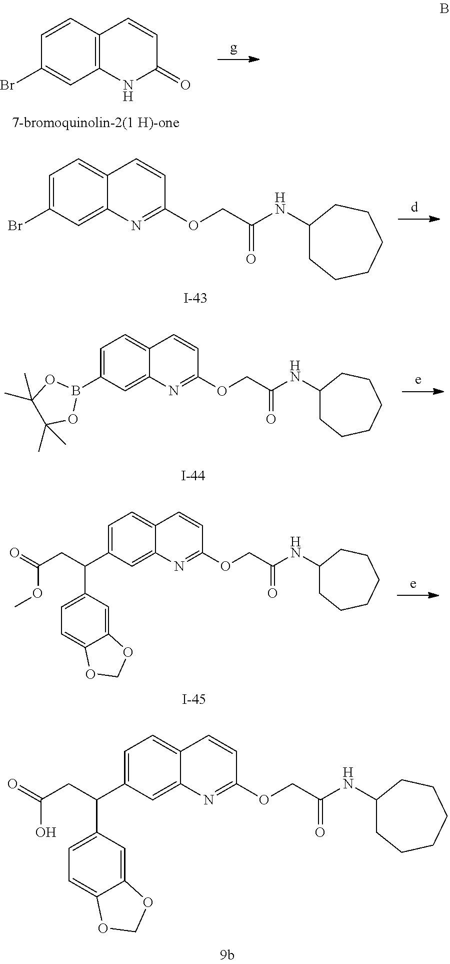

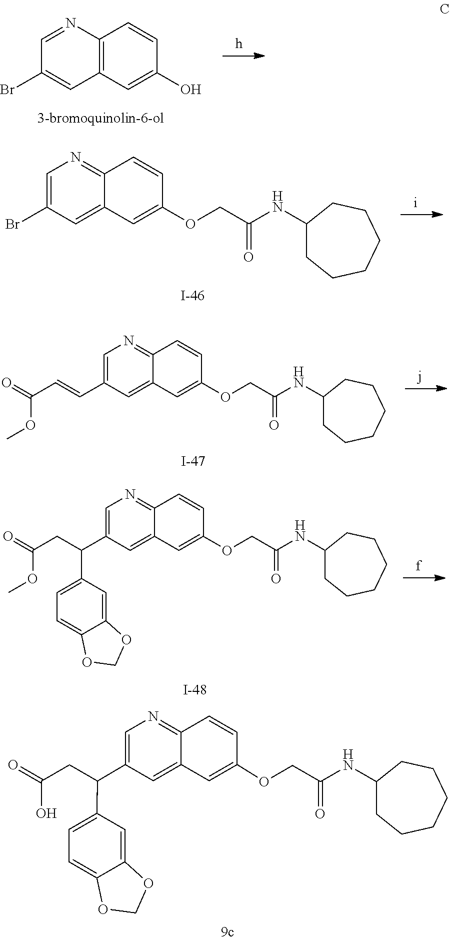

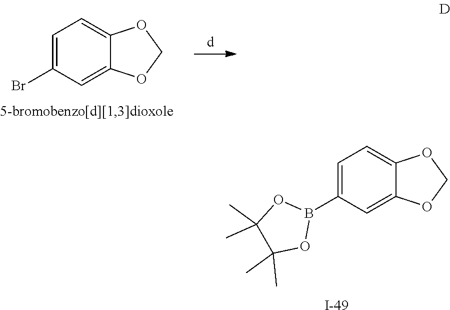

^{a}Reagents and conditions: (a) Ethyl 2-bromopropanoate, K₂CO₃, MeCN, 60° C., 18 h (87%); (b) 1M NaOH, MeOH, RT, 12 h (92%); (c) HATU, DIPEA, cycloheptanamine, DMF, 0° C. to RT, 12 h (45%); (d) PinBBPin, Pd(dppf)Cl₂ KOAc, Dioxane, N₂, 100° C., 4 h (20-90%); (e) I-9m, [Rh(COD)Cl]₂, TEA, dioxane/H₂O, N₂, 150° C. Microwave heating, 50 min (21-26%); (f) 2M NaOH, MeOH, 100° C. Microwave heating, 30 min (9-31%); (g) I-36, K₂CO₃, Pd(Xantphos)Cl₂, Toluene, 100° C., 48 h, (26%); (h) 1-36, K₂CO₃, MeCN, 80° C., 18 h (10%); (i) Methyl acrylate, Pd(OAc)₂, P(o-tol)₃, Et₃N, MeCN, N₂, reflux, 1.5 h (83%); (j) 1-49, [Rh(COD)Cl]₂, TEA, dioxane/H₂O, N₂, 150° C. Microwave heating, 50 min (46%).

### Detailed Synthesis Protocols for Making the Compounds are Described:

Procedures for purification and characterization: ¹H NMR, ¹³C NMR, and 2D NMR (COSY, HSQC, and HMBC) spectra were obtained using either a 600 MHz Bruker Avance III HD instrument equipped with a cryogenically cooled 5 mm dual probe or a 400 MHz Bruker Avance Ill instrument equipped with a 5 mm broad band probe. NMR samples were prepared in DMSO-de. Chemical shift (0) was reported in parts per million (ppm), while coupling constant (J) was reported in Hz. LC-MS analysis was performed on an Agilent 6130 Mass Spectrometer instrument using electron spray ionization (ESI) coupled to an Agilent 1200 HPLC system (ESI-LCMS) with a C₁₈ reverse-phase column (Zorbax Eclipse XBD-C18, 4.6 mm×50 mm), autosampler and diode array detector, using a linear gradient of the binary solvent system of buffer A (Milli-Q H₂O: MeCN: formic acid, 95:5:0.1 v/v %) to buffer B (MeCN: formic acid, 100:0.1 v/v %) with a flow rate of 1 mL/min for the other compounds. All of the following LC-MS date were collected in positive mode (m/z as [M+1]⁺). Normal phase flash chromatography was carried out using prepacked RediSep Rf silica flash cartridges on a CombiFlash® Rf+ apparatus. Reverse-phase flash chromatography was carried out on the CombiFlash® Rf+ apparatus using prepacked RediSep Rf Gold® Reversed-phase C₁₈ cartridge (50 g). Microwave reaction was performed on Biotage® Initiator+apparatus.

General procedure A: To a solution of various acid (1 equiv) in DMF (3.0 mL pr. mmol) cooled to 0° C. was added EDC. HCl (1.5 equiv), HOBt (1.5 equiv) and DIPEA (2 equiv). The mixture was stirred at 0° C. for 30 min. After pre-activation, a solution of various amine (1 equiv) in DMF (2.0 mL pr. mmol) was added by syringe and the mixture stirred under nitrogen at room temperature overnight. Upon reaction completion (as determined by LC-MS), water (10 mL pr. mmol) was added and the mixture extracted with EtOAc (3×10 mL pr. mmol). The combined organic layers were washed with 1 M HCl (10 mL pr. mmol), sat. Na₂CO₃ (10 mL pr. mmol) and sat. brine (10 mL pr. mmol), dried over Na₂SO₄, filtered and evaporated to dryness in vacuo. The crude was purified by silica gel column chromatography to afford the desired product.

General procedure B: A solution of various ester (1 equiv), 1 M aq. NaOH (4 equiv) were added in MeOH (1-3 mL). The mixture was stirred at room temperature for 22 h. Upon reaction completion, the pH of the aq. solution was adjusted to pH to acid with 1 M HCl resulting in compound precipitation, extracted with ethyl acetate three times, washed with brine, and dried over Na₂SO₄, filtered and concentrated under reduced pressure. The crude was purified by silica gel column chromatography to afford the desired product.

General procedure C: A solution of various unsaturated bromide (1 equiv), methyl acrylate (1.5 equiv), Pd(OAc)₂ (0.05 equiv), tri(o-tolyl)phosphine (0.1 equiv), and Et₃N (7.17 equiv) in MeCN (10 ml) was made. The reaction mixture was stirred at reflux (90° C.) and under nitrogen protection for 1.5 h. Upon reaction completion (as determined by LC-MS), water (10 mL pr. mmol) was added and the mixture extracted with EtOAc (3×10 mL pr. mmol). The combined organic layers were washed with 1 M HCl (10 mL pr. mmol), sat. NaHCO₃ (10 mL pr. mmol) and sat. brine (10 mL pr. mmol), dried over Na₂SO₄, filtered concentrated under reduced pressure to provide the crude product. The crude was purified by silica gel column chromatography to afford the desired product.

General procedure D: To a 50 mL round-bottomed flask charged with a magnetic stirring bar was added various acrylate (1 equiv) in MeOH (3 mL). The solution was degassed for 10 min. Then 5% Pd/C (20 w/w % wet-basis) was added against a positive nitrogen flow. The flask was capped and subjected to three vacuum-nitrogen cycles. Then hydrogen was introduced via a balloon, and the mixture stirred at RT for 22 h. Upon reaction completion, the flask was subjected to three vacuum-nitrogen cycles. The mixture was filtered through a compact bed of celite and the filter cake washed thoroughly with EtOAc. The combined filtrates were evaporated to dryness in vacuum without purification to afford the desired product.

General procedure E: A solution of 2-((7-bromonaphthalen-2-yl)oxy) acetic acid (1 equiv) in DMF (4 mL pr. mmol) was made. When the temperature cool to −15° C., then HATU (2 equiv), DIPEA (3 equiv), and amine (1.5 equiv) were added. The mixture was stirred at room temperature for overnight. Upon reaction completion (as determined by LC-MS), water (10 mL pr. mmol) was added and the mixture extracted with EtOAc (3×10 mL pr. mmol). The combined organic layers were washed with 1 M HCl (10 mL pr. mmol), sat. Na₂CO₃ (10 mL pr. mmol) and sat. brine (10 mL pr. mmol), dried over Na₂SO₄, filtered concentrated under reduced pressure to provide the crude product. The crude was purified by silica gel column chromatography to afford the desired product.

General procedure F: To a vial charged with a magnetic stirring bar was added 19 (1.5 equiv), various cinnamate esters (0.05 g, 0.25 mmol), [Rh(COD)Cl]₂ (0.1 equiv) and TEA (3 equiv) in dioxane/H₂O (1.5 mL/0.5 mL). The mixture was degassed and microwaved at 120° C. for 50 min. Upon reaction completion (as determined by LC-MS). After cooling down, the solvent was concentrated under reduced pressure to provide the crude product. The crude was purified by silica gel column chromatography to afford the desired product.

General procedure G: To a vial charged with a magnetic stirring bar were added various ester (1 equiv) in methanol (2 ml). Then a solution of 2 M aq. NaOH (2 equiv)) was added in the mixture. The mixture was microwaved at 100° C. for 30 min. Upon reaction completion, the mixture was concentrated in vacuo to remove MeOH. The pH of the aq. solution was then adjusted to pH to acid with 1 M HCl resulting in compound precipitation, and extracted with EtOAc (3×10 mL pr. mmol). The combined organic layers were washed with sat. brine (10 mL pr. mmol), dried over Na₂SO₄, filtered and concentrated under reduced pressure to provide the crude product. The crude was purified by silica gel column chromatography to afford the desired product.

Ethyl 2-((7-bromonaphthalen-2-yl)oxy)acetate (1-1) A mixture of 7-bromonaphthalen-2-ol (1 g, 4.5 mmol), K₂CO₃ (0.64 g, 4.6 mmol) and ethyl 2-bromoacetate (0.79 g, 4.7 mmol) were added in MeCN (10 mL). The mixture was allowed to stir at 60° C. for 18 h. Upon reaction completion, the mixture was cooled to room temperature, extracted with ethyl acetate three times, washed with brine, and dried over Na₂SO₄, filtered and concentrated under reduced pressure. The crude was purified by silica gel column chromatography to afford the desired product. White solid (1.08 g, 78.3%). ¹H NMR (400 MHz, DMSO-d₆) δ 8.06 (d, J=2.0 Hz, 1H), 7.88 (d, J=8.9 Hz, 1H), 7.82 (d, J=8.7 Hz, 1H), 7.48 (dd, J=8.7, 2.0 Hz, 1H), 7.30 (d, J=2.6 Hz, 1H), 7.26 (dd, J=8.9, 2.6 Hz, 1H), 4.89 (s, 2H), 4.20 (q, J=7.1 Hz, 2H), 1.23 (t, J=7.1 Hz, 3H). LC-MS (ESI): m/z=309.0/311.0, t_{R}=4.97 min.

2-((7-bromonaphthalen-2-yl)oxy) acetic acid (1-2). The reaction was conducted following the general procedure B, using I-1 (1.08 g, 3.50 mmol) and 1 M aq. NaOH (14 ml). White solid (0.78 g, 79.6%). ¹H NMR (600 MHz, DMSO) δ 12.29 (s, 1H), 8.08-8.05 (m, 1H), 7.90-7.85 (m, 1H), 7.83-7.79 (m, 1H), 7.47 (dd, J=8.6, 2.0 Hz, 1H), 7.29 (d, J=2.6 Hz, 1H), 7.24 (dd, J=8.9, 2.6 Hz, 1H), 4.79 (s, 2H). LC-MS (ESI): m/z=281.0/283.0, t_{R}=4.23 min

2-((7-Bromonaphthalen-2-yl)oxy)-N-cyclohexylacetamide (1-3a). The reaction was conducted following the general procedure A, using I-2 (0.30 g, 1.07 mmol), EDC·HCl (0.31 g, 1.61 mmol), HOBt (0.22 g, 1.61 mmol), DIPEA (0.4 mL, 2.14 mmol) and cyclohexanamine (0.32 g, 3.21 mmol). White solid (0.12 g, 31.6%). ¹H NMR (600 MHz, DMSO-d₆) δ 8.03 (d, J=2.0 Hz, 1H), 7.94 (d, J=8.1 Hz, 1H), 7.89 (d, J=8.9 Hz, 1H), 7.82 (d, J=8.7 Hz, 1H), 7.48 (dd, J=8.7, 2.0 Hz, 1H), 7.30 (dd, J=8.9, 2.6 Hz, 1H), 7.28 (d, J=2.5 Hz, 1H), 4.56 (s, 2H), 3.64 (td, J=9.3, 4.9 Hz, 1H), 1.71 (ddt, J=32.8, 8.9, 4.8 Hz, 4H), 1.59-1.54 (m, 1H), 1.30-1.25 (m, 4H), 1.16-1.09 (m, 1H). LC-MS (ESI): m/z=362.1/364.2, t_{R}=5.09 min.

Methyl (E)-3-(7-(2-(cyclohexylamino)-2-oxoethoxy)naphthalen-2-yl) acrylate (I-4a). The reaction was conducted following the General procedure C, using I-3a (0.12 g, 0.33 mmol), methyl acrylate (0.05 ml, 0.50 mmol), Pd(OAc)₂ (0.01 g, 0.02 mmol), tri(o-tolyl)phosphine (0.02 g, 0.04 mmol), and Et₃N (0.34 ml, 2.38 mmol). White solid (0.03 g, 24.8%). ¹H NMR (600 MHz, DMSO) δ 7.95 (d, J=8.2 Hz, 1H), 7.88 (dd, J=8.7, 3.7 Hz, 2H), 7.83-7.73 (m, 1H), 7.53 (tt, J=7.5, 1.5 Hz, 1H), 7.41 (dd, J=7.7, 4.0 Hz, 2H), 6.98 (ddd, J=13.9, 7.7, 1.4 Hz, 1H), 6.75 (d, J=16.0 Hz, 1H), 4.59 (s, 2H), 3.76 (s, 3H), 3.65 (d, J=4.0 Hz, 1H), 1.79-1.63 (m, 4H), 1.57 (m, 4H), 1.12 (d, J=10.7 Hz, 2H). LC-MS (ESI): m/z=3 68.3, t_{R}=4.82 min.

Methyl 3-(7-(2-(cyclohexylamino)-2-oxoethoxy)naphthalen-2-yl)propanoate (I-5a). The reaction was conducted following the general procedure D, using I-4a (0.03 g, 0.08 mmol) and 5% Pd/C (0.01 g). Colorless oil. LC-MS (ESI): m/z=370.2, t_{R}=4.56 min.

3-(7-(2-(Cyclohexylamino)-2-oxoethoxy)naphthalen-2-yl)propanoic acid (1a, UCAB #859). The reaction was conducted following the general procedure B, using I-5a (0.04, 0.10 mmol) and 1 M aq. NaOH (1 ml). White solid (0.013 g, 36.9%). ¹H NMR (600 MHz, DMSO) δ 12.11 (s, 1H), 7.92 (d, J=8.2 Hz, 1H), 7.82-7.74 (m, 2H), 7.57 (d, J=1.7 Hz, 1H), 7.26 (dd, J=8.3, 1.7 Hz, 1H), 7.22-7.17 (m, 2H), 4.55 (s, 2H), 3.67-3.63 (m, 1H), 2.96 (t, J=7.6 Hz, 2H), 2.62 (dq, J=3.7, 1.9 Hz, 2H), 1.81-1.67 (m, 4H), 1.57 (d, J=12.7 Hz, 1H), 1.28-1.24 (m, 4H), 1.12 (d, J=11.8 Hz, 1H). ¹³C NMR (151 MHz, DMSO) δ 173.17, 165.79, 155.27, 138.56, 133.61, 128.43, 126.98, 126.77, 124.60, 124.51, 117.40, 106.40, 66.50, 46.88, 34.53, 31.70 (2C), 29.96, 24.60, 24.11 (2C). LC-MS (ESI): m/z=356.2, t_{R}=4.23 min

2-((7-Bromonaphthalen-2-yl)oxy)-N-(tetrahydro-2H-pyran-4-yl) acetamide (I-3b). The reaction was conducted following the general procedure E, using I-2 (0.37 g, 1.32 mmol), HATU (1.01 g, 2.64 mmol), DIPEA (0.7 mL, 3.95 mmol) and tetrahydro-2H-pyran-4-amine (0.20 g, 1.98 mmol). White solid (0.8 g, 92%). ¹H NMR (600 MHz, DMSO-d₆) δ 8.10 (d, J=7.9 Hz, 1H), 8.05 (d, J=2.0 Hz, 1H), 7.89 (d, J=8.9 Hz, 1H), 7.82 (d, J=8.7 Hz, 1H), 7.48 (dd, J=8.7, 2.0 Hz, 1H), 7.34-7.28 (m, 2H), 4.59 (s, 2H), 3.93-3.86 (m, 1H), 3.84 (ddd, J=11.8, 4.1, 2.2 Hz, 2H), 3.35 (td, J=11.6, 2.2 Hz, 2H), 1.69 (ddd, J=12.5, 4.5, 2.1 Hz, 2H), 1.53 (dtd, J=12.9, 11.4, 4.4 Hz, 2H). LC-MS (ESI): m/z=364.0/366.1, t_{R}=4.41 min.

Methyl (E)-3-(7-(2-oxo-2-((tetrahydro-2H-pyran-4-yl)amino)ethoxy)naphthalen-2-yl) acrylate (I-4b). The reaction was conducted following the General procedure C, using I-3b (0.2 g, 0.5 mmol), methyl acrylate (0.08 ml, 0.83 mmol), Pd(OAc)₂ (0.01 g, 0.03 mmol), tri(o-tolyl)phosphine (0.02 g, 0.06 mmol), and Et₃N (0.55 ml, 3.94 mmol). Light brown solid (0.23 g, 94%). ¹H NMR (600 MHz, DMSO) δ 8.11 (d, J=8.0 Hz, 1H), 8.09-8.04 (m, 1H), 7.87 (dd, J=8.7, 5.4 Hz, 2H), 7.82-7.74 (m, 2H), 7.34-7.28 (m, 2H), 6.74 (d, J=16.0 Hz, 1H), 4.60 (s, 2H), 3.88 (dtd, J=11.1, 7.1, 4.0 Hz, 1H), 3.83 (ddd, J=11.8, 4.4, 2.4 Hz, 2H), 3.75 (s, 3H), 3.34 (td, J=11.7, 2.2 Hz, 2H), 1.68 (ddd, J=9.6, 4.6, 2.5 Hz, 2H), 1.57-1.47 (m, 2H). LC-MS (ESI): m/z=370.2, t_{R}=4.19 min.

Methyl 3-(7-(2-oxo-2-((tetrahydro-2H-pyran-4-yl)amino)ethoxy)naphthalen-2-yl)propanoate (I-5b). The reaction was conducted following the general procedure D, using I-4b (0.23 g, 0.63 mmol) and 5% Pd/C (0.3 g). Light brown solid (0.23 g, 92.8%). ¹H NMR (600 MHz, DMSO) δ 8.09 (d, J=8.0 Hz, 1H), 7.80 (d, J=8.7 Hz, 1H), 7.76 (d, J=8.3 Hz, 1H), 7.59-7.56 (m, 1H), 7.30-7.23 (m, 1H), 7.23-7.18 (m, 2H), 4.58 (s, 2H), 3.94-3.76 (m, 3H), 3.59 (s, 3H), 3.36 (td, J=11.7, 2.2 Hz, 2H), 3.00 (t, J=7.6 Hz, 2H), 2.75-2.68 (m, 2H), 1.69 (ddt, J=12.0, 4.5, 2.3 Hz, 2H), 1.53 (dtd, J=12.9, 11.4, 4.4 Hz, 2H). LC-MS (ESI): m/z=372.2, t_{R}=4.11 min.

3-(7-(2-Oxo-2-((tetrahydro-2H-pyran-4-yl)amino)ethoxy)naphthalen-2-yl)propanoic acid (1b, UCAB #996). The reaction was conducted following the general procedure B, using I-5b (0.13, 0.35 mmol) and 1 M aq. NaOH (2 ml). White solid (0.08 g, 61.6%). ¹H NMR (600 MHz, DMSO) δ 12.12 (s, 1H), 8.09 (d, J=8.0 Hz, 1H), 7.80 (d, J=8.6 Hz, 1H), 7.76 (d, J=8.3 Hz, 1H), 7.58 (d, J=1.7 Hz, 1H), 7.26 (dd, J=8.4, 1.7 Hz, 1H), 7.23-7.18 (m, 2H), 4.57 (s, 2H), 3.95-3.80 (m, 3H), 3.36 (dd, J=11.7, 2.2 Hz, 2H), 2.96 (t, J=7.6 Hz, 2H), 2.62 (t, J=7.6 Hz, 2H), 1.69 (ddt, J=11.3, 4.5, 2.2 Hz, 2H), 1.53 (dtd, J=12.9, 11.4, 4.4 Hz, 2H). ¹³C NMR (151 MHz, DMSO) δ 174.21, 167.15, 156.28, 139.60, 134.64, 129.48, 128.01, 127.82, 125.61 (2C), 118.44, 107.47, 67.53, 66.46 (2C), 45.40, 35.56, 32.77 (2C), 30.99. LC-MS (ESI): m/z=358.1, t_{R}=3.65 min.

2-((7-Bromonaphthalen-2-yl)oxy)-N-morpholinoacetamide (I-3c). The reaction was conducted following the general procedure E, using I-2 (0.3 g, 1.07 mmol), HATU (0.82 g, 2.14 mmol), DIPEA (0.6 mL, 3.21 mmol) and morpholin-4-amine (0.17 g, 1.60 mmol). White solid (0.27 g, 68.9%). ¹H NMR (600 MHz, DMSO-d₆) δ 7.87-7.79 (m, 3H), 7.47 (ddd, J=19.1, 8.7, 2.0 Hz, 2H), 7.30 (d, J=8.2 Hz, 2H), 4.56 (s, 2H), 3.63 (t, J=4.6 Hz, 4H), 2.83-2.78 (m, 4H). LC-MS (ESI): m/z=365.0/367.0, t_{R}=4.17 min.

Methyl (E)-3-(7-(2-(morpholinoamino)-2-oxoethoxy)naphthalen-2-yl) acrylate (I-4c). The reaction was conducted following the General procedure C, using I-3c (0.2 g, 0.55 mmol), methyl acrylate (0.08 ml, 0.83 mmol), Pd(OAc)₂ (0.01 g, 0.03 mmol), tri(o-tolyl)phosphine (0.02 g, 0.06 mmol), and Et₃N (0.55 ml, 3.94 mmol). White solid (0.27 g, 68.9%). ¹H NMR (600 MHz, DMSO) δ 9.37-8.75 (m, 1H), 7.83-7.72 (m, 2H), 7.59 (dd, J=16.4, 1.6 Hz, 2H), 7.28-7.16 (m, 1H), 7.14-7.08 (m, 1H), 4.98-4.55 (s, 2H), 3.63 (t, J=4.7 Hz, 4H), 3.59 (s, 3H), 2.99 (td, J=7.6, 5.4 Hz, 2H), 2.83-2.78 (m, 2H), 2.72 (t, J=7.6 Hz, 2H). LC-MS (ESI): m/z=371.1, t_{R}=3.99 min.

Methyl 3-(7-(2-morpholinoamino-2-oxoethoxy)naphthalen-2-yl)propanoate (I-5c). The reaction was conducted following the general procedure D, using I-4c (0.20 g, 0.56 mmol) and 5% Pd/C (0.4 g). Colorless oil (0.27 g, 68.9%). LC-MS (ESI): m/z=373.2, t_{R}=3.89 min.

3-(7-(2-Morpholinoamino-2-oxoethoxy)naphthalen-2-yl)propanoic acid (1c, UCAB #1001). The reaction was conducted following the general procedure B, using 1-5c (0.17, 0.48 mmol) and 1 M aq. NaOH (5 ml). White solid (0.043 g, 26.4%). ¹H NMR (600 MHz, DMSO) δ 12.12 (s, 1H), 9.37-8.81 (m, 1H), 7.95-7.64 (m, 2H), 7.59 (dd, J=13.5, 1.7 Hz, 1H), 7.25 (ddd, J=16.0, 8.3, 1.7 Hz, 1H), 7.21-7.16 (m, 1H), 7.14-7.08 (m, 1H), 4.98-4.55 (s, 2H), 3.63 (q, J=5.9 Hz, 4H), 2.96 (td, J=7.6, 4.9 Hz, 2H), 2.83-2.71 (m, 4H), 2.62 (td, J=7.6, 1.6 Hz, 2H). ¹³C NMR (151 MHz, DMSO) δ 174.22, 169.75, 156.28, 139.62, 134.66 129.51, 129.38, 127.98, 125.63 (2C), 118.40, 107.39, 67.02, 66.38 (2C), 54.99 (2C), 35.56, 31.02. LC-MS (ESI): m/z=359.2, t_{R}=3.44 min.

2-((7-Bromonaphthalen-2-yl)oxy)-N-cycloheptylacetamide (1-3d). The reaction was conducted following the general procedure E, using I-2 (0.07 g, 0.25 mmol), HATU (0.2 g, 0.5 mmol), DIPEA (0.15 mL, 0.75 mmol) and cycloheptanamine (0.05 g, 0.04 mmol). White solid (0.047 g, 50%). ¹H NMR (600 MHz, DMSO) δ 8.04 (d, J=2.0 Hz, 1H), 7.98 (d, J=8.1 Hz, 1H), 7.89 (d, J=8.9 Hz, 1H), 7.82 (d, J=8.7 Hz, 1H), 7.48 (dd, J=8.6, 2.0 Hz, 1H), 7.30 (dd, J=8.9, 2.5 Hz, 1H), 7.28 (d, J=2.5 Hz, 1H), 4.56 (s, 2H), 3.83 (tq, J=12.6, 4.5 Hz, 1H), 1.82-1.74 (m, 2H), 1.66-1.36 (m, 9H), 1.24 (s, 1H). LC-MS (ESI): m/z=376.0/378.1, t_{R}=5.21 min.

Methyl (E)-3-(7-(2-(cycloheptylamino)-2-oxoethoxy)naphthalen-2-yl) acrylate (I-4d). The reaction was conducted following the General procedure C, using I-3d (0.05 g, 0.13 mmol), methyl acrylate (0.08 ml, 0.83 mmol), Pd(OAc)₂ (0.01 g, 0.03 mmol), tri(o-tolyl)phosphine (0.02 g, 0.06 mmol), and Et₃N (0.55 ml, 3.94 mmol). White solid (0.027 g, 54%). ¹H NMR (600 MHz, DMSO) δ 8.07-8.03 (m, 1H), 7.99 (d, J=8.1 Hz, 1H), 7.89-7.73 (m, 4H), 7.33-7.27 (m, 2H), 6.74 (d, J=16.0 Hz, 1H), 4.57 (s, 2H), 3.87-3.80 (m, 1H), 3.75 (s, 3H), 1.81-1.73 (m, 2H), 1.65-1.26 (m, 10H). LC-MS (ESI): m/z=382.2, t_{R}=4.99 min.

Methyl 3-(7-(2-(cycloheptylamino)-2-oxoethoxy)naphthalen-2-yl)propanoate (I-5d). The reaction was conducted following the general procedure D, using I-4d (0.027 g, 0.07 mmol) and 5% Pd/C (0.31 g). White solid (0.02 g, 74.1%). ¹H NMR (600 MHz, DMSO) δ 7.96 (d, J=8.1 Hz, 1H), 7.79 (d, J=8.7 Hz, 1H), 7.75 (d, J=8.3 Hz, 1H), 7.56 (d, J=1.7 Hz, 1H), 7.25 (dd, J=8.3, 1.7 Hz, 1H), 7.21-7.16 (m, 2H), 4.54 (s, 2H), 3.83 (qt, J=9.1, 4.4 Hz, 1H), 2.96 (t, J=7.6 Hz, 2H), 2.61 (t, J=7.6 Hz, 2H), 1.82-1.73 (m, 2H), 1.66-1.34 (m, 8H). LC-MS (ESI): m/z=384.2, t_{R}=4.92 min.

3-(7-(2-(Cycloheptylamino)-2-oxoethoxy)naphthalen-2-yl)propanoic acid (1d, UCAB #909). The reaction was conducted following the general procedure B, using I-5d (0.02, 0.05 mmol) and 1 M aq. NaOH (1 ml). White solid (0.007 g, 36.8%). ¹H NMR (600 MHz, DMSO) δ 12.11 (s, 1H), 7.96 (d, J=8.1 Hz, 1H), 7.79 (d, J=8.7 Hz, 1H), 7.75 (d, J=8.3 Hz, 1H), 7.56 (d, J=1.7 Hz, 1H), 7.25 (dd, J=8.3, 1.7 Hz, 1H), 7.21-7.16 (m, 2H), 4.54 (s, 2H), 3.83 (qt, J=9.1, 4.4 Hz, 1H), 2.96 (t, J=7.6 Hz, 2H), 2.61 (t, J=7.6 Hz, 2H), 1.82-1.73 (m, 2H), 1.66-1.34 (m, 10H). ¹³C NMR (151 MHz, DMSO) δ 174.01, 166.34, 156.12, 139.39, 134.45, 129.26, 127.81, 127.60, 125.42, 125.34, 118.22, 107.21, 67.34, 49.88, 35.36, 34.50 (2C), 30.79, 27.94 (2C), 24.03 (2C). LC-MS (ESI): m/z=370.2, t_{R}=4.40 min.

2-((7-Bromonaphthalen-2-yl)oxy)-N-(4-methoxyphenyl) acetamide (I-3e). The reaction was conducted following the general procedure E, using I-2 (0.2 g, 0.72 mmol), HATU (0.6 g, 1.42 mmol), DIPEA (0.4 mL, 2.13 mmol) and 4-methoxyaniline (0.1 ml, 1.07 mmol). White solid (0.15 g, 53%). ¹H NMR (600 MHz, DMSO) δ 9.99 (s, 1H), 8.08 (d, J=2.0 Hz, 1H), 7.93-7.89 (m, 1H), 7.83 (d, J=8.7 Hz, 1H), 7.59-7.53 (m, 2H), 7.48 (dd, J=8.7, 2.0 Hz, 1H), 7.38-7.33 (m, 2H), 6.93-6.88 (m, 2H), 4.78 (s, 2H), 3.73 (s, 3H). LC-MS (ESI): m/z=386.0/388.0, t_{R}=4.87 min.

Methyl (E)-3-(7-(2-((4-methoxyphenyl)amino)-2-oxoethoxy)naphthalen-2-yl) acrylate (1-4e). The reaction was conducted following the General procedure C, using I-3e (0.15 g, 0.38 mmol), methyl acrylate (0.06 ml, 0.56 mmol), Pd(OAc)₂ (0.011 g, 0.02 mmol), tri(o-tolyl)phosphine (0.02 g, 0.04 mmol), and Et₃N (0.40 ml, 2.70 mmol). White solid (0.11 g, 30%). ¹H NMR (600 MHz, DMSO) δ 10.01 (s, 1H), 8.10 (d, J=1.7 Hz, 1H), 7.90 (dd, J=12.0, 8.6 Hz, 2H), 7.82-7.74 (m, 2H), 7.59-7.54 (m, 2H), 7.39-7.34 (m, 2H), 6.93-6.88 (m, 2H), 6.75 (d, J=16.0 Hz, 1H), 4.80 (s, 2H), 3.75 (s, 3H), 3.73 (s, 3H). LC-MS (ESI): m/z=392.2, t_{R}=4.60 min.

Methyl 3-(7-(2-((4-methoxyphenyl)amino)-2-oxoethoxy)naphthalen-2-yl)propanoate (I-5e). The reaction was conducted following the general procedure D, using I-4e (0.11 g, 0.29 mmol) and 5% Pd/C (0.03 g). White solid (0.07 g, 61.9%). ¹H NMR (600 MHz, HDMSO) δ 9.99 (s, 1H), 7.85-7.74 (m, 2H), 7.60 (d, J=1.6 Hz, 1H), 7.58-7.55 (m, 2H), 7.26 (tt, J=4.4, 2.3 Hz, 3H), 6.93-6.89 (m, 2H), 4.77 (s, 2H), 3.73 (s, 3H), 3.58 (s, 3H), 2.99 (t, J=7.6 Hz, 2H), 2.72 (t, J=7.6 Hz, 2H). LC-MS (ESI): m/z=394.2, t_{R}=4.55 min.

3-(7-(2-((4-Methoxyphenyl)amino)-2-oxoethoxy)naphthalen-2-yl)propanoic acid (1e, UCAB #908). The reaction was conducted following the general procedure B, using I-5e (0.05, 0.13 mmol) and 1 M aq. NaOH (5 ml). White solid (0.002 g, 4.2%). ¹H NMR (600 MHz, DMSO) δ 12.13 (s, 1H), 9.99 (s, 1H), 7.81 (d, J=9.6 Hz, 1H), 7.76 (d, J=8.3 Hz, 1H), 7.63-7.52 (m, 3H), 7.25 (ddt, J=7.1, 4.5, 2.2 Hz, 3H), 6.93-6.86 (m, 2H), 4.76 (s, 2H), 3.72 (s, 3H), 2.96 (t, J=7.6 Hz, 2H), 2.61 (t, J=7.6 Hz, 2H). ¹³C NMR (151 MHz, DMSO) δ 174.22, 166.43, 156.33, 156.05, 139.63, 134.64, 131.91, 129.56, 128.02, 127.86, 125.70, 125.61, 121.91 (2C), 118.45, 114.32 (2C), 107.41, 67.73, 55.65, 35.56, 31.00. LC-MS (ESI): m/z=380.2, t_{R}=4.07 min.

2-((7-Bromonaphthalen-2-yl)oxy)-N-mesitylacetamide (I-3f). The reaction was conducted following the general procedure E, using I-2 (0.07 g, 0.25 mmol), HATU (0.20 g, 0.50 mmol), DIPEA (0.15 mL, 0.75 mmol) and 2,4,6-trimethylaniline (0.6 ml, 0.38 mmol). White solid (0.05 g, 50%). ¹H NMR (600 MHz, DMSO) δ 9.48 (s, 1H), 8.06 (d, J=2.0 Hz, 1H), 7.92 (d, J=8.9 Hz, 1H), 7.84 (d, J=8.7 Hz, 1H), 7.50 (dd, J=8.7, 2.0 Hz, 1H), 7.40-7.34 (m, 2H), 6.88 (s, 2H), 4.84 (s, 2H), 2.22 (s, 3H), 2.08 (s, 6H). LC-MS (ESI): m/z=398.1/400.1, t_{R}=5.10 min.

Methyl (E)-3-(7-(2-(mesitylamino)-2-oxoethoxy)naphthalen-2-yl) acrylate (I-4f). The reaction was conducted following the General procedure C, using I-3f (0.05 g, 0.16 mmol), methyl acrylate (0.02 ml, 0.15 mmol), Pd(OAc)₂ (0.01 g, 0.01 mmol), tri(o-tolyl)phosphine (0.02 g, 0.02 mmol), and Et₃N (0.30 ml, 0.20 mmol). White solid (0.01 g, 22%). ¹H NMR (600 MHz, DMSO) δ 9.48 (s, 1H), 8.08 (d, J=1.7 Hz, 1H), 7.95-7.75 (m, 4H), 7.39-7.36 (m, 2H), 6.87 (s, 2H), 6.75 (d, J=16.0 Hz, 1H), 4.85 (s, 2H), 3.76 (s, 3H), 2.22 (s, 3H), 2.07 (s, 6H). LC-MS (ESI): m/z=404.2, t_{R}=4.89 min.

Methyl 3-(7-(2-(mesitylamino)-2-oxoethoxy)naphthalen-2-yl)propanoate (I-5f). The reaction was conducted following the general procedure D, using I-4f (0.01 g, 0.03 mmol) and 5% Pd/C (0.03 g). White solid (0.007 g, 61.9%). LC-MS (ESI): m/z=406.2, t_{R}=4.83 min.

3-(7-(2-(Mesitylamino)-2-oxoethoxy)naphthalen-2-yl)propanoic acid (1f, UCAB #910). The reaction was conducted following the general procedure B, using I-5f (0.007, 0.02 mmol) and 1 M aq. NaOH (1 ml). White solid (0.004 g, 59.7%). ¹H NMR (600 MHz, DMSO) δ 9.48 (s, 1H), 7.82 (d, J=8.9 Hz, 1H), 7.77 (d, J=8.3 Hz, 1H), 7.58 (d, J=1.7 Hz, 1H), 7.30-7.23 (m, 3H), 6.87 (s, 2H), 4.82 (s, 2H), 2.97 (t, J=7.6 Hz, 2H), 2.63 (t, J=7.6 Hz, 2H), 2.22 (s, 3H), 2.08 (s, 6H). ¹³C NMR (151 MHz, DMSO) δ 174.21, 166.94, 156.31, 139.65, 136.11, 135.55 (2C), 134.61, 132.25, 129.52, 128.74 (2C), 128.04, 127.87, 125.62, 125.54, 118.57, 107.45, 67.49, 35.54, 30.99, 20.95, 18.45 (2C). LC-MS (ESI): m/z=392.2, t_{R}=4.35 min.

2-((7-Bromonaphthalen-2-yl)oxy)-N-(2,3,5,6-tetramethylphenyl) acetamide (1-3 g). The reaction was conducted following the general procedure E, using I-2 (0.21 g, 0.72 mmol), HATU (0.55 g, 1.44 mmol), DIPEA (0.28 mL, 2.15 mmol) and 2,3,5,6-tetramethylaniline hydrochloride (0.21 g, 0.72 mmol). White solid (0.54 g, 95%). ¹H NMR (400 MHz, DMSO) δ 9.53 (s, 1H), 8.04 (d, J=2.0 Hz, 1H), 7.97-7.77 (m, 2H), 7.48 (dd, J=8.7, 2.0 Hz, 1H), 7.41-7.32 (m, 2H), 6.90 (s, 1H), 4.85 (s, 2H), 4.02 (q, J=7.1 Hz, 1H), 2.16 (s, 6H), 1.97 (s, 6H). LC-MS (ESI): m/z=312.1/414.1, t_{R}=5.27 min.

Methyl (E)-3-(7-(2-oxo-2-((2,3,5,6-tetramethylphenyl)amino)ethoxy)naphthalen-2-yl) acrylate (I-4g). The reaction was conducted following the General procedure C, using I-3g (0.21 g, 0.49 mmol), methyl acrylate (0.07 ml, 0.73 mmol), Pd(OAc)₂ (0.01 g, 0.03 mmol), tri(o-tolyl)phosphine (0.02 g, 0.05 mmol), and Et₃N (0.49 ml, 3.48 mmol). White solid (0.30 g, 97.2%). ¹H NMR (600 MHz, DMSO) δ 9.56 (s, 1H), 8.10-8.07 (m, 1H), 7.94-7.88 (m, 2H), 7.82 (d, J=16.0 Hz, 1H), 7.78 (dd, J=8.6, 1.7 Hz, 1H), 7.41 (d, J=2.6 Hz, 1H), 7.39 (dd, J=8.8, 2.6 Hz, 1H), 6.91 (s, 1H), 6.76 (d, J=16.0 Hz, 1H), 4.88 (s, 2H), 3.76 (s, 3H), 2.17 (s, 6H), 1.98 (s, 6H). LC-MS (ESI): m/z=418.2, t_{R}=5.07 min.

Methyl 3-(7-(2-oxo-2-((2,3,5,6-tetramethylphenyl)amino)ethoxy)naphthalen-2-yl)propanoate (I-5g). The reaction was conducted following the general procedure D, using I-4g (0.30 g, 1.143 mmol) and 5% Pd/C (0.3g). White solid (0.30 g, 98.2%). ¹H NMR (600 MHz, DMSO) δ 9.54 (s, 1H), 7.83 (d, J=8.9 Hz, 1H), 7.78 (d, J=8.3 Hz, 1H), 7.59 (d, J=1.7 Hz, 1H), 7.33-7.23 (m, 3H), 6.91 (s, 1H), 4.85 (s, 2H), 3.59 (s, 3H), 3.01 (t, J=7.6 Hz, 2H), 2.73 (t, J=7.6 Hz, 2H), 2.17 (s, 6H), 1.98 (s, 6H). LC-MS (ESI): m/z=420.3, t_{R}=5.00 min.

3-(7-(2-Oxo-2-((2,3,5,6-tetramethylphenyl)amino)ethoxy)naphthalen-2-yl)propanoic acid (1g, UCAB #997). The reaction was conducted following the general procedure B, using I-5g (0.06, 0.43 mmol) and 1 M aq. NaOH (2 ml). White solid (0.30 g, 96.1%). ¹H NMR (600 MHz, DMSO) δ 9.55 (s, 1H), 7.82 (d, J=8.9 Hz, 1H), 7.77 (d, J=8.3 Hz, 1H), 7.59 (s, 1H), 7.32-7.24 (m, 3H), 6.91 (s, 1H), 4.84 (s, 2H), 2.98 (t, J=7.6 Hz, 2H), 2.63 (t, J=7.6 Hz, 2H), 2.17 (s, 6H), 1.99 (s, 6H). ¹³C NMR (151 MHz, DMSO) δ 174.22, 166.99, 156.36, 139.65, 134.63, 133.65 (2C), 131.58 (2C), 130.16, 129.53, 128.05, 127.88, 125.60, 125.59, 118.58, 107.49, 67.53, 40.41, 35.56, 31.01, 20.20 (2C), 14.76 (2C). LC-MS (ESI): m/z=406.2, t_{R}=4.54 min.

2-((7-bromonaphthalen-2-yl)oxy)-N-phenylacetamide (I-3h). The reaction was conducted following the general procedure E, using I-2 (0.1 g, 0.36 mmol), HATU (0.3 g, 0.71 mmol), DIPEA (0.2 mL, 1.07 mmol) and aniline (0.05 g, 0.53 mmol). White solid (0.1 g, 79.4%). ¹H NMR (400 MHz, DMSO) δ 10.14 (s, 1H), 8.09 (d, J=2.0 Hz, 1H), 7.95-7.89 (m, 1H), 7.84 (d, J=8.7 Hz, 1H), 7.70-7.62 (m, 2H), 7.49 (dd, J=8.7, 2.0 Hz, 1H), 7.35 (qd, J=7.3, 2.1 Hz, 4H), 7.14-7.05 (m, 1H), 4.83 (s, 2H). LC-MS (ESI): m/z=356.1/358.1, t_{R}=4.85 min.

Methyl (E)-3-(7-(2-oxo-3-phenylpropoxy)naphthalen-2-yl) acrylate (1-4h). The reaction was conducted following the General procedure C, using I-3h (0.1 g, 0.28 mmol), methyl acrylate (0.04 ml, 0.42 mmol), Pd(OAc)₂ (0.03 g, 0.02 mmol), tri(o-tolyl)phosphine (0.08 g, 0.03 mmol), and Et₃N (0.3 ml, 2.02 mmol). White solid (0.07 g, 70%). ¹H NMR (400 MHz, DMSO) δ 10.14 (s, 1H), 8.10 (s, 1H), 7.89 (t, J=8.5 Hz, 2H), 7.83-7.72 (m, 2H), 7.66 (d, J=8.0 Hz, 2H), 7.44-7.22 (m, 5H), 7.08 (t, J=7.4 Hz, 1H), 6.74 (d, J=16.0 Hz, 1H), 4.84 (s, 2H), 3.74 (s, 3H). LC-MS (ESI): m/z=361.1, t_{R}=5.25 min.

Methyl 3-(7-(2-oxo-3-phenylpropoxy)naphthalen-2-yl)propanoate (I-5h). The reaction was conducted following the general procedure D, using I-4h (0.07 g, 0.19 mmol) and 5% Pd/C (0.1g). White solid (0.07 g, 98.2%). ¹H NMR (600 MHz, DMSO) δ 10.13 (s, 1H), 7.85-7.80 (m, 1H), 7.77 (d, J=8.3 Hz, 1H), 7.69-7.64 (m, 2H), 7.60 (s, 1H), 7.37-7.30 (m, 2H), 7.27 (ddq, J=9.7, 4.1, 2.3 Hz, 4H), 7.08 (t, J=7.4 Hz, 1H) 4.81 (s, 2H), 3.58 (s, 3H), 2.99 (t, J=7.6 Hz, 2H), 2.72 (t, J=7.6 Hz, 2H). LC-MS (ESI): m/z=363.2, t_{R}=5.11 min.

3-(7-(2-oxo-2-(phenylamino)ethoxy)naphthalen-2-yl)propanoic acid (1h, UCAB #1233). The reaction was conducted following the general procedure B, using 1-5h (0.07 g, 0.19 mmol) and 1 M aq. NaOH (1 ml). White solid (0.01 g, 14.9%). ¹H NMR (600 MHz, DMSO) δ 12.11 (s, 1H), 10.12 (s, 1H), 7.82 (d, J=9.6 Hz, 1H), 7.77 (d, J=8.4 Hz, 1H), 7.69-7.64 (m, 2H), 7.60 (s, 1H), 7.36-7.30 (m, 2H), 7.29-7.22 (m, 3H), 7.09 (t, J=7.4 Hz, 1H), 4.81 (s, 2H), 2.96 (t, J=7.6 Hz, 2H), 2.62 (t, J=7.6 Hz, 2H). ¹³C NMR (151 MHz, DMSO) δ 173.44, 166.13, 155.57, 138.87, 138.10, 133.87, 128.80, 128.43 (2C), 127.25, 127.09, 124.92, 124.84, 123.41, 119.46 (2C), 117.65, 106.63, 66.95, 34.79, 30.23. LC-MS (ESI): m/z=350.1, t_{R}=4.15 min.

N-Cycloheptyl-2-((7-(4,4,5,5-tetramethyl-1,3,2-dioxaborolan-2-yl)naphthalen-2-yl)oxy) acetamide (1-6). A solution of I-3d (0.30 g, 0.83 mmol), PinB-BPin (0.26 g, 0.99 mmol), KOAc (0.25 g, 2.48 mmol), and Pd(dppf)Cl₂ (0.02 g, 0.025 mmol) were added in dioxane (3 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.70 g, 90%). ¹H NMR (400 MHz, DMSO) δ 8.17 (d, J=1.1 Hz, 1H), 7.98 (d, J=8.1 Hz, 1H), 7.84 (dd, J=15.4, 8.6 Hz, 2H), 7.57 (dd, J=8.1, 1.2 Hz, 1H), 7.38 (d, J=2.6 Hz, 1H), 7.32 (dd, J=8.9, 2.6 Hz, 1H), 4.55 (s, 2H), 3.83 (dh, J=13.5, 4.5 Hz, 1H), 1.77 (ddt, J=14.8, 7.4, 3.3 Hz, 2H), 1.63-1.40 (m, 5H), 1.33 (s, 12H). LC-MS (ESI): m/z=424.3, t_{R}=5.59 min.

Methyl 3-(7-(2-(cycloheptylamino)-2-oxoethoxy)naphthalen-2-yl)-3-phenylpropanoate (I-7a). The reaction was conducted following the general procedure F, using I-6 (0.29, 0.71 mmol) and methyl cinnamate (I-9a, 0.08 g, 0.48 mmol), TEA (0.22 ml, 1.42 mmol), and [Rh(COD)Cl]₂ (24 mg, 0.05 mmol). White solid (0.16 g, 51.7%). ¹H NMR (600 MHz, DMSO) δ 7.95 (d, J=8.1 Hz, 1H), 7.78-7.74 (m, 1H), 7.72 (d, J=8.4 Hz, 1H), 7.67 (d, J=1.7 Hz, 1H), 7.38-7.33 (m, 2H), 7.32-7.25 (m, 3H), 7.20-7.13 (m, 3H), 4.58 (t, J=8.0 Hz, 1H), 4.53 (s, 2H), 3.91 (s, 0H), 3.87-3.78 (m, 1H), 3.49 (s, 3H), 3.22 (qd, J=15.9, 8.0 Hz, 2H), 1.75 (ddt, J=14.0, 7.3, 2.5 Hz, 2H), 1.63-1.32 (m, 10H). LC-MS (ESI): m/z=460.4, t_{R}=5.19 min.

3-(7-(2-(Cycloheptylamino)-2-oxoethoxy)naphthalen-2-yl)-3-phenylpropanoic acid (2a, UCAB #944). The reaction was conducted following the general procedure G, using I-7a (0.16, 0.38 mmol) and 2 M aq. NaOH (380 μl). White solid (0.035 g, 22.0%). ¹H NMR (600 MHz, DMSO) δ 12.12 (s, 1H), 7.96 (d, J=8.1 Hz, 1H), 7.79-7.75 (m, 1H), 7.73 (d, J=8.5 Hz, 1H), 7.68 (d, J=1.8 Hz, 1H), 7.38-7.33 (m, 2H), 7.33-7.25 (m, 3H), 7.21-7.14 (m, 3H), 4.55 (d, J=9.2 Hz, 3H), 3.83 (dh, J=13.5, 4.5 Hz, 1H), 3.11 (qd, J=15.9, 8.0 Hz, 2H), 1.77 (dddd, J=14.4, 7.2, 4.9, 2.4 Hz, 2H), 1.65-1.35 (m, 9H), 1.25 (d, J=5.0 Hz, 1H). ¹³C NMR (151 MHz, DMSO) δ 173.16, 166.54, 156.39, 144.46, 142.76, 134.58, 129.40, 128.90, 128.88 (2C), 128.14, 128.08 (2C), 127.84, 126.75, 124.87, 118.74, 107.62, 67.53, 50.07, 47.21, 40.55, 34.68 (2C), 28.16 (2C), 24.22 (2C). LC-MS (ESI): m/z=446.4, t_{R}=4.689 min.

Methyl (E)-3-(4-hydroxyphenyl) acrylate (I-9b). The reaction was conducted following the General procedure C, using 4-bromophenol (0.5 g, 2.89 mmol), methyl acrylate (0.4 ml, 04.34 mmol), Pd(OAc)₂ (0.03 g, 0.14 mmol), tri(o-tolyl)phosphine (0.09 g, 0.29 mmol), and Et₃N (2.9 ml, 20.72 mmol). White solid (0.51 g, 97.1%). ¹H NMR (600 MHz, DMSO) δ 10.00 (s, 1H), 7.58-7.53 (m, 3H), 6.82-6.77 (m, 2H), 6.40 (d, J=16.0 Hz, 1H), 3.69 (s, 3H). LC-MS (ESI): m/z=179.1, t_{R}=3.74 min.

Methyl 3-(7-(2-(cycloheptylamino)-2-oxoethoxy)naphthalen-2-yl)-3-(4-hydroxyphenyl)propanoate (I-7b). The reaction was conducted following the general procedure F, using I-6 (0.3, 0.74 mmol) and I-9b (0.09 g, 0.49 mmol), TEA (V; 0.21 ml, 1.47 mmol), and [Rh(COD)Cl]₂ (25 mg, 0.05 mmol). Colorless oil (0.16 g, 47.6%). ¹H NMR (600 MHz, DMSO) δ 9.20 (s, 1H), 7.95 (d, J=8.1 Hz, 1H), 7.77-7.73 (m, 1H), 7.70 (d, J=8.5 Hz, 1H), 7.61 (d, J=1.7 Hz, 1H), 7.28-7.24 (m, 1H), 7.19-7.14 (m, 2H), 7.14-7.09 (m, 2H), 6.67-6.62 (m, 2H), 4.53 (s, 2H), 4.46 (t, J=8.0 Hz, 1H), 3.82 (dh, J=13.5, 4.4 Hz, 1H), 3.48 (s, 3H), 3.16 (dd, J=15.8, 8.2 Hz, 1H), 3.09 (dd, J=15.8, 7.8 Hz, 1H), 1.76 (dd, J=14.4, 7.6 Hz, 3H), 1.63-1.29 (m, 9H). LC-MS (ESI): m/z=476.3, t_{R}=4.87 min.

3-(7-(2-(Cycloheptylamino)-2-oxoethoxy)naphthalen-2-yl)-3-(4-hydroxyphenyl)propanoic acid (2b, UCAB #964). The reaction was conducted following the general procedure G, using I-7b (0.16, 0.35 mmol) and 2 M aq. NaOH (140 μl). White solid (0.039 g, 25.1%). ¹H NMR (400 MHz, DMSO) δ 12.07 (s, 1H), 9.18 (s, 1H), 7.96 (d, J=8.1 Hz, 1H), 7.79-7.68 (m, 2H), 7.62 (d, J=1.7 Hz, 1H), 7.27 (dd, J=8.4, 1.7 Hz, 1H), 7.21-7.08 (m, 4H), 6.70-6.62 (m, 2H), 4.54 (s, 2H), 4.45 (t, J=7.9 Hz, 1H), 3.84 (dp, J=13.2, 4.5 Hz, 1H), 3.02 (qd, J=15.7, 8.0 Hz, 2H), 1.77 (dq, J=13.8, 3.8 Hz, 2H), 1.67-1.15 (m, 10H). ¹³C NMR (101 MHz, DMSO) δ 173.25, 166.55, 156.34, 156.19, 143.36, 134.65, 134.57, 129.36, 128.96, 128.03 (2C), 127.76, 124.91, 124.62, 118.61, 115.58 (2C), 107.62, 67.53, 50.06, 46.45, 40.67, 34.68 (2C), 28.16 (2C), 24.22 (2C). LC-MS (ESI): m/z=462.3, t_{R}=4.47 min.

Methyl (E)-3-(4-chlorophenyl) acrylate (I-9c). The reaction was conducted following the General procedure C, using 1-bromo-4-chlorobenzene (0.5 g, 2.62 mmol), methyl acrylate (0.36 ml, 3.92 mmol), Pd(OAc)₂ (0.03 g, 0.13 mmol), tri(o-tolyl)phosphine (0.08 g, 0.27 mmol), and Et₃N (2.7 ml, 18.73 mmol). White solid (0.41 g, 79.2%). ¹H NMR (400 MHz, DMSO) δ 7.81-7.73 (m, 2H), 7.66 (d, J=16.1 Hz, 1H), 7.53-7.45 (m, 2H), 6.68 (d, J=16.1 Hz, 1H), 3.73 (s, 3H). LC-MS (ESI): m/z=197.0, t_{R}=4.82 min.

Methyl 3-(4-chlorophenyl)-3-(7-(2-(cycloheptylamino)-2-oxoethoxy)naphthalen-2-yl)propanoate (I-7c). The reaction was conducted following the general procedure F, using I-6 (0.25, 0.61 mmol) and I-9c (0.08 g, 0.41 mmol), TEA (V; 0.18 ml, 1.23 mmol), and [Rh(COD)Cl]₂ (25 mg, 0.05 mmol). Colorless oil (0.21 g, 69.2%). ¹H NMR (600 MHz, DMSO) δ 7.96 (d, J=8.1 Hz, 1H), 7.77 (d, J=8.8 Hz, 1H), 7.74 (d, J=8.5 Hz, 1H), 7.67 (d, J=1.7 Hz, 1H), 7.43-7.37 (m, 2H), 7.36-7.29 (m, 3H), 7.22-7.16 (m, 2H), 4.61 (t, J=7.9 Hz, 1H), 4.55 (s, 2H), 3.83 (ddq, J=13.5, 9.1, 4.4 Hz, 1H), 3.50 (s, 3H), 3.29-3.18 (m, 2H), 1.81-1.72 (m, 2H), 1.67-1.34 (m, 10H). LC-MS (ESI): m/z=494.3, t_{R}=5.48 min.

3-(4-Chlorophenyl)-3-(7-(2-(cycloheptylamino)-2-oxoethoxy)naphthalen-2-yl)propanoic acid (2c, UCAB #965). The reaction was conducted following the general procedure G, using I-7c (0.21, 0.43 mmol) and 2 M aq. NaOH (430 μl). White solid (0.025 g, 12.3%). ¹H NMR (600 MHz, DMSO) δ 12.16 (s, 1H), 7.96 (d, J=8.1 Hz, 1H), 7.79-7.72 (m, 2H), 7.66 (d, J=1.7 Hz, 1H), 7.41-7.29 (m, 5H), 7.19 (d, J=8.5 Hz, 2H), 4.57 (t, J=8.0 Hz, 1H), 4.54 (s, 2H), 3.87-3.79 (m, 1H), 3.16-3.05 (m, 2H), 1.80-1.72 (m, 2H), 1.63-1.13 (m, 10H) ¹³C NMR (151 MHz, DMSO) δ 173.03, 166.51, 156.43, 143.49, 142.31, 134.58, 131.38, 130.02 (2C), 129.41, 128.79 (2C), 128.26, 127.88, 124.91, 124.75, 118.85, 107.63, 67.53, 50.06, 46.49, 40.50, 34.69 (2C), 28.16 (2C), 24.23 (2C). LC-MS (ESI): m/z=480.2, t_{R}=5.10 min.

Methyl 3-(7-(2-(cycloheptylamino)-2-oxoethoxy)naphthalen-2-yl)-3-(4-methoxyphenyl)propanoate (I-7d). The reaction was conducted following the general procedure F, using I-6 (0.31, 0.76 mmol) and methyl (E)-3-(4-methoxyphenyl) acrylate (I-9d, 0.10 g, 0.51 mmol), TEA (0.23 ml, 1.52 mmol), and [Rh(COD)Cl]₂ (24 mg, 0.05 mmol). White solid (0.14 g, 39.7%). ¹H NMR (400 MHz, DMSO) δ 7.96 (d, J=8.1 Hz, 1H), 7.80-7.69 (m, 2H), 7.67-7.62 (m, 1H), 7.32-7.22 (m, 3H), 7.22-7.15 (m, 2H), 6.88-6.79 (m, 2H), 4.54 (d, J=5.0 Hz, 3H), 3.84 (qd, J=9.0, 8.4, 3.9 Hz, 1H), 3.70 (s, 3H), 3.50 (s, 3H), 3.26-3.09 (m, 2H), 1.77 (dt, J=14.4, 5.2 Hz, 2H), 1.67-1.33 (m, 10H). LC-MS (ESI): m/z=490.3, t_{R}=5.23 min.

3-(7-(2-(Cycloheptylamino)-2-oxoethoxy)naphthalen-2-yl)-3-(4-methoxyphenyl)propanoic acid (2d, UCAB #945). The reaction was conducted following the general procedure G, using 25c (0.14, 0.31 mmol) and 2 M aq. NaOH (310 μl). White solid (0.07 g, 50.7%). ¹H NMR (600 MHz, DMSO) δ 12.09 (s, 1H), 7.96 (d, J=8.1 Hz, 1H), 7.80-7.69 (m, 2H), 7.64 (d, J=1.7 Hz, 1H), 7.29-7.23 (m, 3H), 7.21-7.16 (m, 2H), 6.87-6.81 (m, 2H), 4.54 (s, 2H), 4.50 (t, J=8.0 Hz, 1H), 3.88-3.79 (m, 1H), 3.70 (s, 3H), 3.13-2.99 (m, 2H), 1.77 (dddd, J=13.4, 6.6, 4.3, 2.2 Hz, 2H), 1.66-1.35 (m, 10H). ¹³C NMR (151 MHz, DMSO) δ 173.20, 166.53, 158.16, 156.37, 143.15, 136.42, 134.58, 129.38, 129.06 (2C), 128.09, 127.79, 124.87, 124.68, 118.67, 114.26 (2C), 107.61, 67.53, 55.46, 50.05, 46.40, 40.49, 34.68 (2C), 28.16 (2C), 24.22 (2C). LC-MS (ESI): m/z=476.3, t_{R}=4.81 min

Methyl (E)-3-(4-ethoxyphenyl) acrylate (I-9e). The reaction was conducted following the General procedure C, using 1-bromo-4-ethoxybenzene (0.5 g, 2.49 mmol), methyl acrylate (0.40 ml, 3.73 mmol), Pd(OAc)₂ (0.03 g, 0.12 mmol), tri(o-tolyl)phosphine (0.08 g, 0.24 mmol), and Et₃N (2.5 ml, 17.83 mmol). White solid (0.51 g, 99.6%). ¹H NMR (400 MHz, DMSO) δ 7.70-7.57 (m, 3H), 6.99-6.91 (m, 2H), 6.47 (d, J=16.0 Hz, 1H), 4.07 (q, J=7.0 Hz, 2H), 3.70 (s, 3H), 1.33 (t, J=7.0 Hz, 3H). LC-MS (ESI): m/z=207.1, t_{R}=4.71 min.

Methyl 3-(7-(2-(cycloheptylamino)-2-oxoethoxy)naphthalen-2-yl)-3-(4-ethoxyphenyl)propanoate (I-7e). The reaction was conducted following the general procedure F, using I-6 (0.30, 0.73 mmol) and I-9e (0.10 g, 0.49 mmol), TEA (0.22 ml, 1.47 mmol), and [Rh(COD)Cl]₂ (25 mg, 0.05 mmol). Colorless oil (0.16 g, 45.8%). ¹H NMR (600 MHz, DMSO) δ 7.96 (d, J=8.1 Hz, 1H), 7.79-7.74 (m, 1H), 7.72 (d, J=8.5 Hz, 1H), 7.64 (d, J=1.8 Hz, 1H), 7.29 (dd, J=8.5, 1.8 Hz, 1H), 7.27-7.22 (m, 2H), 7.18 (d, J=7.9 Hz, 2H), 6.85-6.79 (m, 2H), 4.53 (d, J=12.1 Hz, 3H), 3.99-3.91 (m, 2H), 3.88-3.79 (m, 1H), 3.49 (s, 3H), 3.24-3.11 (m, 2H), 1.81-1.73 (m, 2H), 1.65-1.34 (m, 10H), 1.28 (t, J=7.0 Hz, 3H). LC-MS (ESI): m/z=504.3, t_{R}=5.39 min.

3-(7-(2-(Cycloheptylamino)-2-oxoethoxy)naphthalen-2-yl)-3-(4-ethoxyphenyl)propanoic acid (2e, UCAB #953). The reaction was conducted following the general procedure G, using I-7e (0.16, 0.38 mmol) and 2 M aq. NaOH (380 μl). White solid (0.07 g, 44.7%). ¹H NMR (600 MHz, DMSO) δ 12.10 (s, 1H), 7.96 (d, J=8.1 Hz, 1H), 7.76 (d, J=9.6 Hz, 1H), 7.71 (d, J=8.5 Hz, 1H), 7.63 (s, 1H), 7.28 (dd, J=8.5, 1.7 Hz, 1H), 7.23 (d, J=8.5 Hz, 2H), 7.18 (dd, J=6.4, 2.7 Hz, 2H), 6.81 (d, J=8.3 Hz, 2H), 4.54 (s, 2H), 4.49 (t, J=8.0 Hz, 1H), 3.95 (q, J=7.0 Hz, 2H), 3.83 (qt, J=9.0, 4.4 Hz, 1H), 3.12-2.99 (m, 2H), 1.76 (ddt, J=14.6, 7.1, 3.6 Hz, 2H), 1.65-1.44 (m, 8H), 1.44-1.34 (m, 2H), 1.28 (t, J=7.0 Hz, 3H). ¹³C NMR (151 MHz, DMSO) δ 173.21, 166.54, 157.43, 156.36, 143.16, 136.28, 134.58, 129.38, 129.05 (2C), 128.09, 127.79, 124.88, 124.68, 118.67, 114.72 (2C), 107.61, 67.53, 63.35, 50.06, 46.40, 40.47, 34.68 (2C), 28.16 (2C), 24.22 (2C), 15.14. LC-MS (ESI): m/z=490.3, t_{R}=4.98 min.

Methyl (E)-3-(4-isopropoxyphenyl) acrylate (I-9f). The reaction was conducted following the General procedure C, using 1-bromo-4-isopropoxybenzene (0.5 g, 2.33 mmol), methyl acrylate (0.32 ml, 3.49 mmol), Pd(OAc)₂ (0.03 g, 0.12 mmol), tri(o-tolyl)phosphine (0.08 g, 0.24 mmol), and Et₃N (2.4 ml, 16.67 mmol). White solid (0.45 g, 87.9%). ¹H NMR (600 MHz, DMSO) δ 7.66-7.63 (m, 2H), 7.60 (d, J=16.0 Hz, 1H), 6.96-6.93 (m, 2H), 6.46 (dd, J=16.0, 0.8 Hz, 1H), 4.68 (hept, J=6.0 Hz, 1H), 3.70 (d, J=0.7 Hz, 3H), 1.27 (dd, J=6.0, 0.8 Hz, 6H). LC-MS (ESI): m/z=221.1, t_{R}=4.94 min.

Methyl 3-(7-(2-(cycloheptylamino)-2-oxoethoxy)naphthalen-2-yl)-3-(4-isopropoxyphenyl)propanoate (I-7f). The reaction was conducted following the general procedure F, using I-6 (0.30, 0.74 mmol) and I-9f (0.11 ml, 0.49 mmol), TEA (0.21 ml, 1.47 mmol), and [Rh(COD)Cl]₂ (25 mg, 0.05 mmol). Colorless oil (0.17 g, 66.1%). ¹H NMR (600 MHz, DMSO) δ 7.96 (d, J=8.1 Hz, 1H), 7.79-7.74 (m, 1H), 7.72 (d, J=8.4 Hz, 1H), 7.67-7.63 (m, 1H), 7.29 (dd, J=8.5, 1.8 Hz, 1H), 7.25-7.21 (m, 2H), 7.21-7.17 (m, 2H), 6.83-6.78 (m, 2H), 4.55 (s, 2H), 4.51 (dd, J=6.9, 5.4 Hz, 2H), 3.83 (ddq, J=13.6, 9.1, 4.5 Hz, 1H), 3.49 (s, 3H), 3.24-3.11 (m, 2H), 1.80-1.73 (m, 2H), 1.66-1.34 (m, 10H), 1.22 (d, J=6.0 Hz, 6H). LC-MS (ESI): m/z=518.3, t_{R}=5.53 min.

3-(7-(2-(Cycloheptylamino)-2-oxoethoxy)naphthalen-2-yl)-3-(4-isopropoxyphenyl)propanoic acid (2f, UCAB #966). The reaction was conducted following the general procedure G, using I-7f (0.15, 0.30 mmol) and 2 M aq. NaOH (300 μl). White solid (0.02 g, 10.6%). ¹H NMR (400 MHz, DMSO) δ 12.08 (s, 1H), 7.95 (d, J=8.1 Hz, 1H), 7.79-7.68 (m, 2H), 7.64 (d, J=1.7 Hz, 1H), 7.28 (dd, J=8.4, 1.7 Hz, 1H), 7.25-7.14 (m, 4H), 6.83-6.75 (m, 2H), 4.60-4.38 (m, 4H), 3.82 (qt, J=9.0, 4.4 Hz, 1H), 3.13-2.96 (m, 2H), 1.76 (ddd, J=12.9, 7.5, 4.0 Hz, 2H), 1.66-1.32 (m, 10H), 1.21 (d, J=6.0 Hz, 6H). ¹³C NMR (151 MHz, DMSO) δ 173.22, 166.53, 156.37, 156.35, 143.16, 136.17, 134.59, 129.38 (2C), 129.07, 128.10, 127.80, 124.89, 124.69, 118.67, 115.84 (2C), 107.61, 69.46, 67.54, 50.06, 46.41, 40.55, 34.69 (2C), 28.16 (2C), 24.23 (2C), 22.34 (2C). LC-MS (ESI): m/z=504.3, t_{R}=4.13 min

Methyl (E)-3-(3-hydroxyphenyl) acrylate (1-9g). The reaction was conducted following the General procedure C, using 3-bromophenol (0.5 g, 2.89 mmol), methyl acrylate (0.40 ml, 4.34 mmol), Pd(OAc)₂ (0.03 g, 0.14 mmol), tri(o-tolyl)phosphine (0.09 g, 0.29 mmol), and Et₃N (2.9 ml, 20.72 mmol). White solid (0.48 g, 91.4%). ¹H NMR (600 MHz, DMSO) δ 9.60 (s, 1H), 7.56 (d, J=16.0 Hz, 1H), 7.21 (t, J=7.8 Hz, 1H), 7.13 (dt, J=7.6, 1.3 Hz, 1H), 7.03 (t, J=2.0 Hz, 1H), 6.83 (ddd, J=8.0, 2.4, 1.0 Hz, 1H), 6.51 (d, J=16.0 Hz, 1H), 3.71 (s, 3H). LC-MS (ESI): m/z=179.1, t_{R}=3.82 min.

Methyl 3-(7-(2-(cycloheptylamino)-2-oxoethoxy)naphthalen-2-yl)-3-(3-hydroxyphenyl)propanoate (I-7g). The reaction was conducted following the general procedure F, using I-6 (0.30, 0.74 mmol) and I-9g (0.09 ml, 0.49 mmol), TEA (0.21 ml, 1.47 mmol), and [Rh(COD)Cl]₂ (25 mg, 0.05 mmol). White solid (0.15 g, 44.1%). ¹H NMR (600 MHz, DMSO) δ 9.26 (s, 1H), 7.95 (d, J=8.2 Hz, 1H), 7.78-7.74 (m, 1H), 7.72 (d, J=8.5 Hz, 1H), 7.64 (d, J=1.8 Hz, 1H), 7.27 (dd, J=8.5, 1.8 Hz, 1H), 7.20-7.16 (m, 2H), 7.05 (t, J=7.9 Hz, 1H), 6.76 (dt, J=7.7, 1.3 Hz, 1H), 6.67 (t, J=2.1 Hz, 1H), 6.55 (ddd, J=8.1, 2.4, 1.0 Hz, 1H), 4.54 (s, 2H), 4.47 (d, J=8.0 Hz, 1H), 3.82 (ddp, J=12.6, 9.0, 4.4 Hz, 1H), 3.49 (s, 3H), 3.15 (qd, J=15.9, 8.0 Hz, 2H), 1.79-1.72 (m, 2H), 1.62-1.31 (m, 10H). LC-MS (ESI): m/z=476.3, t_{R}=5.87 min.

3-(7-(2-(Cycloheptylamino)-2-oxoethoxy)naphthalen-2-yl)-3-(3-hydroxyphenyl)propanoic acid (2g, UCAB #962). The reaction was conducted following the general procedure G, using I-7g (0.15, 0.33 mmol) and 2 M aq. NaOH (330 μl). White solid (0.08 g, 53.8%). ¹H NMR (600 MHz, DMSO) δ 12.11 (s, 1H), 9.26 (s, 1H), 7.97 (d, J=8.1 Hz, 1H), 7.77 (d, J=8.7 Hz, 1H), 7.73 (d, J=8.4 Hz, 1H), 7.65 (d, J=1.7 Hz, 1H), 7.27 (dd, J=8.5, 1.8 Hz, 1H), 7.22-7.17 (m, 2H), 7.06 (t, J=7.9 Hz, 1H), 6.78 (dt, J=7.8, 1.2 Hz, 1H), 6.69 (t, J=2.0 Hz, 1H), 6.56 (ddd, J=8.0, 2.5, 1.0 Hz, 1H), 4.55 (s, 2H), 4.46 (t, J=7.9 Hz, 1H), 3.84 (ddt, J=9.3, 8.1, 4.6 Hz, 1H), 3.10-2.98 (m, 2H), 1.81-1.73 (m, 2H), 1.67-1.35 (m, 10H). ¹³C NMR (151 MHz, DMSO) δ 173.15, 166.53, 157.79, 156.38, 145.83, 142.73, 134.57, 129.79, 129.39, 128.09, 127.84, 124.92, 124.81, 118.71, 118.62, 115.07, 113.72, 107.62, 67.53, 50.06, 47.08, 40.49, 34.69 (2C), 28.16 (2C), 24.22 (2C). LC-MS (ESI): m/z=462.3, t_{R}=4.52 min

Methyl (E)-3-(3-methoxyphenyl) acrylate (1-9h). The reaction was conducted following the General procedure C, using 1-bromo-3-methoxybenzene (0.5 g, 2.67 mmol), methyl acrylate (0.37 ml, 4.01 mmol), Pd(OAc)₂ (0.03 g, 0.13 mmol), tri(o-tolyl)phosphine (0.08 g, 0.28 mmol), and Et₃N (2.7 ml, 19.17 mmol). Yellow oil (0.50 g, 97.5%). ¹H NMR (600 MHz, DMSO) δ 7.63 (d, J=16.0 Hz, 1H), 7.36-7.26 (m, 3H), 7.00 (ddd, J=8.1, 2.6, 1.0 Hz, 1H), 6.67 (d, J=16.0 Hz, 1H), 3.79 (s, 3H), 3.73 (s, 3H). LC-MS (ESI): m/z=193.1, t_{R}=4.52 min.

Methyl 3-(7-(2-(cycloheptylamino)-2-oxoethoxy)naphthalen-2-yl)-3-(3-methoxyphenyl)propanoate (I-7h). The reaction was conducted following the general procedure F, using I-6 (0.30, 0.73 mmol) and I-9h (0.08 ml, 0.49 mmol), TEA (0.21 ml, 1.47 mmol), and [Rh(COD)Cl]₂ (25 mg, 0.05 mmol). Colorless oil (0.17 g, 48.9%). ¹H NMR (600 MHz, DMSO) δ 7.95 (d, J=8.1 Hz, 1H), 7.78-7.74 (m, 1H), 7.72 (d, J=8.5 Hz, 1H), 7.67 (d, J=1.7 Hz, 1H), 7.32 (dd, J=8.5, 1.8 Hz, 1H), 7.18 (dt, J=6.9, 4.4 Hz, 3H), 6.91 (dd, J=7.4, 1.4 Hz, 2H), 6.74 (dt, J=8.2, 1.3 Hz, 1H), 4.54 (d, J=6.0 Hz, 3H), 3.82 (dh, J=13.6, 4.4 Hz, 1H), 3.70 (s, 3H), 3.49 (s, 3H), 3.20 (qd, J=16.0, 8.0 Hz, 2H), 1.75 (dddd, J=14.2, 7.0, 4.7, 2.3 Hz, 2H), 1.65-1.35 (m, 10H). LC-MS (ESI): m/z=490.3, t_{R}=5.27 min. 3-(7-(2-(Cycloheptylamino)-2-oxoethoxy)naphthalen-2-yl)-3-(3-methoxyphenyl)propanoic acid (2h, UCAB #961). The reaction was conducted following the general procedure G, using I-7h (0.17, 0.36 mmol) and 2 M aq. NaOH (360 μl). White solid (0.13 g, 61.4%). ¹H NMR (600 MHz, DMSO) δ 12.11 (s, 1H), 7.95 (d, J=8.1 Hz, 1H), 7.78-7.70 (m, 2H), 7.67 (d, J=1.7 Hz, 1H), 7.31 (dd, J=8.4, 1.8 Hz, 1H), 7.18 (dd, J=7.7, 3.1 Hz, 3H), 6.91 (dd, J=5.0, 3.1 Hz, 2H), 6.76-6.71 (m, 1H), 4.53 (d, J=11.0 Hz, 3H), 3.83 (dh, J=13.5, 4.4 Hz, 1H), 3.70 (s, 3H), 3.08 (qd, J=15.9, 7.9 Hz, 2H), 1.80-1.72 (m, 2H), 1.65-1.17 (m, 10H). ¹³C NMR (151 MHz, DMSO) δ 173.15, 166.53, 159.75, 156.37, 146.03, 142.64, 134.57, 129.90, 129.39, 128.10, 127.85, 124.87 (2C), 120.31, 118.74, 114.17, 111.77, 107.62, 67.54, 55.43, 50.05, 47.16, 40.55, 34.68 (2C), 28.16 (2C), 24.22 (2C). LC-MS (ESI): m/z=476.3, t_{R}=4.86 min.

Methyl (E)-3-(3-chlorophenyl) acrylate (I-9i). The reaction was conducted following the General procedure C, using 1-bromo-3-chlorobenzene (0.5 g, 2.62 mmol), methyl acrylate (0.36 ml, 3.91 mmol), Pd(OAc)₂ (0.03 g, 0.13 mmol), tri(o-tolyl)phosphine (0.08 g, 0.27 mmol), and Et₃N (2.7 ml, 18.73 mmol). Light yellow oil (0.49 g, 95.5%). ¹H NMR (400 MHz, DMSO) δ 7.83 (t, J=1.8 Hz, 1H), 7.73-7.60 (m, 2H), 7.50-7.39 (m, 2H), 6.72 (d, J=16.1 Hz, 1H), 3.73 (s, 3H). LC-MS (ESI): m/z=197.1, t_{R}=4.82 min.

Methyl 3-(3-chlorophenyl)-3-(7-(2-(cycloheptylamino)-2-oxoethoxy)naphthalen-2-yl)propanoate (I-7i). The reaction was conducted following the general procedure F, using I-6 (0.25, 0.61 mmol) and I-9i (0.07 ml, 0.41 mmol), TEA (0.18 ml, 1.23 mmol), and [Rh(COD)Cl]₂ (21 mg, 0.04 mmol). Light yellow oil (0.21 g, 72.0%). ¹H NMR (600 MHz, DMSO) δ 7.96 (d, J=8.1 Hz, 1H), 7.76 (dd, J=17.2, 8.6 Hz, 2H), 7.71 (d, J=1.8 Hz, 1H), 7.45 (t, J=2.0 Hz, 1H), 7.38-7.28 (m, 3H), 7.26-7.16 (m, 3H), 4.61 (t, J=8.0 Hz, 1H), 4.55 (s, 2H), 3.83 (dh, J=13.5, 4.4 Hz, 1H), 3.50 (s, 3H), 3.26 (dd, J=8.0, 1.3 Hz, 2H), 1.80-1.72 (m, 2H), 1.64-1.36 (m, 10H). LC-MS (ESI): m/z=494.3, t_{R}=5.47 min.

3-(3-Chlorophenyl)-3-(7-(2-(cycloheptylamino)-2-oxoethoxy)naphthalen-2-yl)propanoic acid (2i, UCAB #963). The reaction was conducted following the general procedure G, using I-7i (0.21, 0.43 mmol) and 2 M aq. NaOH (240 μl). White solid (0.035 g, 17.2%) ¹H NMR (600 MHz, DMSO) δ 7.97 (d, J=8.1 Hz, 1H), 7.80-7.73 (m, 2H), 7.71 (d, J=1.8 Hz, 1H), 7.44 (t, J=1.9 Hz, 1H), 7.36-7.29 (m, 2H), 7.20 (d, J=7.4 Hz, 2H), 4.59 (t, J=8.0 Hz, 1H), 4.55 (s, 2H), 3.88-3.79 (m, 1H), 3.14 (d, J=8.0 Hz, 2H), 1.77 (ddq, J=14.0, 7.0, 2.2 Hz, 2H), 1.66-1.20 (m, 10H). ¹³C NMR (151 MHz, DMSO) o 173.02, 166.52, 156.44, 147.09, 142.11, 134.59, 133.50, 130.75, 129.43, 128.29, 127.98, 127.92, 126.85, 126.77, 124.98, 124.74, 118.89, 107.64, 67.54, 50.07, 46.79, 40.49, 34.68 (2C), 28.15 (2C), 24.23 (2C). LC-MS (ESI): m/z=480.2, t_{R}=5.10 min.

Methyl (E)-3-(4-methoxy-3-methylphenyl) acrylate (I-9j). The reaction was conducted following the General procedure C, using 4-bromo-1-methoxy-2-methylbenzene (0.5 g, 2.49 mmol), methyl acrylate (0.34 ml, 3.74 mmol), Pd(OAc)₂ (0.03 g, 0.13 mmol), tri(o-tolyl)phosphine (0.08 g, 0.25 mmol), and Et₃N (2.5 ml, 17.84 mmol). Light yellow oil (0.45 g, 87.9%). ¹H NMR (600 MHz, DMSO) δ 7.60-7.50 (m, 3H), 6.97 (d, J=8.4 Hz, 1H), 6.46 (d, J=16.0 Hz, 1H), 3.83 (s, 3H), 3.70 (s, 3H), 2.16 (s, 3H). LC-MS (ESI): m/z=207.1, t_{R}=4.84 min.

Methyl 3-(7-(2-(cycloheptylamino)-2-oxoethoxy)naphthalen-2-yl)-3-(4-methoxy-3-methylphenyl)propanoate (I-7j). The reaction was conducted following the general procedure F, using I-6 (0.30, 0.74 mmol) and I-9j (0.11 g, 0.49 mmol), TEA (0.21 ml, 1.47 mmol), and [Rh(COD)Cl]₂ (25 mg, 0.05 mmol). Light yellow oil (0.17 g, 63.7%). ¹H NMR (400 MHz, DMSO) δ 7.96 (d, J=8.2 Hz, 1H), 7.74 (dd, J=17.4, 9.1 Hz, 2H), 7.63 (d, J=1.7 Hz, 1H), 7.28 (dd, J=8.5, 1.7 Hz, 1H), 7.18 (dd, J=6.4, 2.7 Hz, 2H), 7.16-7.08 (m, 2H), 6.83 (d, J=8.4 Hz, 1H), 4.54 (s, 2H), 4.49 (t, J=8.0 Hz, 1H), 3.92 (s, 3H), 3.83 (qt, J=8.9, 4.3 Hz, 1H), 3.72 (s, 3H), 3.25-3.08 (m, 2H), 2.08 (s, 3H), 1.84-1.71 (m, 2H), 1.67-1.30 (m, 10H) LC-MS (ESI): m/z=504.3, t_{R}=5.46 min.

3-(7-(2-(Cycloheptylamino)-2-oxoethoxy)naphthalen-2-yl)-3-(4-methoxy-3-methylphenyl)propanoic acid (2j, UCAB #967). The reaction was conducted following the general procedure G, using I-7j (0.17, 0.35 mmol) and 2 M aq. NaOH (350 μl). White solid (0.077 g, 46.1%) ¹H NMR (400 MHz, DMSO) δ 12.08 (s, 1H), 7.96 (d, J=8.1 Hz, 1H), 7.80-7.69 (m, 2H), 7.63 (d, J=1.7 Hz, 1H), 7.28 (dd, J=8.4, 1.7 Hz, 1H), 7.23-7.02 (m, 4H), 6.83 (d, J=8.4 Hz, 1H), 4.55 (s, 2H), 4.46 (t, J=7.9 Hz, 1H), 3.84 (qt, J=9.0, 4.4 Hz, 1H), 3.73 (s, 3H), 3.13-2.97 (m, 2H), 2.09 (s, 3H), 1.82-1.72 (m, 2H), 1.71-1.16 (m, 10H). ¹³C NMR (101 MHz, DMSO) δ 173.22, 166.54, 156.35, 156.25, 143.25, 135.96, 134.57, 130.21, 129.37, 128.05, 127.77, 126.24, 125.80, 124.89, 124.64, 118.64, 110.61, 107.62, 67.54, 55.63, 50.04, 46.44, 40.71, 34.68 (2C), 28.16 (2C), 24.22 (2C), 16.59. LC-MS (ESI): m/z=490.3, t_{R}=5.04 min.

Methyl (E)-3-(4-methoxy-2-methylphenyl) acrylate (1-9k). The reaction was conducted following the General procedure C, using 1-bromo-4-methoxy-2-methylbenzene (0.5 g, 2.49 mmol), methyl acrylate (0.34 ml, 3.73 mmol), Pd(OAc)₂ (0.03 g, 0.13 mmol), tri(o-tolyl)phosphine (0.08 g, 0.25 mmol), and Et₃N (2.5 ml, 17.83 mmol). White solid (0.31 g, 60.8%). ¹H NMR (400 MHz, DMSO) δ 7.81 (d, J=15.9 Hz, 1H), 7.71 (d, J=8.6 Hz, 1H), 6.88-6.77 (m, 2H), 6.41 (d, J=15.8 Hz, 1H), 3.78 (s, 3H), 3.71 (s, 3H), 2.38 (s, 3H).LC-MS (ESI): m/z=207.1, t_{R}=4.67 min.

Methyl 3-(7-(2-(cycloheptylamino)-2-oxoethoxy)naphthalen-2-yl)-3-(4-methoxy-2-methylphenyl)propanoate (I-7k). The reaction was conducted following the general procedure F, using I-6 (0.30, 0.73 mmol) and I-9k (0.10 g, 0.49 mmol), TEA (0.21 ml, 1.47 mmol), and [Rh(COD)Cl]₂ (25 mg, 0.05 mmol). Colorless oil (0.21 g, 58.1%). ¹H NMR (600 MHz, DMSO) δ 7.96 (d, J=8.1 Hz, 1H), 7.76 (d, J=8.9 Hz, 1H), 7.72 (d, J=8.5 Hz, 1H), 7.55-7.51 (m, 1H), 7.28 (d, J=8.5 Hz, 1H), 7.24 (dd, J=8.5, 1.8 Hz, 1H), 7.20-7.14 (m, 2H), 6.75 (dd, J=8.5, 2.8 Hz, 1H), 6.72 (d, J=2.8 Hz, 1H), 4.70 (t, J=7.9 Hz, 1H), 4.54 (s, 2H), 3.87-3.78 (m, 1H), 3.71 (s, 3H), 3.51 (s, 3H), 3.18-3.07 (m, 2H), 2.27 (s, 3H), 1.80-1.71 (m, 2H), 1.65-1.33 (m, 10H). LC-MS (ESI): m/z=504.3, t_{R}=5.37 min.

3-(7-(2-(Cycloheptylamino)-2-oxoethoxy)naphthalen-2-yl)-3-(4-methoxy-2-methylphenyl)propanoic acid (2k, UCAB #960). The reaction was conducted following the general procedure G, using I-7k (0.21, 0.36 mmol) and 2M aq. NaOH (360 μl). White solid (0.077 g, 46.7%) ¹H NMR (600 MHz, DMSO) δ 12.11 (s, 1H), 7.96 (d, J=8.1 Hz, 1H), 7.76 (d, J=8.8 Hz, 1H), 7.72 (d, J=8.5 Hz, 1H), 7.53 (d, J=1.8 Hz, 1H), 7.29-7.22 (m, 2H), 7.20-7.14 (m, 2H), 6.76 (dd, J=8.5, 2.8 Hz, 1H), 6.72 (d, J=2.7 Hz, 1H), 4.68 (t, J=7.9 Hz, 1H), 4.54 (s, 2H), 3.87-3.78 (m, 1H), 3.71 (s, 3H), 3.00 (h, J=8.1 Hz, 2H), 2.27 (s, 3H), 1.80-1.72 (m, 2H), 1.65-1.435 (m, 10H) ¹³C NMR (151 MHz, DMSO) δ 173.23, 166.54, 157.89, 156.36, 142.59, 137.51, 134.48, 134.20, 129.36, 128.06, 127.99, 127.73, 125.05 (2C), 118.72, 116.39, 111.63, 107.57, 67.51, 55.34, 50.01, 42.37, 40.55, 34.67 (2C), 28.17 (2C), 24.21 (2C), 20.07. LC-MS (ESI): m/z=490.3, t_{R}=4.95 min.

Methyl (E)-3-(3-chloro-2-methylphenyl) acrylate (I-91). The reaction was conducted following the General procedure C, using 1-bromo-3-chloro-2-methylbenzene (1.0 g, 4.86 mmol), methyl acrylate (0.66 ml, 7.20 mmol), Pd(OAc)₂ (0.06 g, 0.26 mmol), tri(o-tolyl)phosphine (0.15 g, 0.50 mmol), and Et₃N (4.4 ml, 31.40 mmol). Colorless oil (0.79 g, 79.0%). ¹H NMR (400 MHz, DMSO) δ 7.90 (d, J=15.9 Hz, 1H), 7.68 (dd, J=7.8, 1.2 Hz, 1H), 7.50 (dd, J=8.1, 1.2 Hz, 1H), 7.28 (q, J=7.1 Hz, 1H), 6.54 (d, J=15.8 Hz, 1H), 3.75 (s, 3H), 2.42 (s, 3H). LC-MS (ESI): m/z=211.1, t_{R}=4.79 min.

Methyl 3-(3-chloro-2-methylphenyl)-3-(7-(2-(cycloheptylamino)-2-oxoethoxy)naphthalen-2-yl)propanoate (I-71). The reaction was conducted following the general procedure F, using I-6 (0.19, 0.45 mmol) and I-91 (0.06 g, 0.30 mmol), TEA (0.19 ml, 1.35 mmol), and [Rh(COD)Cl]₂ (15 mg, 0.03 mmol). Colorless oil (0.12 g, 52.9%). ¹H NMR (600 MHz, DMSO) δ 7.96 (d, J=8.1 Hz, 1H), 7.76 (dd, J=17.3, 8.7 Hz, 2H), 7.55 (d, J=1.7 Hz, 1H), 7.38 (dd, J=7.8, 1.2 Hz, 1H), 7.31 (dd, J=8.1, 1.2 Hz, 1H), 7.29-7.18 (m, 3H), 7.16 (d, J=2.6 Hz, 1H), 4.86 (t, J=7.8 Hz, 1H), 4.54 (s, 2H), 3.82 (ddq, J=13.5, 9.1, 4.4 Hz, 1H), 3.52 (s, 3H), 3.19 (d, J=7.9 Hz, 2H), 2.37 (s, 3H), 1.80-1.70 (m, 2H), 1.62-1.31 (m, 10H). LC-MS (ESI): m/z=508.2, t_{R}=5.31 min.

3-(3-chloro-2-methylphenyl)-3-(7-(2-(cycloheptylamino)-2-oxoethoxy) naphthalen-2-yl)propanoic acid (21, UCAB #1083). The reaction was conducted following the general procedure G, using I-71 (0.12, 0.24 mmol) and 2M aq. NaOH (1 ml). White solid (0.08 g, 71.0%) ¹H NMR (600 MHz, DMSO) δ 12.23 (s, 1H), 7.96 (d, J=8.1 Hz, 1H), 7.76 (dd, J=17.6, 8.7 Hz, 2H), 7.55 (d, J=1.7 Hz, 1H), 7.39-7.12 (m, 6H), 4.84 (t, J=7.8 Hz, 1H), 4.54 (s, 2H), 3.82 (dh, J=13.5, 4.4 Hz, 1H), 3.07 (d, J=7.8 Hz, 2H), 2.62 (p, J=1.9 Hz, 1H), 2.37 (s, 3H), 1.82-1.70 (m, 2H), 1.65-1.44 (m, 7H), 1.39 (dtt, J=12.8, 9.8, 2.9 Hz, 2H). ¹³C NMR (151 MHz, DMSO) δ 173.04, 166.54, 156.44, 144.60, 141.60, 134.54, 134.50, 134.01, 129.42, 128.30, 127.85, 127.66, 126.22, 125.30, 124.93, 118.95, 107.60, 67.50, 50.03, 43.63, 40.78, 40.54, 34.66 (2C), 28.16 (2C), 24.20 (2C), 16.26. LC-MS (ESI): m/z=404.2, t_{R}=4.94 min.

Methyl (E)-3-(benzo[d][1,3]dioxol-5-yl) acrylate (I-9m). The reaction was conducted following the General procedure C, using 5-bromobenzo[d][1,3]dioxole (0.3 ml, 2.49 mmol), methyl acrylate (0.40 ml, 3.73 mmol), Pd(OAc)₂ (0.03 g, 0.12 mmol), tri(o-tolyl)phosphine (0.08 g, 0.24 mmol), and Et₃N (2.5 ml, 17.83 mmol). White solid (0.45 g, 88.2%). ¹H NMR (400 MHz, DMSO) δ 7.58 (d, J=15.9 Hz, 1H), 7.41 (d, J=1.7 Hz, 1H), 7.20 (dd, J=8.0, 1.7 Hz, 1H), 6.95 (d, J=8.0 Hz, 1H), 6.51 (d, J=15.9 Hz, 1H), 6.08 (s, 2H), 3.70 (s, 3H). LC-MS (ESI): m/z=207.1, t_{R}=4.35 min.

Methyl 3-(benzo[d][1,3]dioxol-5-yl)-3-(7-(2-(cycloheptylamino)-2-oxoethoxy)naphthalen-2-yl)propanoate (I-7m, 8a, UCAB #1133). The reaction was conducted following the general procedure F, using I-6 (0.30, 0.73 mmol) and I-9m (0.10 g, 0.49 mmol), TEA (0.22 ml, 1.47 mmol), and [Rh(COD)Cl]₂ (15 mg, 0.03 mmol). Colorless oil (0.12 g, 33.8%). ¹H NMR (400 MHz, DMSO) δ 7.95 (d, J=8.1 Hz, 1H), 7.79-7.68 (m, 2H), 7.66 (d, J=1.8 Hz, 1H), 7.30 (dd, J=8.4, 1.7 Hz, 1H), 7.18 (d, J=7.5 Hz, 2H), 6.95 (d, J=1.6 Hz, 1H), 6.86-6.76 (m, 2H), 5.93 (dd, J=4.2, 1.0 Hz, 2H), 4.52 (d, J=13.6 Hz, 3H), 3.82 (qt, J=9.0, 4.4 Hz, 1H), 3.49 (s, 3H), 3.26-3.09 (m, 2H), 1.81-1.70 (m, 2H), 1.66-1.32 (m, 10H). LC-MS (ESI): m/z=504.2, t_{R}=5.20 min.

3-(benzo[d][1,3]dioxol-5-yl)-3-(7-(2-(cycloheptylamino)-2-oxoethoxy)naphthalen-2-yl)propanoic acid (2m, UCAB #952). The reaction was conducted following the general procedure G, using I-7m (0.16, 0.38 mmol) and 2M aq. NaOH (380 μl). White solid (0.08 g, 47.8%). ¹H NMR (600 MHz, DMSO) δ 12.11 (s, 1H), 7.96 (d, J=8.1 Hz, 1H), 7.76 (d, J=9.6 Hz, 1H), 7.72 (d, J=8.5 Hz, 1H), 7.66 (d, J=1.7 Hz, 1H), 7.30 (dd, J=8.5, 1.8 Hz, 1H), 7.18 (dq, J=5.3, 2.5 Hz, 2H), 6.95 (d, J=1.6 Hz, 1H), 6.84-6.77 (m, 2H), 5.93 (dd, J=5.3, 1.0 Hz, 2H), 4.54 (s, 2H), 4.48 (t, J=8.0 Hz, 1H), 3.83 (ddt, J=13.4, 9.1, 4.6 Hz, 1H), 3.12-2.98 (m, 2H), 1.76 (dddt, J=15.5, 7.9, 3.7, 1.7 Hz, 2H), 1.65-1.42 (m, 8H), 1.44-1.35 (m, 2H). ¹³C NMR (151 MHz, DMSO) δ 173.17, 166.54, 156.37, 147.74, 146.04, 142.93, 138.46, 134.58, 129.38, 128.10, 127.83, 124.82, 124.69, 121.05, 118.71, 108.55, 108.52, 107.63, 101.24, 67.54, 50.06, 46.87, 40.41, 34.68 (2C), 28.15 (2C), 24.22 (2C). LC-MS (ESI): m/z=490.2, t_{R}=4.79 min.

Methyl (E)-3-(6-methylbenzo[d][1,3]dioxol-5-yl) acrylate (I-9n). The reaction was conducted following the General procedure C, using 5-bromo-6-methylbenzo[d][1,3]dioxole (0.2 g, 0.93 mmol), methyl acrylate (0.13 ml, 1.40 mmol), Pd(OAc)₂ (0.01 g, 0.05 mmol), tri(o-tolyl)phosphine (0.03 g, 0.10 mmol), and Et₃N (0.93 ml, 6.67 mmol). White solid (0.09 g, 44.1%). ¹H NMR (400 MHz, DMSO) δ 7.78 (d, J=15.8 Hz, 1H), 7.37 (s, 1H), 6.87 (s, 1H), 6.46 (d, J=15.7 Hz, 1H), 6.03 (s, 2H), 3.71 (s, 3H), 2.33 (s, 4H). LC-MS (ESI): m/z=221.1, t_{R}=4.50 min.

Methyl 3-(7-(2-(cycloheptylamino)-2-oxoethoxy)naphthalen-2-yl)-3-(6-methylbenzo[d][1,3]dioxol-5-yl)propanoate (I-7n). The reaction was conducted following the general procedure F, using I-6 (0.20, 0.47 mmol) and I-9n (0.07 g, 0.31 mmol), TEA (0.13 ml, 0.93 mmol), and [Rh(COD)Cl]₂ (16 mg, 0.03 mmol). Colorless oil (0.10 g, 65.0%). ¹H NMR (600 MHz, DMSO) δ 7.96 (d, J=8.1 Hz, 1H), 7.77 (d, J=8.7 Hz, 1H), 7.73 (d, J=8.5 Hz, 1H), 7.58 (d, J=1.8 Hz, 1H), 7.27 (dd, J=8.5, 1.8 Hz, 1H), 7.19 (d, J=8.3 Hz, 2H), 6.98 (s, 1H), 6.73 (s, 1H), 5.95 (d, J=1.0 Hz, 1H), 5.91 (d, J=1.0 Hz, 1H), 4.69 (t, J=7.9 Hz, 1H), 4.54 (s, 2H), 3.83 (dh, J=13.5, 4.4 Hz, 1H), 3.51 (s, 3H), 3.13 (h, J=8.1 Hz, 2H), 2.25 (s, 3H), 1.80-1.72 (m, 2H), 1.68-1.33 (m, 10H). LC-MS (ESI): m/z=518.3, t_{R}=5.28 min.

3-(7-(2-(cycloheptylamino)-2-oxoethoxy)naphthalen-2-yl)-3-(6-methylbenzo[d][1,3]dioxol-5-yl)propanoic acid (2n, UCAB #985). The reaction was conducted following the general procedure G, using I-7n (0.10, 0.20 mmol) and 2 M aq. NaOH (80 μl). White solid (0.02 g, 22.0%). ¹H NMR (600 MHz, DMSO) δ 12.38 (s, 1H), 8.21 (d, J=8.1 Hz, 1H), 8.04-8.00 (m, 1H), 7.98 (d, J=8.5 Hz, 1H), 7.83 (d, J=1.7 Hz, 1H), 7.53 (dd, J=8.5, 1.8 Hz, 1H), 7.44 (dd, J=6.7, 2.7 Hz, 2H), 7.22 (s, 1H), 6.98 (s, 1H), 6.20 (d, J=1.0 Hz, 1H), 6.17 (d, J=1.0 Hz, 1H), 4.92 (t, J=7.8 Hz, 1H), 4.80 (s, 2H), 4.09 (dh, J=13.5, 4.4 Hz, 1H), 3.32-3.21 (m, 2H), 2.50 (s, 3H), 2.06-1.97 (m, 2H), 1.93-1.69 (m, 8H), 1.65 (dtd, J=16.9, 9.3, 4.9 Hz, 2H). ¹³C NMR (151 MHz, DMSO) δ 173.22, 166.54, 156.36, 146.01, 145.66, 142.31, 135.28, 134.50, 129.36, 129.13, 128.10, 127.77, 125.04, 124.97, 118.75, 110.84, 107.61 (2C), 101.08, 67.52, 50.03, 42.76, 40.77, 34.67 (2C), 28.16 (2C), 24.21 (2C), 19.79. LC-MS (ESI): m/z=504.2, t_{R}=4.85 min.

Methyl (E)-3-(7-methoxy-1-methyl-1H-benzo[d][1,2,3]triazol-5-yl) acrylate (I-90). The reaction was conducted following the General procedure C, using 5-bromo-7-methoxy-1-methyl-1H-benzo[d][1,2,3]triazol (0.15 g, 0.63 mmol), methyl acrylate (0.10 ml, 0.94 mmol), Pd(OAc)₂ (0.01 g, 0.03 mmol), tri(o-tolyl)phosphine (0.02g, 0.06 mmol), and Et₃N (0.63 ml, 4.48 mmol). White solid (0.15 g, 91.5%). ¹H NMR (400 MHz, DMSO) δ 7.94 (d, J=1.0 Hz, 1H), 7.81 (d, J=16.0 Hz, 1H), 7.38 (d, J=1.1 Hz, 1H), 6.84 (d, J=16.0 Hz, 1H), 4.41 (s, 3H), 4.05 (s, 3H), 3.76 (s, 3H). LC-MS (ESI): m/z=248.1, t_{R}=4.10 min.

Methyl 3-(7-(2-(cycloheptylamino)-2-oxoethoxy)naphthalen-2-yl)-3-(7-methoxy-1-methyl-1H-benzo[d][1,2,3]triazol-5-yl)propanoate (I-70). The reaction was conducted following the general procedure F, using I-6 (0.25, 0.61 mmol) and I-90 (0.10 g, 0.41 mmol), TEA (0.17 ml, 1.22 mmol), and [Rh(COD)Cl]₂ (20 mg, 0.04 mmol). Colorless oil (0.13 g, 60.0%). ¹H NMR (600 MHz, DMSO) δ 7.95 (d, J=8.1 Hz, 1H), 7.84-7.70 (m, 3H), 7.55 (d, J=1.1 Hz, 1H), 7.41 (dd, J=8.5, 1.8 Hz, 1H), 7.22-7.16 (m, 2H), 6.98 (d, J=1.1 Hz, 1H), 4.75 (t, J=8.0 Hz, 1H), 4.54 (s, 2H), 4.34 (s, 3H), 3.92 (s, 3H), 3.87-3.78 (m, 1H), 3.51 (s, 3H), 3.33 (dd, J=8.1, 1.6 Hz, 2H), 1.75 (ddt, J=15.7, 7.7, 4.2 Hz, 2H), 1.65-1.30 (m, 10H). LC-MS (ESI): m/z=545.3, t_{R}=4.93 min.

3-(7-(2-(cycloheptylamino)-2-oxoethoxy)naphthalen-2-yl)-3-(7-methoxy-1-methyl-1H-benzo[d][1,2,3]triazol-5-yl)propanoic acid (20, UCAB #987). The reaction was conducted following the general procedure G, using I-70 (0.01, 0.13 mmol) and 2M aq. NaOH (130 μl). White solid (0.03 g, 52.4%). ¹H NMR (600 MHz, DMSO) δ 12.16 (s, 1H), 7.95 (d, J=8.1 Hz, 1H), 7.78-7.71 (m, 3H), 7.53 (s, 1H), 7.40 (dd, J=8.6, 1.7 Hz, 1H), 7.18 (dq, J=5.5, 2.5 Hz, 2H), 6.98-6.95 (m, 1H), 4.71 (t, J=7.9 Hz, 1H), 4.54 (s, 2H), 4.33 (s, 3H), 3.91 (s, 3H), 3.82 (qt, J=9.0, 4.4 Hz, 1H), 3.23-3.17 (m, 2H), 1.75 (ddq, J=11.9, 7.5, 4.0 Hz, 2H), 1.64-1.17 (m, 10H). ¹³C NMR (151 MHz, DMSO) δ 173.21, 166.54, 156.38, 147.96, 146.20, 142.54, 142.11, 134.59, 129.40, 128.13, 127.90, 125.08, 124.91, 124.33, 118.78, 109.12, 107.89, 107.66, 67.54, 56.66, 50.05, 47.35, 40.53, 37.02, 34.67 (2C), 28.14 (2C), 24.21 (2C). LC-MS (ESI): m/z=531.3, t_{R}=4.55 min.

N-ethyl-3-methyl-2-nitroaniline (I-10). To a 50 mL round-bottomed flask charged with a magnetic stirring bar were added 1-fluoro-3-methyl-2-nitrobenzene (2 g, 12.9 mmol), Ethylamine HCl salt (5.3 g, 64.5 mmol), K₂CO₃ (4.5 g, 32.23 mmol) and TEA (6.52 g, 9 ml) in EtOH (50 ml). The mixture was sealed and stirred at 90° C. for 12 h. Upon reaction completion (as determined by LC-MS). After cooling down, the reaction mixture was diluted with EA (55 ml). The organic layer was washed with water (20 ml), and sat. brine (20 mL), dried over Na₂SO₄, filtered and concentrated under reduced pressure to afford the desired product without purification. Orange oil (0.79 g, 69.0%). ¹H NMR (400 MHz, DMSO) δ 7.27 (t, J=7.9 Hz, 1H), 6.77 (d, J=8.5 Hz, 1H), 6.55 (d, J=7.3 Hz, 1H), 6.32 (t, J=5.6 Hz, 1H), 3.20 (qd, J=7.1, 5.4 Hz, 2H), 2.30 (s, 3H), 1.16 (t, J=7.1 Hz, 3H). LC-MS (ESI): m/z=181.1, t_{R}=4.79 min.

4-bromo-N-ethyl-3-methyl-2-nitroaniline (I-11). To a 50 mL round-bottomed flask charged with a magnetic stirring bar added N-ethyl-3-methyl-2-nitroaniline (I-10, 0.8 g, 4.37 mmol) in DMF (1.5 ml). NBS (0.7 g, 3.93 mmol) in DMF (1.5 ml) was added dropwise at 0° C., and then the mixture was warmed to room temperature for 12 h. Upon reaction completion (as determined by LC-MS), the reaction mixture was diluted with EA (10 ml). The organic layer was washed with sat. brine (5 mL×5), dried over Na₂SO₄, filtered and concentrated under reduced pressure to afford the crude product. The crude was purified by silica gel column chromatography to afford the desired product. Red oil (0.87 g, 76.0%). ¹H NMR (600 MHz, DMSO) δ 7.54 (d, J=9.1 Hz, 1H), 6.73 (d, J=9.1 Hz, 1H), 6.08 (t, J=5.6 Hz, 1H), 3.16 (qd, J=7.1, 5.5 Hz, 2H), 2.25 (s, 3H), 1.11 (t, J=7.1 Hz, 3H). LC-MS (ESI): m/z=259.0/261.0, t_{R}=5.17 min.

4-bromo-N₁-ethyl-3-methylbenzene-1,2-diamine (I-12). A solution of 4-bromo-N-ethyl-3-methyl-2-nitroaniline (I-11, 0.78 g, 3.01 mmol), Fe (1.2 g, 21.1 mmol) and NH₄Cl (1.2 g, 21.1 mmol) in EtOH/H₂O (9/3 ml). Then the mixture was stirred at 80° C. for 1h. Upon reaction completion (as determined by LC-MS). After cooling down, the reaction mixture was under reduced pressure to afford the crude product. The crude was purified by silica gel column chromatography to afford the desired product. Brown solid (0.23 g, 52.3%). ¹H NMR (600 MHz, DMSO) δ 6.71 (d, J=8.5 Hz, 1H), 6.25 (d, J=8.5 Hz, 1H), 4.59 (s, 2H), 4.50 (t, J=5.3 Hz, 1H), 3.02 (qd, J=7.1, 5.2 Hz, 2H), 2.16 (s, 3H), 1.20 (t, J=7.1 Hz, 3H). LC-MS (ESI): m/z=229.1/231.1, t_{R}=3.56 min.

5-bromo-1-ethyl-4-methyl-1H-benzo[d][1,2,3]triazole (1-8p). To a viral charged with a magnetic stirring bar added 4-bromo-N₁-ethyl-3-methylbenzene-1,2-diamine (I-12, 0.23 g, 1.01 mmol) in H₂SO₄ (0.4g). A solution of NaNO₂ (0.11 g, 1.51 mmol) in H₂O (4 ml) was added dropwise at 0° C. . . . Then the mixture was stirred at 0° C. for 2h. Upon reaction completion (as determined by LC-MS), the reaction mixture was concentrated under reduced pressure to afford the crude product. The crude was purified by silica gel column chromatography to afford the desired product. Brown solid (0.13 g, 56.5%). ¹H NMR (600 MHz, DMSO) δ 7.74-7.66 (m, 2H), 4.73 (q, J=7.3 Hz, 2H), 2.72 (s, 3H), 1.50 (t, J=7.3 Hz, 3H). LC-MS (ESI): m/z=240.0/242.0, t_{R}=4.46 min.

Methyl (E)-3-(1-ethyl-4-methyl-1H-benzo[d][1,2,3]triazol-5-yl) acrylate (I-9p). The reaction was conducted following the General procedure C, using I-8p (0.13 g, 0.54 mmol), methyl acrylate (0.10 ml, 0.82 mmol), Pd(OAc)₂ (0.01 g, 0.03 mmol), tri(o-tolyl)phosphine (0.02 g, 0.06 mmol), and Et₃N (0.60 ml, 3.88 mmol). White solid (0.07 g, 56.0%). ¹H NMR (600 MHz, DMSO) δ 8.04 (d, J=15.9 Hz, 1H), 7.97 (d, J=8.8 Hz, 1H), 7.75 (d, J=8.8 Hz, 1H), 6.67 (d, J=15.9 Hz, 1H), 4.73 (q, J=7.3 Hz, 2H), 3.76 (s, 3H), 2.82 (s, 3H), 1.50 (t, J=7.3 Hz, 3H). LC-MS (ESI): m/z=246.1, t_{R}=4.12 min.

Methyl 3-(7-(2-(cycloheptylamino)-2-oxoethoxy)naphthalen-2-yl)-3-(1-ethyl-4-methyl-1H-benzo[d][1,2,3]triazol-5-yl)propanoate (I-7p). The reaction was conducted following the general procedure F, using I-6 (0.19, 0.45 mmol) and I-9p (0.01 g, 0.31 mmol), TEA (0.13 ml, 0.91 mmol), and [Rh(COD)Cl]₂ (15 mg, 0.03 mmol). Colorless oil (0.05 g, 31.6%). ¹H NMR (400 MHz, DMSO) δ 7.95 (d, J=8.1 Hz, 1H), 7.75 (dd, J=14.9, 9.1 Hz, 2H), 7.69-7.59 (m, 2H), 7.52 (d, J=8.7 Hz, 1H), 7.30 (dd, J=8.5, 1.8 Hz, 1H), 7.19 (dq, J=4.5, 2.5 Hz, 2H), 5.02 (t, J=7.8 Hz, 1H), 4.67 (q, J=7.2 Hz, 2H), 4.54 (s, 2H), 3.83 (qt, J=9.1, 4.5 Hz, 1H), 3.51 (s, 3H), 3.26-3.09 (m, 2H), 2.81 (s, 3H), 1.75 (ddd, J=13.0, 7.5, 4.0 Hz, 2H), 1.65-1.32 (m, 13H). LC-MS (ESI): m/z=543.3, t_{R}=5.04 min.

3-(7-(2-(cycloheptylamino)-2-oxoethoxy)naphthalen-2-yl)-3-(1-ethyl-4-methyl-1H-benzo[d][1,2,3]triazol-5-yl)propanoic acid (2p, UCAB #991). The reaction was conducted following the general procedure G, using I-7p (0.05, 0.10 mmol) and 2 M aq. NaOH (100 μl). White solid (0.02 g, 48.6%). ¹H NMR (600 MHz, DMSO) δ 12.14 (s, 1H), 7.95 (d, J=8.1 Hz, 1H), 7.79-7.75 (m, 1H), 7.73 (d, J=8.5 Hz, 1H), 7.66 (d, J=1.8 Hz, 1H), 7.63 (d, J=8.7 Hz, 1H), 7.51 (d, J=8.7 Hz, 1H), 7.30 (dd, J=8.5, 1.8 Hz, 1H), 7.21-7.16 (m, 2H), 5.00 (t, J=7.9 Hz, 1H), 4.67 (q, J=7.3 Hz, 2H), 4.54 (s, 2H), 3.82 (dp, J=13.5, 4.5 Hz, 1H), 3.22 (dd, J=15.9, 7.3 Hz, 1H), 3.12 (dd, J=15.9, 8.4 Hz, 1H), 2.81 (s, 3H), 1.79-1.71 (m, 2H), 1.65-1.47 (m, 8H), 1.46 (d, J=7.2 Hz, 3H), 1.43-1.34 (m, 2H). ¹³C NMR (151 MHz, DMSO) δ 173.24, 166.52, 156.40, 146.52, 142.28, 137.03, 134.54, 131.32, 129.37, 128.19, 127.76, 127.19, 127.04, 124.98, 124.89, 118.81, 108.18, 107.66, 67.52, 50.02, 43.08, 41.98, 40.49, 34.66 (2C), 28.15 (2C), 24.20 (2C), 15.40, 13.38. LC-MS (ESI): m/z=529.3, t_{R}=4.60 min.

Methyl (E)-3-(2,3-dihydrobenzofuran-5-yl) acrylate (I-9q). The reaction was conducted following the General procedure C, using 5-bromo-2,3-dihydrobenzofuran (0.50 g, 2.52 mmol), methyl acrylate (0.40 ml, 3.77 mmol), Pd(OAc)₂ (0.03 g, 0.12 mmol), tri(o-tolyl)phosphine (0.08 g, 0.24 mmol), and Et₃N (2.5 ml, 17.83 mmol). White solid (0.12 g, 23.5%). ¹H NMR (600 MHz, DMSO) δ 7.64 (q, J=1.4 Hz, 1H), 7.59 (d, J=16.0 Hz, 1H), 7.45 (dd, J=8.2, 1.9 Hz, 1H), 6.79 (d, J=8.2 Hz, 1H), 6.43 (d, J=16.0 Hz, 1H), 4.58 (t, J=8.7 Hz, 2H), 3.69 (s, 3H), 3.19 (t, J=8.7 Hz, 2H). LC-MS (ESI): m/z=205.1, t_{R}=4.72 min.

Methyl 3-(7-(2-(cycloheptylamino)-2-oxoethoxy)naphthalen-2-yl)-3-(2,3-dihydrobenzofuran-5-yl)propanoate (I-7q). The reaction was conducted following the general procedure F, using I-6 (0.03, 0.74 mmol) and I-9q (0.1 g, 0.49 mmol), TEA (0.21 ml, 1.47 mmol), and [Rh(COD)Cl]₂ (25 mg, 0.05 mmol). Colorless oil (0.05 g, 21.7%). ¹H NMR (600 MHz, DMSO) δ 7.96 (d, J=8.1 Hz, 1H), 7.76 (d, J=9.6 Hz, 1H), 7.72 (d, J=8.4 Hz, 1H), 7.67-7.63 (m, 1H), 7.29 (dd, J=8.4, 1.8 Hz, 1H), 7.18 (dq, J=4.4, 2.6 Hz, 3H), 7.07 (dd, J=8.4, 2.0 Hz, 1H), 6.65 (d, J=8.2 Hz, 1H), 4.55 (s, 2H), 4.51 (t, J=8.0 Hz, 1H), 4.45 (t, J=8.7 Hz, 2H), 3.83 (ddq, J=13.5, 9.1, 4.5 Hz, 1H), 3.50 (s, 3H), 3.20 (dd, J=15.8, 8.2 Hz, 1H), 3.12 (dt, J=22.2, 8.3 Hz, 3H), 1.80-1.73 (m, 2H), 1.65-1.22 (m, 10H). LC-MS (ESI): m/z=502.3, t_{R}=5.45 min.

3-(7-(2-(cycloheptylamino)-2-oxoethoxy)naphthalen-2-yl)-3-(2,3-dihydrobenzofuran-5-yl)propanoic acid (2q, UCAB #973). The reaction was conducted following the general procedure G, using I-7q (0.05, 0.11 mmol) and 2 M aq. NaOH (110 μl). White solid (0.03 g, 51.0%). ¹H NMR (600 MHz, DMSO) δ 12.09 (s, 1H), 7.96 (d, J=8.1 Hz, 1H), 7.75 (d, J=8.6 Hz, 1H), 7.71 (d, J=8.5 Hz, 1H), 7.64 (d, J=1.7 Hz, 1H), 7.28 (dd, J=8.5, 1.8 Hz, 1H), 7.20-7.15 (m, 3H), 7.06 (dd, J=8.3, 1.9 Hz, 1H), 6.64 (d, J=8.2 Hz, 1H), 4.54 (s, 2H), 4.51-4.41 (m, 3H), 3.88-3.79 (m, 1H), 3.12-3.04 (m, 3H), 3.01 (dd, J=15.7, 7.9 Hz, 1H), 1.80-1.72 (m, 2H), 1.65-1.35 (m, 10H). ¹³C NMR (151 MHz, DMSO) δ 171.12, 164.44, 156.53, 154.25, 141.17, 134.33, 132.48, 127.26, 125.96, 125.70, 125.67, 125.26, 122.81, 122.60, 122.48, 116.53, 106.89, 105.52, 69.16, 65.42, 47.95, 44.57, 38.55, 32.57 (2C), 27.47, 26.04 (2C), 22.11 (2C). LC-MS (ESI): m/z=488.3, t_{R}=5.13 min.

Methyl (E)-3-(2,2-difluorobenzo[d][1,3]dioxol-5-yl) acrylate (I-9r). The reaction was conducted following the General procedure C, using 5-bromo-2,2-difluorobenzo[d][1,3]dioxole (0.50 g, 2.12 mmol), methyl acrylate (0.30 ml, 3.18 mmol), Pd(OAc)₂ (0.02 g, 0.11 mmol), tri(o-tolyl)phosphine (0.07 g, 0.22 mmol), and Et₃N (2.2 ml, 15.2 mmol). White solid (0.45 g, 87.7%). ¹H NMR (400 MHz, DMSO) δ 7.92 (d, J=1.7 Hz, 1H), 7.67 (d, J=16.0 Hz, 1H), 7.58 (dd, J=8.4, 1.7 Hz, 1H), 7.46 (d, J=8.3 Hz, 1H), 6.68 (d, J=16.0 Hz, 1H), 3.72 (s, 3H). LC-MS (ESI): m/z=243.0, t_{R}=4.67 min.

Methyl 3-(7-(2-(cycloheptylamino)-2-oxoethoxy)naphthalen-2-yl)-3-(2,2-difluorobenzo[d][1,3]dioxol-5-yl)propanoate (I-7r). The reaction was conducted following the general procedure F, using I-6 (0.02, 0.45 mmol) and I-9r (0.07 g, 0.30 mmol), TEA (0.19 ml, 1.35 mmol), and [Rh(COD)Cl]₂ (15 mg, 0.03 mmol). Colorless oil (0.10 g, 41.7%). ¹H NMR (400 MHz, DMSO) δ 7.96 (d, J=8.1 Hz, 1H), 7.76 (dd, J=11.6, 8.6 Hz, 2H), 7.70 (d, J=1.8 Hz, 1H), 7.52 (d, J=1.7 Hz, 1H), 7.35 (dd, J=8.5, 1.8 Hz, 1H), 7.30 (d, J=8.3 Hz, 1H), 7.27-7.15 (m, 3H), 4.65 (t, J=8.0 Hz, 1H), 4.55 (s, 2H), 3.83 (dh, J=13.3, 4.4 Hz, 1H), 3.51 (s, 3H), 3.27 (dd, J=8.0, 4.7 Hz, 2H), 1.84-1.69 (m, 2H), 1.67-1.33 (m, 10H). LC-MS (ESI): m/z=540.2, t_{R}=5.30 min.

3-(7-(2-(cycloheptylamino)-2-oxoethoxy)naphthalen-2-yl)-3-(2,2-difluorobenzo[d][1,3]dioxol-5-yl)propanoic acid (2r, UCAB #1082). The reaction was conducted following the general procedure G, using I-7r (0.10, 0.18 mmol) and 2 M aq. NaOH (810 μl). White solid (0.05 g, 53.0%). ¹H NMR (600 MHz, DMSO) δ 12.18 (s, 1H), 7.96 (d, J=8.1 Hz, 1H), 7.76 (dd, J=18.1, 8.6 Hz, 2H), 7.70 (s, 1H), 7.51 (t, J=1.4 Hz, 1H), 7.35 (dt, J=8.5, 1.4 Hz, 1H), 7.30 (dd, J=8.3, 1.0 Hz, 1H), 7.25-7.16 (m, 3H), 4.61 (t, J=7.9 Hz, 1H), 4.55 (s, 2H), 3.83 (tp, J=9.0, 4.4 Hz, 1H), 3.23-3.00 (m, 2H), 1.80-1.69 (m, 2H), 1.68-1.32 (m, 10H). ¹³C NMR (151 MHz, DMSO) δ 173.02, 166.53, 156.44, 143.27, 142.28, 141.71, 141.63, 134.59, 131.63, 129.43, 128.29, 127.91, 124.90, 124.66, 124.12, 118.89, 110.29, 109.90, 107.63, 67.54, 50.06, 46.87, 34.68 (2C), 28.14 (2C), 24.22 (2C). LC-MS (ESI): m/z=526.2, t_{R}=4.87 min.

Methyl (E)-3-(2,3-dihydrobenzo[b][1,4]dioxin-6-yl) acrylate (I-9s). The reaction was conducted following the General procedure C, using 6-bromo-2,3-dihydrobenzo[b][1,4]dioxine (0.50 g, 2.33 mmol), methyl acrylate (0.32 ml, 3.49 mmol), Pd(OAc)₂ (0.03 g, 0.12 mmol), tri(o-tolyl)phosphine (0.07 g, 0.23 mmol), and Et₃N (2.4 ml, 16.7 mmol). White solid (0.41 g, 80.1%). ¹H NMR (400 MHz, DMSO) δ 7.55 (d, J=16.0 Hz, 1H), 7.27 (d, J=2.1 Hz, 1H), 7.20 (dd, J=8.4, 2.1 Hz, 1H), 6.89 (d, J=8.3 Hz, 1H), 6.48 (d, J=16.0 Hz, 1H), 4.32-4.22 (m, 4H), 3.70 (s, 3H). LC-MS (ESI): m/z=221.1, t_{R}=4.19 min.

Methyl 3-(7-(2-(cycloheptylamino)-2-oxoethoxy)naphthalen-2-yl)-3-(2,3-dihydrobenzo[b][1,4]dioxin-6-yl)propanoate (I-7s). The reaction was conducted following the general procedure F, using I-6 (0.03, 0.71 mmol) and I-9s (0.10 g, 0.47 mmol), TEA (0.30 ml, 2.1 mmol), and [Rh(COD)Cl]₂ (23 mg, 0.05 mmol). Colorless oil (0.12 g, 32.7%). ¹H NMR (400 MHz, DMSO) δ 7.96 (d, J=8.2 Hz, 1H), 7.74 (dd, J=17.2, 9.1 Hz, 2H), 7.65 (s, 1H), 7.29 (dd, J=8.4, 1.7 Hz, 1H), 7.22-7.16 (m, 2H), 6.87-6.70 (m, 3H), 4.55 (s, 2H), 4.47 (t, J=8.0 Hz, 1H), 4.17 (s, 4H), 3.84 (ddq, J=13.3, 9.0, 4.3 Hz, 1H), 3.50 (s, 3H), 3.24-3.05 (m, 2H), 1.83-1.70 (m, 2H), 1.68-1.33 (m, 10H). LC-MS (ESI): m/z=518.2, t_{R}=4.89 min.

3-(7-(2-(cycloheptylamino)-2-oxoethoxy)naphthalen-2-yl)-3-(2,3-dihydrobenzo[b][1,4]dioxin-6-yl)propanoic acid (2s, UCAB #1090). The reaction was conducted following the general procedure G, using I-7s (0.12, 0.23 mmol) and 2 M aq. NaOH (1 ml). White solid (0.06 g, 51.2%). ¹H NMR (400 MHz, DMSO) δ 12.10 (s, 1H), 7.97 (d, J=8.1 Hz, 1H), 7.78-7.75 (m, 1H), 7.72 (d, J=8.5 Hz, 1H), 7.65 (d, J=1.7 Hz, 1H), 7.29 (dd, J=8.5, 1.8 Hz, 1H), 7.19 (d, J=7.9 Hz, 2H), 6.83 (d, J=2.1 Hz, 1H), 6.79 (dd, J=8.4, 2.1 Hz, 1H), 6.74 (d, J=8.3 Hz, 1H), 4.55 (s, 2H), 4.44 (t, J=8.0 Hz, 1H), 4.17 (h, J=3.3 Hz, 4H), 3.89-3.79 (m, 1H), 3.04 (qd, J=15.8, 8.0 Hz, 2H), 1.77 (ddtd, J=12.3, 6.2, 3.0, 1.5 Hz, 2H), 1.66-1.45 (m, 8H), 1.44-1.36 (m, 2H). ¹³C NMR (151 MHz, DMSO) δ 171.06, 164.42, 154.24, 141.43, 140.84, 140.15, 135.46, 132.46, 127.26, 125.96, 125.70, 122.75, 122.56, 118.64, 116.57, 115.17, 114.41, 105.50, 65.42, 62.39, 62.29, 47.95, 44.35, 38.42, 32.57 (2C), 26.04 (2C), 22.11 (2C). LC-MS (ESI): m/z=504.2, t_{R}=4.52 min.

Methyl (E)-3-(benzofuran-5-yl) acrylate (I-9t). The reaction was conducted following the General procedure C, using 5-bromobenzofuran (0.5 g, 2.54 mmol), methyl acrylate (0.33 ml, 3.81 mmol), Pd(OAc)₂ (0.03 g, 0.13 mmol), tri(o-tolyl)phosphine (0.08 g, 0.26 mmol), and Et₃N (2.6 ml, 18.2 mmol). White solid (0.50 g, 97.5%). ¹H NMR (600 MHz, DMSO) δ 8.05 (d, J=2.2 Hz, 1H), 8.03 (d, J=1.8 Hz, 1H), 7.78 (d, J=16.0 Hz, 1H), 7.72 (dd, J=8.6, 1.8 Hz, 1H), 7.64 (d, J=8.6 Hz, 1H), 7.00 (dd, J=2.2, 1.0 Hz, 1H), 6.64 (d, J=16.0 Hz, 1H), 3.73 (s, 3H). LC-MS (ESI): m/z=203.1, t_{R}=4.93 min.

Methyl 3-(benzofuran-5-yl)-3-(7-(2-(cycloheptylamino)-2-oxoethoxy) naphthalen-2-yl)propanoate (I-7t). The reaction was conducted following the general procedure F, using I-6 (0.30, 0.74 mmol) and methyl cinnamate (I-9t, 0.1 g, 0.49 mmol), TEA (0.21 ml, 1.47 mmol), and [Rh(COD)Cl]₂ (25 mg, 0.05 mmol). White solid (0.23 g, 92.1%). ¹H NMR (600 MHz, DMSO) δ 7.98-7.91 (m, 2H), 7.79-7.74 (m, 1H), 7.73 (d, J=8.5 Hz, 1H), 7.71-7.68 (m, 1H), 7.64 (d, J=1.8 Hz, 1H), 7.48 (d, J=8.5 Hz, 1H), 7.36-7.24 (m, 2H), 7.19 (dq, J=5.0, 2.5 Hz, 2H), 6.90 (dd, J=2.2, 1.0 Hz, 1H), 4.71 (t, J=8.0 Hz, 1H), 4.54 (s, 2H), 3.83 (tq, J=12.7, 4.5 Hz, 1H), 3.50 (s, 3H), 3.30-3.23 (m, 2H), 1.76 (dtd, J=13.6, 7.3, 3.4 Hz, 2H), 1.66-1.26 (m, 10H). LC-MS (ESI): m/z=500.2, t_{R}=6.57 min.

3-(Benzofuran-5-yl)-3-(7-(2-(cycloheptylamino)-2-oxoethoxy)naphthalen-2-yl)propanoic acid (2t, UCAB #983). The reaction was conducted following the general procedure G, using I-7t (0.23, 0.47 mmol) and 2 M aq. NaOH (470 μl). White solid (0.027 g, 12.1%). ¹H NMR (600 MHz, DMSO) δ 12.12 (s, 1H), 7.98-7.93 (m, 2H), 7.76 (d, J=8.7 Hz, 1H), 7.74-7.68 (m, 2H), 7.63 (d, J=1.8 Hz, 1H), 7.48 (d, J=8.6 Hz, 1H), 7.35-7.27 (m, 2H), 7.19 (d, J=8.2 Hz, 2H), 6.90 (dd, J=2.2, 1.0 Hz, 1H), 4.68 (t, J=7.9 Hz, 1H), 4.54 (s, 2H), 3.83 (dh, J=13.5, 4.5 Hz, 1H), 3.21-3.10 (m, 2H), 1.76 (ddt, J=11.0, 7.5, 3.7 Hz, 2H), 1.66-1.19 (m, 10H). ¹³C NMR (151 MHz, DMSO) δ 173.21, 166.52, 156.38, 153.49, 146.75, 143.14, 139.21, 134.59, 129.38, 128.12, 127.77 (2C), 125.48, 124.75, 124.14, 120.28, 118.72, 111.52, 107.64, 107.17, 67.53, 50.04, 47.03, 40.49, 34.68 (2C), 28.16 (2C), 24.22 (2C). LC-MS (ESI): m/z=486.2, t_{R}=5.24 min.

6-bromo-7-methyl-2,3-dihydrobenzo[b][1,4]dioxine (I-8u). To a vial charged with a magnetic stirring bar was added 6-methyl-2,3-dihydrobenzo[b][1,4]dioxine (0.05 g, 0.33 mmol) dissolved in MeCN (1 ml). To this solution was then added N-bromosuccinimide (0.076 g, 0.43 mmol) under stirring. The mixture was stirred at room temperature for 48 h under N₂ atmosphere. Upon reaction completion (as determined by LC-MS), the mixture is poured into saturated NaHCO₃(the color changes from yellow to colorless) and extracted with EtOAc (3×10 mL pr. mmol). The combined organic layers were dried over Na₂SO₄, filtered concentrated under reduced pressure to provide the crude product. The crude was purified by silica gel column chromatography to afford the desired product. White solid (0.04 g, 54%). ¹H NMR (600 MHz, DMSO) δ 7.06 (s, 1H), 6.87 (s, 1H), 4.24-4.19 (m, 4H), 2.21 (s, 3H). Not easily visible using LC-MS.

Methyl (E)-3-(7-methyl-2,3-dihydrobenzo[b][1,4]dioxin-6-yl) acrylate (I-9u). The reaction was conducted following the General procedure C, using I-8u (0.04 g, 0.17 mmol), methyl acrylate (0.023 ml, 0.26 mmol), Pd(OAc)₂ (0.002 g, 0.009 mmol), tri(o-tolyl)phosphine (0.0053 g, 0.016 mmol), and Et₃N (0.17 ml, 1.18 mmol). Yellow solid (0.005 g, 13%). ¹H NMR (600 MHz, DMSO) δ 7.74 (d, J=15.8 Hz, 1H), 7.27 (s, 1H), 6.76 (d, J=0.8 Hz, 1H), 6.40 (d, J=15.8 Hz, 1H), 4.28-4.20 (m, 4H), 3.71 (s, 3H), 2.28 (d, J=0.7 Hz, 3H). LC-MS (ESI): m/z=235.1, t_{R}=4.27 min.

Methyl 3-(7-(2-(cycloheptylamino)-2-oxoethoxy)naphthalen-2-yl)-3-(7-methyl-2,3-dihydrobenzo[b][1,4]dioxin-6-yl)propanoate (I-7u). The reaction was conducted following the general procedure F, using I-6 (0.075, 0.18 mmol) and methyl cinnamate I-9u (0.028 g, 0.12 mmol), TEA (0.1 ml, 0.54 mmol), and [Rh(COD)Cl]₂ (7 mg, 0.013 mmol). White solid (0.02 g, 22%). ¹H NMR (600 MHz, CDCl₃) δ 7.59 (d, J=8.5 Hz, 1H), 7.44-7.41 (m, 1H), 7.13 (dd, J=8.5, 1.8 Hz, 1H), 7.04 (dd, J=8.9, 2.6 Hz, 1H), 6.99 (d, J=2.6 Hz, 1H), 6.70 (s, 1H), 6.56 (s, 1H), 6.44 (d, J=8.5 Hz, 1H), 4.68 (t, J=7.8 Hz, 1H), 4.48 (s, 2H), 4.14 (s, 4H), 3.99 (tp, J=8.2, 4.2 Hz, 1H), 3.77 (s, 1H), 3.52 (s, 3H), 3.01-2.90 (m, 2H), 2.12 (s, 3H), 1.86 (qd, J=7.7, 3.7 Hz, 2H), 1.46-1.36 (m, 10H). LC-MS (ESI): m/z=532.3, t_{R}=4.99 min.

3-(7-(2-(Cycloheptylamino)-2-oxoethoxy)naphthalen-2-yl)-3-(7-methyl-2,3-dihydrobenzo[b][1,4]dioxin-6-yl)propanoic acid (2u, UCAB #1124). The reaction was conducted following the general procedure G, using I-7u (0.02 g, 0.04 mmol) and 2 M aq. NaOH (200 μl). White solid (0.0073 g, 37%). ¹H NMR (600 MHz, DMSO) δ 12.13 (s, 1H), 7.97 (d, J=8.1 Hz, 1H), 7.79-7.75 (m, 1H), 7.73 (d, J=8.5 Hz, 1H), 7.57-7.54 (m, 1H), 7.25 (dd, J=8.5, 1.8 Hz, 1H), 7.21-7.16 (m, 2H), 6.82 (s, 1H), 6.63 (d, J=0.8 Hz, 1H), 4.61 (t, J=7.9 Hz, 1H), 4.54 (s, 2H), 4.22-4.13 (m, 4H), 3.83 (dtd, J=12.6, 9.0, 4.5 Hz, 1H), 3.02-2.92 (m, 2H), 2.18 (s, 3H), 1.80-1.72 (m, 2H), 1.66-1.35 (m, 10H). ¹³C NMR (151 MHz, DMSO) δ 173.18, 166.56, 158.38, 156.35, 142.36, 141.77, 141.72, 135.04, 134.49, 129.37, 128.82, 128.10, 127.76, 125.03, 118.94, 118.74, 115.72, 107.57, 67.51, 64.51, 64.50, 50.03, 42.54, 40.85, 34.67 (2C), 28.15 (2C), 24.21 (2C), 19.03. LC-MS (ESI): m/z=518.2, t_{R}=4.61 min.

Methyl (E)-3-(5-chloro-2-methylphenyl) acrylate (I-9v). The reaction was conducted following the General procedure C, using 2-bromo-4-chloro-1-methylbenzene (1.0 g, 4.86 mmol), methyl acrylate (0.66 ml, 7.2 mmol), Pd(OAc)₂ (0.06 g, 0.26 mmol), tri(o-tolyl)phosphine (0.15 g, 0.5 mmol), and Et₃N (4.4 ml, 31.4 mmol). Yellow solid (0.12 g, 12%). ¹H NMR (600 MHz, DMSO) δ 7.91-7.71 (m, 2H), 7.45-7.18 (m, 2H), 6.65 (d, J=15.9 Hz, 1H), 3.75 (s, 3H), 2.37 (s, 3H). LC-MS (ESI): m/z=211.0, t_{R}=4.73 min.

Methyl 3-(5-chloro-2-methylphenyl)-3-(7-(2-(cycloheptylamino)-2-oxoethoxy) naphthalen-2-yl)propanoate (I-7v). The reaction was conducted following the general procedure F, using I-6 (0.12 g, 0.28 mmol) and methyl cinnamate (I-9v, 0.042 g, 0.2 mmol), TEA (0.12 ml, 0.8 mmol), and [Rh(COD)Cl]₂ (10 mg, 0.02 mmol). White solid (0.074 g, 52%). ¹H NMR (600 MHz, DMSO) δ 7.97 (d, J=8.1 Hz, 1H), 7.77 (dd, J=15.2, 8.6 Hz, 2H), 7.59 (d, J=1.8 Hz, 1H), 7.41 (d, J=1.8 Hz, 1H), 7.29 (dd, J=8.4, 1.8 Hz, 1H), 7.23-7.16 (m, 4H), 4.76 (t, J=7.8 Hz, 1H), 4.55 (s, 2H), 3.83 (qt, J=8.9, 4.3 Hz, 1H), 3.52 (s, 3H), 3.26-3.16 (m, 2H), 2.31 (s, 3H), 1.76 (dp, J=12.6, 4.4 Hz, 2H), 1.64-1.44 (m, 8H), 1.39 (qt, J=9.9, 4.0 Hz, 2H). LC-MS (ESI): m/z=508.2, t_{R}=5.28 min.

3-(5-chloro-2-methylphenyl)-3-(7-(2-(cycloheptylamino)-2-oxoethoxy)naphthalen-2-yl)propanoic acid (2v, UCAB #1101). The reaction was conducted following the general procedure G, using I-7v (0.08, 0.16 mmol) and 2 M aq. NaOH (720 μl). White solid (0.048 g, 34%). ¹H NMR (600 MHz, DMSO) δ 12.22 (s, 1H), 7.97 (d, J=8.1 Hz, 1H), 7.77 (dd, J=15.4, 8.7 Hz, 2H), 7.58 (d, J=1.7 Hz, 1H), 7.39 (d, J=1.7 Hz, 1H), 7.29 (dd, J=8.5, 1.8 Hz, 1H), 7.22-7.15 (m, 4H), 4.74 (t, J=7.8 Hz, 1H), 4.55 (s, 2H), 3.87-3.78 (m, 1H), 3.13-3.03 (m, 2H), 2.30 (s, 3H), 1.76 (dddd, J=12.9, 7.7, 4.5, 2.5 Hz, 2H), 1.65-1.44 (m, 8H), 1.44-1.34 (m, 2H). ¹³C NMR (151 MHz, DMSO) δ 173.05, 166.54, 156.43, 144.59, 141.37, 135.29, 134.52, 132.59, 131.15, 129.41, 128.29, 127.88, 126.85, 126.61, 125.28, 124.97, 118.94, 107.59, 67.51, 50.02, 43.01, 40.53, 34.66 (2C), 28.16 (2C), 24.20 (2C), 19.35. LC-MS (ESI): m/z=494.2, t_{R}=4.68 min.

Methyl (E)-3-(7-methyl-2,3-dihydrobenzo[b][1,4]dioxin-6-yl) acrylate (I-9w). The reaction was conducted following the General procedure C, using 5-bromo-2,2,6-trimethylbenzo[d][1,3]dioxole (0.05 g, 0.2 mmol), methyl acrylate (0.027 ml, 0.3 mmol), Pd(OAc)₂ (0.0024 g, 0.01 mmol), tri(o-tolyl)phosphine (0.006 g, 0.02 mmol), and Et₃N (0.2 ml, 1.39 mmol). Yellow solid (0.09 g, 30%). ¹H NMR (600 MHz, DMSO) δ 7.87 (s, 1H), 7.81 (d, J=15.8 Hz, 1H), 7.40-7.36 (m, 1H), 6.61 (d, J=15.8 Hz, 1H), 3.74 (s, 3H), 2.42 (d, J=0.6 Hz, 3H). LC-MS (ESI): m/z=257.1, t_{R}=4.82 min.

Methyl 3-(7-(2-(cyclohepten-1-ylamino)-2-oxoethoxy)naphthalen-2-yl)-3-(2,2-difluoro-6-methylbenzo[d][1,3]dioxol-5-yl)propanoate (I-7w). The reaction was conducted following the general procedure F, using I-6 (0.075 g, 0.18 mmol) and methyl cinnamate 1-9w (0.030 g, 0.12 mmol), TEA (0.11 ml, 0.6 mmol), and [Rh(COD)Cl]₂ (8 mg, 0.016 mmol). White solid (0.050 g, 50%). ¹H NMR (600 MHz, DMSO) δ 7.97 (d, J=8.2 Hz, 1H), 7.76 (dd, J=15.6, 8.7 Hz, 2H), 7.57 (d, J=1.7 Hz, 1H), 7.52 (s, 1H), 7.30 (dd, J=8.5, 1.8 Hz, 1H), 7.23 (s, 1H), 7.22-7.15 (m, 2H), 4.77 (t, J=7.8 Hz, 1H), 4.54 (s, 2H), 3.82 (ddp, J=12.6, 9.0, 4.4 Hz, 1H), 3.52 (s, 3H), 3.27-3.16 (m, 2H), 2.34 (s, 3H), 1.75 (qt, J=7.7, 2.5 Hz, 2H), 1.67-1.13 (m, 10H). LC-MS (ESI): m/z=554.2, t_{R}=5.40 min.

3-(7-(2-(cycloheptylamino)-2-oxoethoxy)naphthalen-2-yl)-3-(2,2-difluoro-6-methylbenzo[d][1,3]dioxol-5-yl)propanoic acid (2w, UCAB #1129). The reaction was conducted following the general procedure G, using I-7w (0.05 g, 0.09 mmol) and 2 M aq. NaOH (500 μl). White solid (0.002 g, 4%). ¹H NMR (600 MHz, DMSO) δ 12.20 (s, 1H), 7.96 (d, J=8.1 Hz, 1H), 7.76 (dd, J=16.3, 8.7 Hz, 2H), 7.57 (d, J=1.8 Hz, 1H), 7.51 (s, 1H), 7.31 (dd, J=8.5, 1.8 Hz, 1H), 7.23 (s, 1H), 7.22-7.14 (m, 2H), 4.74 (t, J=7.8 Hz, 1H), 4.54 (s, 2H), 3.83 (qt, J=9.0, 4.4 Hz, 1H), 3.19-3.02 (m, 2H), 2.34 (s, 3H), 1.83-1.69 (m, 2H), 1.69-1.33 (m, 9H), 1.28-1.20 (m, 1H). ¹³C NMR (151 MHz, DMSO) δ 216.15, 173.08, 166.52, 156.42, 141.79, 141.51, 141.34, 138.80, 134.50, 132.82, 129.39, 128.28, 127.85, 125.28, 124.85, 118.94, 112.00, 109.01, 107.60, 67.51, 50.02, 42.93, 40.68, 34.67 (2C), 28.14 (2C), 24.21 (2C), 19.93. LC-MS (ESI): m/z=540.2, t_{R}=5.12 min.

Methyl (E)-3-(6-methyl-2,3-dihydrobenzofuran-5-yl) acrylate (I-9x). The reaction was conducted following the General procedure C, using 5-bromo-6-methyl-2,3-dihydrobenzofuran (0.11 g, 0.52 mmol), methyl acrylate (0.081 ml, 0.78 mmol), Pd(OAc)₂ (0.0057 g, 0.025 mmol), tri(o-tolyl)phosphine (0.0155 g, 0.048 mmol), and Et₃N (0.5 ml, 3.65 mmol). cololess oil (0.03 g, 30%). ¹H NMR (600 MHz, DMSO) δ 7.80 (dd, J=15.9, 1.3 Hz, 1H), 7.52 (d, J=1.4 Hz, 1H), 6.57 (s, 1H), 6.26 (dd, J=15.9, 1.2 Hz, 1H), 4.48 (td, J=8.7, 1.3 Hz, 2H), 4.18 (s, 3H), 3.13 (m, J=1.6 Hz, 2H), 2.48 (s, J=1.9 Hz, 3H). LC-MS (ESI): m/z=219.2, t_{R}=4.40 min.

Methyl 3-(7-(2-(cycloheptylamino)-2-oxoethoxy)naphthalen-2-yl)-3-(6-methyl-2,3-dihydrobenzofuran-5-yl)propanoate (I-7x). The reaction was conducted following the general procedure F, using I-6 (0.090 g, 0.21 mmol) and methyl cinnamate I-9x (0.030 g, 0.14 mmol), TEA (0.13 ml, 0.7 mmol), and [Rh(COD)Cl]₂ (9 mg, 0.019 mmol). White solid (0.026 g, 24%). LC-MS (ESI): m/z=516.3, t_{R}=5.17 min.

3-(7-(2-(cycloheptylamino)-2-oxoethoxy)naphthalen-2-yl)-3-(6-methyl-2,3-dihydrobenzofuran-5-yl)propanoic acid (2x, UCAB #1141). The reaction was conducted following the general procedure G, using I-7x (0.026 g, 0.05 mmol) and 2 M aq. NaOH (240 μl). White solid (0.003 g, 13%). ¹H NMR (600 MHz, DMSO) δ 12.11 (s, 1H), 7.96 (d, J=8.1 Hz, 1H), 7.79-7.74 (m, 1H), 7.72 (d, J=8.5 Hz, 1H), 7.58-7.55 (m, 1H), 7.24 (dd, J=8.5, 1.8 Hz, 1H), 7.21-7.16 (m, 3H), 6.56 (s, 1H), 4.68 (t, J=7.9 Hz, 1H), 4.54 (s, 2H), 4.45 (ddd, J=15.5, 9.1, 7.7 Hz, 2H), 3.83 (tq, J=12.6, 4.5 Hz, 1H), 3.11 (t, J=8.7 Hz, 2H), 3.06-2.93 (m, 2H), 2.26 (s, 3H), 1.80-1.72 (m, 2H), 1.68-1.32 (m, 9H), 1.25 (s, 1H). ¹³C NMR (151 MHz, DMSO) δ 173.28, 166.55, 158.49, 156.35, 142.73, 135.63, 134.50, 134.04, 129.36, 128.06, 127.73, 125.11, 125.09, 124.93, 123.59, 118.68, 111.12, 107.61, 71.27, 67.52, 50.02, 42.45, 40.98, 34.67 (2C), 29.55, 28.17 (2C), 24.21 (2C), 20.24. LC-MS (ESI): m/z=502.4, t_{R}=4.78 min.

Methyl (E)-3-(6-methylbenzofuran-5-yl) acrylate (I-9y). The reaction was conducted following the General procedure C, using 5-bromo-6-methylbenzofuran (0.25 g, 1.19 mmol), methyl acrylate (0.13 ml, 1.8 mmol), Pd(OAc)₂ (0.0014 g, 0.06 mmol), tri(o-tolyl)phosphine (0.04 g, 0.13 mmol), and Et₃N (1.2 ml, 8.53 mmol). Cololess oil (0.15 g, 59.4%). ¹H NMR (600 MHz, DMSO) δ 8.04 (s, 1H), 7.98-7.92 (m, 2H), 7.51 (s, 1H), 6.91 (dd, J=2.2, 1.0 Hz, 1H), 6.53 (d, J=15.8 Hz, 1H), 3.73 (s, 3H), 2.50 (s, 3H). LC-MS (ESI): m/z=217.1, t_{R}=4.66 min.

Methyl 3-(7-(2-(cycloheptylamino)-2-oxoethoxy)naphthalen-2-yl)-3-(6-methylbenzofuran-5-yl)propanoate (I-7y). The reaction was conducted following the general procedure F, using I-6 (0.12 g, 0.35 mmol) and methyl cinnamate I-9y (0.04 g, 0.24 mmol), TEA (0.1 ml, 0.71 mmol), and [Rh(COD)Cl]₂ (12 mg, 0.024 mmol). White solid (0.073 g, 80.1%). ¹H NMR (600 MHz, DMSO) δ 7.95 (d, J=8.2 Hz, 1H), 7.89 (d, J=2.2 Hz, 1H), 7.79-7.71 (m, 2H), 7.64 (s, 1H), 7.55-7.52 (m, 1H), 7.40 (s, 1H), 7.28 (dd, J=8.5, 1.8 Hz, 1H), 7.18 (dd, J=8.9, 2.5 Hz, 1H), 7.15 (d, J=2.6 Hz, 1H), 6.89 (dd, J=2.2, 1.0 Hz, 1H), 4.84 (d, J=7.9 Hz, 1H), 4.53 (s, 2H), 3.81 (dp, J=13.4, 4.5 Hz, 1H), 3.51 (s, 3H), 3.25-3.17 (m, 2H), 2.40 (d, J=5.9 Hz, 3H), 1.74 (dh, J=12.5, 4.1 Hz, 2H), 1.64-1.32 (m, 10H).LC-MS (ESI): m/z=514.3, t_{R}=5.31 min.

3-(7-(2-(cycloheptylamino)-2-oxoethoxy)naphthalen-2-yl)-3-(6-methylbenzo[d][1,3]dioxol-5-yl)propanoic acid (2y, UCAB #1191). The reaction was conducted following the general procedure G, using I-7y (0.073 g, 0.15 mmol) and 2 M aq. NaOH (150 μl). White solid (0.008 g, 11.2%). ¹H NMR (600 MHz, DMSO) δ 12.14 (s, 1H), 7.95 (d, J=8.1 Hz, 1H), 7.88 (d, J=2.2 Hz, 1H), 7.78-7.70 (m, 2H), 7.63 (s, 1H), 7.53 (s, 1H), 7.38 (s, 1H), 7.28 (dd, J=8.5, 1.8 Hz, 1H), 7.20-7.12 (m, 2H), 6.88 (d, J=2.7 Hz, 1H), 4.82 (t, J=7.8 Hz, 1H), 4.52 (s, 2H), 3.81 (ddq, J=13.4, 8.9, 4.4 Hz, 1H), 3.12-3.05 (m, 2H), 2.39 (s, 3H), 1.73 (dtd, J=12.5, 8.0, 3.8 Hz, 2H), 1.63-1.42 (m, 8H), 1.38 (ddt, J=16.2, 10.0, 2.7 Hz, 2H). ¹³C NMR (151 MHz, DMSO) δ 173.22, 166.54, 156.38, 153.58, 145.84, 142.47, 137.28, 134.49, 133.19, 129.37, 128.13, 127.76, 125.66, 125.19, 119.43, 118.79, 112.91, 107.56, 107.09, 67.49, 49.99, 42.99, 41.32, 34.65 (2C), 28.16 (2C), 24.19 (2C), 20.53. LC-MS (ESI): m/z=500.2, t_{R}=4.87 min.

Methyl (E)-3-(6-chlorobenzo[d][1,3]dioxol-5-yl) acrylate (I-9z). The reaction was conducted following the General procedure C, using 5-bromo-6-chlorobenzo[d][1,3]dioxole (0.5 g, 2.23 mmol), methyl acrylate (0.3 ml, 3.19 mmol), Pd(OAc)₂ (0.024 g, 0.11 mmol), tri(o-tolyl)phosphine (0.065 g, 0.22 mmol), and Et₃N (2.2 ml, 15.23 mmol). Cololess oil (0.4 g, 78.4%). ¹H NMR (600 MHz, DMSO) δ 7.86 (d, J=15.9 Hz, 1H), 7.59 (s, 1H), 7.20 (s, 1H), 6.65 (d, J=15.9 Hz, 1H), 6.15 (s, 2H), 3.73 (s, 3H). LC-MS (ESI): m/z=241.1, t_{R}=4.54 min.

Methyl 3-(6-chlorobenzo[d][1,3]dioxol-5-yl)-3-(7-(2-(cycloheptylamino)-2-oxoethoxy)naphthalen-2-yl)propanoate (I-7z). The reaction was conducted following the general procedure F, using I-6 (0.15 g, 0.35 mmol) and methyl cinnamate 1-9z (0.06 g, 0.24 mmol), TEA (0.1 ml, 0.71 mmol), and [Rh(COD)Cl]₂ (12 mg, 0.024 mmol). White solid (0.02 g, 14.9%). ¹H NMR (400 MHz, DMSO) δ 7.99-7.89 (m, 1H), 7.75 (dd, J=11.0, 8.5 Hz, 2H), 7.68-7.56 (m, 1H), 7.31 (dd, J=8.5, 1.8 Hz, 1H), 7.22-7.15 (m, 3H), 7.02 (s, 1H), 6.06 (d, J=1.0 Hz, 1H), 6.01 (d, J=1.0 Hz, 1H), 4.94 (t, J=7.9 Hz, 1H), 4.54 (s, 2H), 3.82 (qt, J=9.0, 4.5 Hz, 1H), 3.51 (s, 3H), 3.29-3.11 (m, 2H), 1.75 (t, J=6.8 Hz, 2H), 1.64-1.23 (m, 10H). LC-MS (ESI): m/z=538.4, t_{R}=5.28 min.

3-(6-chlorobenzo[d][1,3]dioxol-5-yl)-3-(7-(2-(cycloheptylamino)-2-oxoethoxy)naphthalen-2-yl)propanoic acid (2z, UCAB #1221). The reaction was conducted following the general procedure G, using I-7y (0.02 g, 0.04 mmol) and 2 M aq. NaOH (100 μl). White solid (0.007 g, 36.1%). ¹H NMR (600 MHz, DMSO) δ 12.19 (s, 1H), 8.02-7.91 (m, 1H), 7.76 (dd, J=17.3, 8.6 Hz, 2H), 7.62 (s, 1H), 7.33 (dd, J=8.5, 1.8 Hz, 1H), 7.22-7.17 (m, 3H), 7.03 (s, 1H), 6.07-5.98 (m, 2H), 4.92 (t, J=7.9 Hz, 1H), 4.54 (d, J=12.3 Hz, 2H), 3.83 (qt, J=9.0, 4.4 Hz, 1H), 3.15-3.01 (m, 2H), 1.76 (ddd, J=12.1, 8.2, 3.8 Hz, 2H), 1.67-1.18 (m, 10H). ¹³C NMR (151 MHz, DMSO) δ 172.91, 166.53, 156.39, 147.34, 146.90, 141.32, 134.51, 134.47, 129.38, 128.18, 127.88, 125.25, 124.79, 124.67, 118.91, 110.01, 108.66, 107.61, 102.40, 67.53, 50.03, 43.22, 40.43, 34.68 (2C), 28.16 (2C), 24.21 (2C). LC-MS (ESI): m/z=524.3, t_{R}=4.82 min.

Methyl (E)-3-(4-methoxy-2,6-dimethylphenyl) acrylate (I-9aa). The reaction was conducted following the General procedure C, using 2-bromo-5-methoxy-1,3-dimethylbenzene (0.50 g, 2.33 mmol), methyl acrylate (0.32 ml, 3.49 mmol), Pd(OAc)₂ (0.03 g, 0.12 mmol), tri(o-tolyl)phosphine (0.07 g, 0.24 mmol), and Et₃N (2.4 ml, 17.67 mmol). Light yellow solid (0.15 g, 29.9%). ¹H NMR (400 MHz, DMSO) δ 7.74 (d, J=16.3 Hz, 1H), 6.70 (s, 2H), 6.09 (d, J=16.4 Hz, 1H), 3.73 (d, J=8.9 Hz, 6H), 2.31 (s, 6H). LC-MS (ESI): m/z=221.1, t_{R}=4.78 min.

Methyl (E)-3-(2,2-dimethylbenzo[d][1,3]dioxol-5-yl) acrylate (I-9ab) The reaction was conducted following the General procedure C, using 5-bromo-2,2-dimethylbenzo[d][1,3]dioxole (0.10 g, 0.44 mmol), methyl acrylate (0.06 ml, 0.66 mmol), Pd(OAc)₂ (0.005 g, 0.03 mmol), tri(o-tolyl)phosphine (0.015 g, 0.05 mmol), and Et₃N (0.44 ml, 3.13 mmol). White solid (0.06 g, 57.7%). ¹H NMR (400 MHz, DMSO) δ 7.55 (d, J=16.0 Hz, 1H), 7.30 (d, J=1.7 Hz, 1H), 7.14 (dd, J=8.2, 1.8 Hz, 1H), 6.86 (d, J=8.0 Hz, 1H), 6.47 (d, J=15.9 Hz, 1H), 3.69 (s, 3H), 1.65 (s, 6H).LC-MS (ESI): m/z=235.1, t_{R}=4.60 min.

Methyl (E)-3-(5-methoxy-2-methylphenyl) acrylate (I-9ac). The reaction was conducted following the General procedure C, using 2-bromo-4-methoxy-1-methylbenzene (0.5 g, 2.5 mmol), methyl acrylate (0.34 ml, 3.73 mmol), Pd(OAc)₂ (0.03 g, 0.12 mmol), tri(o-tolyl)phosphine (0.08 g, 0.23 mmol), and Et₃N (2.5 ml, 16.7 mmol). White solid (0.50 g, 97.8%). ¹H NMR (600 MHz, DMSO) δ 7.82 (d, J=15.9 Hz, 1H), 7.26 (d, J=2.7 Hz, 1H), 7.18-7.15 (m, 1H), 6.91 (dd, J=8.4, 2.7 Hz, 1H), 6.59 (d, J=15.9 Hz, 1H), 3.75 (d, J=20.3 Hz, 6H), 2.31 (s, 3H). LC-MS (ESI): m/z=207.1, t_{R}=4.50 min.

Methyl (E)-3-(6-methyl-1H-pyrrolo[2,3-b]pyridin-5-yl) acrylate (I-9ad). The reaction was conducted following the General procedure C, using 5-bromo-6-methyl-1H-pyrrolo[2,3-b]pyridine (0.1 g, 0.71 mmol), methyl acrylate (0.1 ml, 1.07 mmol), Pd(OAc)₂ (0.008 g, 0.04 mmol), tri(o-tolyl)phosphine (0.022 g, 0.07 mmol), and Et₃N (0.72 ml, 5.1 mmol). White solid (0.04 g, 39.2%). LC-MS (ESI): m/z=217.2, t_{R}=3.34 min.

N-Cyclohexyl-2-((7-(4,4,5,5-tetramethyl-1,3,2-dioxaborolan-2-yl)naphthalen-2-yl)oxy) acetamide (I-13). A solution of I-3a (0.30 g, 0.80 mmol), PinB-BPin (0.26 g, 1.00 mmol), KOAc (0.24 g, 2.48 mmol), and Pd(dppf)Cl₂ (0.02 g, 0.025 mmol) were added in dioxane (3 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.97 g, 89.1%). ¹H NMR (600 MHz, DMSO) δ 8.18 (d, J=1.1 Hz, 1H), 7.96-7.91 (m, 1H), 7.85 (dd, J=23.6, 8.5 Hz, 2H), 7.58 (dd, J=8.1, 1.1 Hz, 1H), 7.39 (d, J=2.6 Hz, 1H), 7.33 (dd, J=8.9, 2.6 Hz, 1H), 4.56 (s, 2H), 1.72 (ddd, J=28.2, 8.2, 3.8 Hz, 4H), 1.57 (d, J=12.8 Hz, 1H), 1.34 (s, 12H), 1.32-1.22 (m, 4H), 1.12 (d, J=10.5 Hz, 1H). LC-MS (ESI): m/z=410.3, t_{R}=5.41 min.

Methyl 3-(7-(2-(cyclohexylamino)-2-oxoethoxy)naphthalen-2-yl)-3-(4-methoxyphenyl)propanoate (I-14a). The reaction was conducted following the general procedure F, using I-13 (0.16, 0.39 mmol) and methyl (E)-3-(4-methoxyphenyl) acrylate (0.05 g, 0.26 mmol), TEA (0.11 ml, 0.78 mmol), and [Rh(COD)Cl]₂ (13 mg, 0.03 mmol). Colorless oil (0.07 g, 59.1%). ¹H NMR (400 MHz, DMSO) δ 7.91 (d, J=8.2 Hz, 1H), 7.74 (dd, J=17.1, 9.0 Hz, 2H), 7.65 (d, J=1.7 Hz, 1H), 7.27 (t, J=9.1 Hz, 3H), 7.19 (dq, J=4.3, 2.5 Hz, 2H), 6.88-6.79 (m, 2H), 4.54 (d, J=7.1 Hz, 3H), 3.70 (s, 3H), 3.68-3.61 (m, 1H), 3.49 (s, 3H), 3.26-3.09 (m, 2H), 1.77-1.65 (m, 4H), 1.61-1.53 (m, 1H), 1.26 (t, J=10.2 Hz, 5H). LC-MS (ESI): m/z=476.3, t_{R}=5.08 min.

3-(7-(2-(cyclohexylamino)-2-oxoethoxy)naphthalen-2-yl)-3-(4-methoxyphenyl)propanoic acid (3a, UCAB #1022). The reaction was conducted following the general procedure G, using I-14a (0.07, 0.16 mmol) and 2M aq. NaOH (700 μl). White solid (0.005 g, 7.1%). ¹H NMR (600 MHz, DMSO) δ 12.12 (s, 1H), 7.94 (d, J=8.2 Hz, 1H), 7.80-7.76 (d, 1H), 7.74 (d, J=8.5 Hz, 1H), 7.66 (d, J=1.7 Hz, 1H), 7.32-7.27 (m, 3H), 7.21 (d, J=7.5 Hz, 2H), 6.88-6.83 (m, 2H), 4.57 (s, 2H), 4.52 (t, J=8.0 Hz, 1H), 3.71 (s, 3H), 3.67 (dq, J=9.8, 5.9 Hz, 1H), 3.15-3.02 (m, 2H), 1.77-1.69 (m, 4H), 1.58 (m, J=10.3, 6.4 Hz, 1H), 1.29 (m, J=12.1, 8.8, 3.3 Hz, 4H), 1.21-1.11 (m, 1H). ¹³C NMR (151 MHz, DMSO) δ 172.72, 166.33, 157.67, 155.86, 142.66, 135.93, 134.09, 128.89, 128.57 (2C), 127.60, 127.30, 124.38, 124.21, 118.18, 113.77 (2C), 107.16, 67.03, 54.97, 47.40, 45.91, 40.06, 32.23 (2C), 25.14, 24.63 (2C). LC-MS (ESI): m/z=462.2, t_{R}=4.60 min.

Methyl 3-(7-(2-(cyclohexylamino)-2-oxoethoxy)naphthalen-2-yl)-3-(4-methoxy-2-methylphenyl)propanoate (I-14b). The reaction was conducted following the general procedure F, using I-13 (0.12, 0.30 mmol) and I-9k (0.04 g, 0.20 mmol), TEA (0.10 ml, 0.60 mmol), and [Rh(COD)Cl]₂ (10 mg, 0.02 mmol). Colorless oil (0.07 g, 31.9%). ¹H NMR (400 MHz, DMSO) δ 7.95-7.88 (m, 1H), 7.75 (dd, J=16.9, 8.6 Hz, 2H), 7.54 (d, J=1.8 Hz, 1H), 7.31-7.16 (m, 4H), 6.79-6.72 (m, 2H), 4.70 (t, J=7.9 Hz, 1H), 4.55 (s, 2H), 3.71 (s, 3H), 3.64 (m, 1H), 3.51 (s, 3H), 3.13 (dd, J=7.9, 2.9 Hz, 2H), 2.28 (s, 3H), 1.77-1.62 (m, 4H), 1.57 (d, J=12.7 Hz, 1H), 1.26 (s, 5H). LC-MS (ESI): m/z=409.3, t_{R}=5.19 min.

3-(7-(2-(cyclohexylamino)-2-oxoethoxy)naphthalen-2-yl)-3-(4-methoxy-2-methylphenyl)propanoic acid (3b, UCAB #1018). The reaction was conducted following the general procedure G, using I-14b (0.03 g, 0.06 mmol) and 2M aq. NaOH (200 μl). White solid (0.03 g, 88.2%). ¹H NMR (600 MHz, DMSO) δ 12.11 (s, 1H), 7.92 (d, J=8.2 Hz, 1H), 7.76 (d, J=8.7 Hz, 1H), 7.72 (d, J=8.5 Hz, 1H), 7.54 (d, J=1.7 Hz, 1H), 7.27 (d, J=8.6 Hz, 1H), 7.24 (dd, J=8.5, 1.8 Hz, 1H), 7.21-7.15 (m, 2H), 6.76 (dd, J=8.5, 2.8 Hz, 1H), 6.72 (d, J=2.8 Hz, 1H), 4.68 (t, J=7.9 Hz, 1H), 4.54 (s, 2H), 3.71 (s, 3H), 3.64 (dq, J=14.7, 5.4, 4.5 Hz, 1H), 3.01 (h, J=7.7 Hz, 2H), 2.27 (s, 3H), 1.75-1.66 (m, 4H), 1.57 (d, J=13.0 Hz, 1H), 1.26 (ddt, J=12.8, 10.2, 4.2 Hz, 4H), 1.14 (pd, J=13.5 Hz, 1H). ¹³C NMR (151 MHz, DMSO) δ 173.24, 166.83, 157.89, 156.33, 142.58, 137.50, 134.48, 134.21, 129.35, 128.04 (2C), 127.74, 125.06 (2C), 118.72, 116.38, 111.64, 107.61, 67.50, 55.34, 47.86, 42.36, 40.98, 32.71 (2C), 25.64, 25.10 (2C). LC-MS (ESI): m/z=476.3, t_{R}=4.65 min.

Methyl 3-(7-(2-(cyclohexylamino)-2-oxoethoxy)naphthalen-2-yl)-3-(4-methoxy-2,6-dimethylphenyl)propanoate (I-14c). The reaction was conducted following the general procedure F, using I-13 (0.14, 0.35 mmol) and I-9aa (0.05 g, 0.23 mmol), TEA (0.10 ml, 0.68 mmol), and [Rh(COD)Cl]₂ (12 mg, 0.02 mmol). Colorless oil (0.07 g, 59.1%). ¹H NMR (400 MHz, DMSO) δ 7.91 (d, J=8.2 Hz, 1H), 7.74 (dd, J=17.1, 9.0 Hz, 2H), 7.65 (d, J=1.7 Hz, 1H), 7.27 (t, J=9.1 Hz, 3H), 7.19 (dq, J=4.3, 2.5 Hz, 2H), 6.88-6.79 (m, 2H), 4.54 (d, J=7.1 Hz, 3H), 3.70 (s, 3H), 3.68-3.61 (m, 1H), 3.49 (s, 3H), 3.26-3.09 (m, 2H), 1.77-1.65 (m, 4H), 1.61-1.53 (m, 1H), 1.26 (t, J=10.2 Hz, 5H). LC-MS (ESI): m/z=476.3, t_{R}=5.08 min.

3-(7-(2-(cyclohexylamino)-2-oxoethoxy)naphthalen-2-yl)-3-(4-methoxy-2,6-dimethylphenyl)propanoic acid (3c, UCAB #1021). The reaction was conducted following the general procedure G, using I-14c (0.03 g, 0.06 mmol) and 2 M aq. NaOH (300 μl). White solid (0.012 g, 41.1%). ¹H NMR (600 MHz, DMSO) δ 12.25 (s, 1H), 7.92 (d, J=8.2 Hz, 1H), 7.77 (d, J=8.9 Hz, 1H), 7.72 (d, J=8.5 Hz, 1H), 7.46 (d, J=1.6 Hz, 1H), 7.21-7.14 (m, 2H), 7.02 (dd, J=8.5, 1.8 Hz, 1H), 6.60 (s, 2H), 5.05 (t, J=7.5 Hz, 1H), 4.55 (s, 2H), 4.01 (s, 7H), 3.71 (s, 3H), 3.39 (dd, J=15.6, 7.9 Hz, 1H), 2.86 (dd, J=15.6, 7.1 Hz, 1H), 1.70 (ddd, J=16.7, 9.4, 3.6 Hz, 4H), 1.57 (d, J=12.7 Hz, 1H), 1.33-1.19 (m, 4H), 1.13 (dtd, J=15.5, 9.8, 5.9 Hz, 1H). ¹³C NMR (151 MHz, DMSO) δ 174.07, 166.87, 157.69, 156.39, 141.84, 138.47 (3C), 134.39, 133.10, 129.32, 127.94, 127.41, 124.22, 118.66, 107.64 (3C), 67.50, 55.19 (3C), 47.83, 40.50, 37.48, 32.70 (2C), 25.64, 25.08 (2C). LC-MS (ESI): m/z=490.2, t_{R}=4.82 min.

Methyl 3-(3-chlorophenyl)-3-(7-(2-(cyclohexylamino)-2-oxoethoxy)naphthalen-2-yl)propanoate (I-14d). The reaction was conducted following the general procedure F, using I-13 (0.15, 0.40 mmol) and I-9i (0.05 g, 0.25 mmol), TEA (0.11 ml, 0.73 mmol), and [Rh(COD)Cl]₂ (13 mg, 0.03 mmol). Colorless oil (0.08 g, 45.7%). ¹H NMR (600 MHz, DMSO) δ 7.93-7.89 (m, 1H), 7.75 (dd, J=17.8, 8.6 Hz, 2H), 7.72-7.69 (m, 1H), 7.44 (t, J=1.9 Hz, 1H), 7.37-7.29 (m, 3H), 7.25-7.16 (m, 3H), 4.61 (t, J=8.0 Hz, 1H), 4.55 (s, 2H), 3.63 (m, 1H), 3.50 (s, 3H), 3.25 (dd, J=7.9, 1.3 Hz, 2H), 1.76-1.65 (m, 4H), 1.56 (d, J=12.7 Hz, 1H), 1.24 (d, J=11.5 Hz, 5H). LC-MS (ESI): m/z=480.2, t_{R}=5.30 min.

3-(3-chlorophenyl)-3-(7-(2-(cyclohexylamino)-2-oxoethoxy)naphthalen-2-yl)propanoic acid (3d, UCAB #1017). The reaction was conducted following the general procedure G, using I-14d (0.08 g, 0.17 mmol) and 2 M aq. NaOH (200 μl). White solid (0.03 g, 43.2%). ¹H NMR (600 MHz, DMSO) δ 12.16 (s, 1H), 7.91 (d, J=8.2 Hz, 1H), 7.78-7.75 (m, 1H), 7.74 (d, J=8.5 Hz, 1H), 7.70 (d, J=1.7 Hz, 1H), 7.43 (t, J=1.9 Hz, 1H), 7.36-7.32 (m, 2H), 7.30 (t, J=7.8 Hz, 1H), 7.23 (ddd, J=7.9, 2.2, 1.2 Hz, 1H), 7.21-7.18 (m, 2H), 4.57 (t, J=8.0 Hz, 1H), 4.54 (s, 2H), 3.64 (dq, J=10.7, 6.1 Hz, 1H), 3.13 (d, J=7.9 Hz, 2H), 1.75-1.67 (m, 4H), 1.58-1.53 (m, 1H), 1.28-1.25 (m, 4H), 1.12 (t, J=12.2 Hz, 1H). ¹³C NMR (151 MHz, DMSO) δ 172.67, 166.45, 156.06, 146.74, 141.75, 134.23, 133.13, 130.39, 129.07, 127.93, 127.56, 126.50, 126.41, 124.65, 124.39, 118.53, 107.31, 67.18, 47.54, 46.43, 40.20,32.37 (2C), 25.28, 24.77 (2C), LC-MS (ESI): m/z=466.2, t_{R}=4.87 min.

Methyl 3-(3-chloro-2-methylphenyl)-3-(7-(2-(cyclohexylamino)-2-oxoethoxy)naphthalen-2-yl)propanoate (I-14e). The reaction was conducted following the general procedure F, using I-13 (0.25, 0.61 mmol) and I-91 (0.09 g, 0.41 mmol), TEA (0.18 ml, 1.25 mmol), and [Rh(COD)Cl]₂ (21 mg, 0.04 mmol). White solid (0.16 g, 53.3%). ¹H NMR (600 MHz, DMSO) δ 7.92 (d, J=8.2 Hz, 1H), 7.80-7.73 (m, 2H), 7.57 (d, J=1.8 Hz, 1H), 7.38 (dd, J=7.8, 1.3 Hz, 1H), 7.31 (dd, J=8.0, 1.3 Hz, 1H), 7.28-7.16 (m, 4H), 4.87 (t, J=7.8 Hz, 1H), 4.55 (s, 2H), 3.69-3.59 (m, 1H), 3.52 (s, 3H), 3.19 (d, J=7.8 Hz, 2H), 2.37 (s, 3H), 1.75-1.62 (m, 4H), 1.57 (d, J=12.9 Hz, 1H), 1.26 (dh, J=13.6, 3.6 Hz, 5H). LC-MS (ESI): m/z=494.2, t_{R}=5.26 min.

3-(3-chloro-2-methylphenyl)-3-(7-(2-(cyclohexylamino)-2-oxoethoxy)naphthalen-2-yl)propanoic acid (3e, UCAB #1084). The reaction was conducted following the general procedure G, using I-14e (0.16 g, 0.32 mmol) and 2 M aq. NaOH (1.4 ml). White solid (0.07 g, 45.2%). ¹H NMR (600 MHz, DMSO) δ 12.2 (s, 1H), 7.92 (d, J=8.2 Hz, 1H), 7.76 (dd, J=18.6, 8.7 Hz, 2H), 7.57 (d, J=1.8 Hz, 1H), 7.37 (dd, J=7.9, 1.3 Hz, 1H), 7.31 (dd, J=8.0, 1.2 Hz, 1H), 7.29-7.16 (m, 4H), 4.84 (t, J=7.8 Hz, 1H), 4.55 (s, 2H), 3.69-3.59 (m, 1H), 3.07 (d, J=7.8 Hz, 2H), 2.37 (s, 3H), 1.79-1.62 (m, 4H), 1.56 (dd, J=10.5, 6.3 Hz, 1H), 1.26 (dtt, J=10.0, 7.1, 3.5 Hz, 4H), 1.18-1.08 (m, 1H). ¹³C NMR (151 MHz, DMSO) δ 173.04, 166.82, 156.42, 144.60, 141.59, 134.54, 134.01, 129.41, 128.30, 127.86, 127.65, 127.62, 126.23, 125.33, 124.93, 118.95, 107.65, 67.50, 47.88, 43.63, 40.78, 40.54, 32.71 (2C), 25.63, 25.10 (2C). LC-MS (ESI): m/z=480.2, t_{R}=4.871 min.

Methyl 3-(benzo[d][1,3]dioxol-5-yl)-3-(7-(2-(cyclohexylamino)-2-oxoethoxy)naphthalen-2-yl)propanoate (I-14f). The reaction was conducted following the general procedure F, using I-13 (0.15, 0.37 mmol) and I-9m (0.05 g, 0.25 mmol), TEA (0.18 ml, 0.74 mmol), and [Rh(COD)Cl]₂ (12 mg, 0.03 mmol). White solid (0.76 g, 47.8%). ¹H NMR (600 MHz, DMSO) δ 7.92 (d, J=8.1 Hz, 1H), 7.79-7.71 (m, 2H), 7.69-7.66 (m, 1H), 7.32 (dd, J=8.5, 1.8 Hz, 1H), 7.19 (d, J=7.4 Hz, 2H), 6.97 (d, J=1.7 Hz, 1H), 6.85-6.78 (m, 2H), 5.94 (dd, J=6.2, 1.0 Hz, 2H), 4.57-4.49 (m, 3H), 3.68-3.60 (m, 1H), 3.50 (s, 3H), 3.25-3.12 (m, 2H), 1.76-1.66 (m, 4H), 1.57 (d, J=13.0 Hz, 1H), 1.32-1.20 (m, 5H). LC-MS (ESI): m/z=490.2, t_{R}=5.01 min.

3-(benzo[d][1,3]dioxol-5-yl)-3-(7-(2-(cyclohexylamino)-2-oxoethoxy)naphthalen-2-yl)propanoic acid (3f, UCAB #1004). The reaction was conducted following the general procedure G, using I-14f (0.04 g, 0.09 mmol) and 2 M aq. NaOH (30 μl). White solid (0.03 g, 89.6%). ¹H NMR (600 MHz, DMSO) δ 12.08 (s, 1H), 7.92 (d, J=8.2 Hz, 1H), 7.79-7.74 (m, 1H), 7.72 (d, J=8.5 Hz, 1H), 7.67 (d, J=1.7 Hz, 1H), 7.31 (dd, J=8.5, 1.8 Hz, 1H), 7.19 (d, J=7.1 Hz, 2H), 6.95 (d, J=1.6 Hz, 1H), 6.87-6.78 (m, 2H), 5.94 (d, J=5.1 Hz, 2H), 4.55 (s, 2H), 4.48 (t, J=7.9 Hz, 1H), 3.65 (s, 1H), 3.13-3.00 (m, 2H), 1.78-1.53 (m, 6H), 1.33-1.07 (m, 6H). ¹³C NMR (151 MHz, DMSO) δ 173.04, 166.82, 156.42, 144.60, 141.59, 134.54, 134.01, 129.41, 128.30, 127.86, 127.65, 127.62, 126.23, 125.33, 124.93, 118.95, 107.65, 67.50, 47.88, 43.63, 40.54, 32.71, 25.63 (2C), 25.10 (2C), 16.26. LC-MS (ESI): m/z=476.2, t_{R}=4.59 min.

Methyl 3-(7-(2-(cyclohexylamino)-2-oxoethoxy)naphthalen-2-yl)-3-(6-methylbenzo[d][1,3]dioxol-5-yl)propanoate (I-14g). The reaction was conducted following the general procedure F, using I-13 (0.08, 0.21 mmol) and I-9n (0.03 g, 0.14 mmol), TEA (0.11 ml, 0.78 mmol), and [Rh(COD)Cl]₂ (13 mg, 0.03 mmol). White solid (0.03 g, 33.7%). ¹H NMR (600 MHz, DMSO) δ 7.91 (s, 1H), 7.79-7.71 (m, 2H), 7.58 (d, J=1.8 Hz, 1H), 7.27 (dd, J=8.5, 1.8 Hz, 1H), 7.22-7.17 (m, 2H), 6.99 (s, 1H), 6.73 (s, 1H), 5.95 (d, J=1.0 Hz, 1H), 5.91 (d, J=1.0 Hz, 1H), 4.69 (t, J=7.9 Hz, 1H), 4.55 (s, 2H), 3.64 (d, J=11.2 Hz, 1H), 3.51 (s, 3H), 3.13 (h, J=8.1 Hz, 2H), 2.25 (s, 3H), 1.75-1.66 (m, 4H), 1.57 (d, J=12.5 Hz, 1H), 1.29 (dt, J=7.5, 4.1 Hz, 5H). LC-MS (ESI): m/z=504.3, t_{R}=5.16 min.

3-(7-(2-(cyclohexylamino)-2-oxoethoxy)naphthalen-2-yl)-3-(6-methylbenzo[d][1,3]dioxol-5-yl)propanoic acid (3 g, UCAB #1026). The reaction was conducted following the general procedure G, using I-14g (0.03 g, 0.06 mmol) and 2 M aq. NaOH (700 μl). White solid (0.002 g, 6.9%). ¹H NMR (600 MHz, DMSO) δ 12.12 (s, 1H), 7.91 (d, J=8.2 Hz, 1H), 7.79-7.74 (m, 1H), 7.72 (d, J=8.5 Hz, 1H), 7.58 (s, 1H), 7.27 (dd, J=8.5, 1.8 Hz, 1H), 7.18 (dd, J=6.5, 2.6 Hz, 2H), 6.96 (s, 1H), 6.73 (s, 1H), 5.93 (d, J=19.3 Hz, 2H), 4.67 (t, J=7.9 Hz, 1H), 4.54 (s, 1H), 3.01 (qd, J=15.9, 7.9 Hz, 2H), 2.24 (s, 3H), 1.75-1.66 (m, 4H), 1.59-1.54 (m, 1H), 1.32-1.20 (m, 7H), 1.14 (tt, J=12.1, 3.6 Hz, 1H). ¹³C NMR (151 MHz, DMSO) δ 172.87, 166.47, 155.98, 145.65, 145.30, 141.94, 134.94, 134.14, 129.00, 128.77, 127.74, 127.41, 124.66 (2C), 118.39, 110.48, 107.28 (2C), 100.72, 67.16, 47.51, 42.40, 40.39, 32.36 (2C), 25.28, 24.75 (2C), 19.43. LC-MS (ESI): m/z=490.2, t_{R}=4.60 min.

Methyl 3-(7-(2-(cyclohexylamino)-2-oxoethoxy)naphthalen-2-yl)-3-(1-ethyl-4-methyl-1H-benzo[d][1,2,3]triazol-5-yl)propanoate (I-14h). The reaction was conducted following the general procedure F, using I-13 (0.13, 0.31 mmol) and I-9p (0.05 g, 0.21 mmol), TEA (0.09 ml, 0.61 mmol), and [Rh(COD)Cl]₂ (11 mg, 0.02 mmol). Colorless oil (0.07 g, 70.0%). ¹H NMR (600 MHz, DMSO) δ 7.91 (d, J=8.2 Hz, 1H), 7.79-7.75 (m, 1H), 7.73 (d, J=8.5 Hz, 1H), 7.69-7.66 (m, 1H), 7.63 (d, J=8.7 Hz, 1H), 7.53 (d, J=8.7 Hz, 1H), 7.30 (dd, J=8.5, 1.8 Hz, 1H), 7.22-7.17 (m, 2H), 5.02 (t, J=7.9 Hz, 1H), 4.54 (s, 2H), 4.04 (q, J=7.1 Hz, 2H), 3.63 (dd, J=7.3, 3.2 Hz, 1H), 3.51 (s, 3H), 3.26 (dd, J=16.0, 8.5 Hz, 2H), 1.99 (s, 3H), 1.74-1.64 (m, 4H), 1.57 (d, J=12.8 Hz, 1H), 1.47 (t, J=7.2 Hz, 3H), 1.31-1.22 (m, 5H). LC-MS (ESI): m/z=529.3, t_{R}=4.91 min.

3-(7-(2-(cyclohexylamino)-2-oxoethoxy)naphthalen-2-yl)-3-(1-ethyl-4-methyl-1H-benzo[d][1,2,3]triazol-5-yl)propanoic acid (3h, UCAB #1015). The reaction was conducted following the general procedure G, using I-14h (0.07 g, 0.14 mmol) and 2 M aq. NaOH (4 ml). White solid (0.02 g, 28.2%). ¹H NMR (600 MHz, DMSO) δ 12.15 (s, 1H) 7.91 (d, J=8.2 Hz, 1H), 7.77 (d, J=8.6 Hz, 1H), 7.73 (d, J=8.5 Hz, 1H), 7.67 (d, J=1.8 Hz, 1H), 7.63 (d, J=8.7 Hz, 1H), 7.51 (d, J=8.8 Hz, 1H), 7.30 (dd, J=8.5, 1.8 Hz, 1H), 7.22-7.17 (m, 2H), 5.00 (t, J=7.9 Hz, 1H), 4.67 (q, J=7.3 Hz, 2H), 4.54 (s, 2H), 3.7 (m, 1H), 3.22 (dd, J=15.9, 7.3 Hz, 1H), 3.12 (dd, J=15.9, 8.4 Hz, 1H), 2.81 (s, 3H), 1.74-1.65 (m, 4H), 1.47 (t, J=7.3 Hz, 3H), 1.57 (m, 1H), 1.31-1.20 (m, 5H). ¹³C NMR (151 MHz, DMSO) δ 173.24, 166.81, 156.38, 146.52, 142.28, 137.03, 134.55, 131.32, 129.37, 128.19, 127.77, 127.19, 127.04, 124.99, 124.90, 118.81, 108.19, 107.70, 67.51, 47.87, 43.08, 41.98, 40.49, 32.71 (2C), 25.63, 25.10 (2C), 15.41, 13.38. LC-MS (ESI): m/z=515.3, t_{R}=4.47 min.

Methyl 3-(7-(2-(cyclohexylamino)-2-oxoethoxy)naphthalen-2-yl)-3-(2,3-dihydrobenzofuran-5-yl)propanoate (I-14i). The reaction was conducted following the general procedure F, using I-13 (0.15, 0.40 mmol) and I-9q (0.05 g, 0.25 mmol), TEA (0.11 ml, 0.73 mmol), and [Rh(COD)Cl]₂ (13 mg, 0.03 mmol). Colorless oil (0.07 g, 61.5%). ¹H NMR (600 MHz, DMSO) δ 7.92 (t, J=4.1 Hz, 1H), 7.79-7.74 (m, 1H), 7.72 (d, J=8.5 Hz, 1H), 7.67-7.64 (m, 1H), 7.29 (dd, J=8.5, 1.8 Hz, 1H), 7.22-7.15 (m, 3H), 7.07 (dd, J=8.3, 2.0 Hz, 1H), 6.65 (d, J=8.2 Hz, 1H), 4.57-4.48 (m, 3H), 4.45 (t, J=8.7 Hz, 2H), 3.92 (s, 3H), 3.67-3.61 (m, 1H), 3.20 (dd, J=15.8, 8.1 Hz, 1H), 3.17-3.07 (m, 3H), 1.75-1.66 (m, 4H), 1.57 (d, J=12.5 Hz, 1H), 1.26 (d, J=11.0 Hz, 5H). LC-MS (ESI): m/z=488.2, t_{R}=5.10 min.

3-(7-(2-(cyclohexylamino)-2-oxoethoxy)naphthalen-2-yl)-3-(2,3-dihydrobenzofuran-5-yl)propanoic acid (3i, UCAB #1016). The reaction was conducted following the general procedure G, using I-14i (0.07 g, 0.15 mmol) and 2 M aq. NaOH (700 μl). White solid (0.02 g, 32.2%). ¹H NMR (600 MHz, DMSO) δ 12.08 (s, 1H), 7.92 (d, J=8.2 Hz, 1H), 7.76 (d, J=8.7 Hz, 1H), 7.72 (d, J=8.5 Hz, 1H), 7.66 (d, J=1.7 Hz, 1H), 7.29 (dd, J=8.5, 1.8 Hz, 1H), 7.19 (dq, J=8.5, 2.4 Hz, 3H), 7.07 (dd, J=8.3, 2.0 Hz, 1H), 6.65 (d, J=8.2 Hz, 1H), 4.55 (s, 2H), 4.47 (dt, J=17.4, 8.3 Hz, 3H), 3.7 (m, 1H), 3.13-3.05 (m, 3H), 3.02 (dd, J=15.7, 7.9 Hz, 1H), 1.76-1.67 (m, 4H), 1.57 (d, J=13.1 Hz, 1H), 1.26 (q, J=9.6 Hz, 5H). ¹³C NMR (151 MHz, DMSO) δ 172.73, 166.33, 158.14, 155.85, 142.78, 135.95, 134.09, 128.88, 127.58, 127.32, 127.28, 126.88, 124.42, 124.22, 124.11, 118.15, 108.50, 107.16, 70.78, 67.03, 47.40, 46.19, 40.00, 32.23 (2C), 29.08, 25.14, 24.63 (2C). LC-MS (ESI): m/z=474.2, t_{R}=4.64 min.

Methyl 3-(7-(2-(cyclohexylamino)-2-oxoethoxy)naphthalen-2-yl)-3-(2,2-dimethylbenzo[d][1,3]dioxol-5-yl)propanoate (I-14j). The reaction was conducted following the general procedure F, using I-13 (0.15, 0.37 mmol) and I-9ab (0.06 g, 0.25 mmol), TEA (0.11 ml, 0.73 mmol), and [Rh(COD)Cl]₂ (12 mg, 0.03 mmol). Colorless oil (0.04 g, 53.3%). ¹H NMR (600 MHz, DMSO) δ 7.94-7.89 (m, 1H), 7.79-7.70 (m, 2H), 7.69-7.66 (m, 1H), 7.32 (dd, J=8.5, 1.8 Hz, 1H), 7.22-7.17 (m, 2H), 6.86 (d, J=1.8 Hz, 1H), 6.78 (dd, J=8.1, 1.8 Hz, 1H), 6.69 (d, J=8.0 Hz, 1H), 4.55 (s, 2H), 4.48 (t, J=8.0 Hz, 1H), 3.64 (ddd, J=12.5, 8.0, 3.7 Hz, 1H), 3.50 (s, 3H), 3.22 (dd, J=15.9, 8.5 Hz, 1H), 3.13 (dd, J=15.9, 7.5 Hz, 1H), 1.77-1.66 (m, 4H), 1.58 (d, J=5.1 Hz, 7H), 1.33-1.25 (m, 5H). LC-MS (ESI): m/z=518.2, t_{R}=5.12 min.

3-(7-(2-(cyclohexylamino)-2-oxoethoxy)naphthalen-2-yl)-3-(2,2-dimethylbenzo[d][1,3]dioxol-5-yl)propanoic acid (3j, UCAB #1038). The reaction was conducted following the general procedure G, using I-14j (0.04 g, 0.08 mmol) and 2 M aq. NaOH (300 μl). White solid (0.04 g, 86.1%). ¹H NMR (600 MHz, DMSO) δ 7.91 (d, J=8.2 Hz, 1H), 7.79-7.74 (m, 1H), 7.72 (d, J=8.5 Hz, 1H), 7.67 (d, J=1.8 Hz, 1H), 7.31 (dd, J=8.5, 1.8 Hz, 1H), 7.19 (d, J=7.9 Hz, 2H), 6.85 (d, J=1.7 Hz, 1H), 6.77 (dd, J=8.1, 1.8 Hz, 1H), 6.69 (d, J=8.0 Hz, 1H), 4.55 (s, 2H), 4.45 (t, J=7.9 Hz, 1H), 3.68-3.60 (m, 1H), 3.12-2.99 (m, 2H), 1.75-1.66 (m, 4H), 1.58 (d, J=4.9 Hz, 7H), 1.32-1.21 (m, 4H), 1.15-1.08 (m, 1H). ¹³C NMR (151 MHz, DMSO) δ 173.13, 166.76, 156.28, 147.27, 145.62, 142.95, 137.83, 134.52, 129.31, 128.03, 127.77, 124.63, 120.36, 118.63, 118.13, 108.17 (2C), 107.58 (2C), 67.47, 47.84, 46.88, 40.47, 32.66 (2C), 25.99 (2C), 25.56 (2C), 25.06. LC-MS (ESI): m/z=504.2, t_{R}=4.72 min.

Methyl 3-(7-(2-(cyclohexylamino)-2-oxoethoxy)naphthalen-2-yl)-3-(2,2-difluorobenzo[d][1,3]dioxol-5-yl)propanoate (I-14k). The reaction was conducted following the general procedure F, using I-13 (0.10, 0.25 mmol) and I-9r (0.04g, 0.17 mmol), TEA (0.10 ml, 0.53 mmol), and [Rh(COD)Cl]₂ (8 mg, 0.02 mmol). Colorless oil (0.03 g, 34.9%). ¹H NMR (600 MHz, DMSO) δ 7.91 (s, 1H), 7.76 (dd, J=18.0, 8.7 Hz, 2H), 7.70 (d, J=1.7 Hz, 1H), 7.52 (d, J=1.7 Hz, 1H), 7.35 (dd, J=8.4, 1.8 Hz, 1H), 7.30 (d, J=8.4 Hz, 1H), 7.26-7.16 (m, 3H), 4.65 (t, J=8.0 Hz, 1H), 4.56 (s, 2H), 3.73-3.57 (m, 1H), 3.51 (s, 3H), 3.31-3.19 (m, 2H), 1.76-1.65 (m, 4H), 1.57 (d, J=13.0 Hz, 1H), 1.30-1.22 (m, 5H). LC-MS (ESI): m/z=526.2, t_{R}=5.22 min.

3-(7-(2-(cyclohexylamino)-2-oxoethoxy)naphthalen-2-yl)-3-(2,2-difluorobenzo[d][1,3]dioxol-5-yl)propanoic acid (3k, UCAB #1031). The reaction was conducted following the general procedure G, using I-14k (0.03 g, 0.06 mmol) and 2 M aq. NaOH (300 μl). White solid (0.01 g, 59.3%). ¹H NMR (600 MHz, DMSO) δ 12.17 (s, 1H), 7.91 (d, J=8.1 Hz, 1H), 7.80-7.71 (m, 2H), 7.69 (d, J=1.7 Hz, 1H), 7.50 (d, J=1.7 Hz, 1H), 7.34 (dd, J=8.5, 1.8 Hz, 1H), 7.31-7.17 (m, 4H), 4.61 (t, J=8.0 Hz, 1H), 4.55 (s, 2H), 3.64 (dh, J=9.3, 4.1 Hz, 1H), 3.14 (h, J=8.0 Hz, 2H), 1.75-1.65 (m, 4H), 1.55 (dd, J=10.3, 6.5 Hz, 1H), 1.32-1.19 (m, 4H), 1.11 (qt, J=11.7, 4.0 Hz, 1H). ¹³C NMR (151 MHz, DMSO) δ 173.02, 166.81, 156.41, 143.26, 142.27, 141.66 (2C), 134.58, 129.42, 128.28, 127.92, 124.90, 124.66, 124.12, 118.89, 110.29, 109.90, 107.66, 67.53, 47.90, 46.87, 40.42 (2C), 32.72 (2C), 25.62, 25.12 (2C). LC-MS (ESI): m/z=512.2, t_{R}=4.82 min.

Methyl 3-(7-(2-(cyclohexylamino)-2-oxoethoxy)naphthalen-2-yl)-3-(2,3-dihydrobenzo[b][1,4]dioxin-6-yl)propanoate (I-141). The reaction was conducted following the general procedure F, using I-13 (0.10, 0.25 mmol) and I-9s (0.04 g, 0.17 mmol), TEA (0.10 ml, 0.53 mmol), and [Rh(COD)Cl]₂ (8 mg, 0.02 mmol). Colorless oil (0.04 g, 32.8%). ¹H NMR (600 MHz, DMSO) δ 7.92 (d, J=8.1 Hz, 1H), 7.78-7.70 (m, 2H), 7.68-7.65 (m, 1H), 7.29 (dd, J=8.5, 1.8 Hz, 1H), 7.22-7.16 (m, 2H), 6.84 (d, J=2.1 Hz, 1H), 6.80 (dd, J=8.4, 2.2 Hz, 1H), 6.73 (d, J=8.3 Hz, 1H), 4.55 (s, 2H), 4.47 (t, J=8.0 Hz, 1H), 4.20-4.15 (m, J=1.7 Hz, 4H), 3.68-3.61 (m, 1H), 3.50 (s, 3H), 3.21-3.10 (m, 2H), 1.76-1.67 (m, 4H), 1.57 (d, J=13.0 Hz, 1H), 1.25 (t, J=7.1 Hz, 4H), 1.11 (d, J=10.8 Hz, 1H). LC-MS (ESI): m/z=504.3, t_{R}=4.88 min.

3-(7-(2-(cyclohexylamino)-2-oxoethoxy)naphthalen-2-yl)-3-(2,3-dihydrobenzo[b][1,4]dioxin-6-yl)propanoic acid (31, UCAB #1032). The reaction was conducted following the general procedure G, using I-141 (0.04 g, 0.08 mmol) and 2 M aq. NaOH (400 μl). White solid (0.01 g, 17.6%). ¹H NMR (600 MHz, DMSO) δ 12.09 (s, 1H), 7.92 (d, J=8.1 Hz, 1H), 7.77 (d, J=8.6 Hz, 1H), 7.72 (d, J=8.5 Hz, 1H), 7.66 (d, J=1.7 Hz, 1H), 7.29 (dd, J=8.5, 1.8 Hz, 1H), 7.19 (d, J=8.6 Hz, 2H), 6.83 (d, J=2.1 Hz, 1H), 6.79 (dd, J=8.4, 2.1 Hz, 1H), 6.74 (d, J=8.3 Hz, 1H), 4.55 (s, 2H), 4.44 (t, J=7.9 Hz, 1H), 4.21-4.14 (m, 4H), 3.78 (m, 1H), 3.04 (qd, J=15.8, 8.0 Hz, 2H), 1.78-1.65 (m, 4H), 1.57 (d, J=12.6 Hz, 1H), 1.33-1.22 (m, 4H), 1.17-0.98 (m, 1H). ¹³C NMR (151 MHz, DMSO) δ 173.18, 166.82, 156.34, 143.54, 142.96, 142.26, 137.58, 134.58, 129.38, 128.08, 127.82, 124.86, 124.69, 120.76, 118.69, 117.29, 116.54, 107.65, 67.53, 64.50, 64.40, 47.90, 46.47, 40.43, 32.73 (2C), 25.64, 25.13 (2C). LC-MS (ESI): m/z=490.2, t_{R}=4.45 min.

Methyl 3-(7-(2-(cyclohexylamino)-2-oxoethoxy)naphthalen-2-yl)-3-(5-methoxy-2-methylphenyl)propanoate (I-14m). The reaction was conducted following the general procedure F, using I-13 (0.1, 0.25 mmol) and I-9ac (0.04 g, 0.17 mmol), TEA (0.10 ml, 0.53 mmol), and [Rh(COD)Cl]₂ (8 mg, 0.02 mmol). Colorless oil (0.04 g, 42.6%). ¹H NMR (600 MHz, DMSO) δ 7.91 (d, J=8.2 Hz, 1H), 7.78-7.74 (m, 1H), 7.72 (d, J=8.5 Hz, 1H), 7.58-7.55 (m, 1H), 7.27 (dd, J=8.5, 1.8 Hz, 1H), 7.18 (d, J=8.1 Hz, 2H), 7.04 (d, J=8.3 Hz, 1H), 6.92 (d, J=2.7 Hz, 1H), 6.69 (dd, J=8.3, 2.7 Hz, 1H), 4.71 (t, J=7.9 Hz, 1H), 4.54 (s, 2H), 3.71 (s, 3H), 3.66-3.58 (m, 1H), 3.50 (s, 3H), 3.19-3.11 (m, 2H), 2.21 (s, 3H), 1.71 (dd, J=8.2, 4.2 Hz, 4H), 1.25 (ddt, J=13.0, 6.9, 3.3 Hz, 6H). LC-MS (ESI): m/z=490.3. t_{R}=5.09 min.

3-(7-(2-(cyclohexylamino)-2-oxoethoxy)naphthalen-2-yl)-3-(5-methoxy-2-methylphenyl)propanoic acid (3m, UCAB #1033). The reaction was conducted following the general procedure G, using I-14m (0.04 g, 0.08 mmol) and 2M aq. NaOH (300 μl). White solid (0.03 g, 57.9%). ¹H NMR (600 MHz, DMSO) δ 12.12 (s, 1H), 7.91 (d, J=8.2 Hz, 1H), 7.78-7.70 (m, 2H), 7.56 (d, J=1.8 Hz, 1H), 7.27 (dd, J=8.5, 1.8 Hz, 1H), 7.21-7.15 (m, 2H), 7.06-7.02 (m, 1H), 6.91 (d, J=2.7 Hz, jH), 6.69 (dd, J=8.3, 2.7 Hz, 1H), 4.69 (t, J=7.8 Hz, 1H), 4.54 (s, 2H), 3.71 (s, 3H), 3.67-3.57 (m, 1H), 3.03 (dd, J=7.8, 1.9 Hz, 2H), 2.21 (s, 3H), 1.75-1.65 (m, 4H), 1.59-1.53 (m, 1H), 1.30-1.21 (m, 4H), 1.12 (d, J=11.1 Hz, 1H). ¹³C NMR (151 MHz, DMSO) δ 172.97, 166.62, 157.91, 156.13, 143.02, 141.74, 134.28, 131.40, 129.16, 127.90, 127.75, 127.59, 125.09, 124.85, 118.62, 118.58, 113.40, 111.01, 107.41, 67.30, 55.19, 43.05, 40.50, 32.50 (2C), 25.42, 24.89 (2C), 18.79. LC-MS (ESI): m/z=476.3, t_{R}=4.66 min.

Methyl 3-(7-(2-(cyclohexylamino)-2-oxoethoxy)naphthalen-2-yl)-3-(6-methyl-1H-pyrrolo[2,3-b]pyridin-5-yl)propanoate (I-14n). The reaction was conducted following the general procedure F, using I-13 (0.11, 0.26 mmol) and I-9ad (0.04 g, 0.18 mmol), TEA (0.10 ml, 0.53 mmol), and [Rh(COD)Cl]₂ (8 mg, 0.02 mmol). Colorless oil (0.03 g, 32.6%). ¹H NMR (600 MHz, DMSO) δ 11.38 (s, 1H), 7.95-7.84 (m, 2H), 7.79-7.71 (m, 2H), 7.60-7.56 (m, 1H), 7.35-7.26 (m, 2H), 7.19 (d, J=8.6 Hz, 3H), 4.84 (t, J=7.9 Hz, 1H), 4.54 (s, 2H), 3.65-3.61 (m, 1H), 3.52 (d, J=7.4 Hz, 3H), 3.29-3.18 (m, 2H), 2.55 (s, 3H), 1.69-1.64 (m, 4H), 1.56 (d, J=12.8 Hz, 1H), 1.31-1.25 (m, 5H). LC-MS (ESI): m/z=500.2. t_{R}=4.15 min.

3-(7-(2-(cyclohexylamino)-2-oxoethoxy)naphthalen-2-yl)-3-(6-methyl-1H-pyrrolo[2,3-b]pyridin-5-yl)propanoic acid (3n, UCAB #1028). The reaction was conducted following the general procedure G, using I-14n (0.03 g, 0.06 mmol) and 2M aq. NaOH (300 μl). White solid (0.002 g, 6.9%). ¹H NMR (600 MHz, DMSO) δ 12.21 (s, 1H), 11.61 (s, 1H), 8.09 (s, 1H), 7.90 (d, J=8.2 Hz, 1H), 7.76 (dd, J=17.9, 8.6 Hz, 2H), 7.59 (d, J=8.7 Hz, 1H), 7.40 (s, 1H), 7.30 (td, J=8.8, 1.8 Hz, 1H), 7.24-7.15 (m, 2H), 6.44 (s, 1H), 4.83 (t, J=7.8 Hz, 1H), 4.52 (d, J=11.6 Hz, 2H), 3.64 (m, 1H), 3.14 (p, J=8.3 Hz, 2H), 2.59 (s, 3H), 1.72-1.64 (m, 4H), 1.56 (d, J=13.0 Hz, 1H), 1.25 (t, J=7.1 Hz, 4H), 1.11 (d, J=10.8 Hz, 1H). ¹³C NMR (151 MHz, DMSO) δ 173.18, 166.80, 156.39, 142.14, 134.49, 130.1, 129.38, 128.22, 127.82, 125.22, 125.02, 123.71, 122.56, 120.51, 118.85, 117.50, 117.10, 115.59, 107.65, 100.50, 67.49, 47.85, 42.65, 40.49, 32.70 (2C), 25.63 (2C), 25.09. LC-MS (ESI): m/z=486.2, t_{R}=3.72 min.

Methyl 3-(benzofuran-5-yl)-3-(7-(2-(cyclohexylamino)-2-oxoethoxy)naphthalen-2-yl)propanoate (I-140). The reaction was conducted following the general procedure F, using I-13 (0.50, 1.22 mmol) and I-9t (0.16 g, 0.81 mmol), TEA (0.35 ml, 2.47 mmol), and [Rh(COD)Cl]₂ (41 mg, 0.08 mmol). Colorless oil (0.3 g, 51%). ¹H NMR (400 MHz, DMSO) δ 7.95 (d, J=2.2 Hz, 1H), 7.92 (d, J=7.9 Hz, 1H), 7.83-7.68 (m, 3H), 7.64 (d, J=1.8 Hz, 1H), 7.49 (d, J=8.5 Hz, 1H), 7.33 (ddd, J=13.2, 8.5, 1.8 Hz, 2H), 7.25-7.15 (m, 2H), 6.90 (dd, J=2.2, 1.0 Hz, 1H), 4.71 (t, J=8.0 Hz, 1H), 4.55 (s, 2H), 3.66 (d, J=14.7 Hz, 1H), 3.50 (s, 3H), 1.82-1.63 (m, 4H), 1.57 (d, J=12.8 Hz, 1H), 1.26 (d, J=7.2 Hz, 4H), 1.17 (s, 1H), 1.08 (s, 2H). LC-MS (ESI): m/z=486.2. t_{R}=4.99 min.

3-(Benzofuran-5-yl)-3-(7-(2-(cyclohexylamino)-2-oxoethoxy)naphthalen-2-yl)propanoic acid (30, UCAB #1081). The reaction was conducted following the general procedure G, using I-140 (0.10 g, 0.20 mmol) and 2M aq. NaOH (900 μl). White solid (0.038 g, 40%). ¹H NMR (400 MHz, DMSO) δ 12.13 (s, 1H), 7.99-7.85 (m, 2H), 7.80-7.68 (m, 3H), 7.63 (d, J=1.9 Hz, 1H), 7.48 (d, J=8.5 Hz, 1H), 7.31 (ddd, J=14.7, 8.5, 1.8 Hz, 2H), 7.19 (d, J=8.5 Hz, 2H), 6.93-6.84 (m, 1H), 4.68 (t, J=7.9 Hz, 1H), 4.55 (s, 2H), 3.64 (s, 1H), 3.24-3.05 (m, 2H), 1.71 (d, J=19.9 Hz, 4H), 1.57 (d, J=12.7 Hz, 1H), 1.25 (d, J=9.8 Hz, 4H), 1.09 (d, J=25.1 Hz, 1H). ¹³C NMR (101 MHz, DMSO)· 173.22, 166.83, 156.36, 153.49, 146.75, 143.14, 139.21, 134.59, 129.38, 128.12, 127.81, 127.74, 124.95, 124.86, 124.78, 120.28, 118.72, 111.52, 107.67, 107.17, 67.52, 47.89, 47.03, 40.68, 32.71 (2C), 25.62 (2C), 25.11. LC-MS (ESI): m/z=472.2, t_{R}=4.57 min.

Methyl 3-(5-chloro-2-methylphenyl)-3-(7-(2-(cyclohexylamino)-2-oxoethoxy)naphthalen-2-yl)propanoate (I-14p). The reaction was conducted following the general procedure F, using I-13 (0.15, 0.4 mmol) and I-9v (0.06 g, 0.3 mmol), TEA (0.11 ml, 0.8 mmol), and [Rh(COD)Cl]₂ (13 mg, 0.03 mmol). Colorless oil (0.10 g, 53%). ¹H NMR (600 MHz, DMSO) δ 7.93 (d, J=8.2 Hz, 1H), 7.77 (dd, J=16.1, 8.6 Hz, 2H), 7.61-7.58 (m, 1H), 7.41 (d, J=1.8 Hz, 1H), 7.28 (dd, J=8.5, 1.8 Hz, 1H), 7.23-7.14 (m, 4H), 4.76 (t, J=7.8 Hz, 1H), 4.55 (s, 2H), 3.93 (s, 1H), 3.65-3.62 (m, 1H), 3.52 (s, 3H), 3.21 (dd, J=7.9, 2.8 Hz, 2H), 2.31 (s, 3H), 1.75-1.65 (m, 4H), 1.57 (d, J=13.1 Hz, 1H), 1.30-1.21 (m, 4H). LC-MS (ESI): m/z=494.2. t_{R}=5.16 min.

3-(5-Chloro-2-methylphenyl)-3-(7-(2-(cyclohexylamino)-2-oxoethoxy)naphthalen-2-yl)propanoic acid (3p, UCAB #1099). The reaction was conducted following the general procedure G, using I-14p (0.104 g, 0.21 mmol) and 2M aq. NaOH (950 μl). White solid (0.032 g, 32%). ¹H NMR (400 MHz, DMSO) δ 12.21 (s, 1H), 7.92 (d, J=8.2 Hz, 1H), 7.81-7.72 (m, 2H), 7.59 (d, J=1.8 Hz, 1H), 7.39 (d, J=1.7 Hz, 1H), 7.29 (dd, J=8.5, 1.8 Hz, 1H), 7.24-7.13 (m, 4H), 4.74 (t, J=7.8 Hz, 1H), 4.55 (s, 2H), 3.64 (s, 1H), 3.08 (d, J=7.8 Hz, 2H), 2.30 (s, 3H), 1.76-1.66 (m, 4H), 1.57 (d, J=12.6 Hz, 1H), 1.26 (dp, J=10.1, 3.6 Hz, 4H), 1.12 (d, J=11.5 Hz, 1H). ¹³C NMR (151 MHz, DMSO) δ 173.06, 166.82, 156.41, 144.61, 141.36, 135.29, 134.50, 132.59, 131.14, 129.41, 128.29, 127.86, 126.87, 126.61, 125.32, 124.98, 118.95, 107.65, 67.50, 47.87, 43.01, 40.54, 32.70 (2C), 25.63 (2C), 25.10, 19.36. LC-MS (ESI): m/z=480.2, t_{R}=4.77 min.

Methyl 3-(7-(2-(cyclohexylamino)-2-oxoethoxy)naphthalen-2-yl)-3-(7-methyl-2,3-dihydrobenzo[b][1,4]dioxin-6-yl)propanoate (I-14q). The reaction was conducted following the general procedure F, using I-13 (0.08, 0.2 mmol) and I-9u (0.03g, 0.13 mmol), TEA (0.11 ml, 0.6 mmol), and [Rh(COD)Cl]₂ (8 mg, 0.016 mmol). white solid (0.032 g, 31%). ¹H NMR (600 MHz, DMSO) δ 7.93 (d, J=8.2 Hz, 1H), 7.77 (d, J=9.6 Hz, 1H), 7.73 (d, J=8.5 Hz, 1H), 7.58-7.55 (m, 1H), 7.24 (dd, J=8.5, 1.8 Hz, 1H), 7.21-7.17 (m, 2H), 6.83 (s, 1H), 6.63 (d, J=0.8 Hz, 1H), 4.64 (t, J=7.9 Hz, 1H), 4.55 (s, 2H), 4.22-4.13 (m, 4H), 3.51 (s, 3H), 3.15-3.04 (m, 2H), 2.20-2.17 (m, 3H), 1.75-1.66 (m, 4H), 1.57 (d, J=12.9 Hz, 1H), 1.31-1.24 (m, 4H), 1.18 (t, J=7.1 Hz, 1H), 1.08 (s, 1H). LC-MS (ESI): m/z=518.3. t_{R}=4.60 min.

3-(7-(2-(cyclohexylamino)-2-oxoethoxy)naphthalen-2-yl)-3-(7-methyl-2,3-dihydrobenzo[b][1,4]dioxin-6-yl)propanoic acid (3q, UCAB #1125). The reaction was conducted following the general procedure G, using I-14q (0.032 g, 0.062 mmol) and 2M aq. NaOH (300 μl). White solid (0.02 g, 65%). ¹H NMR (600 MHz, DMSO) δ 12.09 (s, 1H), 7.93 (d, J=8.2 Hz, 1H), 7.79-7.75 (m, 1H), 7.73 (d, J=8.5 Hz, 1H), 7.56 (d, J=1.7 Hz, 1H), 7.25 (dd, J=8.4, 1.8 Hz, 1H), 7.21-7.16 (m, 2H), 6.82 (s, 1H), 6.63 (s, 1H), 4.61 (t, J=7.9 Hz, 1H), 4.55 (s, 2H), 4.22-4.13 (m, 4H), 3.65 (tdt, J=10.5, 7.0, 4.1 Hz, 1H), 3.03-2.92 (m, 2H), 2.18 (s, 3H), 1.71 (ddd, J=22.1, 8.4, 3.8 Hz, 3H), 1.60-1.54 (m, 1H), 1.33-1.21 (m, 5H), 1.13 (ddt, J=15.5, 11.8, 5.8 Hz, 1H). ¹³C NMR (151 MHz, DMSO) δ 173.19, 166.85, 156.33, 142.35, 141.77, 141.73, 135.04, 134.49, 129.37, 128.83, 128.10, 127.77, 125.06, 125.03, 118.94, 118.74, 115.73, 107.62, 67.50, 64.51, 64.50, 47.88, 42.54, 40.84, 32.71 (2C), 25.63 (2C), 25.10, 19.03. LC-MS (ESI): m/z=504.2, t_{R}=4.58 min.

Methyl 3-(7-(2-(cyclohexen-1-ylamino)-2-oxoethoxy)naphthalen-2-yl)-3-(2,2-difluoro-6-methylbenzo[d][1,3]dioxol-5-yl)propanoate (I-14r). The reaction was conducted following the general procedure F, using I-13 (0.08, 0.2 mmol) and I-9w (0.03 g, 0.12 mmol), TEA (0.11 ml, 0.6 mmol), and [Rh(COD)Cl]₂ (8 mg, 0.016 mmol). white solid (0.020 g, 21%). ¹H NMR (600 MHz, DMSO) δ 7.93 (d, J=8.2 Hz, 1H), 7.76 (dd, J=16.5, 8.7 Hz, 2H), 7.58 (d, J=1.7 Hz, 1H), 7.53 (s, 1H), 7.30 (dd, J=8.5, 1.8 Hz, 1H), 7.25-7.16 (m, 3H), 4.77 (t, J=7.8 Hz, 1H), 4.55 (s, 2H), 3.93 (s, 1H), 3.52 (s, 3H), 3.27-3.16 (m, 2H), 2.35 (s, 3H), 1.76-1.64 (m, 4H), 1.57 (d, J=13.1 Hz, 1H), 1.30-1.21 (m, 4H), 1.18-1.11 (m, 1H). LC-MS (ESI): m/z=540.2. t_{R}=5.30 min.

3-(7-(2-(cycloheptylamino)-2-oxoethoxy)naphthalen-2-yl)-3-(2,2-difluoro-6-methylbenzo[d][1,3]dioxol-5-yl)propanoic acid (3r, UCAB #1130). The reaction was conducted following the general procedure G, using I-14r (0.05 g, 0.09 mmol) and 2M aq. NaOH (200 μl). White solid (0.008 g, 38%). ¹H NMR (600 MHz, DMSO) δ 12.20 (s, 1H), 7.92 (d, J=8.2 Hz, 1H), 7.77 (dd, J=17.2, 8.6 Hz, 2H), 7.58 (d, J=1.8 Hz, 1H), 7.51 (s, 1H), 7.31 (dd, J=8.5, 1.8 Hz, 1H), 7.24 (s, 1H), 7.22-7.17 (m, 2H), 4.75 (t, J=7.8 Hz, 1H), 4.55 (s, 2H), 3.15-3.04 (m, 2H), 2.34 (s, 3H), 1.76-1.66 (m, 3H), 1.60-1.54 (m, 1H), 1.32-1.20 (m, 6H), 1.13 (td, J=12.2, 3.7 Hz, 1H). ¹³C NMR (151 MHz, DMSO) δ 173.08, 166.80, 156.40, 141.79, 141.50, 141.34, 138.81, 134.49, 132.82, 131.72, 129.39, 128.28, 127.86, 125.30, 124.85, 118.94, 111.99, 109.02, 107.64, 67.50, 47.86, 42.93, 40.56, 32.71 (2C), 25.62 (2C), 25.10, 19.94. LC-MS (ESI): m/z=526.2, t_{R}=4.97 min.

Methyl 3-(7-(2-(cyclohexylamino)-2-oxoethoxy)naphthalen-2-yl)-3-(6-methylbenzofuran-5-yl)propanoate (I-14s). The reaction was conducted following the general procedure F, using I-13 (0.12, 0.29 mmol) and I-9y (0.043 g, 0.2 mmol), TEA (0.1 ml, 0.71 mmol), and [Rh(COD)Cl]₂ (12 mg, 0.024 mmol). colorless oil (0.02 g, 50.5%). ¹H NMR (400 MHz, DMSO) δ 7.95-7.84 (m, 2H), 7.74 (dd, J=14.0, 8.6 Hz, 2H), 7.64 (s, 1H), 7.55 (d, J=1.8 Hz, 1H), 7.38 (s, 1H), 7.27 (dd, J=8.5, 1.8 Hz, 1H), 7.24-7.14 (m, 2H), 6.88 (dd, J=2.3, 0.9 Hz, 1H), 4.85 (t, J=7.8 Hz, 1H), 4.54 (s, 2H), 3.63 (s, 1H), 3.50 (s, 3H), 3.21 (d, J=7.9 Hz, 2H), 2.50 (h, J=1.9 Hz, 2H), 2.40 (s, 3H), 1.75-1.62 (m, 6H), 1.55 (d, J=12.4 Hz, 2H). LC-MS (ESI): m/z=500.2. t_{R}=5.19 min.

3-(7-(2-(cyclohexylamino)-2-oxoethoxy)naphthalen-2-yl)-3-(6-methylbenzofuran-5-yl)propanoic acid (3s, UCAB #1200). The reaction was conducted following the general procedure G, using I-14s (0.05 g, 0.09 mmol) and 2M aq. NaOH (280 μl). White solid (0.021 g, 30.9%). ¹H NMR (600 MHz, DMSO) δ 12.16 (s, 1H), 7.93-7.86 (m, 2H), 7.78-7.71 (m, 2H), 7.63 (s, 1H), 7.54 (d, J=1.8 Hz, 1H), 7.39 (s, 1H), 7.28 (dd, J=8.5, 1.8 Hz, 1H), 7.20-7.14 (m, 2H), 6.89 (dd, J=2.2, 1.0 Hz, 1H), 4.82 (t, J=7.8 Hz, 1H), 4.53 (s, 2H), 3.13-3.05 (m, 2H), 2.62 (p, J=1.9 Hz, 1H), 2.39 (s, 3H), 1.74-1.64 (m, 4H), 1.56 (d, J=12.9 Hz, 1H), 1.30-1.19 (m, 4H), 1.11 (d, J=11.9 Hz, 1H). ¹³C NMR (151 MHz, DMSO) δ 173.23, 166.82, 156.36, 153.58, 145.85, 142.46, 137.30, 134.49, 133.19, 129.37, 128.13, 127.77, 125.66, 125.20, 125.15, 119.44, 118.79, 112.91, 107.61, 107.09, 67.48, 47.84, 42.98, 41.32, 32.70 (2C), 25.64, 25.08, 20.54 (2C). LC-MS (ESI): m/z=486.3, t_{R}=4.73 min.

Methyl 3-(7-(2-(cyclohexylamino)-2-oxoethoxy)naphthalen-2-yl)-3-(6-methyl-2,3-dihydrobenzofuran-5-yl)propanoate (I-14t). The reaction was conducted following the general procedure F, using I-13 (0.12, 0.29 mmol) and I-9x (0.043 g, 0.2 mmol), TEA (0.1 ml, 0.71 mmol), and [Rh(COD)Cl]₂ (12 mg, 0.024 mmol). white solid (0.021 g, 30.9%). ¹H NMR (400 MHz, DMSO) δ 7.91 (d, J=6.3 Hz, 1H), 7.80-7.69 (m, 2H), 7.57 (d, J=1.7 Hz, 1H), 7.26-7.16 (m, 4H), 6.56 (s, 1H), 4.70 (t, J=7.8 Hz, 1H), 4.55 (s, 2H), 4.46 (td, J=8.7, 5.1 Hz, 2H), 4.08 (dd, J=10.9, 5.6 Hz, 1H), 3.51 (s, 3H), 3.10 (td, J=14.0, 7.7 Hz, 4H), 2.26 (s, 3H), 1.79-1.62 (m, 4H), 1.57 (d, J=12.4 Hz, 1H), 1.30-1.19 (m, 4H), 1.11 (d, J=11.9 Hz, 1H). LC-MS (ESI): m/z=502.2. t_{R}=5.09 min.

3-(7-(2-(cyclohexylamino)-2-oxoethoxy)naphthalen-2-yl)-3-(6-methyl-2,3-dihydrobenzofuran-5-yl)propanoic acid (3t, UCAB #1201). The reaction was conducted following the general procedure G, using I-14t (0.02 g, 0.04 mmol) and 2M aq. NaOH (100 μl). White solid (0.001 g, 5.2%). ¹H NMR (600 MHz, DMSO) δ 12.16 (s, 1H), δ 7.92 (d, J=8.2 Hz, 1H), 7.76 (d, J=9.6 Hz, 1H), 7.72 (d, J=8.5 Hz, 1H), 7.58-7.55 (m, 1H), 7.24 (dd, J=8.5, 1.8 Hz, 1H), 7.18 (h, J=2.6 Hz, 3H), 6.56 (s, 1H), 4.68 (t, J=7.8 Hz, 1H), 4.55 (s, 2H), 4.45 (td, J=8.8, 6.9 Hz, 2H), 3.64 (m, 1H), 3.10 (t, J=8.7 Hz, 2H), 3.06-2.93 (m, 2H), 2.26 (s, 3H), 1.84-1.63 (m, 4H), 1.57 (d, J=12.2 Hz, 1H), 1.26 (d, J=10.9 Hz, 4H), 1.12 (d, J=12.5 Hz, 1H). ¹³C NMR (151 MHz, DMSO) δ 173.21, 166.77, 158.42, 156.26, 142.65, 135.56, 134.43, 133.97, 129.28, 127.99, 127.66, 125.03 (2C), 124.87, 123.53, 118.60, 111.05, 107.58, 71.20, 67.44, 47.80, 42.37, 40.90, 32.64 (2C), 29.48, 25.57, 25.03 (2C), 20.17. LC-MS (ESI): m/z=488.3, t_{R}=4.61 min.

2-((7-Bromonaphthalen-2-yl)oxy)-N-(cyclohex-2-en-1-yl) acetamide (I-15). The reaction was conducted following the general procedure E, using I-2 (0.20 g, 0.72 mmol), HATU (0.55 g, 1.43 mmol), DIPEA (0.4 mL, 2.14 mmol) and cyclohex-2-en-1-amine hydrogenchoride (0.11 ml, 1.07 mmol). White solid (0.25 g, 97.7%). ¹H NMR (600 MHz, DMSO) δ 8.12 (d, J=8.2 Hz, 1H), 8.03 (d, J=2.0 Hz, 1H), 7.87 (d, J=8.9 Hz, 1H), 7.81 (d, J=8.6 Hz, 1H), 7.47 (dd, J=8.6, 2.0 Hz, 1H), 7.32-7.25 (m, 2H), 5.81 (dtd, J=9.7, 3.7, 2.0 Hz, 1H), 5.54 (dq, J=10.1, 2.4 Hz, 1H), 4.58 (s, 2H), 4.37 (ddp, J=7.9, 5.3, 2.7 Hz, 1H), 1.97 (dtt, J=11.2, 5.7, 2.5 Hz, 2H), 1.82-1.65 (m, 2H), 1.59-1.47 (m, 2H). LC-MS (ESI): m/z=360.1/362.1, t_{R}=5.05 min.

N-(cyclohex-2-en-1-yl)-2-((7-(4,4,5,5-tetramethyl-1,3,2-dioxaborolan-2-yl)naphthalen-2-yl)oxy) acetamide (1-16). A solution of I-15 (0.15 g, 0.42 mmol), PinB-BPin (0.13 g, 0.05 mmol), KOAc (0.125 g, 1.24 mmol), and Pd(dppf)Cl₂ (0.01 g, 0.012 mmol) were added in dioxane (5 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.15 g, 88.7%). ¹H NMR (400 MHz, DMSO) δ 8.18 (d, J=1.2 Hz, 1H), 8.12 (d, J=8.2 Hz, 1H), 7.84 (dd, J=14.3, 8.6 Hz, 2H), 7.57 (dd, J=8.1, 1.2 Hz, 1H), 7.38 (d, J=2.5 Hz, 1H), 7.32 (dd, J=8.9, 2.5 Hz, 1H), 5.81 (dtd, J=9.7, 3.7, 2.0 Hz, 1H), 5.54 (dq, J=10.0, 2.4 Hz, 1H), 4.59 (s, 2H), 4.43-4.33 (m, 1H), 1.98 (q, J=5.2, 4.2 Hz, 2H), 1.86-1.67 (m, 2H), 1.58-1.48 (m, 2H), 1.33 (s, 12H).LC-MS (ESI): m/z=408.2, t_{R}=5.33 min.

Methyl 3-(benzo[d][1,3]dioxol-5-yl)-3-(7-(2-(cyclohex-2-en-1-ylamino)-2-oxoethoxy)naphthalen-2-yl)propanoate (I-17a). The reaction was conducted following the general procedure F, using I-16 (0.15 g, 0.37 mmol) and I-9m (0.05 g, 0.25 mmol), TEA (0.11 ml, 0.74 mmol), and [Rh(COD)Cl]₂ (13 mg, 0.03 mmol). White solid (0.023 g, 31.2%). ¹H NMR (600 MHz, DMSO) δ 8.10 (d, J=8.2 Hz, 1H), 7.79-7.74 (m, 1H), 7.73 (d, J=8.5 Hz, 1H), 7.69-7.65 (m, 1H), 7.31 (dd, J=8.5, 1.8 Hz, 1H), 7.19 (d, J=7.8 Hz, 2H), 6.97 (d, J=1.7 Hz, 1H), 6.85-6.78 (m, 2H), 5.94 (dd, J=5.8, 1.0 Hz, 2H), 5.82 (ddq, J=9.3, 3.6, 1.8 Hz, 1H), 5.55 (dq, J=10.0, 2.6 Hz, 1H), 4.58 (s, 2H), 4.51 (t, J=8.0 Hz, 1H), 4.38 (ddt, J=9.6, 7.0, 3.5 Hz, 1H), 3.50 (s, 3H), 3.22 (dd, J=15.9, 8.1 Hz, 1H), 3.15 (dd, J=15.9, 7.9 Hz, 1H), 1.82-1.67 (m, 1H), 1.59-1.48 (m, 2H), 1.31-1.21 (m, 3H). LC-MS (ESI): m/z=488.2, t_{R}=4.94 min.

3-(Benzo[d][1,3]dioxol-5-yl)-3-(7-(2-(cyclohex-2-en-1-ylamino)-2-oxoethoxy)naphthalen-2-yl)propanoic acid (4a, UCAB #1010). The reaction was conducted following the general procedure G, using I-17a (0.08 g, 0.16 mmol) and 2 M aq. NaOH (300 μl). White solid (0.023 g, 31.2%). ¹H NMR (600 MHz, DMSO) δ 12.12 (s, 1H), 8.10 (d, J=8.2 Hz, 1H), 7.78-7.74 (m, 1H), 7.72 (d, J=8.5 Hz, 1H), 7.67 (d, J=1.8 Hz, 1H), 7.31 (dd, J=8.5, 1.8 Hz, 1H), 7.21-7.16 (m, 2H), 6.95 (d, J=1.6 Hz, 1H), 6.84-6.78 (m, 2H), 5.94 (d, J=4.7 Hz, 2H), 5.82 (ddp, J=9.6, 3.7, 1.8 Hz, 1H), 5.55 (dq, J=10.0, 2.5 Hz, 1H), 4.58 (s, 2H), 4.48 (t, J=7.9 Hz, 1H), 4.38 (th, J=8.3, 2.6 Hz, 1H), 3.09 (dd, J=15.8, 8.1 Hz, 1H), 3.03 (dd, J=15.9, 7.9 Hz, 1H), 2.05-1.92 (m, 2H), 1.83-1.66 (m, 2H), 1.54 (tdd, J=14.0, 7.9, 2.6 Hz, 2H). ¹³C NMR (151 MHz, DMSO) δ 172.96, 167.02, 156.18, 147.53, 145.83, 142.71, 138.25, 134.36, 129.91, 129.17, 128.63, 127.89, 127.61, 124.48 (2C), 120.85, 118.51, 108.34 (2C), 107.35, 101.03, 67.18, 46.66, 44.35, 40.28, 29.02, 24.59, 20.06. LC-MS (ESI): m/z=474.2, t_{R}=4.55 min.

Methyl 3-(7-(2-(cyclohex-2-en-1-ylamino)-2-oxoethoxy)naphthalen-2-yl)-3-(6-methylbenzo[d][1,3]dioxol-5-yl)propanoate (I-17b). The reaction was conducted following the general procedure F, using I-16 (0.09 g, 0.37 mmol) and I-9n (0.032 g, 0.14 mmol), TEA (0.10 ml, 0.742 mmol), and [Rh(COD)Cl]₂ (10 mg, 0.02 mmol). White solid (0.030 g, 30%). ¹H NMR (600 MHz, DMSO) δ 8.11 (d, J=8.2 Hz, 1H), 7.75 (dd, J=20.7, 9.1 Hz, 2H), 7.58 (s, 1H), 7.27 (dd, J=8.5, 1.8 Hz, 1H), 7.22-7.17 (m, 2H), 6.98 (d, J=1.0 Hz, 1H), 6.73 (s, 1H), 5.95 (d, J=1.0 Hz, 1H), 5.91 (d, J=1.0 Hz, 1H), 5.82 (dtd, J=9.5, 3.6, 1.8 Hz, 1H), 5.54 (dt, J=9.9, 2.8 Hz, 1H), 4.69 (t, J=7.9 Hz, 1H), 4.58 (s, 2H), 4.42-4.32 (m, OH), 3.51 (s, 3H), 3.13 (h, J=8.1 Hz, 2H), 2.25 (s, 3H), 1.98 (d, J=9.1 Hz, 2H), 1.81-1.66 (m, 1H), 1.60-1.44 (m, 1H), 1.24 (s, 1H). LC-MS (ESI): m/z=502.2, t_{R}=4.85 min.

3-(7-(2-(Cyclohex-2-en-1-ylamino)-2-oxoethoxy)naphthalen-2-yl)-3-(6-methylbenzo[d][1,3]dioxol-5-yl)propanoic acid (4b, UCAB #1118). The reaction was conducted following the general procedure G, using I-17b (0.03 g, 0.06 mmol) and 2 M aq. NaOH (310 μl). White solid (0.006 g, 21%). ¹H NMR (600 MHz, DMSO) δ 12.14 (s, 1H), 8.11 (d, J=8.2 Hz, 1H), 7.80-7.71 (m, 2H), 7.58 (s, 1H), 7.27 (dd, J=8.5, 1.8 Hz, 1H), 7.22-7.16 (m, 2H), 6.96 (d, J=1.7 Hz, 1H), 6.73 (s, 1H), 5.93 (dd, J=17.2, 1.0 Hz, 2H), 5.82 (ddp, J=9.4, 3.7, 1.8 Hz, 1H), 5.54 (dp, J=8.2, 2.6 Hz, 1H), 4.67 (t, J=7.8 Hz, 1H), 4.58 (s, 2H), 4.37 (dtq, J=7.9, 5.3, 2.6 Hz, 1H), 3.07-2.95 (m, 2H), 2.25 (s, 3H), 2.02-1.93 (m, 2H), 1.81-1.67 (m, 2H), 1.53 (ddddd, J=22.1, 19.2, 9.8, 4.7, 2.1 Hz, 2H). ¹³C NMR (151 MHz, DMSO) δ 173.23, 167.25, 156.38, 146.01, 145.66, 142.31, 135.29, 134.50, 130.16, 129.37, 129.14, 128.78, 128.11, 127.76, 125.04, 124.98, 118.77, 110.84, 107.62, 107.53, 101.08, 67.36, 44.52, 42.77, 40.77, 29.21, 24.80, 20.22, 19.79. LC-MS (ESI): m/z=488.2, t_{R}=4.45 min.

Methyl 3-(3-chloro-2-methylphenyl)-3-(7-(2-(cyclohexen-2-ylamino)-2-oxoethoxy)naphthalen-2-yl)propanoate (I-17c). The reaction was conducted following the general procedure F, using I-16 (0.12 g, 0.3 mmol) and I-91 (0.044 g, 0.21 mmol), TEA (0.14 ml, 0.99 mmol), and [Rh(COD)Cl]₂ (11 mg, 0.022 mmol). White solid (0.070 g, 48%). ¹H NMR (600 MHz, DMSO) δ 8.11 (d, J=8.3 Hz, 1H), 7.76 (dd, J=15.7, 8.7 Hz, 2H), 7.59-7.54 (m, 1H), 7.38 (d, J=7.9 Hz, 1H), 7.32 (dd, J=8.0, 1.2 Hz, 1H), 7.29-7.15 (m, 4H), 5.85-5.79 (m, 1H), 5.53 (ddt, J=9.8, 4.5, 2.4 Hz, 1H), 4.87 (t, J=7.8 Hz, 1H), 4.58 (s, 2H), 4.36 (s, 1H), 3.52 (s, 3H), 3.19 (d, J=7.9 Hz, 2H), 2.37 (s, 3H), 1.98 (s, 1H), 1.77 (dq, J=12.5, 5.9 Hz, 1H), 1.58-1.48 (m, 2H), 1.27 (dt, J=17.2, 6.3 Hz, 2H). LC-MS (ESI): m/z=492.2, t_{R}=5.11 min.

3-(3-Chloro-2-methylphenyl)-3-(7-(2-(cyclohex-2-en-1-ylamino)-2-oxoethoxy)naphthalen-2-yl)propanoic acid (4c, UCAB #1109). The reaction was conducted following the general procedure G, using I-17c (0.07 g, 0.142 mmol) and 2 M aq. NaOH (700 μl). White solid (0.033 g, 50%). ¹H NMR (600 MHz, DMSO) δ 12.23 (s, 1H), 8.11 (d, J=8.2 Hz, 1H), 7.76 (dd, J=16.0, 8.7 Hz, 2H), 7.55 (d, J=2.1 Hz, 1H), 7.37 (d, J=7.8 Hz, 1H), 7.31 (dd, J=8.0, 1.2 Hz, 1H), 7.29-7.16 (m, 4H), 5.82 (ddq, J=9.4, 3.6, 1.8 Hz, 1H), 5.53 (ddq, J=9.6, 4.7, 2.3 Hz, 1H), 4.84 (t, J=7.8 Hz, 1H), 4.58 (s, 2H), 4.39-4.33 (m, 1H), 3.07 (d, J=7.9 Hz, 2H), 2.37 (s, 3H), 2.04-1.93 (m, 2H), 1.81-1.66 (m, 2H), 1.59-1.46 (m, 2H). ¹³C NMR (151 MHz, DMSO) δ 173.05, 167.23, 156.46, 144.60, 141.60, 134.54, 134.50, 134.01, 130.17, 129.41, 128.77, 128.30, 127.85, 127.66, 127.63, 126.23, 125.31, 124.95, 118.96, 107.53, 67.35, 44.52, 43.64, 40.53, 29.21, 24.81, 20.20, 16.27. LC-MS (ESI): m/z=478.2, t_{R}=4.70 min.

Methyl 3-(7-(2-(cyclohex-2-en-amino)-2-oxoethoxy)naphthalen-2-yl)-3-(2,3-dihydrobenzo[b][1,4]dioxin-6-yl)propanoate (I-17d). The reaction was conducted following the general procedure F, using I-16 (0.12 g, 0.3 mmol) and I-9s (0.05 g, 0.22 mmol), TEA (0.14 ml, 0.99 mmol), and [Rh(COD)Cl]₂ (11 mg, 0.02 mmol). White solid (0.048 g, 34%). ¹H NMR (600 MHz, DMSO) δ 8.11 (d, J=8.2 Hz, 1H), 7.79-7.74 (m, 1H), 7.72 (d, J=8.5 Hz, 1H), 7.69-7.64 (m, 1H), 7.29 (dd, J=8.5, 1.8 Hz, 1H), 7.19 (tt, J=4.9, 2.7 Hz, 2H), 6.84 (d, J=2.1 Hz, 1H), 6.80 (dd, J=8.4, 2.2 Hz, 1H), 6.74 (d, J=8.3 Hz, 1H), 5.85-5.79 (m, 1H), 5.55 (dq, J=10.0, 2.6 Hz, 1H), 4.58 (s, 2H), 4.47 (t, J=8.0 Hz, 1H), 4.37 (dtq, J=7.7, 5.0, 2.5 Hz, 1H), 4.20-4.14 (m, 4H), 3.50 (s, 3H), 3.16 (qd, J=15.9, 8.0 Hz, 3H), 2.55 (s, 2H), 1.76-1.68 (m, 1H), 1.57-1.48 (m, 2H). LC-MS (ESI): m/z=502.2, t_{R}=4.68 min.

3-(7-(2-(cyclohex-2-en-1-ylamino)-2-oxoethoxy)naphthalen-2-yl)-3-(2,3-dihydrobenzo[b][1,4]dioxin-6-yl)propanoic acid (4d, UCAB #1100). The reaction was conducted following the general procedure G, using I-17d (0.048 g, 0.10 mmol) and 2 M aq. NaOH (450 μl). White solid (0.023 g, 48%). ¹H NMR (600 MHz, DMSO) δ 12.11 (s, 1H), 8.11 (d, J=8.2 Hz, 1H), 7.76 (d, J=8.6 Hz, 1H), 7.72 (d, J=8.4 Hz, 1H), 7.68-7.64 (m, 1H), 7.29 (dd, J=8.5, 1.8 Hz, 1H), 7.19 (d, J=8.2 Hz, 2H), 6.90-6.64 (m, 3H), 5.82 (ddt, J=9.0, 3.8, 2.1 Hz, 1H), 5.55 (dp, J=10.1, 2.5 Hz, 1H), 4.58 (s, 2H), 4.50-4.31 (m, 2H), 4.24-4.11 (m, 3H), 3.04 (qd, J=15.8, 8.0 Hz, 2H), 2.05-1.92 (m, 2H), 1.83-1.67 (m, 2H), 1.54 (ddtd, J=21.7, 11.9, 10.0, 7.6 Hz, 2H), 1.24 (s, 1H). ¹³C NMR (151 MHz, DMSO) δ 173.18, 167.25, 156.38, 143.55, 142.96, 142.27, 137.59, 134.58, 130.13, 129.38, 128.84, 128.09, 127.82, 124.87, 124.68, 120.77, 118.70, 117.30, 116.54, 107.55, 67.38, 64.51, 64.41, 46.47, 44.56, 40.54, 29.23, 24.81, 20.26. LC-MS (ESI): m/z=488.2, t_{R}=4.29 min.

Methyl 3-(7-(2-(cyclohex-2-en-1-ylamino)-2-oxoethoxy)naphthalen-2-yl)-3-(2,3-dihydrobenzofuran-5-yl)propanoate (I-17e). The reaction was conducted following the general procedure F, using I-16 (0.11 g, 0.3 mmol) and I-9q (0.042 g, 0.2 mmol), TEA (0.13 ml, 0.90 mmol), and [Rh(COD)Cl]₂ (10 mg, 0.02 mmol). White solid (0.030 g, 22%). ¹H NMR (400 MHz, DMSO) δ 8.10 (d, J=8.2 Hz, 1H), 7.74 (dd, J=15.8, 9.1 Hz, 2H), 7.65 (s, 1H), 7.29 (dd, J=8.5, 1.8 Hz, 1H), 7.19 (h, J=2.7 Hz, 3H), 7.07 (d, J=8.3 Hz, 1H), 6.65 (d, J=8.2 Hz, 1H), 5.82 (d, J=10.1 Hz, 1H), 5.58-5.51 (m, 1H), 4.58 (s, 2H), 4.48 (dt, J=24.1, 8.3 Hz, 3H), 4.37 (s, 1H), 3.50 (s, 2H), 3.26-3.06 (m, 6H), 1.80-1.68 (m, 2H), 1.62-1.46 (m, 3H), 1.24 (s, 1H).

3-(7-(2-(Cyclohex-2-en-1-ylamino)-2-oxoethoxy)naphthalen-2-yl)-3-(2,3-dihydrobenzofuran-5-yl)propanoic acid (4e, UCAB #1117). The reaction was conducted following the general procedure G, using I-17e (0.03 g, 0.06 mmol) and 2 M aq. NaOH (310 μl). White solid (0.017 g, 59%). ¹H NMR (600 MHz, DMSO) δ 12.10 (s, 1H), 8.11 (d, J=8.2 Hz, 1H), 7.74 (dd, J=24.1, 8.5 Hz, 2H), 7.68-7.64 (m, 1H), 7.29 (dd, J=8.5, 1.8 Hz, 1H), 7.21-7.18 (m, 3H), 7.07 (dd, J=8.3, 2.0 Hz, 1H), 6.65 (d, J=8.2 Hz, 1H), 5.82 (dtd, J=9.5, 3.5, 1.6 Hz, 1H), 5.55 (dp, J=10.0, 2.4 Hz, 1H), 4.58 (s, 2H), 4.45 (t, J=8.7 Hz, 3H), 4.38 (dtq, J=7.9, 5.3, 2.6 Hz, 1H), 3.13-3.05 (m, 3H), 3.02 (dd, J=15.8, 7.9 Hz, 1H), 2.06-1.91 (m, 2H), 1.82-1.68 (m, 2H), 1.60-1.48 (m, 2H). ¹³C NMR (151 MHz, DMSO) o 173.24, 167.25, 158.64, 156.38, 143.27, 136.44, 134.58, 130.14, 129.38, 128.82, 128.08, 127.83, 127.77, 127.37, 124.92, 124.73, 124.59, 118.66, 109.01, 107.55, 71.28, 67.37, 46.68, 44.55, 40.53, 29.58, 29.23, 24.80, 20.26. LC-MS (ESI): m/z=472.2, t_{R}=4.37 min.

Methyl 3-(7-(2-(cyclohex-2-en-1-ylamino)-2-oxoethoxy)naphthalen-2-yl)-3-(2,2-difluorobenzo[d][1,3]dioxol-5-yl)propanoate (I-17f). The reaction was conducted following the general procedure F, using I-16 (0.13 g, 0.32 mmol) and I-9r (0.05 g, 0.21 mmol), TEA (0.14 ml, 0.99 mmol), and [Rh(COD)Cl]₂ (11 mg, 0.02 mmol). White solid (0.070 g, 42%). ¹H NMR (600 MHz, DMSO) δ 8.14-8.08 (m, 1H), 7.76 (dd, J=15.9, 8.7 Hz, 2H), 7.72-7.68 (m, 1H), 7.52 (d, J=1.8 Hz, 1H), 7.35 (dd, J=8.5, 1.8 Hz, 1H), 7.30 (d, J=8.4 Hz, 1H), 7.26-7.16 (m, 3H), 5.81 (dtt, J=9.8, 3.9, 2.2 Hz, 1H), 5.54 (dq, J=9.2, 2.8 Hz, 1H), 4.65 (t, J=8.0 Hz, 1H), 4.59 (s, 2H), 4.37 (ddp, J=8.3, 5.5, 2.8 Hz, 2H), 3.51 (s, 3H), 3.26 (h, J=8.2 Hz, 3H), 1.83-1.75 (m, 1H), 1.71 (ddtd, J=12.8, 7.5, 5.3, 2.1 Hz, 1H), 1.59-1.47 (m, 2H). LC-MS (ESI): m/z=524.2, t_{R}=5.04 min.

3-(7-(2-(Cyclohex-2-en-1-ylamino)-2-oxoethoxy)naphthalen-2-yl)-3-(2,2-difluorobenzo[d][1,3]dioxol-5-yl)propanoic acid (4f, UCAB #1095). The reaction was conducted following the general procedure G, using I-17f (0.07 g, 0.13 mmol) and 2 M aq. NaOH (599 μl). White solid (0.023 g, 34%). ¹H NMR (600 MHz, DMSO) δ 12.19 (s, 1H), 8.11 (dd, J=8.2, 1.8 Hz, 1H), 7.81-7.67 (m, 3H), 7.51 (d, J=1.8 Hz, 1H), 7.35 (dd, J=8.5, 1.8 Hz, 1H), 7.30 (d, J=8.3 Hz, 1H), 7.26-7.16 (m, 3H), 5.81 (ddp, J=9.7, 3.7, 2.2 Hz, 1H), 5.54 (dt, J=9.9, 2.9 Hz, 1H), 4.60 (d, J=18.6 Hz, 3H), 4.38 (ddp, J=8.2, 5.5, 2.7 Hz, 1H), 3.14 (h, J=8.0 Hz, 2H), 2.05-1.91 (m, 2H), 1.86-1.65 (m, 2H), 1.63-1.44 (m, 2H). ¹³C NMR (151 MHz, DMSO) δ 173.02, 167.22, 156.45, 143.26, 142.26, 141.70, 141.62, 134.58, 130.11, 129.42, 128.83, 128.29, 127.90, 124.89, 124.67, 124.64, 124.12, 118.89, 110.29, 109.91, 107.56, 67.38, 46.87, 44.56, 40.53, 40.42, 40.28, 40.14, 40.00, 39.86, 39.72, 39.58, 29.22, 24.79, 20.26. LC-MS (ESI): m/z=510.1, t_{R}=4.67 min.

Methyl 3-(benzofuran-5-yl)-3-(7-(2-(cyclohex-2-en-1-ylamino)-2-oxoethoxy)naphthalen-2-yl)propanoate (I-17g). The reaction was conducted following the general procedure F, using I-16 (0.11 g, 0.30 mmol) and I-9t (0.04 g, 0.2 mmol), TEA (0.13 ml, 0.90 mmol), and [Rh(COD)Cl]₂ (10 mg, 0.02 mmol). White solid (0.053 g, 37%). ¹H NMR (600 MHz, DMSO) δ 8.11 (d, J=8.2 Hz, 1H), 7.94 (d, J=2.2 Hz, 1H), 7.79-7.66 (m, 3H), 7.64 (d, J=1.8 Hz, 1H), 7.49 (d, J=8.6 Hz, 1H), 7.32 (ddd, J=20.5, 8.5, 1.8 Hz, 2H), 7.23-7.16 (m, 2H), 6.90 (dd, J=2.2, 0.9 Hz, 1H), 5.81 (ddt, J=10.2, 4.5, 2.8 Hz, 1H), 5.54 (ddq, J=9.6, 4.6, 2.3 Hz, 1H), 4.71 (t, J=8.0 Hz, 1H), 4.58 (s, 2H), 4.36 (d, J=8.6 Hz, 1H), 3.50 (s, 3H), 1.98 (d, J=13.6 Hz, 2H), 1.82-1.73 (m, 1H), 1.70 (dq, J=7.3, 5.4 Hz, OH), 1.58-1.47 (m, 2H), 1.26 (dd, J=18.4, 6.3 Hz, 2H). LC-MS (ESI): m/z=484.2, t_{R}=4.85 min.

3-(Benzofuran-5-yl)-3-(7-(2-(cyclohex-2-en-1-ylamino)-2-oxoethoxy)naphthalen-2-yl)propanoic acid (4g, UCAB #1119). The reaction was conducted following the general procedure G, using I-17g (0.053 g, 0.11 mmol) and 2 M aq. NaOH (600 μl). White solid (0.034 g, 66%). ¹H NMR (600 MHz, DMSO) δ 12.14 (s, 1H), 8.11 (d, J=8.2 Hz, 1H), 7.94 (d, J=2.2 Hz, 1H), 7.79-7.67 (m, 3H), 7.63 (d, J=1.8 Hz, 1H), 7.49 (d, J=8.6 Hz, 1H), 7.32 (ddd, J=22.5, 8.5, 1.8 Hz, 2H), 7.19 (d, J=8.7 Hz, 2H), 6.90 (dd, J=2.2, 1.0 Hz, 1H), 5.85-5.78 (m, 1H), 5.57-5.51 (m, 1H), 4.68 (t, J=7.9 Hz, 1H), 4.58 (s, 2H), 4.37 (d, J=8.6 Hz, 1H), 3.16 (qd, J=15.9, 8.0 Hz, 2H), 2.04-1.92 (m, 2H), 1.82-1.74 (m, 1H), 1.74-1.65 (m, 1H), 1.53 (tqd, J=10.2, 6.3, 2.4 Hz, 2H). ¹³C NMR (151 MHz, DMSO) δ 173.24, 167.25, 156.41, 153.50, 146.76, 143.15, 139.21, 134.59, 130.15, 129.40, 128.81, 128.13, 127.80, 127.76, 124.88, 120.30, 118.74, 111.54, 107.56, 107.19, 67.37, 47.03, 44.54, 40.53, 29.22, 24.80, 20.23. LC-MS (ESI): m/z=470.2, t_{R}=4.46 min.

Methyl 3-(5-chloro-2-methylphenyl)-3-(7-(2-(cyclohex-2-en-1-ylamino)-2-oxoethoxy)naphthalen-2-yl)propanoate (I-17h). The reaction was conducted following the general procedure F, using I-16 (0.11 g, 0.30 mmol) and I-9v (0.04 g, 0.2 mmol), TEA (0.13 ml, 0.90 mmol), and [Rh(COD)Cl]₂ (10 mg, 0.02 mmol). Colorless oil (0.03 g, 21%). ¹H NMR (600 MHz, DMSO) δ 8.11 (d, J=8.2 Hz, 1H), 7.77 (dd, J=13.6, 8.6 Hz, 2H), 7.59 (d, J=2.0 Hz, 1H), 7.41 (d, J=1.8 Hz, 1H), 7.29 (dd, J=8.5, 1.8 Hz, 1H), 7.23-7.14 (m, 4H), 5.82 (ddp, J=9.6, 3.8, 2.1 Hz, 1H), 5.53 (ddq, J=10.1, 5.0, 2.3 Hz, 1H), 4.76 (t, J=7.8 Hz, 1H), 4.58 (s, 2H), 4.37 (d, J=6.0 Hz, 1H), 3.52 (s, 3H), 3.26-3.16 (m, 2H), 2.31 (s, 2H), 2.01-1.95 (m, 2H), 1.81-1.74 (m, 1H), 1.70 (dddd, J=14.7, 9.3, 5.4, 3.1 Hz, 1H), 1.58-1.46 (m, 3H). LC-MS (ESI): m/z=492.2, t_{R}=5.08 min.

3-(5-Chloro-2-methylphenyl)-3-(7-(2-(cyclohex-2-en-1-ylamino)-2-oxoethoxy)naphthalen-2-yl)propanoic acid (4h, UCAB #1102). The reaction was conducted following the general procedure G, using I-17g (0.03 g, 0.06 mmol) and 2 M aq. NaOH (300 μl). White solid (0.012 g, 61%). ¹H NMR (400 MHz, DMSO) δ 12.26 (s, 1H), 8.11 (d, J=8.3 Hz, 1H), 7.77 (t, J=9.0 Hz, 2H), 7.58 (s, 1H), 7.39 (s, 1H), 7.29 (dd, J=8.5, 1.8 Hz, 1H), 7.20 (dd, J=11.4, 3.2 Hz, 4H), 5.82 (dtd, J=9.8, 3.6, 1.8 Hz, 1H), 5.54 (dt, J=10.0, 3.0 Hz, 1H), 4.74 (t, J=7.8 Hz, 1H), 4.58 (s, 2H), 4.36 (s, 1H), 3.08 (d, J=7.8 Hz, 2H), 2.30 (s, 3H), 1.98 (t, J=4.9 Hz, 2H), 1.81-1.67 (m, 2H), 1.54 (tq, J=10.3, 6.4 Hz, 2H). ¹³C NMR (151 MHz, DMSO) δ 173.05, 167.22, 156.44, 144.59, 141.37, 135.30, 134.50, 132.60, 131.14, 130.17, 129.41, 128.77, 128.30, 127.85, 126.87, 126.62, 125.29, 124.97, 118.96, 107.52, 67.36, 44.51, 43.03, 29.21, 29.15, 24.81, 20.20, 19.35.LC-MS (ESI): m/z=478.2, t_{R}=4.72 min.

Methyl 3-(7-(2-(cyclohex-2-en-1-ylamino)-2-oxoethoxy)naphthalen-2-yl)-3-(7-methyl-2,3-dihydrobenzo[b][1,4]dioxin-6-yl)propanoate (I-17i). The reaction was conducted following the general procedure F, using I-16 (0.07 g, 0.18 mmol) and I-9u (0.03 g, 0.12 mmol), TEA (0.1 ml, 0.54 mmol), and [Rh(COD)Cl]₂ (7 mg, 0.013 mmol). White solid (0.02 g, 22%). ¹H NMR (600 MHz, DMSO) δ 8.11 (d, J=8.2 Hz, 1H), 7.75 (dd, J=22.0, 9.1 Hz, 2H), 7.57 (s, 1H), 7.24 (dd, J=8.4, 1.8 Hz, 1H), 7.22-7.16 (m, 2H), 6.83 (d, J=1.7 Hz, 1H), 6.63 (s, 1H), 5.82 (dt, J=9.9, 2.9 Hz, 1H), 5.54 (dq, J=10.1, 2.5 Hz, 1H), 4.64 (t, J=7.9 Hz, 1H), 4.58 (s, 2H), 4.17 (q, J=1.7 Hz, 4H), 3.51 (s, 3H), 3.15-3.04 (m, 2H), 2.19 (s, 3H), 1.98 (s, 2H), 1.83-1.64 (m, 1H), 1.58-1.48 (m, 2H), 1.31-1.23 (m, 2H). LC-MS (ESI): m/z=516.3, t_{R}=4.80 min.

3-(7-(2-(cyclohex-2-en-1-ylamino)-2-oxoethoxy)naphthalen-2-yl)-3-(7-methyl-2,3-dihydrobenzo[b][1,4]dioxin-6-yl)propanoic acid (4i, UCAB #1123). The reaction was conducted following the general procedure G, using I-17i (0.02 g, 0.06 mmol) and 2 M aq. NaOH (200 μl). White solid (0.014 g, 70%). ¹H NMR (600 MHz, DMSO) δ 12.13 (s, 1H), 8.11 (d, J=8.2 Hz, 1H), 7.80-7.74 (m, 1H), 7.73 (d, J=8.5 Hz, 1H), 7.56 (d, J=1.6 Hz, 1H), 7.25 (dd, J=8.5, 1.8 Hz, 1H), 7.22-7.16 (m, 2H), 6.82 (d, J=2.2 Hz, 1H), 6.63 (d, J=0.8 Hz, 1H), 5.82 (dtd, J=9.7, 3.7, 1.9 Hz, 1H), 5.54 (dp, J=10.1, 2.5 Hz, 1H), 4.61 (t, J=7.8 Hz, 1H), 4.58 (s, 2H), 4.37 (dtq, J=7.5, 4.9, 2.5 Hz, 1H), 4.18 (t, J=1.4 Hz, 4H), 3.03-2.92 (m, 2H), 2.18 (s, 3H), 2.05-1.95 (m, 2H), 1.83-1.67 (m, 2H), 1.53 (ddddt, J=16.9, 12.1, 7.5, 5.1, 2.6 Hz, 2H). ¹³C NMR (151 MHz, DMSO) δ 173.21, 167.27, 156.38, 142.36, 141.78, 141.73, 135.06, 134.50, 130.17, 129.39, 128.84, 128.80, 128.12, 127.77, 125.06, 118.95, 118.76, 115.74, 107.52, 67.36, 64.51, 64.50, 44.52, 42.56, 40.85, 40.53, 40.41, 40.39, 40.27, 40.13, 39.99, 39.85, 39.72, 39.58, 29.22, 24.81, 20.23, 19.03. LC-MS (ESI): m/z=502.2, t_{R}=4.41 min.

methyl 3-(3-chlorophenyl)-3-(7-(2-(cyclohex-2-en-1-ylamino)-2-oxoethoxy)naphthalen-2-yl)propanoate (I-17j). The reaction was conducted following the general procedure F, using I-16 (0.10 g, 0.25 mmol) and I-9i (0.033 g, 0.17 mmol), TEA (0.14 ml, 0.8 mmol), and [Rh(COD)Cl]₂ (10 mg, 0.02 mmol). White solid (0.05 g, 42%). ¹H NMR (600 MHz, DMSO) δ 8.11 (d, J=8.2 Hz, 1H), 7.81-7.70 (m, 3H), 7.45 (t, J=1.9 Hz, 1H), 7.39-7.29 (m, 3H), 7.27-7.18 (m, 3H), 5.82 (dd, J=8.6, 3.8 Hz, 1H), 5.54 (dt, J=10.0, 2.6 Hz, 1H), 4.62 (t, J=8.0 Hz, 1H), 4.59 (s, 2H), 4.37 (d, J=6.3 Hz, 1H), 3.51 (s, 2H), 3.26 (d, J=8.2 Hz, 2H), 1.98 (s, 2H), 1.78 (dd, J=12.9, 4.9 Hz, 1H), 1.74-1.67 (m, 1H), 1.60-1.48 (m, 2H), 1.30-1.21 (m, 1H). LC-MS (ESI): m/z=478.2, t_{R}=5.08 min.

3-(3-chlorophenyl)-3-(7-(2-(cyclohex-2-en-1-ylamino)-2-oxoethoxy)naphthalen-2-yl)propanoic acid (4j, UCAB #1126). The reaction was conducted following the general procedure G, using I-17j (0.05 g, 0.1 mmol) and 2 M aq. NaOH (500 μl). White solid (0.036 g, 78%). ¹H NMR (600 MHz, DMSO) δ 12.19 (s, 1H), 8.14-8.09 (m, 1H), 7.81-7.69 (m, 3H), 7.44 (t, J=1.9 Hz, 1H), 7.38-7.29 (m, 3H), 7.24 (ddd, J=7.8, 2.1, 1.2 Hz, 1H), 7.21 (dd, J=6.7, 2.7 Hz, 2H), 5.82 (ddt, J=8.9, 3.9, 2.1 Hz, 1H), 5.55 (dp, J=10.0, 2.4 Hz, 1H), 4.59 (s, 3H), 4.37 (dtq, J=7.9, 5.2, 2.5 Hz, 1H), 3.14 (d, J=8.1 Hz, 2H), 2.05-1.95 (m, 2H), 1.78 (dtt, J=11.9, 5.1, 2.4 Hz, 1H), 1.71 (ddd, J=15.7, 8.5, 3.6 Hz, 1H), 1.60-1.47 (m, 2H). ¹³C NMR (151 MHz, DMSO) o 173.03, 167.23, 156.45, 147.08, 142.10, 134.58, 133.49, 130.76, 130.14, 129.43, 128.81, 128.29, 127.98, 127.91, 126.86, 126.78, 124.98, 124.75, 124.73, 118.90, 107.56, 67.38, 46.79, 44.56, 29.22, 24.80, 20.26. LC-MS (ESI): m/z=464.2, t_{R}=4.69 min.

Methyl 3-(7-(2-(cyclohex-2-en-1-ylamino)-2-oxoethoxy) naphthalen-2-yl)-3-(4-methoxy-2-methylphenyl)propanoate (I-17k). The reaction was conducted following the general procedure F, using I-16 (0.08 g, 0.2 mmol) and I-9k (0.027 g, 0.13 mmol), TEA (0.11 ml, 0.6 mmol), and [Rh(COD)Cl]₂ (8 mg, 0.016 mmol). White solid (0.03 g, 31%). ¹H NMR (600 MHz, DMSO) δ 8.11 (d, J=8.2 Hz, 1H), 7.76 (d, J=8.9 Hz, 1H), 7.72 (d, J=8.4 Hz, 1H), 7.53 (s, 1H), 7.28 (dd, J=8.5, 1.1 Hz, 1H), 7.24 (dd, J=8.5, 1.8 Hz, 1H), 7.21-7.15 (m, 2H), 6.78-6.71 (m, 2H), 5.82 (dtt, J=9.7, 3.8, 2.2 Hz, 1H), 5.53 (ddq, J=9.8, 4.8, 2.3 Hz, 1H), 4.70 (t, J=7.9 Hz, 1H), 4.57 (s, 2H), 4.36 (q, J=5.2 Hz, 1H), 3.71 (s, 3H), 3.51 (s, 3H), 3.18-3.07 (m, 2H), 2.27 (s, 3H), 2.02-1.92 (m, 2H), 1.81-1.67 (m, 1H), 1.59-1.46 (m, 1H), 1.52 (s, 1H), 1.25 (d, J=6.2 Hz, 1H). LC-MS (ESI): m/z=488.3, t_{R}=4.97 min.

3-(7-(2-(cyclohex-2-en-1-ylamino)-2-oxoethoxy)naphthalen-2-yl)-3-(4-methoxy-2-methylphenyl)propanoic acid (4k, UCAB #1127). The reaction was conducted following the general procedure G, using I-17k (0.03 g, 0.1 mmol) and 2 M aq. NaOH (300 μl). White solid (0.0052 g, 17%). ¹H NMR (600 MHz, DMSO) δ 12.11 (s, 1H), 8.11 (d, J=8.2 Hz, 1H), 7.76 (d, J=8.8 Hz, 1H), 7.72 (d, J=8.5 Hz, 1H), 7.53 (d, J=1.7 Hz, 1H), 7.26 (ddd, J=17.3, 8.5, 1.7 Hz, 2H), 7.21-7.15 (m, 2H), 6.76 (dd, J=8.5, 2.8 Hz, 1H), 6.73 (d, J=2.8 Hz, 1H), 5.82 (ddq, J=9.9, 3.8, 1.9 Hz, 1H), 5.54 (ddq, J=9.7, 4.7, 2.3 Hz, 1H), 4.68 (t, J=7.9 Hz, 1H), 4.57 (s, 2H), 4.36 (tdt, J=7.2, 4.7, 2.4 Hz, 1H), 3.83 (s, 1H), 3.71 (s, 3H), 3.00 (h, J=8.1 Hz, 2H), 2.27 (s, 3H), 2.05-1.92 (m, 2H), 1.81-1.66 (m, 2H), 1.60-1.44 (m, 1H), 1.33-1.22 (m, 1H). ¹³C NMR (151 MHz, DMSO) δ 173.24, 167.24, 157.89, 156.38, 142.58, 137.51, 134.48, 134.20, 130.15, 129.36, 128.79, 128.07, 128.01, 127.73, 125.04 (2C), 118.73, 116.38, 111.65, 107.49, 67.35, 55.35, 44.51, 42.38, 41.00, 29.22, 24.81, 20.21, 20.08. LC-MS (ESI): m/z=474.3, t_{R}=4.57 min.

Methyl 3-(7-(2-(cyclohex-2-en-1-ylamino)-2-oxoethoxy)naphthalen-2-yl)-3-(2,2-difluoro-6-methylbenzo[d][1,3]dioxol-5-yl)propanoate (I-171). The reaction was conducted following the general procedure F, using I-16 (0.075 g, 0.2 mmol) and I-9w (0.030 g, 0.12 mmol), TEA (0.11 ml, 0.6 mmol), and [Rh(COD)Cl]₂ (8 mg, 0.016 mmol). White solid (0.03 g, 30%). ¹H NMR (600 MHz, DMSO) δ 8.11 (d, J=8.2 Hz, 1H), 7.76 (dd, J=14.3, 8.7 Hz, 2H), 7.58 (t, J=2.3 Hz, 1H), 7.53 (s, 1H), 7.30 (dd, J=8.5, 1.8 Hz, 1H), 7.25-7.22 (m, 1H), 7.22-7.16 (m, 2H), 5.81 (dddd, J=9.6, 5.6, 3.7, 2.0 Hz, 1H), 5.53 (ddq, J=9.8, 5.0, 2.3 Hz, 1H), 4.77 (t, J=7.8 Hz, 1H), 4.58 (s, 2H), 4.36 (s, 1H), 3.52 (s, 3H), 3.27-3.16 (m, 2H), 2.35 (s, 3H), 2.04-1.95 (m, 2H), 1.81-1.73 (m, 1H), 1.73-1.66 (m, 1H), 1.58-1.46 (m, 1H), 1.32-1.21 (m, 1H). LC-MS (ESI): m/z=538.2, t_{R}=5.24 min.

3-(7-(2-(cyclohex-2-en-1-ylamino)-2-oxoethoxy)naphthalen-2-yl)-3-(2,2-difluoro-6-methylbenzo[d][1,3]dioxol-5-yl)propanoic acid (41, UCAB #1128). The reaction was conducted following the general procedure G, using I-171 (0.03 g, 0.06 mmol) and 2 M aq. NaOH (300 μl). White solid (0.0018 g, 58%). ¹H NMR (600 MHz, DMSO) δ 12.20 (s, 1H), 8.10 (d, J=8.2 Hz, 1H), 7.76 (dd, J=14.9, 8.6 Hz, 2H), 7.62-7.56 (m, 1H), 7.51 (s, 1H), 7.31 (dd, J=8.5, 1.8 Hz, 1H), 7.25-7.23 (m, 1H), 7.23-7.16 (m, 2H), 5.81 (dqd, J=9.7, 3.8, 2.0 Hz, 1H), 5.54 (ddd, J=9.4, 6.0, 2.8 Hz, 1H), 4.75 (t, J=7.8 Hz, 1H), 4.58 (s, 2H), 4.40-4.34 (m, 1H), 3.15-3.04 (m, 2H), 2.34 (s, 3H), 2.04-1.93 (m, 2H), 1.83-1.64 (m, 2H), 1.60-1.45 (m, 1H), 1.30-1.18 (m, 1H). ¹³C NMR (151 MHz, DMSO) δ 173.08, 167.21, 156.44, 141.79, 141.49, 141.34, 138.81, 134.49, 132.83, 131.72, 130.12, 129.39, 128.80, 128.29, 127.85, 125.29, 124.86, 118.95, 112.00, 109.03, 107.53, 67.35, 44.51, 42.94, 40.68, 29.22, 24.79, 20.23, 19.94. LC-MS (ESI): m/z=524.2, t_{R}=4.90 min.

Methyl 3-(7-(2-(cyclohex-2-en-1-ylamino)-2-oxoethoxy)naphthalen-2-yl)-3-(6-methylbenzofuran-5-yl)propanoate (I-17m). The reaction was conducted following the general procedure F, using I-16 (0.12 g, 0.35 mmol) and I-9y (0.040 g, 0.24 mmol), TEA (0.1 ml, 0.71 mmol), and [Rh(COD)Cl]₂ (12 mg, 0.024 mmol). White solid (0.04 g, 44%). ¹H NMR (600 MHz, DMSO) δ 8.08 (d, J=8.3 Hz, 1H), 7.88 (d, J=2.2 Hz, 1H), 7.74 (dd, J=17.9, 8.7 Hz, 2H), 7.63 (s, 1H), 7.57-7.51 (m, 1H), 7.39 (s, 1H), 7.27 (dd, J=8.4, 1.7 Hz, 1H), 7.20-7.13 (m, 2H), 6.88 (d, J=2.2 Hz, 1H), 5.82-5.73 (m, 1H), 5.51 (td, J=9.7, 2.6 Hz, 1H), 4.84 (t, J=7.8 Hz, 1H), 4.56 (s, 2H), 4.39-4.27 (m, 1H), 3.50 (s, 3H), 3.24-3.17 (m, 2H), 2.39 (s, 3H), 2.01-1.90 (m, 2H), 1.81-1.40 (m, 4H). LC-MS (ESI): m/z=498.2, t_{R}=5.11 min.

3-(7-(2-(cycloheptylamino)-2-oxoethoxy)naphthalen-2-yl)-3-(6-methylbenzo[d][1,3]dioxol-5-yl)propanoic acid (4m, UCAB #1192). The reaction was conducted following the general procedure G, using I-17m (0.04 g, 0.08 mmol) and 2 M aq. NaOH (500 μl). White solid (0.017 g, 58%). ¹H NMR (600 MHz, DMSO) δ 12.16 (s, 1H), 8.10 (d, J=8.2 Hz, 1H), 7.89 (d, J=2.2 Hz, 1H), 7.75 (dd, J=17.9, 8.7 Hz, 2H), 7.64 (s, 1H), 7.53 (t, J=2.4 Hz, 1H), 7.40 (s, 1H), 7.29 (dd, J=8.5, 1.7 Hz, 1H), 7.21-7.14 (m, 2H), 6.91-6.88 (m, 1H), 5.84-5.76 (m, 1H), 5.52 (td, J=9.7, 2.7 Hz, 1H), 4.83 (t, J=7.9 Hz, 1H), 4.56 (s, 2H), 4.35 (s, 1H), 3.10 (dd, J=7.9, 1.3 Hz, 2H), 2.39 (d, J=2.6 Hz, 3H), 2.04-1.92 (m, 2H), 1.80-1.65 (m, 1H), 1.57-1.44 (m, 1H), 1.25 (s, 2H). ¹³C NMR (151 MHz, DMSO) δ 173.23, 167.22, 156.40, 153.58, 145.84, 142.48, 137.27, 134.49, 133.21, 130.15, 129.37, 128.74, 128.13, 127.76, 125.67, 125.18, 125.16, 119.44, 118.80, 112.91, 107.48, 107.11, 67.33, 44.47, 42.99, 41.33, 29.20, 24.81, 20.54, 20.18. LC-MS (ESI): m/z=484.2, t_{R}=4.65 min.

Methyl 3-(7-(2-(cyclohex-2-en-1-ylamino)-2-oxoethoxy)naphthalen-2-yl)-3-(6-methyl-2,3-dihydrobenzofuran-5-yl)propanoate (I-17n). The reaction was conducted following the general procedure F, using I-16 (0.12 g, 0.35 mmol) and I-9x (0.040 g, 0.24 mmol), TEA (0.1 ml, 0.71 mmol), and [Rh(COD)Cl]₂ (12 mg, 0.024 mmol). Colorless oil (0.03 g, 35%). ¹H NMR (400 MHz, DMSO) δ 8.09 (d, J=8.1 Hz, 1H), 7.74 (dd, J=15.7, 9.0 Hz, 2H), 7.56 (s, 1H), 7.26-7.15 (m, 4H), 6.56 (s, 1H), 5.84-5.75 (m, 1H), 5.54 (d, J=10.2 Hz, 1H), 4.71 (t, J=7.8 Hz, 1H), 4.58 (s, 2H), 4.45 (td, J=8.7, 4.7 Hz, 2H), 4.37 (s, 1H), 3.51 (s, 3H), 3.25-3.07 (m, 4H), 2.26 (s, 3H), 1.87-1.64 (m, 2H), 1.54 (d, J=8.1 Hz, 4H). LC-MS (ESI): m/z=500.2, t_{R}=5.02 min.

3-(7-(2-(cyclohex-2-en-1-ylamino)-2-oxoethoxy)naphthalen-2-yl)-3-(6-methyl-2,3-dihydrobenzofuran-5-yl)propanoic acid (4n, UCAB #1194). The reaction was conducted following the general procedure G, using I-17n (0.03 g, 0.06 mmol) and 2 M aq. NaOH (500 μl). White solid (0.006 g, 20.7%). ¹H NMR (600 MHz, DMSO) δ 12.09 (s, 1H), 8.09 (d, J=8.3 Hz, 1H), 7.78-7.69 (m, 2H), 7.55 (s, 1H), 7.23 (dd, J=8.5, 1.8 Hz, 1H), 7.17 (h, J=2.6 Hz, 3H), 6.55 (s, 1H), 5.84-5.78 (m, 1H), 5.53 (dd, J=10.1, 2.7 Hz, 1H), 4.67 (t, J=7.8 Hz, 1H), 4.57 (s, 2H), 4.44 (td, J=8.7, 6.5 Hz, 2H), 4.36 (dtt, J=8.2, 5.5, 2.7 Hz, 1H), 3.09 (t, J=8.7 Hz, 2H), 3.05-2.91 (m, 2H), 2.25 (s, 3H), 2.02-1.92 (m, 2H), 1.80-1.66 (m, 2H), 1.52 (dddd, J=15.3, 12.0, 8.2, 2.8 Hz, 2H). ¹³C NMR (151 MHz, DMSO) δ 173.28, 167.24, 158.47, 156.38, 142.72, 135.64, 134.50, 134.05, 130.14, 129.36, 128.80, 128.07, 127.72, 125.09 (2C), 124.92, 123.61, 118.69, 111.12, 107.54, 71.27, 67.36, 44.51, 42.46, 40.97, 29.55, 29.22, 24.81, 20.24 (2C). LC-MS (ESI): m/z=486.2, t_{R}=4.54 min.

N-((1S,2S,4R)-bicyclo[2.2.1]heptan-2-yl)-2-((7-bromonaphthalen-2-yl)oxy) acetamide (I-18). The reaction was conducted following the general procedure E, using I-2 (0.15 g, 0.53 mmol), HATU (0.41 g, 1.07 mmol), DIPEA (0.28 mL, 1.60 mmol) and (1S,2S,4R)-bicyclo[2.2.1]heptan-2-amine (0.1 ml, 0.80 mmol). White solid (0.16 g, 79.6%). ¹H NMR (400 MHz, DMSO) δ 8.03 (d, J=2.0 Hz, 1H), 7.89 (d, J=9.1 Hz, 2H), 7.82 (d, J=8.7 Hz, 1H), 7.48 (dd, J=8.7, 2.1 Hz, 1H), 7.33-7.24 (m, 2H), 4.57 (s, 2H), 3.64-3.57 (m, 1H), 2.22 (s, 1H), 2.11 (s, 1H), 1.62 (ddd, J=12.7, 8.1, 2.2 Hz, 1H), 1.50-1.34 (m, 4H), 1.11 (dd, J=15.3, 8.3 Hz, 3H). LC-MS (ESI): m/z=374.1/376.1, t_{R}=5.10 min.

N-((1S,2S,4R)-bicyclo[2.2.1]heptan-2-yl)-2-((7-(4,4,5,5-tetramethyl-1,3,2-dioxaborolan-2-yl)naphthalen-2-yl)oxy) acetamide (I-19). A solution of 1-18 (0.16 g, 0.43 mmol), PinB-BPin (0.13 g, 0.51 mmol), KOAc (0.13 g, 1.31 mmol), and Pd(dppf)Cl₂ (0.01 g, 0.013 mmol) were added in dioxane (5 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.154 g, 85.6%). ¹H NMR (400 MHz, DMSO) δ 8.18 (s, 1H), 7.94-7.80 (m, 3H), 7.57 (dd, J=8.1, 1.1 Hz, 1H), 7.38 (d, J=2.6 Hz, 1H), 7.32 (dd, J=8.9, 2.5 Hz, 1H), 4.56 (s, 2H), 3.60 (d, J=3.7 Hz, 1H), 2.22 (s, 1H), 2.11 (d, J=3.9 Hz, 1H), 1.67-1.57 (m, 1H), 1.51-1.37 (m, 2H), 1.34 (s, 12H), 1.20-1.09 (m, 4H), 1.08 (s, 1H). LC-MS (ESI): m/z=422.3, t_{R}=5.35 min.

Methyl 3-(benzo[d][1,3]dioxol-5-yl)-3-(7-(2-(((1S,2S,4R)-bicyclo[2.2.1]heptan-2-yl)amino)-2-oxoethoxy)naphthalen-2-yl)propanoate (I-20a). The reaction was conducted following the general procedure F, using I-19 (0.15 g, 0.36 mmol) and I-9m (0.05 g, 0.25 mmol), TEA (0.1 ml, 0.71 mmol), and [Rh(COD)Cl]₂ (12 mg, 0.03 mmol). White solid (0.07 g, 56.9%). ¹H NMR (600 MHz, DMSO) δ 7.90-7.87 (m, 1H), 7.79-7.71 (m, 2H), 7.66 (s, 1H), 7.32 (dd, J=8.4, 1.6 Hz, 1H), 7.21-7.16 (m, 2H), 6.97 (d, J=1.7 Hz, 1H), 6.85-6.78 (m, 2H), 5.94 (dd, J=6.0, 1.0 Hz, 2H), 4.57-4.49 (m, 3H), 3.92 (s, 3H), 3.63-3.56 (m, 1H), 3.23-3.09 (m, 2H), 2.22 (s, 1H), 2.10 (d, J=5.0 Hz, 1H), 1.64-1.56 (m, 1H), 1.48-1.40 (m, 4H), 1.37 (m, 3H). LC-MS (ESI): m/z=502.3, t_{R}=4.98 min.

3-(benzo[d][1,3]dioxol-5-yl)-3-(7-(2-(((1S,2S,4R)-bicyclo[2.2.1]heptan-2-yl)amino)-2-oxoethoxy)naphthalen-2-yl)propanoic acid (5a, UCAB #1182). The reaction was conducted following the general procedure G, using I-20a (0.07 g, 0.14 mmol) and 2 M aq. NaOH (140 μl). White solid (0.012 g, 17.6%). ¹H NMR (600 MHz, DMSO) δ 12.13 (s, 1H), 7.88 (dd, J=7.2, 3.0 Hz, 1H), 7.79-7.75 (m, 1H), 7.73 (d, J=8.5 Hz, 1H), 7.66 (s, 1H), 7.31 (dt, J=8.6, 1.4 Hz, 1H), 7.19 (dd, J=6.6, 2.7 Hz, 2H), 6.95 (s, 1H), 6.85-6.78 (m, 2H), 5.94 (d, J=4.9 Hz, 2H), 4.55 (s, 2H), 4.49 (t, J=7.9 Hz, 1H), 3.59 (td, J=7.7, 3.6 Hz, 1H), 3.14-2.88 (m, 2H), 2.22 (d, J=4.2 Hz, 1H), 2.09 (q, J=5.5 Hz, 1H), 1.61 (ddt, J=12.6, 8.2, 2.1 Hz, 1H), 1.49-1.34 (m, 4H), 1.17-1.05 (m, 3H). ¹³C NMR (151 MHz, DMSO) δ 173.16, 167.11, 156.44, 147.74, 146.04, 142.92, 138.46, 138.44, 134.57, 129.37, 128.10, 127.81, 124.81, 124.66, 121.05, 118.72, 108.55, 108.53, 107.57, 101.24, 67.48, 52.55, 46.86, 42.34, 38.74, 35.57, 35.36, 28.46, 26.60. LC-MS (ESI): m/z=488.3, t_{R}=4.57 min.

Methyl 3-(7-(2-(((1S,2S,4R)-bicyclo[2.2.1]heptan-2-yl)amino)-2-oxoethoxy)naphthalen-2-yl)-3-(6-chlorobenzo[d][1,3]dioxol-5-yl)propanoate (I-20b). The reaction was conducted following the general procedure F, using I-19 (0.11 g, 0.26 mmol) and I-9z (0.04 g, 0.17 mmol), TEA (0.07 ml, 0.52 mmol), and [Rh(COD)Cl]₂ (9 mg, 0.02 mmol). White solid (0.05 g, 34%). ¹H NMR (400 MHz, DMSO) δ 7.88 (d, J=7.2 Hz, 1H), 7.74 (q, J=8.7 Hz, 2H), 7.61 (d, 1H), 7.31 (d, J=8.3 Hz, 1H), 7.19 (d, J=13.0 Hz, 3H), 7.03 (s, 1H), 6.04 (d, J=19.0 Hz, 2H), 4.95 (t, J=7.9 Hz, 1H), 4.55 (s, 2H), 3.64-3.56 (m, 1H), 3.51 (d, J=7.8 Hz, 3H), 3.18 (td, J=13.8, 7.9 Hz, 2H), 2.21 (s, 1H), 2.08 (s, 1H), 1.60 (t, J=10.6 Hz, 1H), 1.49-1.32 (m, 4H), 1.10 (q, J=12.2 Hz, 3H). LC-MS (ESI): m/z=536.2, t_{R}=5.35 min.

3-(7-(2-(((1S,2S,4R)-bicyclo[2.2.1]heptan-2-yl)amino)-2-oxoethoxy)naphthalen-2-yl)-3-(6-chlorobenzo[d][1,3]dioxol-5-yl)propanoic acid (5b, UCAB #1245). The reaction was conducted following the general procedure G, using I-20b (0.031 g, 0.06 mmol) and 2 M aq. NaOH (100 μl). White solid (0.012 g, 37.5%) ¹H NMR (600 MHz, DMSO) δ 12.18 (s, 1H), 7.88 (d, J=7.2 Hz, 1H), 7.75 (m, 2H), 7.60 (d, J=2.0 Hz, 1H), 7.32 (dd, J=8.5, 1.8 Hz, 1H), 7.18 (m, 3H), 7.02 (s, 1H), 6.04 (d, J=22.8, 2H), 4.91 (t, J=7.9 Hz, 1H), 4.55 (s, J=1.5 Hz, 2H), 3.59-3.56 (m, 1H), 3.13-3.02 (m, 2H), 2.22-2.20 (m, 1H), 2.08 (m, 1H), 1.62-1.57 (m, 1H), 1.47-1.34 (m, 4H), 1.15-1.05 (m, 3H). ¹³C NMR (151 MHz, DMSO) δ 172.41, 166.59, 155.98, 146.85, 146.41, 140.83, 133.99 (2C), 128.88, 127.68, 127.36, 124.73, 124.31, 124.17, 118.43, 109.53, 108.17, 107.06, 101.91, 66.98, 52.04, 42.73, 41.84, 39.94, 38.24, 35.07, 34.85, 27.97, 26.10. LC-MS (ESI): m/z=522.2, t_{R}=4.94 min.

Methyl 3-(7-(2-(((1S,2S,4R)-bicyclo[2.2.1]heptan-2-yl)amino)-2-oxoethoxy)naphthalen-2-yl)-3-(6-methylbenzo[d][1,3]dioxol-5-yl)propanoate (I-20c). The reaction was conducted following the general procedure F, using I-19 (0.204 g, 0.48 mmol) and I-9n (0.07 g, 0.32 mmol), TEA (0.14 ml, 0.97 mmol), and [Rh(COD)Cl]₂ (15.8 mg, 0.032 mmol). White solid (0.087 g, 52.4%). ¹H NMR (600 MHz, DMSO) δ 7.88-7.86 (m, 1H), 7.76 (d, J=8.7 Hz, 1H), 7.72 (d, J=8.5 Hz, 1H), 7.56 (s, 1H), 7.27 (dd, J=8.5, 1.8 Hz, 1H), 7.18-7.15 (m, 2H), 6.98 (d, J=3.1 Hz, 1H), 6.72 (s, 1H), 5.93 (dd, J=25.7, 1.0 Hz, 2H), 4.68 (t, J=7.9 Hz, 1H), 4.54 (s, 2H), 3.58 (m, 1H), 3.51 (s, 3H), 3.13 (m, 2H), 2.24 (s, 3H), 2.21 (m, 1H), 2.07 (m, 1H), 1.61-1.57 (m, 1H), 1.46-1.33 (m, 4H), 1.16-1.05 (m, 3H). LC-MS (ESI): m/z=516.3, t_{R}=5.11 min.

3-(7-(2-(((1S,2S,4R)-bicyclo[2.2.1]heptan-2-yl)amino)-2-oxoethoxy)naphthalen-2-yl)-3-(6-methylbenzo[d][1,3]dioxol-5-yl)propanoic acid (5c, UCAB #1256). The reaction was conducted following the general procedure G, using I-20c (0.087 g, 0.17 mmol) and 2 M aq. NaOH (170 μl). White solid (0.054 g, 63.6%). ¹H NMR (600 MHz, DMSO) δ 12.56-11.81 (s, 1H), 7.87 (d, J=7.1 Hz, 1H), 7.76 (d, J=8.7 Hz, 1H), 7.72 (d, J=8.4 Hz, 1H), 7.55 (s, 1H), 7.27 (dd, J=8.6, 1.8 Hz, 1H), 7.17 (m, 2H), 6.95 (d, J=4.0 Hz, 1H), 6.73 (s, 1H), 5.93 (d, 2H), 4.66 (t, J=7.8 Hz, 1H), 4.54 (s, 2H), 3.58 (m, 1H), 3.05-2.96 (m, 2H), 2.25-2.20 (m, 4H), 2.07 (m, 1H), 1.63-1.57 (m, 1H), 1.47-1.34 (m, 4H), 1.15-1.04 (m, 3H). ¹³C NMR (151 MHz, DMSO) δ 172.72, 166.60, 155.94, 145.51, 145.16, 141.82, 141.80, 134.78, 133.99, 128.85, 128.64, 127.60, 127.25, 124.54, 124.48, 118.28, 110.35, 107.11, 107.05, 100.59, 66.97, 52.03, 42.27, 41.84, 40.06, 38.23, 35.07, 34.83, 27.97, 26.09, 19.29. LC-MS (ESI): m/z=502.2, t_{R}=4.72 min.

Methyl 3-(7-(2-(((1S,2S,4R)-bicyclo[2.2.1]heptan-2-yl)amino)-2-oxoethoxy)naphthalen-2-yl)-3-(7-methyl-2,3-dihydrobenzo[b][1,4]dioxin-6-yl)propanoate (I-20d). The reaction was conducted following the general procedure F, using I-19 (0.202 g, 0.48 mmol) and I-9u (0.071 g, 0.32 mmol), TEA (0.14 ml, 0.96 mmol), and [Rh(COD)Cl]₂ (15.8 mg, 0.032 mmol). White solid (0.11 g, 67.5%). ¹H NMR (600 MHz, DMSO) δ 7.87 (d, J=7.1 Hz, 1H), 7.76 (d, J=8.6 Hz, 1H), 7.72 (d, J=8.5 Hz, 1H), 7.54 (s, 1H), 7.24 (dd, J=8.5, 1.8 Hz, 1H), 7.20-7.15 (m, 2H), 6.82 (d, J=3.6 Hz, 1H), 6.62 (d, J=0.7 Hz, 1H), 4.63 (t, J=7.8 Hz, 1H), 4.54 (s, 2H), 4.22-4.13 (m, 4H), 3.58 (m, 1H), 3.51 (s, 3H), 3.12-3.06 (m, 2H), 2.21 (m, 1H), 2.18 (s, 3H), 2.08 (m, 1H), 1.60 (m, 1H), 1.46-1.23 (m, 4H), 1.16-1.05 (m, 3H). LC-MS (ESI): m/z=530.3, t_{R}=5.12 min.

3-(7-(2-(((1S,2S,4R)-bicyclo[2.2.1]heptan-2-yl)amino)-2-oxoethoxy)naphthalen-2-(5d, yl)-3-(7-methyl-2,3-dihydrobenzo[b][1,4]dioxin-6-yl)propanoic acid UCAB #1257). The reaction was conducted following the general procedure G, using I-20d (0.114 g, 0.22 mmol) and 2 M aq. NaOH (220 μl). White solid (0.059 g, 53.0%). ¹H NMR (600 MHz, DMSO) δ 12.14 (s, 1H), 7.87 (d, J=7.1 Hz, 1H), 7.77-7.74 (m, 1H), 7.72 (d, J=8.4 Hz, 1H), 7.54 (s, 1H), 7.24 (dd, J=8.5, 1.8 Hz, 1H), 7.18-7.15 (m, 2H), 6.81 (d, J=4.2 Hz, 1H), 6.62 (s, 1H), 4.61 (t, J=7.8 Hz, 1H), 4.54 (s, 2H), 4.17 (s, 4H), 3.57 (m, 1H), 2.96 (m, 2H), 2.21 (m, 1H), 2.17 (s, 3H), 2.08 (m, 1H), 1.63-1.57 (m, 1H), 1.48-1.34 (m, 4H), 1.09 (m, 3H). ¹³C NMR (151 MHz, DMSO) δ 172.68, 166.61, 155.94, 141.86, 141.28, 141.23, 134.54, 133.99, 128.86, 128.33, 127.60, 127.25, 124.54, 124.51, 118.45, 118.27, 115.23, 107.03, 66.97, 64.01 (2C), 52.04, 42.06, 41.84, 40.37, 38.22, 35.07, 34.83, 27.97, 26.09, 18.54. LC-MS (ESI): m/z=516.3, t_{R}=4.62 min.

2-((7-Bromonaphthalen-2-yl)oxy)-N-(cyclohex-3-en-1-yl) acetamide (I-21a). The reaction was conducted following the general procedure E, using I-2 (0.30 g, 1.07 mmol), HATU (0.82 g, 2.14 mmol), DIPEA (0.6 mL, 3.21 mmol) and cyclohex-3-en-1-amine hydrogen chloride (0.17 g, 1.60 mmol). White solid (0.30 g, 78.9%). ¹H NMR (600 MHz, DMSO) δ 8.05-7.99 (m, 2H), 7.88 (d, J=8.6 Hz, 1H), 7.81 (d, J=8.7 Hz, 1H), 7.47 (dd, J=8.7, 2.0 Hz, 1H), 7.30 (d, J=8.4 Hz, 2H), 5.68-5.58 (m, 2H), 4.58 (s, 2H), 3.93-3.86 (m, 1H), 2.24-2.16 (m, 1H), 2.09-2.05 (m, 2H), 2.05-1.96 (m, 1H), 1.76 (ddt, J=11.7, 5.4, 3.9 Hz, 1H), 1.54 (ddt, J=12.4, 11.2, 8.1 Hz, 1H). LC-MS (ESI): m/z=360.1/362.1, t_{R}=5.07 min.

N-(Cyclohex-3-en-1-yl)-2-((7-(4,4,5,5-tetramethyl-1,3,2-dioxaborolan-2-yl)naphthalen-2-yl)oxy) acetamide (I-22a). A solution of I-21a (0.30 g, 0.84 mmol), PinB-BPin (0.26 g, 1.00 mmol), KOAc (0.25 g, 2.48 mmol), and Pd(dppf)Cl₂ (0.02 g, 0.025 mmol) were added in dioxane (9 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.15 g, 44.1%). ¹H NMR (600 MHz, DMSO) δ 9.35 (d, 1H), 9.19 (d, J=8.0 Hz, 1H), 9.06-8.97 (m, 2H), 8.74 (dd, J=8.1, 1.2 Hz, 1H), 8.56 (d, J=2.6 Hz, 1H), 8.49 (dd, J=8.9, 2.6 Hz, 1H), 6.85-6.75 (m, 2H), 5.75 (s, 2H), 5.11-5.02 (m, 1H), 3.41-3.33 (m, 1H), 3.31-3.22 (m, 2H), 3.18 (ddq, J=17.3, 9.0, 2.8 Hz, 1H), 2.97-2.89 (m, 1H), 2.72 (ddt, J=12.5, 11.3, 8.1 Hz, 1H), 2.50 (s, 12H). LC-MS (ESI): m/z=408.2, t_{R}=5.29 min.

Methyl 3-(benzo[d][1,3]dioxol-5-yl)-3-(7-(2-(cyclohex-3-en-1-ylamino)-2-oxoethoxy)naphthalen-2-yl)propanoate (I-23a). The reaction was conducted following the general procedure F, using I-22a (0.15 g, 0.37 mmol) and I-9m (0.05 g, 0.25 mmol), TEA (0.11 ml, 0.74 mmol), and [Rh(COD)Cl]₂ (13 mg, 0.03 mmol). White solid (0.08 g, 67.8%). ¹H NMR (600 MHz, DMSO) δ 8.00 (d, J=8.0 Hz, 1H), 7.78-7.74 (m, 1H), 7.72 (d, J=8.5 Hz, 1H), 7.69-7.66 (m, 1H), 7.31 (dd, J=8.5, 1.8 Hz, 1H), 7.19 (d, J=7.7 Hz, 2H), 6.96 (d, J=1.7 Hz, 1H), 6.84-6.77 (m, 2H), 5.93 (dd, J=6.3, 1.0 Hz, 2H), 5.67-5.58 (m, 2H), 4.57 (s, 2H), 4.50 (t, J=8.0 Hz, 1H), 3.89 (dddd, J=16.6, 10.6, 6.8, 4.3 Hz, 1H), 3.49 (s, 3H), 3.21 (dd, J=15.9, 8.1 Hz, 1H), 3.15 (dd, J=15.9, 7.9 Hz, 1H), 2.22-2.16 (m, 1H), 2.11-2.05 (m, 2H), 2.04-1.96 (m, 1H), 1.75 (dt, J=12.1, 3.9 Hz, 1H), 1.59-1.49 (m, 1H). LC-MS (ESI): m/z=488.2, t_{R}=4.91 min.

3-(Benzo[d][1,3]dioxol-5-yl)-3-(7-(2-(cyclohex-3-en-1-ylamino)-2-oxoethoxy)naphthalen-2-yl)propanoic acid (6a, UCAB #1000). The reaction was conducted following the general procedure G, using I-23a (0.08 g, 0.16 mmol) and 2M aq. NaOH (350 μl). White solid (0.018 g, 23.1%). ¹H NMR (600 MHz, DMSO) δ 12.12 (s, 1H), 8.01 (d, J=8.1 Hz, 1H), 7.77 (d, J=8.7 Hz, 1H), 7.73 (d, J=8.5 Hz, 1H), 7.68 (d, J=1.7 Hz, 1H), 7.31 (dd, J=8.5, 1.8 Hz, 1H), 7.23-7.17 (m, 2H), 6.96 (d, J=1.6 Hz, 1H), 6.86-6.78 (m, 2H), 5.94 (dd, J=5.4, 1.0 Hz, 2H), 5.69-5.59 (m, 2H), 4.58 (s, 2H), 4.49 (t, J=7.9 Hz, 1H), 3.90 (dddd, J=17.0, 11.2, 5.4, 3.2 Hz, 1H), 3.10 (dd, J=15.8, 8.1 Hz, 1H), 3.03 (dd, J=15.8, 7.9 Hz, 1H), 2.21 (d, J=17.5 Hz, 1H), 2.12-2.06 (m, 2H), 2.06-1.96 (m, 1H), 1.79-1.73 (m, 1H), 1.60-1.51 (m, 1H). ¹³C NMR (151 MHz, DMSO) δ 172.68, 166.70, 155.83, 147.24, 145.55, 142.45, 137.98, 134.09, 128.91, 127.60, 127.36, 126.58, 124.96, 124.36, 124.22, 120.56, 118.20, 108.05 (2C), 107.21, 100.74, 67.06, 46.38, 44.22, 40.07, 30.89, 28.00, 24.06. LC-MS (ESI): m/z=474.2, t_{R}=4.49 min.

2-((7-bromonaphthalen-2-yl)oxy)-N-(4,4-difluorocyclohexyl) acetamide (I-21b). The reaction was conducted following the general procedure E, using I-2 (0.5 g, 1.77 mmol),

HATU (1.37 g, 3.61 mmol), DIPEA (2 mL, 11.48 mmol) and 4,4-Difluorocyclohexanamine Hydrochloride (0.46 g, 2.66 mmol). White solid (0.63 g, 90%). ¹H NMR (600 MHz, DMSO) δ 8.13 (d, J=8.0 Hz, 1H), 8.05 (d, J=2.0 Hz, 1H), 7.89 (d, J=8.9 Hz, 1H), 7.82 (d, J=8.7 Hz, 1H), 7.48 (dd, J=8.6, 2.0 Hz, 1H), 7.33-7.27 (m, 2H), 4.59 (s, 2H), 3.92-3.83 (m, 1H), 2.07-1.85 (m, 3H), 1.80 (dd, J=14.5, 3.8 Hz, 3H), 1.65-1.55 (m, 2H). LC-MS (ESI): m/z=398.1/400.1, t_{R}=4.76 min.

N-(4,4-difluorocyclohexyl)-2-((7-(4,4,5,5-tetramethyl-1,3,2-dioxaborolan-2-yl)naphthalen-2-yl)oxy) acetamide (I-22b). A solution of I-21b (0.62 g, 1.6 mmol), PinB-BPin (0.5 g, 1.90 mmol), KOAc (0.52 g, 5.3 mmol), and Pd(dppf)Cl₂ (0.04 g, 0.055 mmol) were added in dioxane (18 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.50 g, 70.0%). ¹H NMR (400 MHz, DMSO) δ 8.19 (d, J=1.1 Hz, 1H), 8.12 (d, J=8.0 Hz, 1H), 7.85 (dd, J=16.6, 8.5 Hz, 2H), 7.58 (dd, J=8.1, 1.2 Hz, 1H), 7.39 (d, J=2.6 Hz, 1H), 7.33 (dd, J=8.9, 2.6 Hz, 1H), 4.59 (s, 2H), 3.95-3.83 (m, 1H), 2.12-1.73 (m, 6H), 1.60 (qd, J=12.9, 4.3 Hz, 2H), 1.34 (s, 12H). LC-MS (ESI): m/z=446.2, t_{R}=5.02 min.

Methyl 3-(benzo[d][1,3]dioxol-5-yl)-3-(7-(2-((4,4-difluorocyclohexyl)amino)-2-oxoethoxy)naphthalen-2-yl)propanoate (I-23b). The reaction was conducted following the general procedure F, using I-22b (0.12 g, 0.27 mmol) and I-9m (0.04 g, 0.18 mmol), TEA (0.15 ml, 0.81 mmol), and [Rh(COD)Cl]₂ (11 mg, 0.02 mmol). White solid (0.05 g, 31%). ¹H NMR (600 MHz, DMSO) δ 8.11 (d, J=8.0 Hz, 1H), 7.80-7.75 (m, 1H), 7.73 (d, J=8.5 Hz, 1H), 7.70-7.67 (m, 1H), 7.32 (dd, J=8.5, 1.8 Hz, 1H), 7.20 (d, J=8.3 Hz, 2H), 6.97 (d, J=1.7 Hz, 1H), 6.86-6.78 (m, 2H), 5.94 (dd, J=6.4, 1.0 Hz, 2H), 4.58 (s, 2H), 4.52 (t, J=8.0 Hz, 1H), 3.88 (q, J=9.9 Hz, 1H), 3.50 (s, 3H), 3.22 (dd, J=15.9, 8.1 Hz, 1H), 3.16 (dd, J=15.9, 7.9 Hz, 1H), 2.11-1.85 (m, 2H), 1.85-1.72 (m, 2H), 1.65-1.55 (m, 2H), 1.31-1.22 (m, 2H). LC-MS (ESI): m/z=526.2, t_{R}=4.73 min.

3-(benzo[d][1,3]dioxol-5-yl)-3-(7-(2-((4,4-difluorocyclohexyl)amino)-2-oxoethoxy)naphthalen-2-yl)propanoic acid (6b, UCAB #1131). The reaction was conducted following the general procedure G, using I-23b (0.05 g, 0.084 mmol) and 2M aq. NaOH (420 μl). White solid (0.007 g, 17.0%). ¹H NMR (600 MHz, DMSO) δ 12.09 (s, 1H), 8.11 (d, J=8.0 Hz, 1H), 7.80-7.75 (m, 1H), 7.73 (d, J=8.5 Hz, 1H), 7.68 (d, J=1.7 Hz, 1H), 7.31 (dd, J=8.5, 1.8 Hz, 1H), 7.20 (dq, J=4.7, 2.6 Hz, 2H), 6.96 (d, J=1.6 Hz, 1H), 6.85-6.78 (m, 2H), 5.94 (dd, J=5.6, 1.0 Hz, 2H), 4.58 (s, 2H), 4.49 (t, J=7.9 Hz, 1H), 3.89 (tdt, J=9.3, 7.0, 4.6 Hz, 1H), 3.10 (dd, J=15.8, 8.1 Hz, 1H), 3.04 (dd, J=15.8, 7.9 Hz, 1H), 2.11-1.86 (m, 4H), 1.83-1.75 (m, 2H), 1.65-1.55 (m, 2H). ¹³C NMR (151 MHz, DMSO) δ 173.17, 167.35, 156.32, 147.73, 146.04, 142.94, 138.48, 134.57, 129.39, 128.10, 127.85, 124.85, 124.70, 121.05, 118.71, 108.56, 108.52, 107.63, 101.23, 67.46, 46.87, 45.44, 40.55, 31.94, 28.32 (2C), 28.25 (2C). LC-MS (ESI): m/z=512.2, t_{R}=4.33 min.

2-((7-bromonaphthalen-2-yl)oxy)-N-isobutylacetamide (I-21c). The reaction was conducted following the general procedure E, using I-2 (0.25 g, 0.89 mmol), HATU (0.67 g, 1.78 mmol), DIPEA (0.5 mL, 2.67 mmol) and 2-methylpropan-1-amine (0.46 g, 2.66 mmol). Light yellow solid (0.25 g, 83.6%). ¹H NMR (400 MHz, DMSO) õ=8.14 (t, J=5.9, 1H), 8.04 (d, J=2.0, 1H), 7.93-7.85 (m, 1H), 7.83 (d, J=8.7, 1H), 7.48 (dd, J=8.7, 2.0, 1H), 7.35-7.24 (m, 2H), 4.61 (s, 2H), 2.98 (dd, J=6.9, 6.0, 2H), 1.77 (qt, J=13.5, 6.6, 1H), 0.83 (d, J=6.7, 6H). LC-MS (ESI): m/z=336.1/338.1, t_{R}=4.77 min.

N-isobutyl-2-((7-(4,4,5,5-tetramethyl-1,3,2-dioxaborolan-2-yl)naphthalen-2-yl)oxy) acetamide (1-22c). A solution of I-21c (0.25 g, 0.74 mmol), PinB-BPin (0.35 g, 1.39 mmol), KOAc (0.34 g, 3.48 mmol), and Pd(dppf)Cl₂ (0.03 g, 0.01 mmol) were added in dioxane (5 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. Light yellow solid (0.21 g, 75.0%). ¹H NMR (600 MHz, DMSO) δ 8.18 (d, J=1.1 Hz, 1H), 8.14 (t, J=6.1 Hz, 1H), 7.90-7.81 (m, 2H), 7.58 (dd, J=8.1, 1.2 Hz, 1H), 7.40 (d, J=2.6 Hz, 1H), 7.33 (dd, J=8.9, 2.6 Hz, 1H), 4.61 (s, 2H), 2.98 (dd, J=6.9, 6.0 Hz, 2H), 1.75 (hept, J=6.8 Hz, 1H), 1.34 (s, 12H), 0.83 (d, J=6.7 Hz, 6H). LC-MS (ESI): m/z=384.3, t_{R}=5.06 min.

Methyl 3-(benzo[d][1,3]dioxol-5-yl)-3-(7-(2-(isobutylamino)-2-oxoethoxy)naphthalen-2-yl)propanoate (I-23c). The reaction was conducted following the general procedure F, using I-22c (0.1 g, 0.26 mmol) and I-9m (0.04 g, 0.17 mmol), TEA (0.1 ml, 0.52 mmol), and [Rh(COD)Cl]₂ (8 mg, 0.02 mmol). White solid (0.03 g, 22.5%). ¹H NMR (600 MHz, DMSO) δ 8.11 (t, J=6.0 Hz, 1H), 7.80-7.75 (m, 1H), 7.73 (d, J=8.5 Hz, 1H), 7.71-7.66 (m, 1H), 7.32 (dd, J=8.5, 1.8 Hz, 1H), 7.19 (dd, J=6.7, 2.7 Hz, 2H), 6.96 (d, J=1.7 Hz, 1H), 6.85-6.78 (m, 2H), 5.94 (dd, J=6.1, 1.0 Hz, 2H), 4.59 (s, 2H), 4.51 (t, J=8.0 Hz, 1H), 3.50 (s, 3H), 3.22 (dd, J=15.9, 8.1 Hz, 1H), 3.15 (dd, J=15.9, 7.9 Hz, 1H), 2.97 (t, J=6.5 Hz, 2H), 1.74 (dp, J=13.4, 6.7 Hz, 1H), 0.89-0.80 (m, 6H). LC-MS (ESI): m/z=464.3, t_{R}=4.72 min.

3-(benzo[d][1,3]dioxol-5-yl)-3-(7-(2-(isobutylamino)-2-oxoethoxy)naphthalen-2-yl)propanoic acid (6c, UCAB #1164). The reaction was conducted following the general procedure G, using I-23c (0.03 g, 0.059 mmol) and 2M aq. NaOH (100 μl). White solid (0.011 g, 30.3%). ¹H NMR (600 MHz, DMSO) δ 12.09 (s, 1H), 8.11 (t, J=6.0 Hz, 1H), 7.76 (d, J=8.7 Hz, 1H), 7.71 (d, J=8.5 Hz, 1H), 7.66 (d, J=1.7 Hz, 1H), 7.30 (dd, J=8.5, 1.8 Hz, 1H), 7.21-7.16 (m, 2H), 6.94 (d, J=1.6 Hz, 1H), 6.83-6.77 (m, 2H), 5.95-5.91 (m, 2H), 4.58 (s, 2H), 4.47 (t, J=7.9 Hz, 1H), 3.12-2.94 (m, 4H), 1.78-1.69 (m, J=6.8 Hz, 1H), 0.82 (d, J=6.7 Hz, 6H). ¹³C NMR (151 MHz, DMSO) δ 172.39, 167.12, 155.54, 146.97, 145.27, 142.17, 137.71, 133.80, 128.64, 127.32, 127.09, 124.10, 123.91, 120.28, 117.94, 107.78, 107.74, 106.87, 100.47, 66.76, 46.10, 45.48, 39.66, 27.77, 19.75 (2C). LC-MS (ESI): m/z=450.3, t_{R}=4.29 min.

2-((7-bromonaphthalen-2-yl)oxy)-N-cyclopentylacetamide (I-21d). The reaction was conducted following the general procedure E, using I-2 (0.15 g, 0.53 mmol), HATU (0.41 g, 1.07 mmol), DIPEA (0.3 mL, 1.60 mmol) and cyclopentanamine (0.08 g, 0.80 mmol). Light yellow solid (0.15 g, 81%). ¹H NMR (400 MHz, DMSO) δ 8.07-8.00 (m, 2H), 7.89 (d, J=8.8 Hz, 1H), 7.82 (d, J=8.7 Hz, 1H), 7.48 (dd, J=8.7, 2.1 Hz, 1H), 7.35-7.26 (m, 2H), 4.57 (s, 2H), 4.11 (p, J=7.0 Hz, 1H), 1.81 (dd, J=11.2, 6.5 Hz, 4H), 1.71-1.41 (m, 4H). LC-MS (ESI): m/z=348.1/350.1, t_{R}=4.92 min.

N-cyclopentyl-2-((7-(4,4,5,5-tetramethyl-1,3,2-dioxaborolan-2-yl)naphthalen-2-yl)oxy) acetamide (I-22d). A solution of I-21d (0.15 g, 0.4 mmol), PinB-BPin (0.23 g, 0.88 mmol), KOAc (0.22 g, 2.2 mmol), and Pd(dppf)Cl₂ (0.015 g, 0.02 mmol) were added in dioxane (5 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. Light yellow solid (0.15 g, 97.4%). ¹H NMR (600 MHz, DMSO) δ 8.17 (d, J=1.2 Hz, 1H), 8.02 (d, J=7.5 Hz, 1H), 7.86 (d, J=9.0 Hz, 1H), 7.82 (d, J=8.1 Hz, 1H), 7.57 (dd, J=8.1, 1.2 Hz, 1H), 7.38 (d, J=2.6 Hz, 1H), 7.32 (dd, J=8.9, 2.6 Hz, 1H), 4.55 (s, 2H), 4.10 (h, J=7.1 Hz, 1H), 1.86-1.78 (m, 2H), 1.69-1.60 (m, 2H), 1.55-1.41 (m, 4H), 1.33 (s, 12H). LC-MS (ESI): m/z=396.2, t_{R}=5.20 min.

Methyl 3-(benzo[d][1,3]dioxol-5-yl)-3-(7-(2-(cyclopentylamino)-2-oxoethoxy)naphthalen-2-yl)propanoat (I-23d). The reaction was conducted following the general procedure F, using I-22d (0.1 g, 0.25 mmol) and I-9m (0.04 g, 0.16 mmol), TEA (0.1 ml, 0.47 mmol), and [Rh(COD)Cl]₂ (9 mg, 0.02 mmol). Colorless oil (0.04 g, 33.3%). ¹H NMR (400 MHz, DMSO) δ 8.01 (d, J=7.5 Hz, 1H), 7.81-7.65 (m, 3H), 7.32 (dd, J=8.4, 1.8 Hz, 1H), 7.23-7.16 (m, 2H), 6.97 (d, J=1.6 Hz, 1H), 6.87-6.77 (m, 2H), 5.97-5.91 (m, 2H), 4.53 (d, J=14.0 Hz, 3H), 4.17-3.99 (m, 1H), 3.51 (s, 3H), 3.28-3.10 (m, 2H), 1.87-1.76 (m, 2H), 1.69-1.59 (m, 2H), 1.52-1.39 (m, 4H). LC-MS (ESI): m/z=476.2, t_{R}=4.79 min.

3-(benzo[d][1,3]dioxol-5-yl)-3-(7-(2-(cyclopentylamino)-2-oxoethoxy)naphthalen-2-yl)propanoic acid (6d, UCAB #1165). The reaction was conducted following the general procedure G, using I-23d (0.04 g, 0.084 mmol) and 2M aq. NaOH (100 μl). White solid (0.02 g, 52.3%). ¹H NMR (600 MHz, DMSO) δ 12.10 (s, 1H), 8.01 (d, J=7.5 Hz, 1H), 7.79-7.75 (m, 1H), 7.73 (d, J=8.5 Hz, 1H), 7.69-7.66 (m, 1H), 7.31 (dd, J=8.5, 1.8 Hz, 1H), 7.22-7.17 (m, 2H), 6.96 (d, J=1.6 Hz, 1H), 6.85-6.78 (m, 2H), 5.94 (dd, J=5.1, 1.0 Hz, 2H), 4.55 (s, 2H), 4.49 (t, J=8.0 Hz, 1H), 4.10 (h, J=7.1 Hz, 1H), 3.13-3.00 (m, 2H), 1.87-1.78 (m, 2H), 1.66 (tdd, J=10.8, 6.3, 2.2 Hz, 2H), 1.56-1.41 (m, 4H). ¹³C NMR (151 MHz, DMSO) δ 173.16, 167.30, 156.38, 147.74, 146.04, 142.93, 138.47, 134.58, 129.38, 128.09, 127.83, 124.82, 124.69, 121.05, 118.72, 108.55, 108.52 107.62, 101.23, 67.53, 50.59, 46.87, 40.55, 32.55 (2C), 23.95 (2C). LC-MS (ESI): m/z=462.2, t_{R}=4.35 min.

N-((1S,3s)-adamantan-1-yl)-2-((7-bromonaphthalen-2-yl)oxy) acetamide (I-21e). The reaction was conducted following the general procedure E, using I-2 (0.15 g, 0.53 mmol), HATU (0.41 g, 1.07 mmol), DIPEA (0.3 mL, 1.60 mmol) and (1S,3s)-adamantan-1-amine (0.12 g, 0.80 mmol). Light yellow solid (0.20 g, 90.9%). ¹H NMR (400 MHz, DMSO) δ 8.04 (d, J=2.0 Hz, 1H), 7.89 (d, J=8.7 Hz, 1H), 7.82 (d, J=8.7 Hz, 1H), 7.48 (dd, J=8.7, 2.0 Hz, 1H), 7.39 (s, 1H), 7.32-7.24 (m, 2H), 4.51 (s, 2H), 2.06-1.96 (m, 8H), 1.88-1.81 (m, 2H), 1.67-1.57 (m, 5H). LC-MS (ESI): m/z=414.1/416.1, t_{R}=5.36 min.

N-((1S,3s)-adamantan-1-yl)-2-((7-(4,4,5,5-tetramethyl-1,3,2-dioxaborolan-2-yl)naphthalen-2-yl)oxy) acetamide (I-22e). A solution of I-21e (0.2 g, 0.53 mmol), PinB-BPin (0.16 g, 0.64 mmol), KOAc (0.16 g, 1.6 mmol), and Pd(dppf)Cl₂ (0.012 g, 0.02 mmol) were added in dioxane (5 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.2 g, 90.9%). ¹H NMR (600 MHz, DMSO) δ 8.18 (d, J=1.1 Hz, 1H), 7.89-7.81 (m, 2H), 7.58 (dd, J=8.1, 1.1 Hz, 1H), 7.37 (d, J=2.2 Hz, 2H), 7.31 (dd, J=8.9, 2.6 Hz, 1H), 4.51 (s, 2H), 2.03 (t, J=3.1 Hz, 3H), 1.99 (d, J=2.9 Hz, 6H), 1.64 (t, J=3.1 Hz, 6H), 1.34 (s, 12H). LC-MS (ESI): m/z=462.3, t_{R}=5.91 min.

Methyl 3-(7-(2-(((1S,3s)-adamantan-1-yl)amino)-2-oxoethoxy)naphthalen-2-yl)-3-(benzo[d][1,3]dioxol-5-yl)propanoate (I-23e). The reaction was conducted following the general procedure F, using I-22e (0.15 g, 0.33 mmol) and I-9m (0.045 g, 0.22 mmol), TEA (0.1 ml, 0.65 mmol), and [Rh(COD)Cl]₂ (11 mg, 0.02 mmol). White solid (0.063 g, 36.0%). ¹H NMR (600 MHz, DMSO) δ 7.79-7.71 (m, 2H), 7.69-7.64 (m, 1H), 7.36 (s, 1H), 7.31 (dd, J=8.5, 1.8 Hz, 1H), 7.17 (d, J=8.0 Hz, 2H), 6.97 (d, J=1.7 Hz, 1H), 6.86-6.77 (m, 2H), 5.94 (dd, J=6.3, 1.0 Hz, 2H), 4.51 (d, J=12.8 Hz, 3H), 3.50 (s, 3H), 3.26-3.12 (m, 2H), 2.03 (s, 3H), 1.98 (d, J=2.9 Hz, 6H), 1.64 (d, J=3.1 Hz, 6H). LC-MS (ESI): m/z=542.3, t_{R}=5.03 min.

3-(7-(2-(((1S,3s)-adamantan-1-yl)amino)-2-oxoethoxy)naphthalen-2-yl)-3-(benzo[d][1,3]dioxol-5-yl)propanoic acid (6e, UCAB #1167). The reaction was conducted following the general procedure G, using I-23e (0.063 g, 0.12 mmol) and 2M aq. NaOH (200 μl). White solid (0.015 g, 26.2%). ¹H NMR (600 MHz, DMSO) δ 12.13 (s, 1H), 7.78-7.70 (m, 2H), 7.66 (d, J=1.7 Hz, 1H), 7.35 (s, 1H), 7.31 (dd, J=8.5, 1.8 Hz, 1H), 7.20-7.14 (m, 2H), 6.95 (d, J=1.7 Hz, 1H), 6.84-6.77 (m, 2H), 5.93 (dd, J=5.4, 1.0 Hz, 2H), 4.49 (d, J=3.6 Hz, 3H), 3.11-3.00 (m, 2H), 2.04-1.95 (m, 9H), 1.63 (d, J=3.2 Hz, 6H). ¹³C NMR (151 MHz, DMSO) δ 173.17, 166.89, 156.36, 147.74, 146.04, 142.93, 138.47, 134.59, 129.40, 128.10, 127.82, 124.80, 124.67, 121.05, 118.60, 108.55, 108.52, 107.71, 101.24, 67.66, 51.52, 46.87, 41.42 (3C), 40.54, 36.42 (3C), 29.28 (3C). LC-MS (ESI): m/z=528.2, t_{R}=5.05 min.

2-((7-bromonaphthalen-2-yl)oxy)-N-(1-methylcyclohexyl) acetamide (I-21f). The reaction was conducted following the general procedure E, using I-2 (0.15 g, 0.53 mmol), HATU (0.41 g, 1.07 mmol), DIPEA (0.3 mL, 1.60 mmol) and 1-methylcyclohexan-1-amine (0.1 g, 0.80 mmol). White solid (0.19 g, 95%). ¹H NMR (400 MHz, DMSO) δ 8.01 (d, J=2.0 Hz, 1H), 7.92-7.76 (m, 2H), 7.48 (dd, J=8.7, 2.0 Hz, 1H), 7.33-7.23 (m, 3H), 4.58 (s, 2H), 2.07 (d, J=12.4 Hz, 2H), 1.49-1.22 (m, 11H). LC-MS (ESI): m/z=376.0/378.1, t_{R}=5.36 min.

N-(1-methylcyclohexyl)-2-((7-(4,4,5,5-tetramethyl-1,3,2-dioxaborolan-2-yl)naphthalen-2-yl)oxy) acetamide (I-22f). A solution of I-21f (0.19 g, 0.53 mmol), PinB-BPin (0.16 g, 0.64 mmol), KOAc (0.16 g, 1.6 mmol), and Pd(dppf)Cl₂ (0.012 g, 0.02 mmol) were added in dioxane (5 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. Colorless oil (0.2 g, 95.2%). ¹H NMR (600 MHz, DMSO) δ 8.17 (d, J=1.1 Hz, 1H), 7.87 (d, J=9.0 Hz, 1H), 7.83 (d, J=8.1 Hz, 1H), 7.57 (dd, J=8.1, 1.1 Hz, 1H), 7.40 (d, J=2.6 Hz, 1H), 7.31 (dd, J=8.9, 2.6 Hz, 1H), 7.25 (s, 1H), 4.58 (s, 2H), 1.34 (m, 25H). LC-MS (ESI): m/z=424.3, t_{R}=5.61 min.

Methyl 3-(benzo[d][1,3]dioxol-5-yl)-3-(7-(2-((1-methylcyclohexyl)amino)-2-oxoethoxy)naphthalen-2-yl)propanoate (I-23f). The reaction was conducted following the general procedure F, using I-22f (0.1 g, 0.24 mmol) and I-9m (0.022 g, 0.16 mmol), TEA (0.1 ml, 0.47 mmol), and [Rh(COD)Cl]₂ (8 mg, 0.016 mmol). Colorless oil (0.066 g, 55.9%). ¹H NMR (600 MHz, DMSO) δ 7.83-7.72 (m, 2H), 7.64 (d, J=1.7 Hz, 1H), 7.31 (dd, J=8.5, 1.8 Hz, 1H), 7.23-7.15 (m, 3H), 6.95 (d, J=1.6 Hz, 1H), 6.85-6.77 (m, 2H), 5.94 (dd, J=6.3, 1.0 Hz, 2H), 4.57 (s, 2H), 4.52 (t, J=8.0 Hz, 1H), 3.50 (s, 3H), 3.25-3.11 (m, 2H), 2.07 (d, J=12.9 Hz, 2H), 1.45-1.11 (m, 11H). LC-MS (ESI): m/z=504.3, t_{R}=5.21 min.

3-(benzo[d][1,3]dioxol-5-yl)-3-(7-(2-((1-methylcyclohexyl)amino)-2-oxoethoxy)naphthalen-2-yl)propanoic acid (6f, UCAB #1168). The reaction was conducted following the general procedure G, using I-23f (0.066 g, 0.13 mmol) and 2M aq. NaOH (200 μl). White solid (0.02 g, 31.3%). ¹H NMR (600 MHz, DMSO) δ 12.11 (s, 1H), 7.77 (d, J=8.9 Hz, 1H), 7.73 (d, J=8.4 Hz, 1H), 7.64 (d, J=1.7 Hz, 1H), 7.31 (dd, J=8.5, 1.8 Hz, 1H), 7.23-7.15 (m, 3H), 6.94 (d, J=1.4 Hz, 1H), 6.84-6.78 (m, 2H), 5.94 (dd, J=5.4, 1.0 Hz, 2H), 4.56 (s, 2H), 4.49 (t, J=8.0 Hz, 1H), 3.12-2.99 (m, 2H), 2.07 (d, J=12.8 Hz, 2H), 1.45-1.13 (m, 11H). ¹³C NMR (151 MHz, DMSO) δ 173.14, 167.15, 156.30, 147.74, 146.04, 142.95, 138.43, 134.56, 129.45, 128.10, 127.81, 124.84, 124.59, 121.04, 118.60, 108.55, 108.52, 107.64, 101.24, 67.63, 53.10, 46.83, 40.49, 36.35 (2C), 26.98, 25.55, 21.93 (2C). LC-MS (ESI): m/z=490.2, t_{R}=4.77 min.

2-((7-bromonaphthalen-2-yl)oxy)-N-(4,4-dimethylcyclohexyl) acetamide (I-21g). The reaction was conducted following the general procedure E, using I-2 (0.15 g, 0.53 mmol), HATU (0.41 g, 1.07 mmol), DIPEA (0.3 mL, 1.60 mmol) and 4,4-dimethylcyclohexan-1-amine (0.12 ml, 0.80 mmol). White solid (0.17 g, 85%). ¹H NMR (400 MHz, DMSO) δ 8.03 (d, J=2.0 Hz, 1H), 7.90 (dd, J=17.7, 8.5 Hz, 2H), 7.81 (d, J=8.7 Hz, 1H), 7.47 (dd, J=8.7, 2.0 Hz, 1H), 7.34-7.25 (m, 2H), 4.55 (s, 2H), 3.66-3.51 (m, 1H), 1.60-1.39 (m, 4H), 1.35 (d, J=13.1 Hz, 2H), 1.20 (td, J=13.0, 4.2 Hz, 2H), 0.89 (d, J=4.8 Hz, 6H). LC-MS (ESI): m/z=390.1/391.1, t_{R}=5.36 min.

N-(4,4-dimethylcyclohexyl)-2-((7-(4,4,5,5-tetramethyl-1,3,2-dioxaborolan-2-yl)naphthalen-2-yl)oxy) acetamide (I-22g). A solution of I-21g (0.17 g, 0.44 mmol), PinB-BPin (0.13 g, 0.52 mmol), KOAc (0.13 g, 1.31 mmol), and Pd(dppf)Cl₂ (0.01 g, 0.013 mmol) were added in dioxane (5 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.16 g, 84.2%). ¹H NMR (600 MHz, DMSO) δ 8.19 (d, J=1.1 Hz, 1H), 7.95 (d, J=8.2 Hz, 1H), 7.89-7.81 (m, 2H), 7.58 (dd, J=8.1, 1.1 Hz, 1H), 7.40 (d, J=2.6 Hz, 1H), 7.33 (dd, J=8.9, 2.6 Hz, 1H), 4.56 (s, 2H), 3.66-3.57 (m, 1H), 1.57 (dt, J=12.9, 4.2 Hz, 2H), 1.53-1.43 (m, 2H), 1.34 (s, 12H), 1.29-1.15 (m, 4H), 0.91 (d, J=10.2 Hz, 6H). LC-MS (ESI): m/z=438.2, t_{R}=5.62 min.

Methyl 3-(benzo[d][1,3]dioxol-5-yl)-3-(7-(2-((4,4-dimethylcyclohexyl)amino)-2-oxoethoxy)naphthalen-2-yl)propanoate (I-23g). The reaction was conducted following the general procedure F, using I-22g (0.1 g, 0.23 mmol) and I-9m (0.022 g, 0.16 mmol), TEA (0.1 ml, 0.47 mmol), and [Rh(COD)Cl]₂ (9 mg, 0.016 mmol). White solid (0.06 g, 50.8%). ¹H NMR (400 MHz, DMSO) δ 7.92 (d, J=8.2 Hz, 1H), 7.81-7.66 (m, 3H), 7.32 (dd, J=8.4, 1.8 Hz, 1H), 7.24-7.17 (m, 2H), 6.97 (d, J=1.6 Hz, 1H), 6.87-6.77 (m, 2H), 5.98-5.91 (m, 2H), 4.54 (d, J=16.0 Hz, 3H), 3.61 (m, 1H), 3.50 (s, 3H), 3.27-3.10 (m, 2H), 1.68-1.14 (m, 8H), 1.08 (s, 6H). LC-MS (ESI): m/z=518.3, t_{R}=5.17 min.

3-(benzo[d][1,3]dioxol-5-yl)-3-(7-(2-((4,4-dimethylcyclohexyl)amino)-2-oxoethoxy)naphthalen-2-yl)propanoic acid (6g, UCAB #1176). The reaction was conducted following the general procedure G, using I-23g (0.06 g, 0.11 mmol) and 2M aq. NaOH (200 μl). White solid (0.014 g, 23.8%). ¹H NMR (600 MHz, DMSO) δ 12.08 (s, 1H), 7.92 (d, J=8.2 Hz, 1H), 7.80-7.75 (m, 1H), 7.73 (d, J=8.5 Hz, 1H), 7.68 (d, J=1.7 Hz, 1H), 7.31 (dd, J=8.5, 1.8 Hz, 1H), 7.20 (d, J=7.7 Hz, 2H), 6.95 (d, J=1.6 Hz, 1H), 6.85-6.78 (m, 2H), 5.94 (dd, J=5.4, 1.0 Hz, 2H), 4.55 (s, 2H), 4.48 (t, J=8.0 Hz, 1H), 3.61 (dddd, J=15.3, 10.8, 8.5, 4.4 Hz, 1H), 3.12-3.00 (m, 2H), 1.60-1.53 (m, 2H), 1.48 (tdd, J=12.9, 10.8, 3.6 Hz, 2H), 1.40-1.33 (m, 2H), 1.22 (td, J=13.1, 4.0 Hz, 2H), 0.91 (d, J=7.0 Hz, 6H). ¹³C NMR (151 MHz, DMSO) δ 173.16, 166.93, 156.31, 147.74, 146.04, 142.93, 138.48, 134.58, 129.39, 128.09, 127.85, 124.84, 124.71, 121.05, 118.71, 108.55, 108.52, 107.70, 101.23, 67.57, 47.93, 46.87, 40.49, 37.85, 32.14, 29.78 (2C), 28.32 (2C), 25.12. LC-MS (ESI): m/z=504.2, t_{R}=4.76 min.

N-((1S,2R,4R)-bicyclo[2.2.1]heptan-2-yl)-2-((7-bromonaphthalen-2-yl)oxy) acetamide (I-21h). The reaction was conducted following the general procedure E, using I-2 (0.15 g, 0.53 mmol), HATU (0.41 g, 1.07 mmol), DIPEA (0.3 mL, 1.60 mmol) and (1S,2R,4R)-bicyclo[2.2.1]heptan-2-amine (0.1 ml, 0.80 mmol). White solid (0.15 g, 75%). ¹H NMR (600 MHz, DMSO) δ 8.03 (d, J=2.0 Hz, 1H), 7.89 (t, J=7.7 Hz, 2H), 7.82 (d, J=8.6 Hz, 1H), 7.48 (dd, J=8.6, 2.0 Hz, 1H), 7.32-7.25 (m, 2H), 4.57 (d, J=1.6 Hz, 2H), 3.60 (td, J=7.7, 3.6 Hz, 1H), 2.22 (d, J=4.7 Hz, 1H), 2.11 (d, J=4.2 Hz, 1H), 1.62 (ddd, J=12.6, 8.2, 2.3 Hz, 1H), 1.49-1.34 (m, 4H), 1.17-1.06 (m, 3H). LC-MS (ESI): m/z=374.0/376.0, t_{R}=5.06 min.

N-((1S,2R,4R)-bicyclo[2.2.1]heptan-2-yl)-2-((7-(4,4,5,5-tetramethyl-1,3,2-dioxaborolan-2-yl)naphthalen-2-yl)oxy) acetamide (I-22h). A solution of I-21h (0.15 g, 0.43 mmol), PinB-BPin (0.13 g, 0.51 mmol), KOAc (0.13 g, 1.31 mmol), and Pd(dppf)Cl₂ (0.01 g, 0.013 mmol) were added in dioxane (5 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.15 g, 85.6%). ¹H NMR (600 MHz, DMSO) δ 8.18 (d, J=1.1 Hz, 1H), 7.94-7.81 (m, 3H), 7.57 (dd, J=8.1, 1.1 Hz, 1H), 7.37 (d, J=2.6 Hz, 1H), 7.32 (dd, J=8.9, 2.6 Hz, 1H), 4.56 (d, J=2.3 Hz, 2H), 3.60 (td, J=7.5, 3.3 Hz, 1H), 2.22 (t, J=4.2 Hz, 1H), 2.11 (d, J=4.1 Hz, 1H), 1.62 (ddd, J=12.6, 8.1, 2.3 Hz, 1H), 1.50-1.40 (m, 4H), 1.34 (s, 12H), 1.12-1.07 (m, 3H). LC-MS (ESI): m/z=422.2, t_{R}=5.30 min.

Methyl 3-(benzo[d][1,3]dioxol-5-yl)-3-(7-(2-(((1S,2R,4R)-bicyclo[2.2.1]heptan-2-yl)amino)-2-oxoethoxy)naphthalen-2-yl)propanoate (I-23h). The reaction was conducted following the general procedure F, using I-22h (0.15 g, 0.36 mmol) and I-9m (0.051 g, 0.24 mmol), TEA (0.1 ml, 0.71 mmol), and [Rh(COD)Cl]₂ (12 mg, 0.024 mmol). White solid (0.07 g, 56.9%). ¹H NMR (600 MHz, DMSO) δ 7.88 (dd, J=7.3, 2.7 Hz, 1H), 7.79-7.71 (m, 2H), 7.66 (s, 1H), 7.32 (dt, J=8.6, 1.5 Hz, 1H), 7.21-7.15 (m, 2H), 6.97 (d, J=1.6 Hz, 1H), 6.85-6.78 (m, 2H), 5.94 (dd, J=6.0, 1.0 Hz, 2H), 4.59-4.49 (m, 3H), 3.59 (td, J=7.6, 3.6 Hz, 1H), 3.51 (s, 3H), 3.25-3.12 (m, 2H), 2.22 (s, 1H), 2.09 (s, 1H), 1.64-1.57 (m, 1H), 1.49-1.33 (m, 3H), 1.21-1.05 (m, 4H). LC-MS (ESI): m/z=502.3, t_{R}=4.93 min.

3-(benzo[d][1,3]dioxol-5-yl)-3-(7-(2-(((1S,2R,4R)-bicyclo[2.2.1]heptan-2-yl)amino)-2-oxoethoxy)naphthalen-2-yl)propanoic acid (6 h, UCAB #1188). The reaction was conducted following the general procedure G, using I-23h (0.06 g, 0.14 mmol) and 2M aq. NaOH (140 μl). White solid (0.03 g, 44.8%). ¹H NMR (600 MHz, DMSO) δ 12.12 (s, 1H), 7.88 (dd, J=7.1, 3.0 Hz, 1H), 7.79-7.75 (m, 1H), 7.73 (d, J=8.5 Hz, 1H), 7.66 (s, 1H), 7.31 (dt, J=8.5, 1.4 Hz, 1H), 7.21-7.16 (m, 2H), 6.95 (d, J=1.5 Hz, 1H), 6.85-6.78 (m, 2H), 5.94 (dd, J=5.1, 1.0 Hz, 2H), 4.56 (s, 2H), 4.49 (t, J=7.9 Hz, 1H), 3.60 (td, J=7.8, 3.5 Hz, 1H), 3.14-3.00 (m, 2H), 2.22 (d, J=4.3 Hz, 1H), 2.09 (q, J=5.8 Hz, 1H), 1.61 (ddt, J=12.5, 8.3, 2.1 Hz, 1H), 1.52-1.32 (m, 4H), 1.18-1.05 (m, 3H). ¹³C NMR (151 MHz, DMSO) δ 173.17, 167.10, 156.45, 147.74, 146.04, 142.92, 138.46, 134.57, 129.37, 128.10, 127.81, 124.81, 124.67, 121.05, 118.72, 108.54 (2C), 107.58, 101.24, 67.49, 52.54, 46.86, 42.35, 40.55, 38.75, 35.57, 35.36, 28.46, 26.61. LC-MS (ESI): m/z=504.2, t_{R}=4.76 min.

2-((7-bromonaphthalen-2-yl)oxy)-N-trans-(4-(trifluoromethyl)cyclohexyl) acetamide (I-21i). The reaction was conducted following the general procedure E, using I-2 (0.15 g, 0.53 mmol), HATU (0.41 g, 1.07 mmol), DIPEA (0.3 mL, 1.60 mmol) and 4-(trifluoromethyl)cyclohexan-1-amine (0.14 g, 0.80 mmol). Colorless oil (0.1 g, 90.1%). ¹H NMR (400 MHz, DMSO) δ 8.08-8.02 (m, 2H), 7.89 (d, J=8.8 Hz, 1H), 7.82 (d, J=8.7 Hz, 1H), 7.48 (dd, J=8.8, 2.0 Hz, 1H), 7.34-7.26 (m, 2H), 4.57 (s, 2H), 3.65 (s, 1H), 2.24 (m, 2H), 1.88 (d, J=8.8 Hz, 4H), 1.34 (dd, J=11.2, 8.7 Hz, 3H). ¹⁹F NMR (376 MHz, DMSO) δ −72.06. LC-MS (ESI): m/z=430.1/.432.0, t_{R}=5.05 min.

2-((7-(4,4,5,5-tetramethyl-1,3,2-dioxaborolan-2-yl)naphthalen-2-yl)oxy)-N-trans-(4-(trifluoromethyl)cyclohexyl) acetamide (I-22i). A solution of I-21i (0.1 g, 0.28 mmol), PinB-BPin (0.1 g, 0.34 mmol), KOAc (0.1 g, 0.84 mmol), and Pd(dppf)Cl₂ (0.01 g, 0.01 mmol) were added in dioxane (5 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.045 g, 33.8%). ¹H NMR (400 MHz, DMSO) δ 8.18 (s, 1H), 8.04 (d, J=7.9 Hz, 1H), 7.85 (dd, J=15.9, 8.5 Hz, 2H), 7.58 (dd, J=8.0, 1.2 Hz, 1H), 7.39 (d, J=2.6 Hz, 1H), 7.32 (dd, J=8.9, 2.6 Hz, 1H), 4.57 (s, 2H), 3.65 (s, 1H), 2.23 (s, 1H), 1.89 (d, J=9.1 Hz, 4H), 1.34 (s, 16H). ¹⁹F NMR (376 MHz, DMSO) δ −72.06. LC-MS (ESI): m/z=478.3, t_{R}=5.25 min.

Methyl 3-(benzo[d][1,3]dioxol-5-yl)-3-(7-(2-oxo-2-((trans-4-(trifluoromethyl)cyclohexyl)amino)ethoxy)naphthalen-2-yl)propanoate (I-23i). The reaction was conducted following the general procedure F, using I-22i (0.06 g, 0.13 mmol) and I-9m (0.026 g, 0.2 mmol), TEA (0.04 ml, 0.25 mmol), and [Rh(COD)Cl]₂ (10 mg, 0.01 mmol). Colorless oil (0.012 g, 17.1%). ¹H NMR (600 MHz, DMSO) δ 8.03 (d, J=8.0 Hz, 1H), 7.80-7.71 (m, 2H), 7.70-7.64 (m, 1H), 7.32 (dd, J=8.5, 1.8 Hz, 1H), 7.20 (dd, J=6.6, 2.7 Hz, 2H), 6.97 (d, J=1.7 Hz, 1H), 6.87-6.78 (m, 2H), 5.94 (dd, J=5.9, 1.0 Hz, 2H), 4.56 (s, 2H), 4.52 (q, J=7.7 Hz, 1H), 3.67-3.62 (m, 1H), 3.50 (s, 3H), 3.26-3.13 (m, 2H), 2.23 (d, J=10.2 Hz, 1H), 1.89 (d, J=12.6 Hz, 4H), 1.41-1.27 (m, 4H). ¹⁹F NMR (376 MHz, DMSO) δ −72.07 (d, J=9.6 Hz). LC-MS (ESI): m/z=558.2, t_{R}=4.91 min.

3-(benzo[d][1,3]dioxol-5-yl)-3-(7-(2-oxo-2-((trans-4-(trifluoromethyl)cyclohexyl)amino)ethoxy)naphthalen-2-yl)propanoic acid (6i, UCAB #1202_P1). The reaction was conducted following the general procedure G, using I-23h (0.012 g, 0.022 mmol) and 2M aq. NaOH (60 μl). White solid (0.003 g, 27.3%). ¹H NMR (600 MHz, DMSO) δ 12.16 (s, 1H), δ 8.03 (d, J=8.0 Hz, 1H), 7.79-7.66 (m, 3H), 7.31 (d, J=8.6 Hz, 1H), 7.18 (d, J=8.0 Hz, 2H), 6.95 (s, 1H), 6.81 (q, J=7.9 Hz, 2H), 5.93 (d, J=4.9 Hz, 2H), 4.56 (s, 2H), 4.48 (t, J=7.9 Hz, 1H), 3.58 (m, 1H), 3.13-3.00 (m, 2H), 2.22 (s, 1H), 1.87 (t, J=9.7 Hz, 4H), 1.37-1.31 (m, 4H). ¹³C NMR (151 MHz, DMSO) δ 173.16, 167.13, 156.33, 147.73, 146.04, 142.93, 138.48, 134.58, 129.41, 129.39, 128.09, 127.85, 124.84, 124.71, 121.06, 118.70, 108.54 (2C), 107.66, 101.23, 67.53, 47.33, 46.87, 40.43 (2C), 30.67 (2C), 24.09 (2C). ¹⁹F NMR (376 MHz, DMSO) δ −72.07. ¹⁹F NMR (376 MHz, DMSO) δ -72.04 LC-MS (ESI): m/z=488.3, t_{R}=4.61 min.

2-((7-bromonaphthalen-2-yl)oxy)-N-cis-(4-(trifluoromethyl)cyclohexyl) acetamide (I-21j). The reaction was conducted following the general procedure E, using I-2 (0.15 g, 0.53 mmol), HATU (0.41 g, 1.07 mmol), DIPEA (0.3 mL, 1.60 mmol) and 4-(trifluoromethyl)cyclohexan-1-amine (0.14 g, 0.80 mmol). Colorless oil (0.12 g, 92%). ¹H NMR (400 MHz, DMSO) δ 8.08-7.99 (m, 2H), 7.92-7.85 (m, 1H), 7.82 (d, J=8.7 Hz, 1H), 7.48 (dd, J=8.6, 2.0 Hz, 1H), 7.28 (d, J=7.6 Hz, 2H), 4.65 (s, 2H), 3.95 (s, 1H),, 1.78 (d, J=9.3 Hz, 2H), 1.69-1.51 (m, 5H), 1.24 (s, 1H). ¹⁹F NMR (376 MHz, DMSO) δ −71.34. LC-MS (ESI): m/z=430.1/.432.0, t_{R}=5.04 min.

2-((7-(4,4,5,5-tetramethyl-1,3,2-dioxaborolan-2-yl)naphthalen-2-yl)oxy)-N-cis-4-(trifluoromethyl)cyclohexyl) acetamide (I-22j). A solution of I-21j (0.12 g, 0.28 mmol), PinB-BPin (0.1 g, 0.34 mmol), KOAc (0.1 g, 0.84 mmol), and Pd(dppf)Cl₂ (0.01 g, 0.01 mmol) were added in dioxane (5 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.043 g, 33.5%). ¹H NMR (400 MHz, DMSO) δ 8.16 (s, 1H), 8.09 (d, J=7.0 Hz, 1H), 7.85 (dd, J=14.3, 8.6 Hz, 2H), 7.57 (dd, J=8.1, 1.2 Hz, 1H), 7.37 (d, J=2.6 Hz, 1H), 7.29 (dd, J=8.9, 2.6 Hz, 1H), 4.66 (s, 2H), 3.98-3.93 (m, 1H), 2.30 (dd, J=9.7, 4.7 Hz, 1H), 1.77 (d, J=11.4 Hz, 2H), 1.72-1.48 (m, 6H), 1.34 (s, 12H). ¹⁹F NMR (376 MHz, DMSO) δ -71.35. LC-MS (ESI): m/z=478.3, t_{R}=5.26 min.

Methyl 3-(benzo[d][1,3]dioxol-5-yl)-3-(7-(2-oxo-2-((cis-4-(trifluoromethyl)cyclohexyl)amino)ethoxy)naphthalen-2-yl)propanoate (I-23j). The reaction was conducted following the general procedure F, using I-22j (0.06 g, 0.13 mmol) and I-9m (0.026 g, 0.2 mmol), TEA (0.04 ml, 0.25 mmol), and [Rh(COD)Cl]₂ (10 mg, 0.01 mmol). Colorless oil (0.012 g, 17.1%). ¹H NMR (600 MHz, DMSO) δ 8.05 (d, J=7.1 Hz, 1H), 7.77 (d, J=8.7 Hz, 1H), 7.73 (d, J=8.5 Hz, 1H), 7.64 (d, J=1.8 Hz, 1H), 7.31 (dd, J=8.4, 1.8 Hz, 1H), 7.20-7.14 (m, 2H), 6.95 (d, J=1.6 Hz, 1H), 6.84-6.77 (m, 2H), 5.94 (dd, J=6.3, 1.0 Hz, 2H), 4.64 (s, 2H), 4.51 (t, J=8.0 Hz, 1H), 3.97-3.93 (m, 1H), 3.50 (s, 3H), 3.18 (qd, J=15.9, 8.0 Hz, 2H), 2.31 (d, J=9.9 Hz, 1H), 1.77 (d, J=11.3 Hz, 2H), 1.69-1.53 (m, 6H). ¹⁹F NMR (376 MHz, DMSO) δ −71.33. LC-MS (ESI): m/z=558.2, t_{R}=4.90 min.

3-(benzo[d][1,3]dioxol-5-yl)-3-(7-(2-oxo-2-((cis-4-(trifluoromethyl)cyclohexyl)amino)ethoxy)naphthalen-2-yl)propanoic acid (6j, UCAB #1202_P2). The reaction was conducted following the general procedure G, using I-23h (0.012 g, 0.022 mmol) and 2M aq. NaOH (60 μl). White solid (0.002 g, 17.1%). ¹H NMR (600 MHz, DMSO) δ 12.10 (s, 1H), 8.06 (d, J=7.0 Hz, 1H), 7.79-7.70 (m, 2H), 7.64 (d, J=1.7 Hz, 1H), 7.31 (dd, J=8.5, 1.8 Hz, 1H), 7.20-7.14 (m, 2H), 6.93 (d, J=1.5 Hz, 1H), 6.84-6.77 (m, 2H), 5.94 (dd, J=5.4, 1.0 Hz, 2H), 4.64 (s, 2H), 4.48 (t, J=8.0 Hz, 1H), 3.95 (dq, J=7.4, 3.8 Hz, 1H), 3.06 (qd, J=15.8, 8.0 Hz, 2H), 2.30 (q, J=8.4 Hz, 1H), 1.77 (d, J=12.7 Hz, 2H), 1.70-1.53 (m, 6H). ¹³C NMR (151 MHz, DMSO) δ 173.14, 167.49, 156.53, 147.74, 146.04, 142.92, 138.45, 134.57, 129.49 (2C), 128.12, 127.80, 124.81, 124.54, 121.00, 118.64, 108.51 (2C), 107.41, 101.24, 67.32, 46.87, 43.94, 40.41 (2C), 28.14 (2C), 20.05 (2C). ¹⁹F NMR (376 MHz, DMSO) δ -71.33. LC-MS (ESI): m/z=488.3, t_{R}=4.47 min.

2-((7-bromonaphthalen-2-yl)oxy)-1-(2-propylpiperidin-1-yl) ethan-1-one (I-21k). The reaction was conducted following the general procedure E, using I-2 (0.15 g, 0.53 mmol), HATU (0.41 g, 1.07 mmol), DIPEA (0.3 mL, 1.60 mmol) and Propylpiperidine hydrochloride (0.14 g, 0.80 mmol). Colorless oil (0.12 g, 92%). ¹H NMR (600 MHz, DMSO) (minor diastereomer in brackets) δ 8.04-7.98 (m, 1H), 7.87 (t, J=6.4 Hz, 1H), 7.81 (d, J=8.7 Hz, 1H), 7.46 (dd, J=8.7, 2.1 Hz, 1H), 7.28-7.21 (m, 2H), 4.98-4.87 (m, 2H), 4.86-4.80 (m, 1H), 4.57 (dt, J=10.6, 5.8 Hz, 1H), [4.29-4.24 (m, 1H)], 3.94 (d, J=9.5 Hz, 1H), 3.69-3.63 (m, 1H), 3.09 (t, J=13.3 Hz, 1H), [2.68-2.61 (m, 1H)], 1.79-1.41 (m, 5H), 1.35 (dq, J=15.3, 7.3 Hz, 1H), 1.30-1.21 (m, 1H), 1.16 (dh, J=13.9, 7.5 Hz, 1H), 0.90-0.81 (m, 3H). LC-MS (ESI): m/z=390.1/.392.1, t_{R}=5.30 min.

1-(2-propylpiperidin-1-yl)-2-((7-(4,4,5,5-tetramethyl-1,3,2-dioxaborolan-2-yl)naphthalen-2-yl)oxy) ethan-1-one (I-22k). A solution of I-21k (0.12 g, 0.3 mmol), PinB-BPin (0.1 g, 0.37 mmol), KOAc (0.1 g, 0.92 mmol), and Pd(dppf)Cl₂ (0.01 g, 0.01 mmol) were added in dioxane (5 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.1 g, 76.9%). ¹H NMR (600 MHz, DMSO) (minor diastereomer in brackets) δ 8.17 (d, J=7.9 Hz, 1H), 7.83 (dd, J=14.6, 8.4 Hz, 2H), 7.59-7.54 (m, 1H), 7.41-7.30 (m, 1H), 7.25 (dd, J=8.9, 2.6 Hz, 1H), 4.96-4.86 (m, 2H), 4.83 (d, J=14.5 Hz, 1H), 4.58 (s, 1H), [4.28 (d, J=13.3 Hz, 1H)], 3.96 (s, 1H), 3.68 (d, J=13.8 Hz, 1H), 3.11 (t, J=13.1 Hz, 1H), [2.70-2.63 (m, 1H)], 1.92-1.41 (m, 8H), 1.34 (s, 12H), 0.86 (q, J=7.4 Hz, 3H). LC-MS (ESI): m/z=438.3, t_{R}=5.53 min.

Methyl 3-(benzo[d][1,3]dioxol-5-yl)-3-(7-(2-oxo-2-(2-propylpiperidin-1-yl) ethoxy)naphthalen-2-yl)propanoate (I-23k). The reaction was conducted following the general procedure F, using I-22k (0.1 g, 0.22 mmol) and I-9m (0.032 g, 0.15 mmol), TEA (0.1 ml, 0.46 mmol), and [Rh(COD)Cl]₂ (10 mg, 0.01 mmol). Colorless oil (0.010 g, 12.5%). ¹H NMR (400 MHz, DMSO) (minor diastereomer in brackets) δ 7.74 (dd, J=11.7, 8.7 Hz, 2H), 7.64 (s, 1H), 7.31 (dd, J=8.5, 1.7 Hz, 1H), 7.20-7.09 (m, 2H), 6.96 (d, J=4.7 Hz, 1H), 6.86-6.77 (m, 2H), 5.94 (dd, J=3.8, 1.0 Hz, 2H), 4.91-4.78 (m, 3H), 4.60-4.47 (m, 2H), [4.27 (d, J=13.5 Hz, 1H)], 3.97 (s, 1H), 3.69 (d, J=13.4 Hz, 1H), 3.50 (s, 3H), 3.27-3.02 (m, 2H), [2.63 (d, J=13.9 Hz, 1H)], 1.82-1.22 (m, 8H), 0.83 (t, J=7.3 Hz, 3H). LC-MS (ESI): m/z=518.4, t_{R}=5.13 min.

3-(benzo[d][1,3]dioxol-5-yl)-3-(7-(2-oxo-2-(2-propylpiperidin-1-yl)ethoxy)naphthalen-2-yl)propanoic acid (6k, UCAB #1224). The reaction was conducted following the general procedure G, using I-23k (0.01 g, 0.02 mmol) and 2M aq. NaOH (200 μl). White solid (0.005 g, 51.5%). ¹H NMR (600 MHz, DMSO) (minor diastereomer in brackets) δ 12.10 (s, 1H), 7.74 (dd, J=17.2, 8.6 Hz, 2H), 7.64 (d, J=11.1 Hz, 1H), 7.30 (dd, J=8.5, 1.8 Hz, 1H), 7.19 (d, J=9.5 Hz, 1H), 7.12 (dd, J=8.9, 2.5 Hz, 1H), 6.95 (d, J=9.0 Hz, 1H), 6.81 (d, J=2.8 Hz, 2H), 5.94 (dd, J=4.8, 1.0 Hz, 2H), 4.93-4.79 (m, 3H), 4.58 (s, 1H), 4.48 (t, J=8.0 Hz, 1H), [4.28 (d, J=13.3 Hz, 1H)], 3.97 (s, 1H), 3.72-3.66 (m, 1H), 3.28-2.88 (m, 3H), [2.66 (d, J=13.1 Hz, 1H)], 1.85-0.89 (m, 8H), 0.85 (dt, J=14.9, 7.0 Hz, 3H). ¹³C NMR (151 MHz, DMSO) (minor diastereomer in brackets) δ 173.15, 166.00 (d, J=15.0 Hz), 156.60, 147.74, 146.03, 142.86, 138.48, 134.59, 129.33, 128.09, 127.73, 124.66 (2C), 121.03, 118.55, [108.55], 108.52, 107.64, [107.48], 101.23, 66.84, [52.22], [47.60], 46.87, 40.43, 36.55, [32.17], 31.33, [28.93], 28.55, 26.37, 19.67, 19.21 (2C), [14.49], 14.29. LC-MS (ESI): m/z=504.4, t_{R}=4.71 min.

2-((7-bromonaphthalen-2-yl)oxy)-N-phenylacetamide (I-211). The reaction was conducted following the general procedure E, using I-2 (0.1 g, 0.36 mmol), HATU (0.3 g, 0.71 mmol), DIPEA (0.2 mL, 1.07 mmol) and aniline (0.05 g, 0.53 mmol). Colorless oil (0.1 g, 79.4%). ¹H NMR (400 MHz, DMSO) δ 10.14 (s, 1H), 8.09 (d, J=2.0 Hz, 1H), 7.95-7.89 (m, 1H), 7.84 (d, J=8.7 Hz, 1H), 7.70-7.62 (m, 2H), 7.49 (dd, J=8.7, 2.0 Hz, 1H), 7.35 (qd, J=7.3, 2.1 Hz, 4H), 7.14-7.05 (m, 1H), 4.83 (s, 2H). LC-MS (ESI): m/z=356.1/.358.0, t_{R}=4.85 min.

N-phenyl-2-((7-(4,4,5,5-tetramethyl-1,3,2-dioxaborolan-2-yl)naphthalen-2-yl)oxy) acetamide (1-221). A solution of I-211 (0.15 g, 0.42 mmol), PinB-BPin (0.13 g, 0.51 mmol), KOAc (0.13 g, 1.26 mmol), and Pd(dppf)Cl₂ (0.01 g, 0.013 mmol) were added in dioxane (5 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.06 g, 35.5%). ¹H NMR (400 MHz, DMSO) δ 10.13 (s, 1H), 8.21 (s, 1H), 7.90 (d, J=9.0 Hz, 1H), 7.84 (d, J=8.2 Hz, 1H), 7.71-7.63 (m, 2H), 7.58 (dd, J=8.1, 1.1 Hz, 1H), 7.46 (d, J=2.6 Hz, 1H), 7.43-7.29 (m, 3H), 7.14-7.05 (m, 1H), 4.82 (s, 2H), 1.34 (s, 12H). LC-MS (ESI): m/z=404.2, t_{R}=5.39 min.

Methyl 3-(benzo[d][1,3]dioxol-5-yl)-3-(7-(2-oxo-2-(phenylamino)ethoxy) naphthalen-2-yl)propanoate (I-231). The reaction was conducted following the general procedure F, using I-221 (0.06 g, 0.15 mmol) and I-9m (0.03 g, 0.1 mmol), TEA (0.04 ml, 0.28 mmol), and [Rh(COD)Cl]₂ (5 mg, 0.01 mmol). Colorless oil (0.012 g, 16.9%). ¹H NMR (400 MHz, DMSO) δ 10.12 (s, 1H), 7.91-7.58 (m, 5H), 7.39-7.29 (m, 3H), 7.29-7.17 (m, 2H), 7.14-7.05 (m, 1H), 6.98 (d, J=1.7 Hz, 1H), 6.87-6.76 (m, 2H), 6.01-5.91 (m, 2H), 4.82 (s, 2H), 4.51 (t, J=8.0 Hz, 1H), 3.50 (s, 3H), 3.29-3.10 (m, 2H). LC-MS (ESI): m/z=584.2, t_{R}=5.37 min.

3-(benzo[d][1,3]dioxol-5-yl)-3-(7-(2-oxo-2-(phenylamino)ethoxy)naphthalen-2-yl)propanoic acid (61, UCAB #1247). The reaction was conducted following the general procedure G, using I-231 (0.012 g, 0.022 mmol) and 2M aq. NaOH (60 μl). White solid (0.002 g, 18.2%). ¹H NMR (400 MHz, DMSO) δ 12.10 (s, 1H), 10.12 (s, 1H), 7.80 (d, J=8.6 Hz, 1H), 7.77-7.64 (m, 4H), 7.38-7.21 (m, 5H), 7.09 (t, J=7.3 Hz, 1H), 6.96 (d, J=1.6 Hz, 1H), 6.88-6.76 (m, 2H), 5.94 (d, J=3.0 Hz, 2H), 4.81 (s, 2H), 4.48 (t, J=7.9 Hz, 1H), 3.07 (qd, J=15.8, 8.0 Hz, 2H). ¹³C NMR (151 MHz, DMSO) δ 173.18, 166.90, 156.41, 147.72, 146.03, 142.98, 138.87, 138.50, 134.58, 129.49, 129.21 (2C), 128.10, 127.90, 124.89, 124.75, 124.19, 121.06, 120.23 (2C), 118.70, 108.54 (2C), 107.62, 101.22, 67.70, 46.89, 40.42. LC-MS (ESI): m/z=470.2, t_{R}=4.48 min.

Ethyl 2-((7-bromonaphthalen-2-yl)oxy) propanoate (I-24). A mixture of 7-bromonaphthalen-2-ol (5.0 g, 22.4 mmol), K₂CO₃ (3.16 g, 22.9 mmol) and ethyl 2-bromopropanoate (4.24 g, 23.5 mmol) were added in MeCN (50 mL). The mixture was allowed to stir at 60° C. for 18 h. Upon reaction completion, the mixture was cooled to room temperature, extracted with ethyl acetate three times, washed with brine, and dried over Na₂SO₄, filtered and concentrated under reduced pressure. The crude was purified by silica gel column chromatography to afford the desired product. White solid (7.0 g, 95.7%). ¹H NMR (400 MHz, DMSO) δ 8.04 (d, J=2.0 Hz, 1H), 7.87 (d, J=8.8 Hz, 1H), 7.80 (d, J=8.7 Hz, 1H), 7.46 (dd, J=8.7, 2.0 Hz, 1H), 7.26-7.18 (m, 2H), 5.09 (q, J=6.7 Hz, 1H), 4.16 (q, J=7.1 Hz, 2H), 1.57 (d, J=6.7 Hz, 3H), 1.20-1.15 (m, 3H). LC-MS (ESI): m/z=323.0/325.0, t_{R}=5.25 min.

2-((7-Bromonaphthalen-2-yl)oxy)propanoic acid (1-25). The reaction was conducted following the general procedure B, using I-24 (7.0 g, 22.4 mmol) and 1 M aq. NaOH (30 ml). White solid (6.3 g, 98.6%). LC-MS (ESI): m/z=295.0/297.0, t_{R}=4.48 min.

2-((7-Bromonaphthalen-2-yl)oxy)-N-cycloheptylpropanamide (I-26). The reaction was conducted following the general procedure E, using I-25 (0.30 g, 1.02 mmol), HATU (0.78 g, 2.03 mmol), DIPEA (0.6 mL, 3.1 mmol) and cycloheptanamine (0.16 g, 1.41 mmol). White solid (0.08 g, 20.4%). ¹H NMR (600 MHz, DMSO) δ 8.04 (d, J=8.1 Hz, 1H), 7.97 (d, J=2.0 Hz, 1H), 7.87 (d, J=8.9 Hz, 1H), 7.81 (d, J=8.7 Hz, 1H), 7.47 (dd, J=8.6, 2.0 Hz, 1H), 7.28-7.20 (m, 2H), 4.83 (q, J=6.6 Hz, 1H), 3.74 (tq, J=12.7, 4.5 Hz, 1H), 1.89-1.82 (m, 1H), 1.76-1.26 (m, 14H). LC-MS (ESI): m/z=390.1/392.1, t_{R}=5.39 min.

N-Cycloheptyl-2-((7-(4,4,5,5-tetramethyl-1,3,2-dioxaborolan-2-yl)naphthalen-2-yl)oxy) propanamide (1-27). A solution of 1-26 (0.20 g, 0.54 mmol), PinB-BPin (0.17 g, 0.64 mmol), KOAc (0.16 g, 1.61 mmol), and Pd(dppf)Cl₂ (0.02 g, 0.016 mmol) were added in dioxane (3 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.27 g, 68.9%). ¹H NMR (600 MHz, DMSO) δ 8.13 (d, J=1.2 Hz, 1H), 8.02 (d, J=8.1 Hz, 1H), 7.87-7.79 (m, 2H), 7.56 (dd, J=8.1, 1.1 Hz, 1H), 7.32 (d, J=2.5 Hz, 1H), 7.26 (dd, J=8.9, 2.5 Hz, 1H), 4.82 (q, J=6.5 Hz, 1H), 3.76 (dh, J=13.4, 4.5 Hz, 1H), 1.75-1.27 (m, 27H). LC-MS (ESI): m/z=438.3, t_{R}=5.62 min.

Methyl 3-(benzo[d][1,3]dioxol-5-yl)-3-(7-((1-(cycloheptylamino)-1-oxopropan-2-yl)oxy)naphthalen-2-yl)propanoate (I-28). The reaction was conducted following the general procedure F, using I-27 (0.1 g, 0.24 mmol) and I-9m (0.04 g, 0.16 mmol), TEA (0.1 ml, 0.48 mmol), and [Rh(COD)Cl]₂ (10 mg, 0.02 mmol). White solid (0.04 g, 35.2%). ¹H NMR (600 MHz, DMSO) δ 8.01 (dd, J=8.2, 3.4 Hz, 1H), 7.77-7.69 (m, 2H), 7.59 (t, J=2.4 Hz, 1H), 7.31 (dt, J=8.5, 1.6 Hz, 1H), 7.15-7.09 (m, 2H), 6.95 (t, J=1.7 Hz, 1H), 6.85-6.78 (m, 2H), 5.94 (dt, J=6.5, 1.0 Hz, 2H), 4.81-4.75 (m, 1H), 4.51 (t, J=7.9 Hz, 1H), 3.79-3.71 (m, 1H), 3.50 (s, 3H), 3.25-3.10 (m, 2H), 1.75-1.64 (m, 1H), 1.60-1.21 (m, 14H). LC-MS (ESI): m/z=518.2, t_{R}=5.21 min.

3-(Benzo[d][1,3]dioxol-5-yl)-3-(7-((1-(cycloheptylamino)-1-oxopropan-2-yl)oxy)naphthalen-2-yl)propanoic acid (7a, UCAB #1002). The reaction was conducted following the general procedure G, using I-25 (0.04 g, 0.09 mmol) and 2 M aq. NaOH (300 μl). White solid (0.015 g, 35.7%). ¹H NMR (600 MHz, DMSO) δ 12.11 (s, 1H), 8.01 (dd, J=8.2, 4.7 Hz, 1H), 7.77-7.69 (m, 2H), 7.58 (t, J=2.2 Hz, 1H), 7.30 (dt, J=8.5, 1.4 Hz, 1H), 7.15-7.10 (m, 2H), 6.93 (t, J=1.8 Hz, 1H), 6.84-6.77 (m, 2H), 5.96-5.91 (m, 2H), 4.78 (q, J=6.5 Hz, 1H), 4.48 (td, J=8.0, 3.2 Hz, 1H), 3.75 (tqd, J=9.6, 4.5, 2.4 Hz, 1H), 3.12-2.98 (m, 2H), 1.76-1.57 (m, 2H), 1.49-1.40 (m, 13H). ¹³C NMR (151 MHz, DMSO) δ 173.14, 170.11, 155.90, 147.74, 146.04, 142.9, 142.8, 138.42, 134.52, 129.37, 128.08, 127.74, 124.84, 124.51, 121.03, 119.01, 108.62, 108.49, 108.23, 101.24, 74.00, 49.93, 46.82, 40.48, 34.58 (2C), 28.12, 24.17 (2C), 19.18 (2C). LC-MS (ESI): m/z=504.2, t_{R}=4.78 min.

Methyl 3-(benzo[d][1,3]dioxol-5-yl)-3-(7-(2-(cycloheptyl(methyl)amino)-2-oxoethoxy)naphthalen-2-yl)propanoate (I-29). To a vial charged with a magnetic stirring bar were added 1-7m (0.100 g, 0.20 mmol) and DMF (3 mL). At 0° C. was then added NaH (0.0053 g, 0.22 mmol). After 1 h stirring at RT, the Mel was added (0.014 mL, 0.22 mmol) cooling at 0° C. and the mixture was stirred at RT for other 60 min. Upon reaction completion (as determined by LC-MS), the solvent was concentrated and the crude was purified by silica gel column chromatography to afford the desired product. White solid (0.06 g, 60%). ¹H NMR (600 MHz, DMSO) (minor diastereomer in brackets) δ 7.78-7.64 (m, 3H), 7.31 (td, J=8.7, 1.8 Hz, 1H), 7.22-7.09 (m, 2H), 6.98 (dd, J=7.8, 1.7 Hz, 1H), 6.86-6.78 (m, 2H), 5.96-5.92 (m, 2H), 4.92 (s, 2H), [4.86 (s, 2H)], 4.51 (td, J=8.0, 3.2 Hz, 1H), 4.37 (tt, J=9.8, 4.8 Hz, 1H), [3.83 (tt, J=9.7, 4.2 Hz, 1H)], 3.50 (d, J=1.0 Hz, 3H), 3.27-3.12 (m, 2H), 2.91 (s, 3H), [2.73 (s, 3H)], 1.84-1.22 (m, 12H). LC-MS (ESI): m/z=518.2, t_{R}=5.08 min.

3-(benzo[d][1,3]dioxol-5-yl)-3-(7-(2-(cycloheptyl(methyl)amino)-2-oxoethoxy)naphthalen-2-yl)propanoic acid (7b, UCAB #1132). The reaction was conducted following the general procedure G, using I-29 (0.06 g, 0.12 mmol) and 2 M aq. NaOH (580 μl). White solid (0.036 g, 60%). ¹H NMR (600 MHz, DMSO) (minor diastereomer in brackets) δ 12.08 (s, 1H), 7.77-7.70 (m, 2H), 7.70-7.66 (m, 1H), 7.30 (td, J=8.1, 1.8 Hz, 1H), 7.18 (d, J=2.5 Hz, 1H), 7.12 (ddd, J=8.5, 5.8, 2.5 Hz, 1H), 6.96 (dd, J=7.4, 1.6 Hz, 1H), 6.85-6.78 (m, 2H), 5.94 (dd, J=5.2, 1.0 Hz, 2H), [4.91 (s, 2H)], 4.85 (s, 2H), 4.48 (td, J=8.0, 3.2 Hz, 1H), 4.37 (tt, J=9.8, 4.9 Hz, 1H), [3.83 (ddt, J=14.2, 9.6, 4.2 Hz, 1H)],3.14-3.00 (m, 2H), 2.91 (s, 3H), [2.73 (s, 3H)], 1.85-1.19 (m, 12H). ¹³C NMR (151 MHz, DMSO) δ 173.17, 166.49, 158.72, 156.71, 147.73, 146.03, 142.82, 138.53, 134.61, 129.29, 128.06, 127.71, 124.65, 121.04, 118.60, 108.52, 107.69, 101.23, [67.17], 66.53, 57.71, 54.58, 46.90, 40.55, [33.03 (2C)], 31.90 (2C), 28.77, 27.64 (2C), [27.55 (2C)], 25.18 (2C), [25.08 (2C)]. LC-MS (ESI): m/z=504.2, t_{R}=4.70 min.

7-(4,4,5,5-tetramethyl-1,3,2-dioxaborolan-2-yl)naphthalen-2-ol (1-30). A solution of 7-bromonaphthalen-2-ol (0.3 g, 1.4 mmol), PinB-BPin (0.41 g, 1.6 mmol), KOAc (0.4 g, 4.1 mmol), and Pd(dppf)Cl₂ (0.03 g, 0.04 mmol) were added in dioxane (5 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.3 g, 83.3%). ¹H NMR (600 MHz, DMSO) δ 9.76 (s, 1H), 8.07 (d, J=1.2 Hz, 1H), 7.76 (t, J=8.3 Hz, 2H), 7.48 (dd, J=8.1, 1.2 Hz, 1H), 7.20 (d, J=2.4 Hz, 1H), 7.14 (dd, J=8.8, 2.4 Hz, 1H), 1.33 (s, 12H). ¹³C NMR (151 MHz, DMSO) δ 155.74, 134.49, 134.30, 129.63, 129.50, 127.28, 127.19, 120.33, 109.70 (2C), 84.05 (2C), 25.13 (4C). LC-MS (ESI): m/z=271.1, t_{R}=4.65 min.

Methyl 3-(benzo[d][1,3]dioxol-5-yl)-3-(7-hydroxynaphthalen-2-yl)propanoat (I-31). The reaction was conducted following the general procedure F, using I-30 (0.15 g, 0.6 mmol) and I-9m (0.08 g, 0.4 mmol), TEA (0.2 ml, 1.21 mmol), and [Rh(COD)Cl]₂ (20 mg, 0.04 mmol). White solid (0.03 g, 19.6%). ¹H NMR (400 MHz, DMSO) δ 9.66 (s, 1H), 7.69-7.58 (m, 3H), 7.19 (dd, J=8.4, 1.8 Hz, 1H), 7.08-6.93 (m, 3H), 6.86-6.75 (m, 2H), 5.97-5.90 (m, 2H), 4.47 (t, J=8.0 Hz, 1H), 3.50 (s, 3H), 3.25-3.09 (m, 2H). ¹³C NMR (101 MHz, DMSO) δ 171.92, 155.67, 147.45, 145.78, 141.89, 138.07, 134.86, 129.11, 127.86, 126.60, 123.60, 123.24, 120.71, 118.33, 108.81, 108.21, 100.95, 73.70, 51.49, 46.56, 25.14. LC-MS (ESI): m/z=351.1, t_{R}=4.36 min.

Methyl 3-(benzo[d][1,3]dioxol-5-yl)-3-(7-((tert-butyldimethylsilyl)oxy)naphthalen-2-yl)propanoate (I-32). A solution of 1-31 (0.15 g, 0.43 mmol), tert-butyldimethylsilyl chloride (0.1 g, 0.64 mmol), imidazole (0.1 g, 1.28 mmol) and N,N-dimethyl-4-aminopyridine (0.03, 0.02 mmol) were added in DMF (6 ml). This mixture was stirred at RT for 30 min. Upon reaction completion (as determined by LC-MS), water (10 mL pr. mmol) was added and the mixture extracted with EtOAc (3×10 mL pr. mmol). The combined organic layers were washed with sat. brine (10 mL pr. mmol), dried over Na₂SO₄, filtered and evaporated to dryness in vacuo. The crude was purified by silica gel column chromatography to afford the desired product. Colorless oil (0.11 g, 75.9%). ¹H NMR (600 MHz, DMSO) δ 7.78-7.70 (m, 3H), 7.32 (dd, J=8.5, 1.8 Hz, 1H), 7.23 (d, J=2.4 Hz, 1H), 7.05 (dd, J=8.8, 2.4 Hz, 1H), 6.98 (d, J=1.7 Hz, 1H), 6.87-6.79 (m, 2H), 5.95 (dd, J=6.7, 1.0 Hz, 2H), 4.51 (t, J=8.0 Hz, 1H), 3.51 (s, 3H), 3.20 (qd, J=15.9, 8.0 Hz, 2H), 1.02-0.93 (m, 9H), 0.26-0.23 (m, 6H). ¹³C NMR (151 MHz, DMSO) δ 171.95, 153.37, 147.54, 145.88, 142.22, 138.02, 134.63, 129.36, 127.88, 127.83, 124.57, 124.30, 121.64, 120.80, 114.68, 108.33, 108.25, 101.04, 51.57, 46.61, 40.21, 25.85 (3C), 18.24, −4.20 (2C). LC-MS (ESI): m/z=465.2, t_{R}=5.51 min.

Methyl 3-(benzo[d][1,3]dioxol-5-yl)-3-(7-((tert-butyldimethylsilyl)oxy)naphthalen-2-yl)-2-methylpropanoate (I-33a). A solution of 1-32 (0.11 g, 0.24 mmol) in THF (6 ml) was added lithium diisopropylamide (0.2 ml. 0.37 mmol) drop-wise with stirring at −78° C. under a nitrogen atmosphere. After 30 min, iodomethane (0.04 g, 0.28 mmol) was added at-78° C., and the reaction mixture was warmed slowly to room temperature and stirred for 1h. Upon reaction completion (as determined by LC-MS), The resulting solution was extracted with ethyl acetate (3×30 ml) water (10 mL pr. mmol) was added and the mixture extracted with EtOAc (3×10 mL pr. mmol). The combined organic layers were combined, dried over Na₂SO₄, filtered and evaporated to dryness in vacuo. The crude was purified by silica gel column chromatography to afford the desired product. Colorless oil (0.032 g, 28.3%). ¹H NMR (600 MHz, DMSO) δ 7.76-7.65 (m, 3H), 7.36 (dd, J=8.5, 1.8 Hz, 1H), 7.19 (d, J=2.5 Hz, 1H), 7.09 (d, J=1.7 Hz, 1H), 7.02 (dd, J=8.8, 2.4 Hz, 1H), 6.92 (dd, J=8.1, 1.8 Hz, 1H), 6.80 (d, J=8.0 Hz, 1H), 5.93 (dd, J=17.6, 1.0 Hz, 2H), 4.11 (d, J=11.7 Hz, 1H), 3.58 (dq, J=11.5, 6.8 Hz, 1H), 3.36 (s, 3H), 1.06 (d, J=6.9 Hz, 3H), 0.98 (s, 9H), 0.23 (s, 6H). ¹³C NMR (151 MHz, DMSO) δ 177.33, 153.51, 147.41, 146.17, 139.91, 135.31, 134.60, 129.42, 127.92, 127.49, 126.97, 126.67, 123.53, 122.00, 115.08, 110.46, 108.19, 101.32, 59.01, 52.04, 46.68, 26.07 (3C), 25.05, 24.64, 18.46, −3.98 (2C). LC-MS (ESI): m/z=479.2, t_{R}=5.65 min.

Methyl 3-(benzo[d][1,3]dioxol-5-yl)-3-(7-hydroxynaphthalen-2-yl)-2-methylpropanoate (I-34a). A mixture of I-33a (0.032 g, 0.07 mmol) and TBAF (0.02 g, 0.07 mmol) in dry THF (3 ml) was stirred at RT overnight. Upon reaction completion (as determined by LC-MS), The resulting solution was concentrated in vacuo. The crude was purified by silica gel column chromatography to afford the desired product. Colorless oil (0.025 g, 100%). ¹H NMR (600 MHz, DMSO) δ 9.64 (s, 1H), 7.65-7.58 (m, 3H), 7.24 (dd, J=8.5, 1.8 Hz, 1H), 7.08 (d, J=1.7 Hz, 1H), 7.03 (d, J=2.4 Hz, 1H), 6.99 (dd, J=8.7, 2.4 Hz, 1H), 6.91 (dd, J=8.1, 1.8 Hz, 1H), 6.79 (d, J=8.0 Hz, 1H), 5.93 (dd, J=17.4, 1.1 Hz, 2H), 4.08 (d, J=11.7 Hz, 1H), 3.56 (dq, J=11.6, 6.8 Hz, 1H), 3.36 (s, 3H), 1.05 (d, J=6.9 Hz, 3H). ¹³C NMR (151 MHz, DMSO) δ 175.96, 155.89, 147.89, 146.12, 142.03, 136.91, 135.09, 129.35, 127.95, 126.84, 123.85, 123.63, 121.86, 118.54, 109.05, 108.69, 108.58, 101.24, 54.70, 51.70, 43.48, 17.44. LC-MS (ESI): m/z=365.1, t_{R}=4.49 min.

2-bromo-N-cycloheptylacetamide (1-36). A solution of cycloheptanamine (0.91 ml, 7.13 mmol) and DIPEA (1.3 ml, 7.13 mmol) in dry DCM (5 ml) was added 2-bromoacetyll chloride (0.5 ml, 5.94 mmol) drop-wise with stirring at −10° C. under a nitrogen atmosphere. After 20 min, the reaction mixture was warmed slowly to room temperature and stirred for 30 min. Upon reaction completion (as determined by LC-MS), The resulting solution was extracted with ethyl acetate (3×30 ml) water (10 mL pr. mmol) was added and the mixture extracted with EtOAc (3×10 mL pr. mmol). The combined organic layers were combined, dried over Na₂SO₄, filtered and evaporated to dryness in vacuo. The crude was purified by silica gel column chromatography to afford the desired product. White solid (0.7 g, 94.6%). ¹H NMR (400 MHz, DMSO) δ 8.16 (d, J=7.8 Hz, 1H), 3.79 (s, 2H), 3.69 (tt, J=8.4, 4.1 Hz, 1H), 1.76 (ddd, J=10.6, 7.6, 4.8 Hz, 2H), 1.63-1.32 (m, 10H). ¹³C NMR (101 MHz, DMSO) δ 165.11, 50.57, 34.47 (2C), 30.31, 28.20 (2C), 24.09 (2C). LC-MS (ESI): m/z=234.2/236.2, t_{R}=3.96 min.

Methyl 3-(benzo[d][1,3]dioxol-5-yl)-3-(7-(2-(cycloheptylamino)-2-oxoethoxy)naphthalen-2-yl)-2-methylpropanoate (I-35a) A mixture of I-34a (0.025 g, 0.07 mmol), K₂CO₃ (0.01 g, 0.07 mmol) and I-36 (0.017 g, 0.072 mmol) were added in MeCN (5 mL). The mixture was allowed to stir at 60° C. for 18 h. Upon reaction completion, the mixture was cooled to room temperature, extracted with ethyl acetate three times, washed with brine, and dried over Na₂SO₄, filtered and concentrated under reduced pressure. The crude was purified by silica gel column chromatography to afford the desired product. White solid (0.011 g, 31.4%). ¹H NMR (600 MHz, DMSO) δ 7.96 (d, J=8.1 Hz, 1H), 7.76-7.72 (m, 1H), 7.69 (d, J=8.6 Hz, 2H), 7.36 (dd, J=8.5, 1.8 Hz, 1H), 7.17 (d, J=8.1 Hz, 2H), 7.08 (d, J=1.6 Hz, 1H), 6.92 (dd, J=8.1, 1.7 Hz, 1H), 6.80 (dd, J=8.0, 1.2 Hz, 1H), 5.93 (dt, J=17.3, 1.2 Hz, 2H), 4.55 (s, 2H), 4.13 (d, J=11.7 Hz, 1H), 3.83 (qt, J=9.0, 4.5 Hz, 1H), 3.57 (dq, J=13.5, 6.8 Hz, 1H), 3.36 (d, J=1.2 Hz, 3H), 1.76 (ddt, J=14.1, 7.3, 3.7 Hz, 2H), 1.65-1.34 (m, 8H), 1.31-1.22 (m, 2H), 1.06 (d, J=6.8 Hz, 3H). ¹³C NMR (151 MHz, DMSO) δ 175.82, 166.48, 156.27, 147.86, 146.09, 142.39, 136.68, 134.46, 129.30, 127.90, 127.74, 124.68, 124.57, 121.79, 118.61, 108.59, 108.55, 107.53, 101.20, 67.48, 54.57, 51.64, 50.00, 43.48, 34.61 (2C), 28.06 (2C), 24.16 (2C), 17.37. LC-MS (ESI): m/z=518.3, t_{R}=5.08 min.

3-(benzo[d][1,3]dioxol-5-yl)-3-(7-(2-(cycloheptylamino)-2-oxoethoxy)naphthalen-2-yl)-2-methylpropanoic acid (7c, UCAB #1227). The reaction was conducted following the general procedure G, using I-35a (0.011 g, 0.022 mmol) and 2 M aq. NaOH (220 μl). White solid (0.003 g, 30%). ¹H NMR (600 MHz, DMSO) (minor diastereomer in brackets) δ 11.99 (s, 1H), 7.96 (dd, J=8.1, 3.4 Hz, 1H), 7.79-7.65 (m, 3H), 7.39 (ddd, J=10.3, 8.5, 1.7 Hz, 1H), 7.22-7.15 (m, 2H), 7.05 (t, J=2.2 Hz, 1H), 6.89 (ddd, J=8.4, 6.6, 1.8 Hz, 1H), 6.79 (dd, J=15.4, 8.0 Hz, 1H), 5.96-5.90 (m, 1H), 4.57-4.52 (m, 2H), 4.09 (dd, J=11.7, 3.6 Hz, 1H), 3.84 (ddq, J=13.5, 9.0, 4.4 Hz, 2H), 3.43 (dq, J=11.7, 6.9 Hz, 1H), 1.77 (dq, J=12.5, 4.2 Hz, 2H), 1.68-1.35 (m, 12H), 1.06 (d, J=6.9 Hz, 3H), [1.00 (d, J=7.0 Hz, 3H]. ¹³C NMR (151 MHz, DMSO) δ 177.0, [176.97], 166.54, 156.39, [156.33], 147.90, [147.54], 146.08, [145.95], 142.87, [141.58], [138.29], 137.15, [134.68], 134.55, 129.42, [129.34], 128.36, 127.88, [127.82], [125.79], 125.06, 124.71, [124.60], 121.80, [121.15], [118.71], 118.63, 108.65, 108.66, [108.56], [108.42], 107.57, 101.24, [101.17], 67.54, [54.73], 54.65, 50.08, 43.65, 34.70 (2C), [34.68 (2C)], 28.15 (2C), 24.23 (2C), [17.56], 17.52. LC-MS (ESI): m/z=504.3, t_{R}=4.61 min.

Methyl 3-(benzo[d][1,3]dioxol-5-yl)-3-(7-((tert-butyldimethylsilyl)oxy)naphthalen-2-yl)-2,2-dimethylpropanoate (I-33b). A solution of 1-32 (0.05 g, 0.11 mmol) in THF (6 ml) was added lithium diisopropylamide (0.2 ml. 0.37 mmol) drop-wise with stirring at −78° C. under a nitrogen atmosphere. After 30 min, iodomethane (0.04 g, 0.28 mmol) was added at −78° C., and the reaction mixture was warmed slowly to room temperature and stirred for 1h. Upon reaction completion (as determined by LC-MS), The resulting solution was extracted with ethyl acetate (3×30 ml) water (10 mL pr. mmol) was added and the mixture extracted with EtOAc (3×10 mL pr. mmol). The combined organic layers were combined, dried over Na₂SO₄, filtered and evaporated to dryness in vacuo. The crude was purified by silica gel column chromatography to afford the desired product. Colorless oil (0.01 g, 18.9%). ¹H NMR (600 MHz, DMSO) δ 7.78-7.72 (m, 2H), 7.70 (d, J=8.5 Hz, 1H), 7.31-7.25 (m, 2H), 7.04 (dd, J=8.7, 2.4 Hz, 1H), 6.97 (d, J=1.7 Hz, 1H), 6.86-6.79 (m, 2H), 5.95 (dd, J=10.2, 1.0 Hz, 2H), 4.40 (s, 1H), 3.45 (s, 3H), 1.24 (d, J=1.8 Hz, 6H), 0.98 (s, 9H), 0.23 (s, 6H). ¹³C NMR (151 MHz, DMSO) δ 177.33, 153.51, 147.41, 146.17, 139.91, 135.31, 134.60, 129.42, 127.92, 127.49, 126.97, 126.67, 123.53, 122.00, 115.08, 110.46, 108.19, 101.32, 59.01, 52.04, 46.68, 26.07 (3C), 25.05, 24.64, 18.46, −3.98 (2C). LC-MS (ESI): m/z=493.2, t_{R}=5.87 min.

Methyl 3-(benzo[d][1,3]dioxol-5-yl)-3-(7-hydroxynaphthalen-2-yl)-2,2-dimethylpropanoate (I-34b). A mixture of I-33b (0.01 g, 0.02 mmol) and TBAF (0.05 g, 0.02 mmol) in dry THF (3 ml) was stirred at RT overnight. Upon reaction completion (as determined by LC-MS), The resulting solution was concentrated in vacuo. The crude was purified by silica gel column chromatography to afford the desired product. Colorless oil (0.008 g, 100%). ¹H NMR (600 MHz, DMSO) δ 9.66 (s, 1H), 7.67 (d, J=8.8 Hz, 1H), 7.65-7.60 (m, 2H), 7.18 (dd, J=8.5, 1.8 Hz, 1H), 7.07 (d, J=2.4 Hz, 1H), 7.02 (dd, J=8.8, 2.4 Hz, 1H), 6.94 (d, J=1.6 Hz, 1H), 6.86-6.79 (m, 2H), 5.96 (dd, J=9.1, 1.0 Hz, 2H), 4.39 (s, 1H), 3.45 (s, 3H), 1.24 (d, J=2.0 Hz, 6H). ¹³C NMR (151 MHz, DMSO) δ 177.40, 155.90, 147.37, 146.13, 139.72, 135.44, 134.85, 129.27, 127.54, 126.76, 126.35, 125.35, 123.47, 118.79, 110.49, 109.19, 108.16, 101.30, 58.93, 52.03, 46.60, 25.12, 24.65. LC-MS (ESI): m/z=379.2, t_{R}=4.54 min.

Methyl 3-(benzo[d][1,3]dioxol-5-yl)-3-(7-(2-(cycloheptylamino)-2-oxoethoxy)naphthalen-2-yl)-2,2-dimethylpropanoate (I-35b) A mixture of 1-34b (0.008 g, 0.02 mmol), K₂CO₃ (0.003 g, 0.02 mmol) and I-36 (0.005 g, 0.02 mmol) were added in MeCN (5 mL). The mixture was allowed to stir at 60° C. for 18 h. Upon reaction completion, the mixture was cooled to room temperature, extracted with ethyl acetate three times, washed with brine, and dried over Na₂SO₄, filtered and concentrated under reduced pressure. The crude was purified by silica gel column chromatography to afford the desired product. White solid (0.006 g, 69.8%). ¹H NMR (600 MHz, DMSO) δ 7.97 (d, J=8.1 Hz, 1H), 7.76 (d, J=8.9 Hz, 1H), 7.71 (dd, J=5.3, 3.3 Hz, 2H), 7.26 (dd, J=8.6, 1.8 Hz, 1H), 7.22 (d, J=2.6 Hz, 1H), 7.19 (dd, J=8.8, 2.5 Hz, 1H), 6.93 (d, J=1.5 Hz, 1H), 6.85-6.79 (m, 2H), 5.95 (dd, J=9.7, 1.0 Hz, 2H), 4.55 (s, 2H), 4.42 (s, 1H), 3.82 (ddp, J=12.6, 9.0, 4.5 Hz, 1H), 3.45 (s, 3H), 1.80-1.70 (m, 2H), 1.66-1.34 (m, 10H), 1.24 (d, J=7.6 Hz, 6H). ¹³C NMR (151 MHz, DMSO) & 176.85, 166.05, 155.84, 146.91, 145.68, 139.68, 134.83, 133.80, 128.76, 127.22, 127.02, 126.40, 126.20, 123.01, 118.44, 109.96, 107.68, 107.29, 100.84, 67.04, 58.29, 51.55, 49.55, 46.07, 34.19 (2C), 27.64 (2C), 24.71, 24.02, 23.72 (2C). LC-MS (ESI): m/z=532.3, t_{R}=5.24 min.

3-(benzo[d][1,3]dioxol-5-yl)-3-(7-(2-(cycloheptylamino)-2-oxoethoxy)naphthalen-2-yl)-2,2-dimethylpropanoic acid (7d, UCAB #1228). The reaction was conducted following the general procedure G, using I-35b (0.006 g, 0.011 mmol) and 2 M aq. NaOH (110 μl). White solid (0.002 g, 35.7%). ¹H NMR (600 MHz, DMSO) δ 12.06 (s, 1H), 7.97 (d, J=8.2 Hz, 1H), 7.79-7.68 (m, 3H), 7.31-7.18 (m, 3H), 6.92 (d, 1H), 6.82 (q, J=8.2 Hz, 2H), 5.95 (d, J=8.0 Hz, 2H), 4.55 (s, 2H), 4.46 (s, 1H), 3.83 (m, 1H), 1.80-1.74 (m, 2H), 1.69-1.33 (m, 10H), 1.22 (d, J=5.9 Hz, 6H). ¹³C NMR (151 MHz, DMSO) δ 178.28, 166.07, 155.82, 146.85, 145.57, 140.19, 135.31, 133.80, 128.76, 127.20, 126.95, 126.48, 126.45, 123.09, 118.39, 110.10, 107.65, 107.27, 100.81, 67.05, 57.79, 49.56, 45.48, 34.20 (2C), 27.67 (2C), 24.79, 24.18, 23.73 (2C). LC-MS (ESI): m/z=518.3, t_{R}=4.80 min.

Ethyl (E)-3-(6-methylbenzo[d][1,3]dioxol-5-yl) acrylate (1-37). The reaction was conducted following the General procedure C, using 5-bromo-6-methylbenzo[d][1,3]dioxole (0.3 ml, 2.33 mmol), ethyl acrylate (0.4 ml, 3.49 mmol), Pd(OAc)₂ (0.027 g, 0.12 mmol), tri(o-tolyl)phosphine (0.08 g, 0.24 mmol), and Et₃N (2.5 ml, 17.83 mmol). White solid (0.36 g, 64%). ¹H NMR (600 MHz, DMSO) δ 7.77 (d, J=15.7 Hz, 1H), 7.36 (s, 1H), 6.85 (s, 1H), 6.44 (d, J=15.7 Hz, 1H), 6.02 (s, 2H), 4.17 (q, J=7.1 Hz, 2H), 2.32 (s, 3H), 1.25 (t, J=7.1 Hz, 3H). ¹³C NMR (151 MHz, DMSO) δ 166.78, 149.35, 146.46, 141.24, 133.16, 126.10, 116.87, 110.75, 106.01, 101.59, 60.12, 19.33, 14.49. LC-MS (ESI): m/z=235.1, t_{R}=4.62 min.

Ethyl 3-(7-(2-(cyclohex-2-en-1-ylamino)-2-oxoethoxy)naphthalen-2-yl)-3-(6-methylbenzo[d][1,3]dioxol-5-yl)propanoate (8b UCAB #1193). The reaction was conducted following the general procedure F, using I-16 (0.15 g, 0.37 mmol) and I-37 (0.06 g, 0.25 mmol), TEA (0.11 ml, 0.74 mmol), and [Rh(COD)Cl]₂ (13 mg, 0.03 mmol). White solid (0.058 g, 45%). ¹H NMR (600 MHz, DMSO) δ 8.10 (d, J=8.2 Hz, 1H), 7.79-7.70 (m, 2H), 7.58 (s, 1H), 7.27 (dd, J=8.5, 1.8 Hz, 1H), 7.21-7.17 (m, 2H), 6.99 (d, J=1.2 Hz, 1H), 6.72 (s, 1H), 5.93 (dd, J=24.9, 1.0 Hz, 2H), 5.81 (ddq, J=10.0, 3.7, 1.8 Hz, 1H), 5.53 (dq, J=9.9, 2.8 Hz, 1H), 4.69 (t, J=7.9 Hz, 1H), 4.58 (s, 2H), 4.37 (ddp, J=8.2, 5.5, 2.7 Hz, 1H), 3.96 (q, J=7.1 Hz, 2H), 3.16-3.05 (m, 2H), 2.25 (s, 3H), 2.01-1.92 (m, 2H), 1.73 (ddt, J=38.0, 14.5, 5.0 Hz, 2H), 1.53 (tt, J=15.1, 4.5 Hz, 2H), 1.04 (t, J=7.1 Hz, 3H). ¹³C NMR (151 MHz, DMSO) δ 171.70, 167.22,156.40, 146.05, 145.71, 141.97, 134.97, 134.49, 130.12, 129.36, 129.13, 128.80, 128.12, 127.79, 127.07, 125.42, 124.86, 118.78, 110.80, 107.59 (2C), 101.09, 67.37, 60.27, 44.53, 42.74, 29.22, 24.80, 20.23, 19.77, 14.43. LC-MS (ESI): m/z=516.2, t_{R}=5.13 min

N-cycloheptyl-2-((7-(1-(6-methylbenzo[d][1,3]dioxol-5-yl)-3-oxo-3-(2-oxothiazolidin-3-yl)propyl)naphthalen-2-yl)oxy) acetamide (8c UCAB #1229). A mixture of 2n (0.043 g, 0.11 mmol), DCC (0.04 g, 0.19 mmol) and DMAP (0.02 g, 0.2 mmol) were added in DMF (1 mL). The mixture was allowed to stir at RT for 30 min. Then thiazolidin-2-one (0.04 g, 0.40 mmol) was added and the mixture was stirred overnight. Upon reaction completion, the mixture was extracted with ethyl acetate three times, washed with brine, and dried over Na₂SO₄, filtered and concentrated under reduced pressure. The crude was purified by silica gel column chromatography to afford the desired product. White solid (0.0016 g, 6.4%). ¹H NMR (600 MHz, DMSO) δ 7.96 (d, J=8.1 Hz, 1H), 7.74 (dd, J=21.4, 8.6 Hz, 2H), 7.54 (d, J=1.8 Hz, 1H), 7.26 (dd, J=8.5, 1.8 Hz, 1H), 7.18 (d, J=8.2 Hz, 2H), 6.89 (s, 1H), 6.73 (s, 1H), 5.92 (dd, J=22.4, 1.0 Hz, 2H), 4.75 (t, J=7.4 Hz, 1H), 4.54 (s, 2H), 4.03-3.93 (m, 2H), 3.82 (qt, J=9.0, 4.5 Hz, 1H), 3.64-3.53 (m, 2H), 3.31 (s, 2H), 2.24 (s, 3H), 1.80-1.22 (m, 12H). ¹³C NMR (151 MHz, DMSO) δ 172.92, 170.27, 166.04, 155.86, 145.50, 145.12, 141.84, 134.76, 134.02, 128.85, 128.59, 127.62, 127.25, 124.58, 124.53, 118.27, 110.37, 107.12, 107.03, 100.59, 67.02, 49.52, 47.00, 42.02, 41.36, 34.17, 27.67 (2C), 24.77 (2C), 23.71 (2C), 19.31. LC-MS (ESI): m/z=589.3, t_{R}=5.19 min

N-cycloheptyl-2-((7-(1-7-methoxy-1-methyl-1H-benzo[d][1,2,3]triazol-5-yl)-3-oxo-3-(2-oxothiazolidin-3-yl)propyl)naphthalen-2-yl)oxy) acetamide (8d UCAB #1231). A mixture of 20 (0.06 g, 0.11 mmol), DCC (0.05 g, 0.25 mmol) and DMAP (0.03 g, 0.25 mmol) were added in DMF (1 mL). The mixture was allowed to stir at RT for 30 min. Then thiazolidin-2-one (0.05 g, 0.44 mmol) was added and the mixture was stirred overnight. Upon reaction completion, the mixture was extracted with ethyl acetate three times, washed with brine, and dried over Na₂SO₄, filtered and concentrated under reduced pressure. The crude was purified by silica gel column chromatography to afford the desired product. White solid (0.005 g, 14%). ¹H NMR (600 MHz, DMSO) δ 7.95 (d, J=8.1 Hz, 1H), 7.75 (dd, J=15.4, 8.6 Hz, 2H), 7.68 (s, 1H), 7.47 (s, 1H), 7.37 (dd, J=8.4, 1.8 Hz, 1H), 7.18 (d, J=8.6 Hz, 2H), 6.95 (s, 1H), 4.80 (t, J=7.5 Hz, 1H), 4.53 (s, 2H), 4.34 (s, 3H), 3.97 (t, J=7.2 Hz, 2H), 3.91 (s, 3H), 3.86-3.78 (m, 2H), 3.72 (dd, J=17.4, 7.3 Hz, 1H), 3.30 (t, J=7.3 Hz, 2H), 1.80-1.31 (m, 12H). ¹³C NMR (151 MHz, DMSO) δ 173.47, 170.74, 166.53, 156.38, 147.96, 146.19, 142.51, 142.04, 134.59, 129.39, 128.16, 127.87, 125.07 (2C), 124.32, 118.81, 109.09, 108.04, 107.68, 67.52, 56.65, 50.03, 47.51, 46.56, 42.18, 37.03, 34.66, 28.14 (2C), 25.25 (2C), 24.20 (2C). LC-MS (ESI): m/z=616.5, t_{R}=4.88 min

Ethyl 2-((6-bromoquinolin-3-yl)oxy)acetate (1-38) A mixture of 6-bromoquinolin-3-ol (0.1 g, 0.45 mmol), K₂CO₃ (0.063 g, 0.46 mmol) and ethyl 2-bromoacetate (0.079, 0.47 mmol) were added in MeCN (4 mL). The mixture was allowed to stir at 60° C. for 18 h. Upon reaction completion, the mixture was cooled to room temperature, extracted with ethyl acetate three times, washed with brine, and dried over Na₂SO₄, filtered and concentrated under reduced pressure. The crude was purified by silica gel column chromatography to afford the desired product. White solid (0.12 g, 87.0%). ¹H NMR (600 MHz, DMSO) δ 8.73 (d, J=2.9 Hz, 1H), 8.13 (d, J=2.2 Hz, 1H), 7.90 (d, J=8.9 Hz, 1H), 7.76-7.69 (m, 2H), 4.97 (s, 2H), 4.20 (q, J=7.1 Hz, 2H), 1.23 (t, J=7.1 Hz, 4H). LC-MS (ESI): m/z=310.0/312.0, t_{R}=4.40 min.

2-((6-bromoquinolin-3-yl)oxy) acetic acid (1-39). The reaction was conducted following the general procedure B, using I-38 (12, 0.39 mmol) and 1 M aq. NaOH (1 ml). White solid (0.10 g, 91.7%). ¹H NMR (400 MHz, DMSO) δ 13.20 (s, 1H), 8.72 (d, J=2.9 Hz, 1H), 8.16 (d, J=2.2 Hz, 1H), 7.91 (d, J=8.9 Hz, 1H), 7.77-7.68 (m, 2H), 4.89 (s, 2H). LC-MS (ESI): m/z=282.0/284.0, t_{R}=3.55 min

2-((6-bromoquinolin-3-yl)oxy)-N-cycloheptylacetamide (1-40). The reaction was conducted following the general procedure E, using I-39 (0.13 g, 0.45 mmol), HATU (0.35 g, 0.92 mmol), DIPEA (0.24 mL, 1.38 mmol) and cycloheptanamine (0.053 g, 0.67 mmol). White solid (0.06 g, 45.1%). ¹H NMR (600 MHz, DMSO) δ 8.76 (d, J=2.9 Hz, 1H), 8.18-8.09 (m, 1H), 8.06 (d, J=8.1 Hz, 1H), 7.91 (d, J=8.9 Hz, 1H), 7.75-7.67 (m, 2H), 4.64 (s, 2H), 3.84 (dtd, J=12.5, 9.1, 4.5 Hz, 1H), 1.78 (ddd, J=13.7, 7.4, 3.3 Hz, 2H), 1.66-1.35 (m, 8H), 1.26-1.16 (m, 2H). LC-MS (ESI): m/z=377.1/379.1, t_{R}=4.71 min.

N-cycloheptyl-2-((6-(4,4,5,5-tetramethyl-1,3,2-dioxaborolan-2-yl) quinolin-3-yl)oxy) acetamide (1-41). A solution of 1-40 (0.06 g, 0.16 mmol), PinB-BPin (0.05 g, 0.19 mmol), KOAc (0.05 g, 0.5 mmol), and Pd(dppf)Cl₂ (0.004 g, 0.005 mmol) were added in dioxane (3 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.06 g, 89.6%). ¹H NMR (600 MHz, DMSO) δ 8.78 (d, J=2.9 Hz, 1H), 8.26 (d, J=1.4 Hz, 1H), 8.06 (d, J=8.1 Hz, 1H), 7.97-7.91 (m, 1H), 7.82-7.76 (m, 2H), 4.63 (s, 2H), 3.84 (ddp, J=12.6, 8.9, 4.5 Hz, 1H), 1.82-1.74 (m, 2H), 1.66-1.37 (m, 9H), 1.35 (s, 12H), 1.27-1.21 (m, 1H). LC-MS (ESI): m/z=425.3, t_{R}=5.02 min.

Methyl 3-(benzo[d][1,3]dioxol-5-yl)-3-(3-(2-(cycloheptylamino)-2-oxoethoxy) quinolin-6-yl)propanoate (I-42). The reaction was conducted following the general procedure F, using I-41 (0.06 g, 0.14 mmol) and I-9m (0.022 g, 0.1 mmol), TEA (0.1 ml, 0.28 mmol), and [Rh(COD)Cl]₂ (5 mg, 0.01 mmol). White solid (0.012 g, 25.5%). LC-MS (ESI): m/z=505.3, t_{R}=4.66 min

3-(benzo[d][1,3]dioxol-5-yl)-3-(3-(2-(cycloheptylamino)-2-oxoethoxy) quinolin-6-yl)propanoic acid (9a, UCAB #1222). The reaction was conducted following the general procedure G, using I-42 (0.012 g, 0.011 mmol) and 2 M aq. NaOH (110 μl). White solid (0.001 g, 9%). ¹H NMR (600 MHz, DMSO) δ 12.13 (s, 1H), 8.65 (d, J=2.9 Hz, 1H), 8.04 (d, J=8.1 Hz, 1H), 7.84 (d, J=8.6 Hz, 1H), 7.75 (d, J=2.0 Hz, 1H), 7.59 (d, J=2.9 Hz, 1H), 7.54 (dd, J=8.7, 2.0 Hz, 1H), 6.97 (d, J=1.6 Hz, 1H), 6.85-6.78 (m, 2H), 5.94 (dd, J=5.3, 1.0 Hz, 2H), 4.61 (d, J=11.3 Hz, 2H), 4.53 (t, J=7.9 Hz, 1H), 3.83 (qt, J=9.1, 4.4 Hz, 1H), 3.15-3.01 (m, 2H), 1.80-1.73 (m, 2H), 1.67-1.29 (m, 10H). ¹³C NMR (151 MHz, DMSO) δ 173.09, 166.09, 152.01, 147.79, 146.13,144.16, 143.6, 142.46, 129.04, 128.77, 127.90, 138.18, 129.04, 128.80, 127.87, 124.92, 121.11, 114.27, 108.57, 101.27, 67.56, 50.14, 34.69 (2C), 28.15 (2C), 24.23 (2C). LC-MS (ESI): m/z=491.2, t_{R}=4.28 min.

2-((7-bromoquinolin-2-yl)oxy)-N-cycloheptylacetamide (1-43) A mixture of 7-bromoquinolin-2 (1H)-one (0.07 g, 0.31 mmol), K₂CO₃ (0.05 g, 0.34 mmol), Pd(Xantphos)Cl₂ (0.006 g, 0.008 mmol) and I-36 (0.0073 g, 0.47 mmol) were added in Toluene (5 mL). The mixture was allowed to stir at 100° C. for 48 h. Upon reaction completion, the mixture was cooled to room temperature, extracted with ethyl acetate three times, washed with brine, and dried over Na₂SO₄, filtered and concentrated under reduced pressure. The crude was purified by silica gel column chromatography to afford the desired product. White solid (0.030 g, 25.6%). ¹H NMR (600 MHz, DMSO) δ 8.31 (d, J=8.9 Hz, 1H), 7.98 (dd, J=13.5, 6.2 Hz, 1H), 7.92-7.86 (m, 2H), 7.61 (dd, J=8.5, 2.0 Hz, 1H), 7.16 (d, J=8.9 Hz, 1H), 4.81 (s, 2H), 3.79 (qq, J=8.1, 4.1 Hz, 1H), 1.80-1.72 (m, 2H), 1.68-1.14 (m, 10H). LC-MS (ESI): m/z=377.1/379.1, t_{R}=4.32 min.

N-cycloheptyl-2-((7-(4,4,5,5-tetramethyl-1,3,2-dioxaborolan-2-yl) quinolin-2-yl)oxy) acetamide (1-44). A solution of I-43 (0.03 g, 0.08 mmol), PinB-BPin (0.03 g, 0.1 mmol), KOAc (0.03 g, 0.3 mmol), and Pd(dppf)Cl₂ (0.002 g, 0.003 mmol) were added in dioxane (3 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. White solid (0.06 g, 89.6%). ¹H NMR (600 MHz, DMSO) δ 8.31-8.24 (m, 1H), 8.07 (d, J=1.2 Hz, 1H), 8.01-7.95 (m, 1H), 7.87 (d, J=8.0 Hz, 1H), 7.64 (dd, J=7.9, 1.2 Hz, 1H), 7.17-7.08 (m, 1H), 4.80 (d, J=7.8 Hz, 2H), 3.79 (qp, J=8.7, 4.3 Hz, 1H), 1.79-1.72 (m, 2H), 1.63-1.38 (m, 8H), 1.33 (s, 12H), 1.24 (d, J=2.9 Hz, 2H). LC-MS (ESI): m/z=425.3, t_{R}=5.30 min.

Methyl 3-(benzo[d][1,3]dioxol-5-yl)-3-(2-(2-(cycloheptylamino)-2-oxoethoxy) quinolin-7-yl)propanoate (I-44). The reaction was conducted following the general procedure F, using I-43 (0.06 g, 0.14 mmol) and I-9m (0.02 g, 0.1 mmol), TEA (0.1 ml, 0.28 mmol), and [Rh(COD)Cl]₂ (5 mg, 0.01 mmol). White solid (0.01 g, 21.1%). ¹H NMR (400 MHz, DMSO) δ 8.18 (d, J=8.8 Hz, 1H), 7.94 (d, J=14.8 Hz, 1H), 7.78 (d, J=8.3 Hz, 1H), 7.60-7.46 (m, 1H), 7.46-7.34 (m, 1H), 7.07-6.92 (m, 2H), 6.87-6.77 (m, 2H), 6.00-5.92 (m, 2H), 4.78 (s, 2H), 4.64-4.51 (m, 1H), 3.78 (tt, J=8.6, 4.6 Hz, 1H), 3.50 (s, 3H), 3.28-3.07 (m, 2H), 1.75 (t, J=8.6 Hz, 2H), 1.67-1.32 (m, 10H). LC-MS (ESI): m/z=505.3, t_{R}=4.90 min

3-(benzo[d][1,3]dioxol-5-yl)-3-(2-(2-(cycloheptylamino)-2-oxoethoxy) quinolin-7-yl)propanoic acid (9b, UCAB #1235). The reaction was conducted following the general procedure G, using I-43 (0.01 g, 0.02 mmol) and 2 M aq. NaOH (110 μl). White solid (0.002 g, 20.6%). ¹H NMR (600 MHz, DMSO) δ 12.13 (s, 1H), 8.19-8.14 (m, 1H), 7.96 (d, J=7.97 Hz, 1H), 7.77 (d, J=8.2 Hz, 1H), 7.56 (s, 1H), 7.40 (d, J=8.3 Hz, 1H), 7.04-6.98 (m, 1H), 6.94 (s, 1H), 6.84-6.77 (m, 2H), 6.01-5.89 (m, 2H), 4.79-4.71 (m, 2H), 4.51 (t, J=7.9 Hz, 1H), 3.77 (s, 1H), 3.16-2.94 (m, 2H), 1.79-1.68 (m, 2H), 1.63-1.31 (m, 10H). ¹³C NMR (151 MHz, DMSO) δ 173.07, 166.82, 161.35, 147.77, 146.68, 146.12 (2C), 139.49, 138.10, 128.19, 125.18, 124.84, 123.95, 121.08, 112.91, 108.57 (2C), 101.28, 64.71, 49.94, 46.77, 40.34, 34.69 (2C), 28.17 (2C), 24.23 (2C). LC-MS (ESI): m/z=491.2, t_{R}=4.46 min.

2-((3-bromoquinolin-6-yl)oxy)-N-cycloheptylacetamide (1-46) A mixture of 3-bromoquinolin-6-ol (0.15 g, 0.67 mmol), K₂CO₃ (0.1 g, 0.7 mmol), and I-36 (0.1 g, 0.7 mmol) were added in MeCN (5 mL). The mixture was allowed to stir at 80° C. for 18 h. Upon reaction completion, the mixture was cooled to room temperature, extracted with ethyl acetate three times, washed with brine, and dried over Na₂SO₄, filtered and concentrated under reduced pressure. The crude was purified by silica gel column chromatography to afford the desired product. White solid (0.025 g, 10%). ¹H NMR (400 MHz, DMSO) δ 8.79 (d, J=2.3 Hz, 1H), 8.57 (d, J=2.3 Hz, 1H), 7.99 (dd, J=17.2, 8.6 Hz, 2H), 7.55 (dd, J=9.2, 2.8 Hz, 1H), 7.31 (d, J=2.8 Hz, 1H), 4.59 (s, 2H), 3.84 (dtd, J=12.8, 8.5, 4.0 Hz, 1H), 1.83-1.73 (m, 2H), 1.66-1.19 (m, 10H). LC-MS (ESI): m/z=377.2/379.2, t_{R}=5.31 min.

Methyl (E)-3-(6-(2-(cycloheptylamino)-2-oxoethoxy) quinolin-3-yl) acrylate (I-47). The reaction was conducted following the General procedure C, using I-46 (0.03 g, 0.08 mmol), methyl acrylate (0.01 ml, 0.12 mmol), Pd(OAc)₂ (0.01 g, 0.004 mmol), tri(o-tolyl)phosphine (0.01 g, 0.008 mmol), and Et₃N (0.1 ml, 0.57 mmol). White solid (0.025 g, 83.3%). ¹H NMR (400 MHz, DMSO) δ 9.12 (d, J=2.1 Hz, 1H), 8.55 (d, J=2.1 Hz, 1H), 8.06-7.94 (m, 2H), 7.85 (d, J=16.1 Hz, 1H), 7.54 (dd, J=9.2, 2.8 Hz, 1H), 7.29 (d, J=2.8 Hz, 1H), 6.94 (d, J=16.2 Hz, 1H), 4.61 (s, 2H), 3.89-3.79 (m, 1H), 3.77 (s, 3H), 1.83-1.73 (m, 2H), 1.67-1.26 (m, 10H). LC-MS (ESI): m/z=383.3, t_{R}=4.36 min.

2-(benzo[d][1,3]dioxol-5-yl)-4,4,5,5-tetramethyl-1,3,2-dioxaborolane (1-49). A solution of 5-bromobenzo[d][1,3]dioxole (0.3 g, 1.5 mmol), PinB-BPin (0.46 g, 1.8 mmol), KOAc (0.44 g, 4.5 mmol), and Pd(dppf)Cl₂ (0.04 g, 0.05 mmol) were added in dioxane (5 ml). This mixture was stirred at 100° C. for 4 h under N₂ atmosphere. Upon reaction completion (as determined by LC-MS). After cooling down, the salts were filtered out, the resulting filtrate was concentrated. The crude was purified by silica gel column chromatography to afford the desired product. Colorless oil (0.08 g, 21.6%). ¹H NMR (600 MHz, DMSO) δ 7.23 (dd, J=7.7, 1.2 Hz, 1H), 7.06 (d, J=1.1 Hz, 1H), 6.93 (d, J=7.7 Hz, 1H), 6.02 (s, 2H), 1.27 (s, 12H). ¹³C NMR (151 MHz, DMSO) δ 149.96, 146.97, 129.48 (2C), 113.04, 108.39, 100.80, 83.50 (2C), 24.61 (4C). LC-MS (ESI): m/z=425.3, t_{R}=5.30 min.

Methyl 3-(benzo[d][1,3]dioxol-5-yl)-3-(3-(2-(cycloheptylamino)-2-oxoethoxy) quinolin-6-yl)propanoate (I-48). The reaction was conducted following the general procedure F, using I-46 (0.025 g, 0.065 mmol) and I-49 (0.025 g, 0.1 mmol), TEA (0.03 ml, 0.2 mmol), and [Rh(COD)Cl]₂ (4 mg, 0.01 mmol). White solid (0.015 g, 45.5%). LC-MS (ESI): m/z=505.3, t_{R}=4.38 min

3-(benzo[d][1,3]dioxol-5-yl)-3-(6-(2-(cycloheptylamino)-2-oxoethoxy) quinolin-3-yl)propanoic acid (9c, UCAB #1258). The reaction was conducted following the general procedure G, using I-43 (0.015 g, 0.03 mmol) and 2 M aq. NaOH (300 μl). White solid (0.0045 g, 31%). ¹H NMR (600 MHz, DMSO) δ 12.25 (s, 1H), 8.89-8.85 (m, 1H), 8.36 (s, 1H), 8.01 (d, J=8.1 Hz, 1H), 7.95 (d, J=9.2 Hz, 1H), 7.53 (dd, J=9.2, 2.8 Hz, 1H), 7.32 (d, J=2.8 Hz, 1H), 7.05 (d, J=1.7 Hz, 1H), 6.94-6.77 (m, 2H), 5.96 (dd, J=6.4, 1.0 Hz, 2H), 4.62 (d, J=11.4 Hz, 3H), 3.83 (dh, J=13.6, 4.5 Hz, 1H), 3.33-2.97 (m, 2H), 1.77 (ddd, J=12.4, 7.2, 4.1 Hz, 2H), 1.63-1.18 (m, 10H). ¹³C NMR (151 MHz, DMSO) δ 172.94, 166.16, 158.69, 158.45, 156.97, 147.96, 146.37, 138.58, 137.32, 129.55, 129.41, 128.07, 123.63, 121.23, 108.73, 108.55, 107.50, 101.38, 67.72, 50.13, 44.22, 40.42, 34.68 (2C), 28.14 (2C), 24.22 (2C). LC-MS (ESI): m/z=491.2, t_{R}=3.98 min.

### Example 6. Binding Activity of the Compounds by FP and X-Ray Crystallography

The final compounds from Example 5 (Scheme 2-7) were tested in the FP assay (Table 1). Thereby it was shown that they bind the Keap1 Kelch domain and inhibit the Keap1-Nrf2 interaction. Several compounds showed very high affinity by FP (Table 1; FIG. 3A), and the binding mode of several compounds to the Keap1 Kelch domain was characterized by X-ray crystallography (FIG. 3B).

TABLE 1
Exemplified compounds of the invention.

| | | | |
|---|---|---|---|
| | | | Activity |
| | | Formula | in FP |
| Cpd | Structure | MW (g/mol) | (Kᵢ/uM) |

Scheme 2 compounds

| | | | |
|---|---|---|---|
| 1a UCAB#859 | 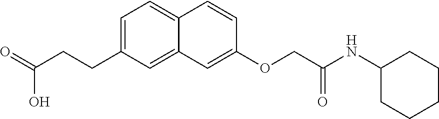 [SMILES: O=C(O)CCc1ccc2ccc(OCC(=O)NC3CCCCC3)cc2c1] | C₂₁H₂₅NO₄ 355.43 | 14 ± 0.8 (n ≥ 3) |
| 1b UCAB#996 | 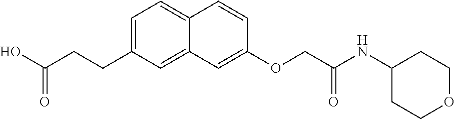 [SMILES: O=C(O)CCc1ccc2ccc(OCC(=O)NC3CCOCC3)cc2c1] | C₂₀H₂₃NO₅ 357.41 | 38 ± 3 (n ≥ 3) |
| 1c UCAB#1001 | 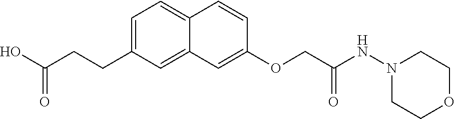 [SMILES: O=C(O)CCc1ccc2ccc(OCC(=O)NN3CCOCC3)cc2c1] | C₁₉H₂₂N₂O₅ 358.39 | 268 ± 29 (n ≥ 3) |
| 1d UCAB#909 | 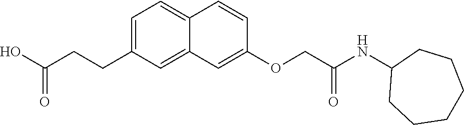 [SMILES: O=C(O)CCc1ccc2ccc(OCC(=O)NC3CCCCCC3)cc2c1] | C₂₂H₂₇NO₄ 369.46 | 6.0 ± 0.3 (n ≥ 3) |
| 1e UCAB#908 | 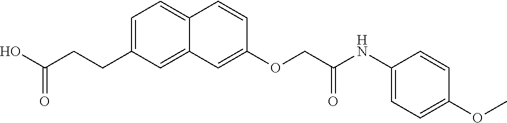 [SMILES: COc1ccc(NC(=O)COc2ccc3ccc(CCC(=O)O)cc3c2)cc1] | C₂₂H₂₁NO₅ 379.41 | 75 ± 10 (n ≥ 3) |
| 1f UCAB#910 | 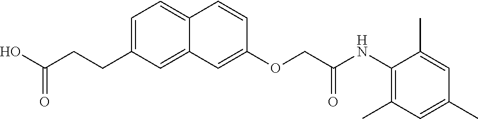 [SMILES: Cc1cc(C)c(NC(=O)COc2ccc3ccc(CCC(=O)O)cc3c2)c(C)c1] | C₂₄H₂₅NO₄ 391.47 | 22 ± 0.8 (n ≥ 3) |
| 1g UCAB#997 | 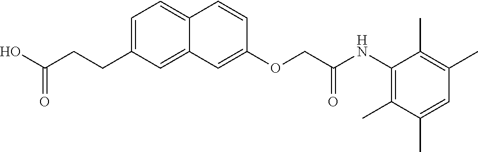 [SMILES: Cc1cc(C)c(C)c(NC(=O)COc2ccc3ccc(CCC(=O)O)cc3c2)c1C] | C₂₅H₂₇NO₄ 405.49 | 196 ± 24 (n ≥ 3) |
| 1h UCAB#1233 | 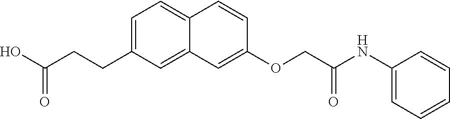 [SMILES: O=C(O)CCc1ccc2ccc(OCC(=O)Nc3ccccc3)cc2c1] | C₂₁H₁₉NO₄ 349.39 | 7.8 ± 0.1 (n = 2) |

Scheme 3 compounds

| | | | |
|---|---|---|---|
| 2a UCAB#944 | 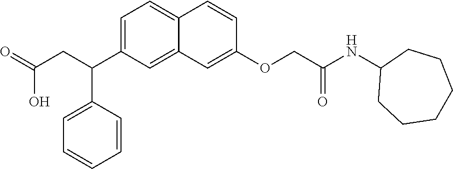 [SMILES: O=C(O)CC(c1ccccc1)c1ccc2ccc(OCC(=O)NC3CCCCCC3)cc2c1] | C₂₈H₃₁NO₄ 445.56 | 1.1 ± 0.1 (n ≥ 3) |
| 2b UCAB#964 | 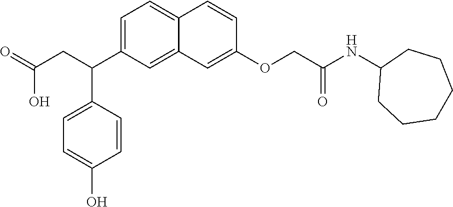 [SMILES: O=C(O)CC(c1ccc(O)cc1)c1ccc2ccc(OCC(=O)NC3CCCCCC3)cc2c1] | C₂₈H₃₁NO₅ 461.56 | 0.181 ± 0.02 (n ≥ 3) |
| 2c UCAB#965 | 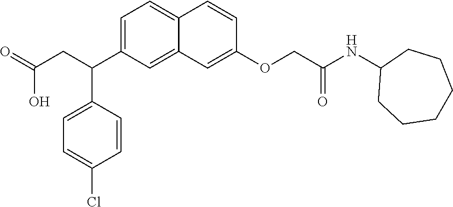 [SMILES: O=C(O)CC(c1ccc(Cl)cc1)c1ccc2ccc(OCC(=O)NC3CCCCCC3)cc2c1] | C₂₈H₃₀ClNO₄ 480.00 | 0.294 ± 0.03 (n ≥ 3) |
| 2d UCAB#945 | 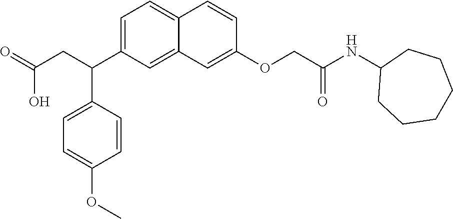 [SMILES: COc1ccc(C(CC(=O)O)c2ccc3ccc(OCC(=O)NC4CCCCCC4)cc3c2)cc1] | C₂₉H₃₃NO₅ 475.59 | 0.032 ± 0.03 (n ≥ 3) |
| 2e UCAB#953 | 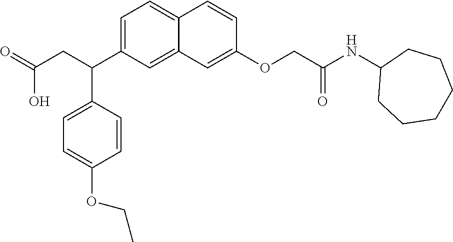 [SMILES: CCOc1ccc(C(CC(=O)O)c2ccc3ccc(OCC(=O)NC4CCCCCC4)cc3c2)cc1] | C₃₀H₃₅NO₅ 489.61 | 0.041 ± 0.005 (n ≥ 3) |
| 2f UCAB#966 | 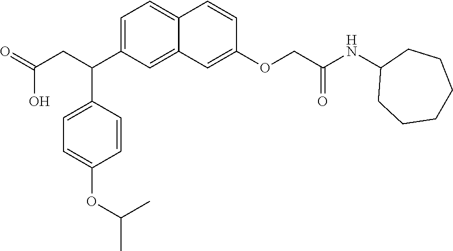 [SMILES: CC(C)Oc1ccc(C(CC(=O)O)c2ccc3ccc(OCC(=O)NC4CCCCCC4)cc3c2)cc1] | C₃₁H₃₇NO₅ 503.64 | 0.101 ± 0.005 (n ≥ 3) |
| 2g UCAB#962 | 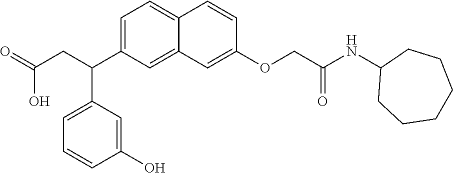 [SMILES: O=C(O)CC(c1cccc(O)c1)c1ccc2ccc(OCC(=O)NC3CCCCCC3)cc2c1] | C₂₈H₃₁NO₅ 461.56 | 0.203 ± 0.03 (n ≥ 3) |
| 2h UCAB#961 | 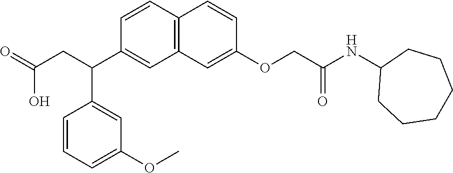 [SMILES: COc1cccc(C(CC(=O)O)c2ccc3ccc(OCC(=O)NC4CCCCCC4)cc3c2)c1] | C₂₉H₃₃NO₅ 475.59 | 0.284 ± 0.03 (n ≥ 3) |
| 2i UCAB#963 | 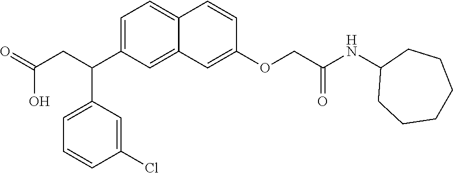 [SMILES: O=C(O)CC(c1cccc(Cl)c1)c1ccc2ccc(OCC(=O)NC3CCCCCC3)cc2c1] | C₂₈H₃₀ClNO₄ 480.00 | 0.054 ± 0.006 (n ≥ 3) |
| 2j UCAB#967 | 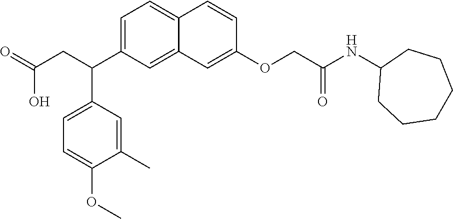 [SMILES: COc1ccc(C(CC(=O)O)c2ccc3ccc(OCC(=O)NC4CCCCCC4)cc3c2)cc1C] | C₃₀H₃₅NO₅ 489.61 | 0.610 ± 0.06 (n ≥ 3) |
| 2k UCAB#960 | 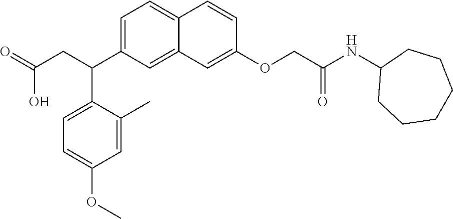 [SMILES: COc1ccc(C(CC(=O)O)c2ccc3ccc(OCC(=O)NC4CCCCCC4)cc3c2)c(C)c1] | C₃₀H₃₅NO₅ 489.61 | 0.027 ± 0.003 (n ≥ 3) |
| 2l UCAB#1083 | 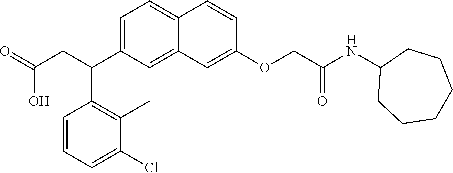 [SMILES: Cc1c(Cl)cccc1C(CC(=O)O)c1ccc2ccc(OCC(=O)NC3CCCCCC3)cc2c1] | C₂₉H₃₂ClNO₄ 494.03 | 0.06 ± 0.008 (n ≥ 3) |
| 2m UCAB#952 | 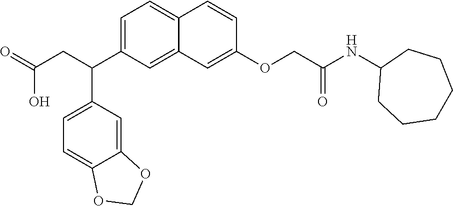 [SMILES: O=C(O)CC(c1ccc2c(c1)OCO2)c1ccc2ccc(OCC(=O)NC3CCCCCC3)cc2c1] | C₂₉H₃₁NO₆ 489.57 | 0.023 ± 0.002 (n ≥ 3) |
| 2n UCAB#985 | 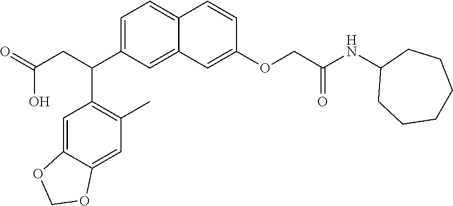 [SMILES: Cc1cc2c(cc1C(CC(=O)O)c1ccc3ccc(OCC(=O)NC4CCCCCC4)cc3c1)OCO2] | C₃₀H₃₃NO₆ 503.60 | 0.008 ± 0.002 (n ≥ 3) |
| 2o UCAB#987 | 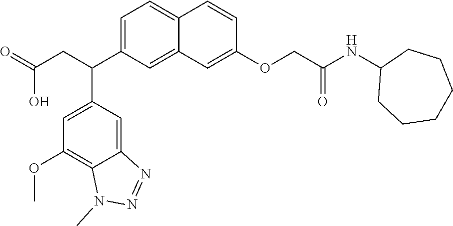 [SMILES: COc1cc(C(CC(=O)O)c2ccc3ccc(OCC(=O)NC4CCCCCC4)cc3c2)cc2nnn(C)c12] | C₃₀H₃₄N₄O₅ 530.63 | 0.0035 ± 0.0006 (n ≥ 3) |
| 2p UCAB#991 | 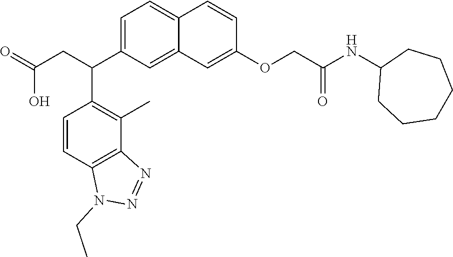 [SMILES: CCn1nnc2c(C)c(C(CC(=O)O)c3ccc4ccc(OCC(=O)NC5CCCCCC5)cc4c3)ccc21] | C₃₁H₃₆N₄O₄ 528.65 | 0.0059 ± 0.0007 (n ≥ 3) |
| 2q UCAB#973 | 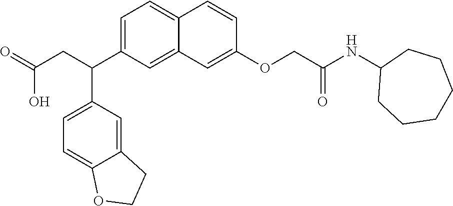 [SMILES: O=C(O)CC(c1ccc2c(c1)CCO2)c1ccc2ccc(OCC(=O)NC3CCCCCC3)cc2c1] | C₃₀H₃₃NO₅ 487.60 | 0.036 ± 0.003 (n ≥ 3) |
| 2r UCAB#1082 | 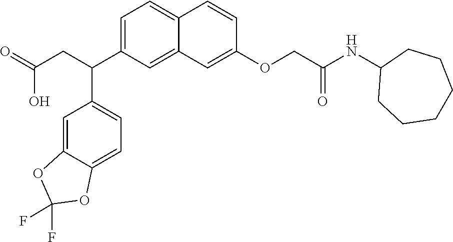 [SMILES: O=C(O)CC(c1ccc2c(c1)OC(F)(F)O2)c1ccc2ccc(OCC(=O)NC3CCCCCC3)cc2c1] | C₂₉H₂₉F₂NO₆ 525.55 | 0.034 ± 0.005 (n ≥ 3) |
| 2s UCAB#1090 | 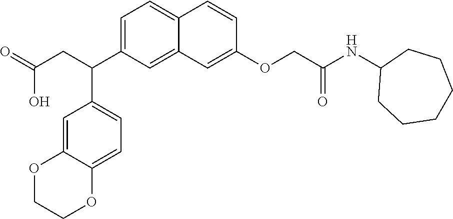 [SMILES: O=C(O)CC(c1ccc2c(c1)OCCO2)c1ccc2ccc(OCC(=O)NC3CCCCCC3)cc2c1] | C₃₀H₃₃NO₆ 503.60 | 0.015 ± 0.003 (n ≥ 3) |
| 2t UCAB#983 | 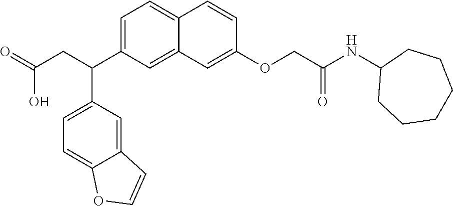 [SMILES: O=C(O)CC(c1ccc2ccc(OCC(=O)NC3CCCCCC3)cc2c1)c1ccc2occc2c1] | C₃₀H₃₁NO₅ 485.58 | 0.038 ± 0.002 (n ≥ 3) |
| 2u UCAB#1124 | 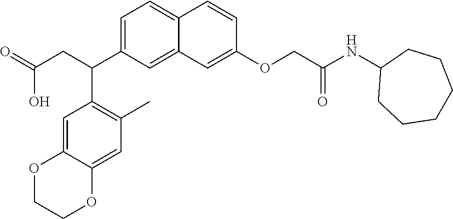 [SMILES: Cc1cc2c(cc1C(CC(=O)O)c1ccc3ccc(OCC(=O)NC4CCCCCC4)cc3c1)OCCO2] | C₃₁H₃₅NO₆ 517.62 | 0.009 ± 0.003 (n ≥ 3) |
| 2v UCAB#1101 | 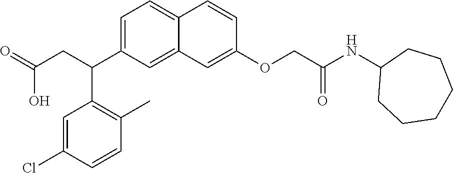 [SMILES: Cc1ccc(Cl)cc1C(CC(=O)O)c1ccc2ccc(OCC(=O)NC3CCCCCC3)cc2c1] | C₂₉H₃₂ClNO₄ 494.03 | 0.097 ± 0.013 (n ≥ 3) |
| 2w UCAB#1129 | 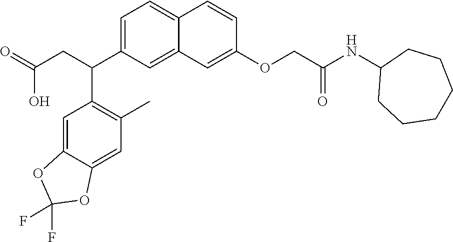 [SMILES: Cc1cc2c(cc1C(CC(=O)O)c1ccc3ccc(OCC(=O)NC4CCCCCC4)cc3c1)OC(F)(F)O2] | C₃₀H₃₁F₂NO₆ 539.58 | 0.014 ± 0.002 (n ≥ 3) |
| 2x UCAB#1141 | 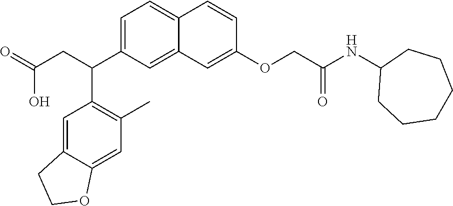 [SMILES: Cc1cc2c(cc1C(CC(=O)O)c1ccc3ccc(OCC(=O)NC4CCCCCC4)cc3c1)CCO2] | C₃₀H₃₅NO₅ 501.62 | 0.019 ± 0.002 (n ≥ 3) |
| 2y UCAB#1191 | 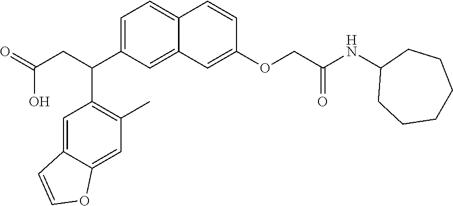 [SMILES: Cc1cc2occc2cc1C(CC(=O)O)c1ccc2ccc(OCC(=O)NC3CCCCCC3)cc2c1] | C₃₁H₃₃NO₅ 499.61 | 0.016 ± 0.003 (n ≥ 3) |
| 2z UCAB#1221 | 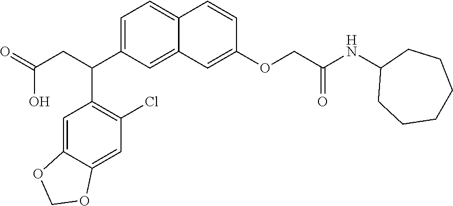 [SMILES: O=C(O)CC(c1ccc2ccc(OCC(=O)NC3CCCCCC3)cc2c1)c1cc2c(cc1Cl)OCO2] | C₂₉H₃₀ClNO₆ 524.01 | 0.009 ± 0.002 (n = 2) |

Scheme 4 compounds

| | | | |
|---|---|---|---|
| 3a UCAB#1022 | 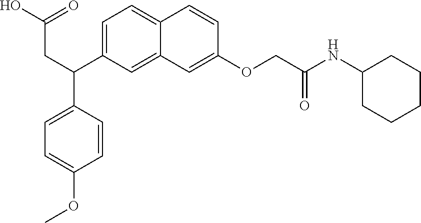 [SMILES: COc1ccc(C(CC(=O)O)c2ccc3ccc(OCC(=O)NC4CCCCC4)cc3c2)cc1] | C₂₈H₃₁NO₅ 461.56 | 0.07 ± 0.003 (n ≥ 3) |
| 3b UCAB#1018 | 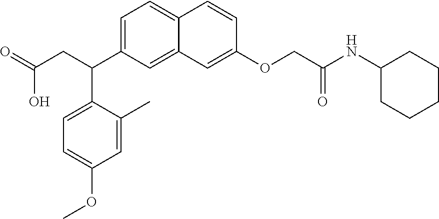 [SMILES: COc1ccc(C(CC(=O)O)c2ccc3ccc(OCC(=O)NC4CCCCC4)cc3c2)c(C)c1] | C₂₉H₃₃NO₅ 475.59 | 0.054 0.004 (n ≥ 3) |
| 3c UCAB#1021 | 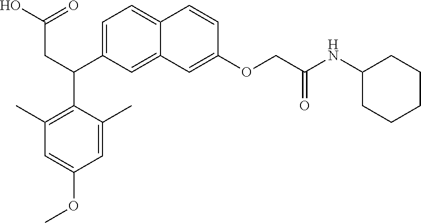 [SMILES: COc1cc(C)c(C(CC(=O)O)c2ccc3ccc(OCC(=O)NC4CCCCC4)cc3c2)c(C)c1] | C₃₀H₃₅NO₅ 489.61 | 0.168 ± 0.003 (n ≥ 3) |
| 3d UCAB#1017 | 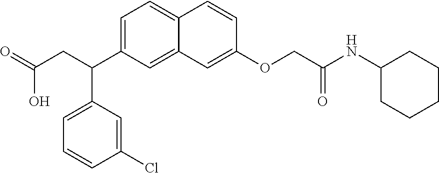 [SMILES: O=C(O)CC(c1cccc(Cl)c1)c1ccc2ccc(OCC(=O)NC3CCCCC3)cc2c1] | C₂₇H₂₈ClNO₄ 465.97 | 0.470 ± 0.047 (n ≥ 3) |
| 3e UCAB#1084 | 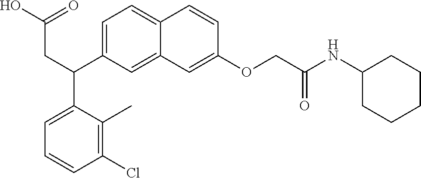 [SMILES: Cc1c(Cl)cccc1C(CC(=O)O)c1ccc2ccc(OCC(=O)NC3CCCCC3)cc2c1] | C₂₈H₃₀ClNO₄ 480.00 | 0.066 ± 0.006 (n ≥ 3) |
| 3f UCAB#1004 | 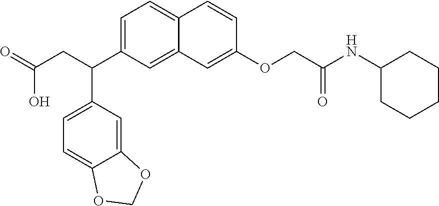 [SMILES: O=C(O)CC(c1ccc2c(c1)OCO2)c1ccc2ccc(OCC(=O)NC3CCCCC3)cc2c1] | C₂₈H₂₉NO₆ 475.54 | 0.059 ± 0.006 (n ≥ 3) |
| 3g UCAB#1026 | 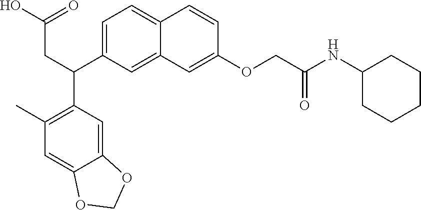 [SMILES: Cc1cc2c(cc1C(CC(=O)O)c1ccc3ccc(OCC(=O)NC4CCCCC4)cc3c1)OCO2] | C₂₉H₃₁NO₆ 489.57 | 0.018 ± 0.006 (n ≥ 3) |
| 3h UCAB#1015 | 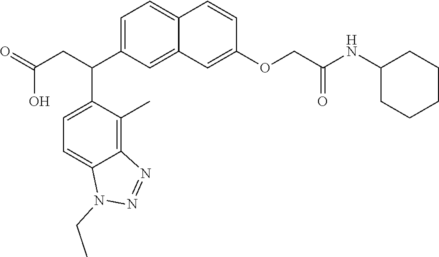 [SMILES: CCn1nnc2c(C)c(C(CC(=O)O)c3ccc4ccc(OCC(=O)NC5CCCCC5)cc4c3)ccc21] | C₃₀H₃₄N₄O₄ 514.63 | 0.0048 ± 0.001 (n ≥ 3) |
| 3i UCAB#1016 | 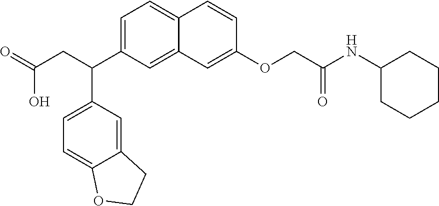 [SMILES: O=C(O)CC(c1ccc2c(c1)CCO2)c1ccc2ccc(OCC(=O)NC3CCCCC3)cc2c1] | C₂₉H₃₁NO₅ 473.57 | 0.082 ± 0.008 (n ≥ 3) |
| 3j UCAB#1038 | 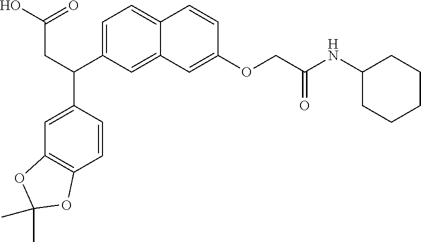 [SMILES: CC1(C)Oc2ccc(C(CC(=O)O)c3ccc4ccc(OCC(=O)NC5CCCCC5)cc4c3)cc2O1] | C₃₀H₃₃NO₆ 503.60 | 0.140 ± 0.011 (n ≥ 3) |
| 3k UCAB#1031 | 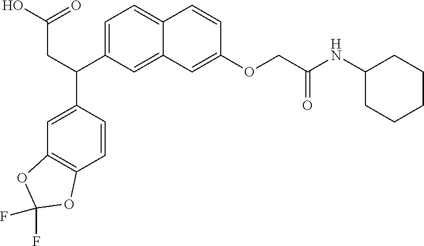 [SMILES: O=C(O)CC(c1ccc2c(c1)OC(F)(F)O2)c1ccc2ccc(OCC(=O)NC3CCCCC3)cc2c1] | C₂₈H₂₇F₂NO₆ 511.52 | 0.052 ± 0.003 (n ≥ 3) |
| 3l UCAB#1032 | 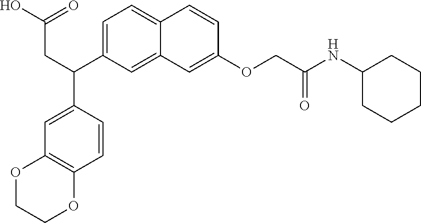 [SMILES: O=C(O)CC(c1ccc2c(c1)OCCO2)c1ccc2ccc(OCC(=O)NC3CCCCC3)cc2c1] | C₂₉H₃₁NO₆ 489.57 | 0.029 ± 0.001 (n ≥ 3) |
| 3m UCAB#1033 | 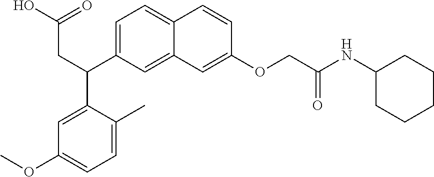 [SMILES: COc1ccc(C)c(C(CC(=O)O)c2ccc3ccc(OCC(=O)NC4CCCCC4)cc3c2)c1] | C₂₉H₃₃NO₅ 475.59 | 0.150 ± 0.014 (n ≥ 3) |
| 3n UCAB#1028 | 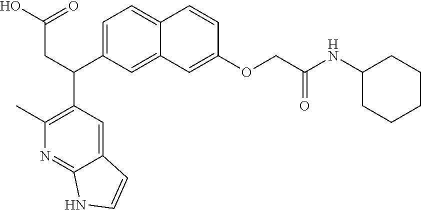 [SMILES: Cc1nc2[nH]ccc2cc1C(CC(=O)O)c1ccc2ccc(OCC(=O)NC3CCCCC3)cc2c1] | C₂₉H₃₁N₃O₄ 485.58 | 0.129 ± 0.013 (n ≥ 3) |
| 3o UCAB#1081 | 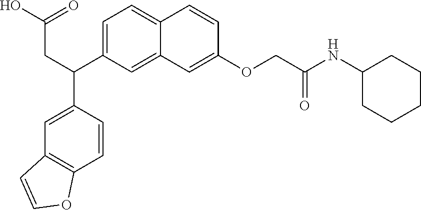 [SMILES: O=C(O)CC(c1ccc2ccc(OCC(=O)NC3CCCCC3)cc2c1)c1ccc2occc2c1] | C₂₉H₂₉NO₅ 471.55 | 0.068 ± 0.012 (n ≥ 3) |
| 3p UCAB#1099 | 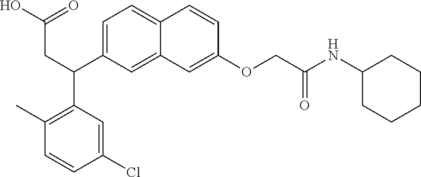 [SMILES: Cc1ccc(Cl)cc1C(CC(=O)O)c1ccc2ccc(OCC(=O)NC3CCCCC3)cc2c1] | C₂₈H₃₀ClNO₄ 480.00 | 0.234 ± 0.04 (n ≥ 3) |
| 3q UCAB#1125 | 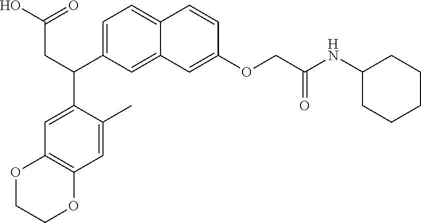 [SMILES: Cc1cc2c(cc1C(CC(=O)O)c1ccc3ccc(OCC(=O)NC4CCCCC4)cc3c1)OCCO2] | C₃₀H₃₃NO₆ 503.60 | 0.015 ± 0.001 (n ≥ 3) |
| 3r UCAB#1130 | 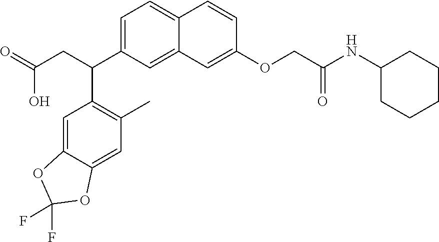 [SMILES: Cc1cc2c(cc1C(CC(=O)O)c1ccc3ccc(OCC(=O)NC4CCCCC4)cc3c1)OC(F)(F)O2] | C₂₉H₂₉F₂NO₆ 525.55 | 0.007 ± 0.001 (n ≥ 3) |
| 3s UCAB#1200 | 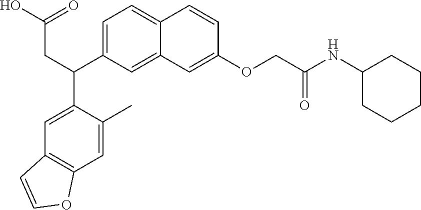 [SMILES: Cc1cc2occc2cc1C(CC(=O)O)c1ccc2ccc(OCC(=O)NC3CCCCC3)cc2c1] | C₃₀H₃₁NO₅ 485.58 | 0.024 ± 0.001 (n ≥ 3) |
| 3t UCAB#1201 | 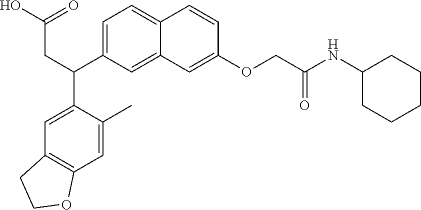 [SMILES: Cc1cc2c(cc1C(CC(=O)O)c1ccc3ccc(OCC(=O)NC4CCCCC4)cc3c1)CCO2] | C₃₀H₃₃NO₅ 487.60 | 0.032 ± 0.001 (n ≥ 3) |

Scheme 5 compounds

| | | | |
|---|---|---|---|
| 4a UCAB#1010 | 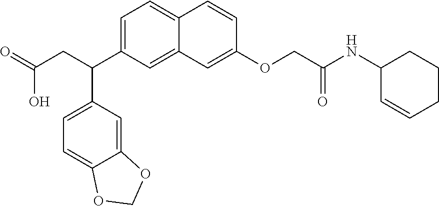 [SMILES: O=C(O)CC(c1ccc2c(c1)OCO2)c1ccc2ccc(OCC(=O)NC3C=CCCC3)cc2c1] | C₂₈H₂₇NO₆ 473.53 | 0.025 ± 0.002 (n ≥ 3) |
| 4b UCAB#1118 | 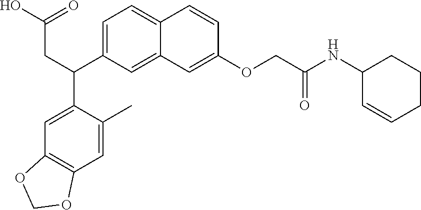 [SMILES: Cc1cc2c(cc1C(CC(=O)O)c1ccc3ccc(OCC(=O)NC4C=CCCC4)cc3c1)OCO2] | C₂₉H₂₉NO₆ 487.55 | 0.004 ± 0.001 (n ≥ 3) |
| 4c UCAB#1109 | 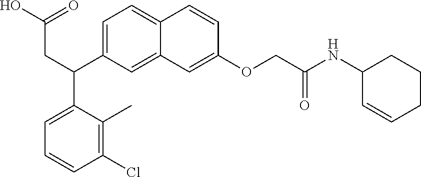 [SMILES: Cc1c(Cl)cccc1C(CC(=O)O)c1ccc2ccc(OCC(=O)NC3C=CCCC3)cc2c1] | C₂₈H₂₈ClNO₄ 477.99 | 0.038 ± 0.006 (n ≥ 3) |
| 4d UCAB#1100 | 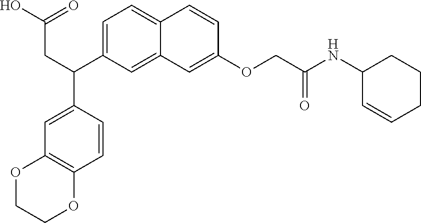 [SMILES: O=C(O)CC(c1ccc2c(c1)OCCO2)c1ccc2ccc(OCC(=O)NC3C=CCCC3)cc2c1] | C₂₉H₂₉NO₆ 487.55 | 0.020 ± 0.002 (n ≥ 3) |
| 4e UCAB#1117 | 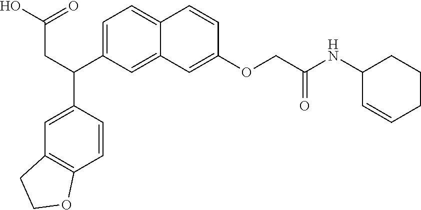 [SMILES: O=C(O)CC(c1ccc2c(c1)CCO2)c1ccc2ccc(OCC(=O)NC3C=CCCC3)cc2c1] | C₂₉H₂₉NO₅ 471.55 | 0.027 ± 0.004 (n ≥ 3) |
| 4f UCAB#1095 | 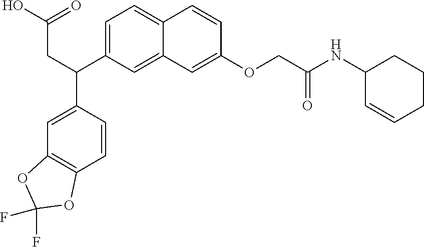 [SMILES: O=C(O)CC(c1ccc2c(c1)OC(F)(F)O2)c1ccc2ccc(OCC(=O)NC3C=CCCC3)cc2c1] | C₂₈H₂₅F₂NO₆ 509.51 | 0.030 ± 0.002 (n ≥ 3) |
| 4g UCAB#1119 | 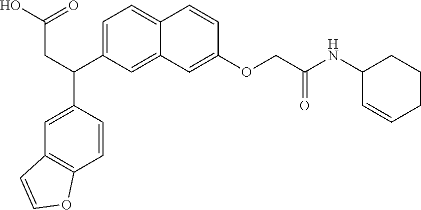 [SMILES: O=C(O)CC(c1ccc2ccc(OCC(=O)NC3C=CCCC3)cc2c1)c1ccc2occc2c1] | C₂₉H₂₇NO₅ 469.54 | 0.021 ± 0.002 (n ≥ 3) |
| 4h UCAB#1102 | 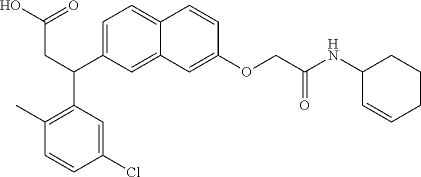 [SMILES: Cc1ccc(Cl)cc1C(CC(=O)O)c1ccc2ccc(OCC(=O)NC3C=CCCC3)cc2c1] | C₂₈H₂₈ClNO₄ 477.99 | 0.048 ± 0.005 (n ≥ 3) |
| 4i UCAB#1123 | 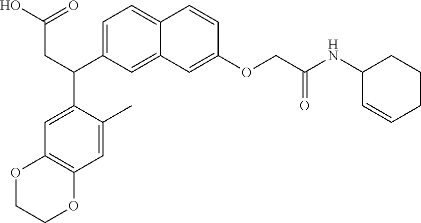 [SMILES: Cc1cc2c(cc1C(CC(=O)O)c1ccc3ccc(OCC(=O)NC4C=CCCC4)cc3c1)OCCO2] | C₃₀H₃₁NO₆ 501.58 | 0.017 ± 0.003 (n ≥ 3) |
| 4j UCAB#1126 | 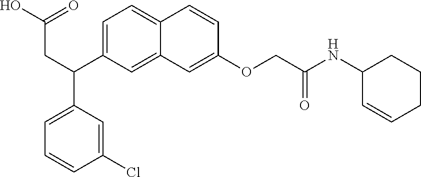 [SMILES: O=C(O)CC(c1cccc(Cl)c1)c1ccc2ccc(OCC(=O)NC3C=CCCC3)cc2c1] | C₂₇H₂₆ClNO₄ 463.96 | 0.206 ± 0.015 (n ≥ 3) |
| 4k UCAB#1127 | 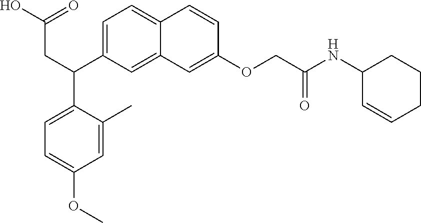 [SMILES: COc1ccc(C(CC(=O)O)c2ccc3ccc(OCC(=O)NC4C=CCCC4)cc3c2)c(C)c1] | C₂₉H₃₁NO₅ 473.57 | 0.017 ± 0.004 (n ≥ 3) |
| 4l UCAB#1128 | 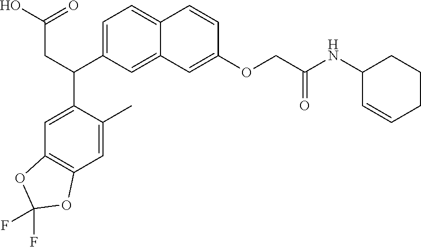 [SMILES: Cc1cc2c(cc1C(CC(=O)O)c1ccc3ccc(OCC(=O)NC4C=CCCC4)cc3c1)OC(F)(F)O2] | C₂₉H₂₇F₂NO₆ 523.53 | 0.016 ± 0.005 (n ≥ 3) |
| 4m UCAB#1192 | 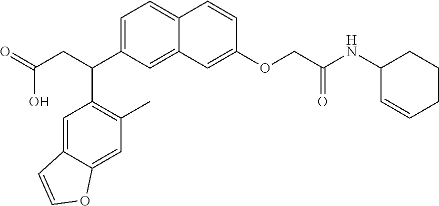 [SMILES: Cc1cc2occc2cc1C(CC(=O)O)c1ccc2ccc(OCC(=O)NC3C=CCCC3)cc2c1] | C₃₀H₂₉NO₅ 483.56 | 0.018 ± 0.003 (n ≥ 3) |
| 4n UCAB#1194 | 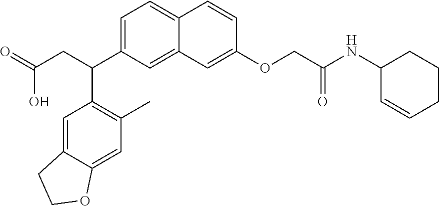 [SMILES: Cc1cc2c(cc1C(CC(=O)O)c1ccc3ccc(OCC(=O)NC4C=CCCC4)cc3c1)CCO2] | C₃₀H₃₁NO₅ 485.58 | 0.018 ± 0.002 (n ≥ 3) |

Scheme 6 compounds

| | | | |
|---|---|---|---|
| 5a UCAB#1182 | 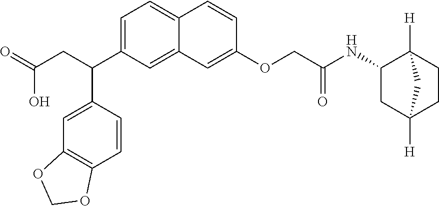 [SMILES: O=C(O)CC(c1ccc2c(c1)OCO2)c1ccc2ccc(OCC(=O)N[C@H]3C[C@@H]4CC[C@H]3C4)cc2c1] | C₂₉H₂₉NO₆ 487.55 | 0.020 ± 0.003 (n ≥ 3) |
| 5b UCAB#1245 | 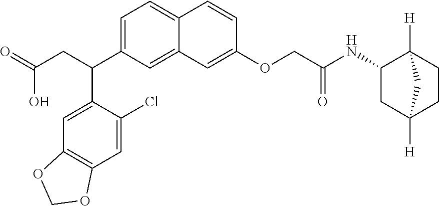 [SMILES: O=C(O)CC(c1ccc2ccc(OCC(=O)N[C@H]3C[C@@H]4CC[C@H]3C4)cc2c1)c1cc2c(cc1Cl)OCO2] | C₂₉H₂₈ClNO₆ 521.99 | 0.004 (n = 1) |
| 5c UCAB#1256 | 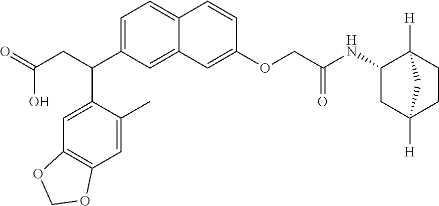 [SMILES: Cc1cc2c(cc1C(CC(=O)O)c1ccc3ccc(OCC(=O)N[C@H]4C[C@@H]5CC[C@H]4C5)cc3c1)OCO2] | C₃₀H₃₁NO₆ 501.58 | 0.007 (n = 1) |
| 5d UCAB#1257 | 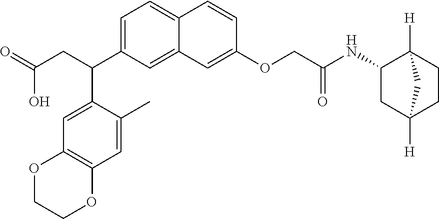 [SMILES: Cc1cc2c(cc1C(CC(=O)O)c1ccc3ccc(OCC(=O)N[C@H]4C[C@@H]5CC[C@H]4C5)cc3c1)OCCO2] | C₃₁H₃₃NO₆ 515.61 | ~0.001 (n = 1) |

Scheme 7 compounds

| | | | |
|---|---|---|---|
| 6a UCAB#1000 | 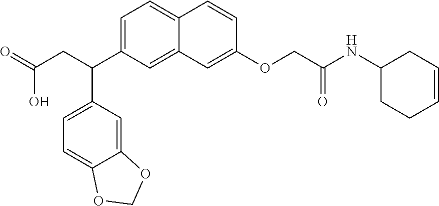 [SMILES: O=C(O)CC(c1ccc2c(c1)OCO2)c1ccc2ccc(OCC(=O)NC3CC=CCC3)cc2c1] | C₂₈H₂₇NO₆ 473.53 | 0.076 ± 0.009 (n ≥ 3) |
| 6b UCAB#1131 | 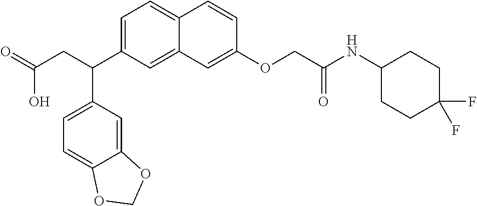 [SMILES: O=C(O)CC(c1ccc2c(c1)OCO2)c1ccc2ccc(OCC(=O)NC3CCC(F)(F)CC3)cc2c1] | C₂₈H₂₇F₂NO₆ 511.52 | 0.080 ± 0.01 (n ≥ 3) |
| 6c UCAB#1164 | 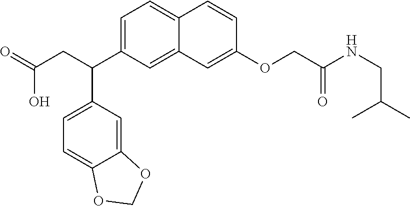 [SMILES: CC(C)CNC(=O)COc1ccc2ccc(C(CC(=O)O)c3ccc4c(c3)OCO4)cc2c1] | C₂₆H₂₇NO₆ 449.50 | 0.138 ± 0.024 (n ≥ 3) |
| 6d UCAB#1165 | 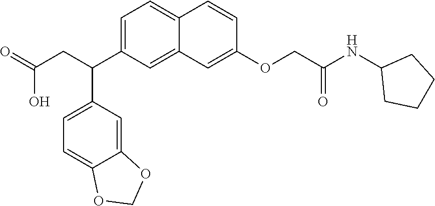 [SMILES: O=C(O)CC(c1ccc2c(c1)OCO2)c1ccc2ccc(OCC(=O)NC3CCCC3)cc2c1] | C₂₇H₂₇NO₆ 461.51 | 0.062 ± 0.005 (n ≥ 3) |
| 6e UCAB#1167 | 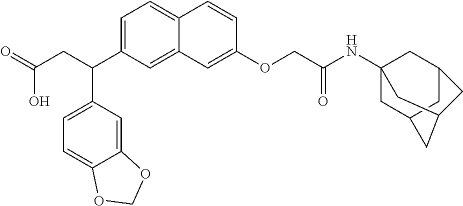 [SMILES: O=C(O)CC(c1ccc2c(c1)OCO2)c1ccc2ccc(OCC(=O)NC34CC5CC(CC(C5)C3)C4)cc2c1] | C₃₂H₃₃NO₆ 527.62 | 0.516 ± 0.115 (n ≥ 3) |
| 6f UCAB#1168 | 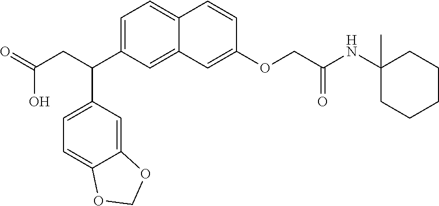 [SMILES: CC1(NC(=O)COc2ccc3ccc(C(CC(=O)O)c4ccc5c(c4)OCO5)cc3c2)CCCCC1] | C₂₉H₃₁NO₆ 489.57 | 0.477 ± 0.07 (n ≥ 3) |
| 6g UCAB#1176 | 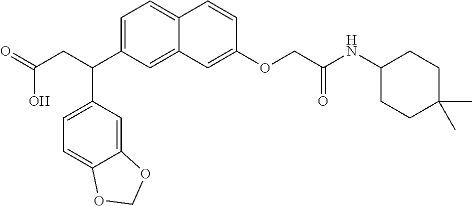 [SMILES: CC1(C)CCC(NC(=O)COc2ccc3ccc(C(CC(=O)O)c4ccc5c(c4)OCO5)cc3c2)CC1] | C₃₀H₃₃NO₆ 503.60 | 0.056 ± 0.007 (n ≥ 3) |
| 6h UCAB#1188 | 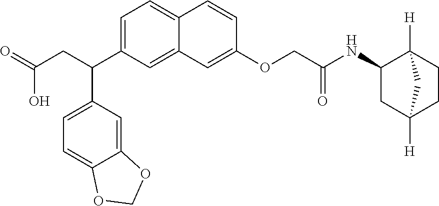 [SMILES: O=C(O)CC(c1ccc2c(c1)OCO2)c1ccc2ccc(OCC(=O)N[C@@H]3C[C@@H]4CC[C@H]3C4)cc2c1] | C₂₉H₂₉NO₆ 487.55 | 0.019 ± 0.003 (n ≥ 3) |
| 6i UCAB#1202-P1 | 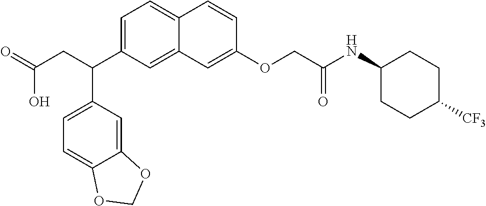 [SMILES: O=C(O)CC(c1ccc2c(c1)OCO2)c1ccc2ccc(OCC(=O)N[C@H]3CC[C@H](C(F)(F)F)CC3)cc2c1] | C₂₉H₂₈F₃NO₆ 543.54 | 0.397 ± 0.041 (n ≥ 3) |
| 6j UCAB#1202-P2 | 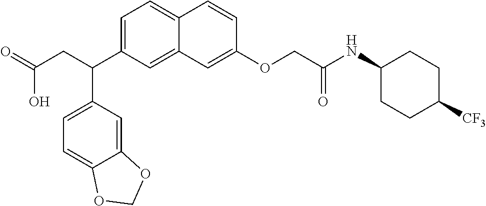 [SMILES: O=C(O)CC(c1ccc2c(c1)OCO2)c1ccc2ccc(OCC(=O)N[C@H]3CC[C@@H](C(F)(F)F)CC3)cc2c1] | C₂₉H₂₈F₃NO₆ 543.54 | 0.045 ± 0.001 (n ≥ 3) |
| 6k UCAB#1224 | 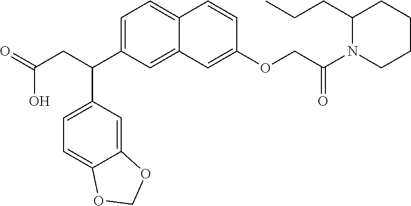 [SMILES: CCCC1CCCCN1C(=O)COc1ccc2ccc(C(CC(=O)O)c3ccc4c(c3)OCO4)cc2c1] | C₃₀H₃₃NO₆ 503.60 | 0.063 ± 0.003 (n = 3) |
| 6l UCAB#1247 | 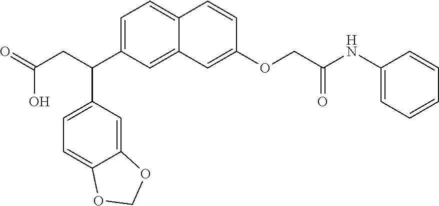 [SMILES: O=C(O)CC(c1ccc2c(c1)OCO2)c1ccc2ccc(OCC(=O)Nc3ccccc3)cc2c1] | C₂₈H₂₃NO₆ 469.49 | 1.88 (n = 1) |

Scheme 8 compounds

| | | | |
|---|---|---|---|
| 7a UCAB#1002 | 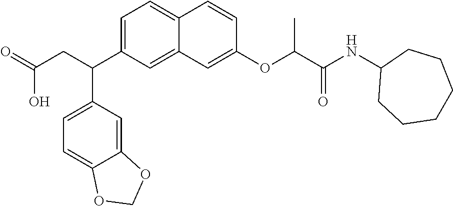 [SMILES: CC(Oc1ccc2ccc(C(CC(=O)O)c3ccc4c(c3)OCO4)cc2c1)C(=O)NC1CCCCCC1] | C₃₀H₃₃NO₆ 503.60 | 0.153 ± 0.012 (n ≥ 3) |
| 7b UCAB#1132 | 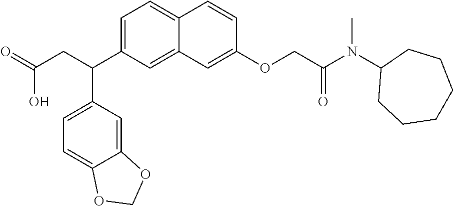 [SMILES: CN(C(=O)COc1ccc2ccc(C(CC(=O)O)c3ccc4c(c3)OCO4)cc2c1)C1CCCCCC1] | C₃₀H₃₃NO₆ 503.60 | 0.129 ± 0.009 (n ≥ 3) |
| 7c UCAB#1227 | 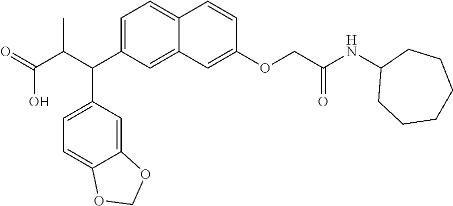 [SMILES: CC(C(=O)O)C(c1ccc2c(c1)OCO2)c1ccc2ccc(OCC(=O)NC3CCCCCC3)cc2c1] | C₃₀H₃₃NO₆ 503.60 | 0.015 ± 0.001 (n = 2) |
| 7d UCAB#1228 | 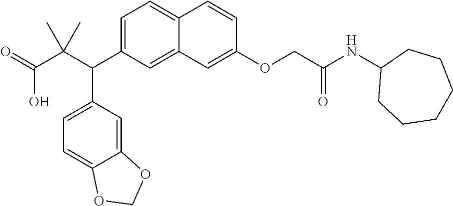 [SMILES: CC(C)(C(=O)O)C(c1ccc2c(c1)OCO2)c1ccc2ccc(OCC(=O)NC3CCCCCC3)cc2c1] | C₃₁H₃₅NO₆ 517.62 | 0.003 ± 0.001 (n = 2) |

Scheme 9 compounds

| | | | |
|---|---|---|---|
| 8a UCAB#1133 | 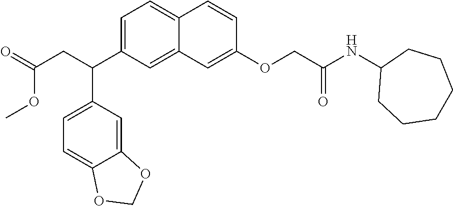 [SMILES: COC(=O)CC(c1ccc2c(c1)OCO2)c1ccc2ccc(OCC(=O)NC3CCCCCC3)cc2c1] | C₃₀H₃₃NO₆ 503.60 | — |
| 8b UCAB#1193 | 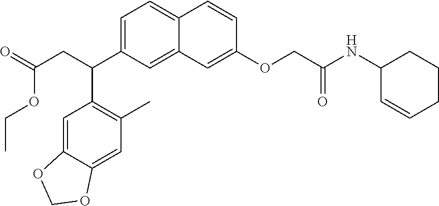 [SMILES: CCOC(=O)CC(c1ccc2ccc(OCC(=O)NC3C=CCCC3)cc2c1)c1cc2c(cc1C)OCO2] | C₃₁H₃₃NO₆ 515.61 | — |
| 8c UCAB#1229 | 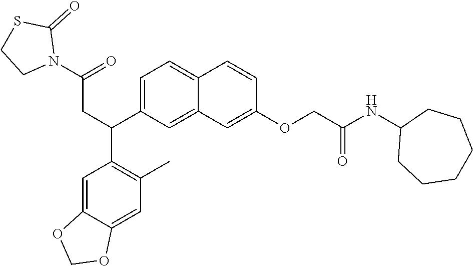 [SMILES: Cc1cc2c(cc1C(CC(=O)N1CCSC1=O)c1ccc3ccc(OCC(=O)NC4CCCCCC4)cc3c1)OCO2] | C₃₃H₃₆N₂O₆S 588.72 | — |
| 8d UCAB#1231 | 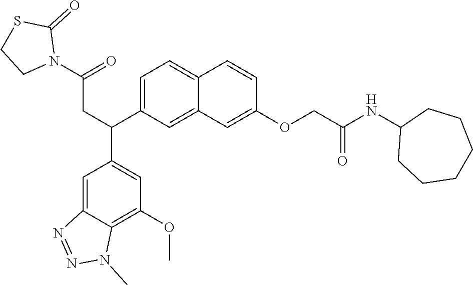 [SMILES: COc1cc(C(CC(=O)N2CCSC2=O)c2ccc3ccc(OCC(=O)NC4CCCCCC4)cc3c2)cc2nnn(C)c12] | C₃₃H₃₇N₅O₅S 615.75 | — |

Scheme 10 compounds

| | | | |
|---|---|---|---|
| 9a UCAB#1222 | 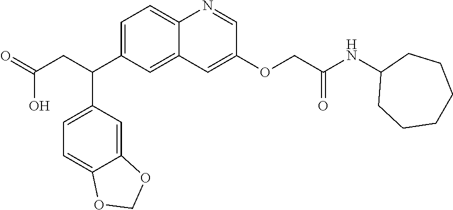 [SMILES: O=C(O)CC(c1ccc2c(c1)OCO2)c1ccc2ncc(OCC(=O)NC3CCCCCC3)cc2c1] | C₂₈H₃₀N₂O₆ 490.56 | 0.522 ± 0.067 (n = 2) |
| 9b UCAB#1235 | 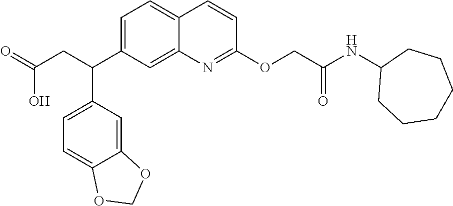 [SMILES: O=C(O)CC(c1ccc2c(c1)OCO2)c1ccc2ccc(OCC(=O)NC3CCCCCC3)nc2c1] | C₂₈H₃₀N₂O₆ 490.56 | 0.083 ± 0.037 (n = 2) |
| 9c UCAB#1258 | 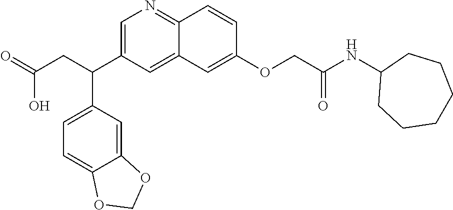 [SMILES: O=C(O)CC(c1ccc2c(c1)OCO2)c1cnc2ccc(OCC(=O)NC3CCCCCC3)cc2c1] | C₂₈H₃₀N₂O₆ 490.56 | 0.064 (n = 1) |

### REFERENCES

1. Bach, A., Targeting Oxidative Stress in Stroke. In Neuroprotective Therapy for Stroke and Ischemic Disease, Lapchak, P. A.; Zhang, J. H., Eds. Springer: 2017; pp 203-250.
2. Casas, A. I.; Nogales, C.; Mucke, H. A. M.; Petraina, A.; Cuadrado, A.; Rojo, A. I.; Ghezzi, P.; Jaquet, V.; Augsburger, F.; Dufrasne, F.; Soubhye, J.; Deshwal, S.; Di Sante, M.; Kaludercic, N.; Di Lisa, F.; Schmidt, H., On the Clinical Pharmacology of Reactive Oxygen Species. Pharmacol Rev 2020, 72 (4), 801-828.
3. Baird, L.; Yamamoto, M., The Molecular Mechanisms Regulating the KEAP1-NRF2 Pathway. Mol Cell Biol 2020, 40 (13).
4. Pallesen, J. S.; Tran, K. T.; Bach, A., Non-covalent Small-Molecule Kelch-like ECH-Associated Protein 1-Nuclear Factor Erythroid 2-Related Factor 2 (Keap1-Nrf2) Inhibitors and Their Potential for Targeting Central Nervous System Diseases. J Med Chem 2018, 61 (18), 8088-8103.
5. Horie, Y.; Suzuki, T.; Inoue, J.; Iso, T.; Wells, G.; Moore, T. W.; Mizushima, T.; Dinkova-Kostova, A. T.; Kasai, T.; Kamei, T.; Koshiba, S.; Yamamoto, M., Molecular basis for the disruption of Keap1-Nrf2 interaction via Hinge & Latch mechanism. Commun Biol 2021, 4 (1), 576.
6. Cuadrado, A.; Rojo, A. I.; Wells, G.; Hayes, J. D.; Cousin, S. P.; Rumsey, W. L.; Attucks, O. C.; Franklin, S.; Levonen, A. L.; Kensler, T. W.; Dinkova-Kostova, A. T., Therapeutic targeting of the NRF2 and KEAP1 partnership in chronic diseases. Nat Rev Drug Discov 2019, 18 (4), 295-317.
7. Barreca, M.; Qin, Y.; Cadot, M. E. H.; Barraja, P.; Bach, A., Advances in developing noncovalent small molecules targeting Keap1. Drug Discov Today 2023, 28 (12), 103800.
8. Magesh, S.; Chen, Y.; Hu, L., Small molecule modulators of Keap1-Nrf2-ARE pathway as potential preventive and therapeutic agents. Med Res Rev 2012, 32 (4), 687-726.
9. Lu, M. C.; Ji, J. A.; Jiang, Z. Y.; You, Q. D., The Keap1-Nrf2-ARE Pathway As a Potential Preventive and Therapeutic Target: An Update. Med Res Rev 2016, 36 (5), 924-963.
10. Tu, J.; Zhang, X.; Zhu, Y.; Dai, Y.; Li, N.; Yang, F.; Zhang, Q.; Brann, D. W.; Wang, R., Cell-Permeable Peptide Targeting the Nrf2-Keap1 Interaction: A Potential Novel Therapy for Global Cerebral Ischemia. J Neurosci 2015, 35 (44), 14727-39.
11. Zhao, X.; Sun, G.; Zhang, J.; Ting, S. M.; Gonzales, N.; Aronowski, J., Dimethyl Fumarate Protects Brain From Damage Produced by Intracerebral Hemorrhage by Mechanism Involving Nrf2. Stroke 2015, 46 (7), 1923-1928.
12. Shih, A. Y.; Li, P.; Murphy, T. H., A small-molecule-inducible Nrf2-mediated antioxidant response provides effective prophylaxis against cerebral ischemia in vivo. Journal of Neuroscience 2005, 25 (44), 10321-10335.
13. Bach, A., Targeting Oxidative Stress in Stroke. In Neuroprotective Therapy for Stroke and Ischemic Disease, Lapchak, P. Z. L., Ed. Springer Series in Translational Stroke Research, 2017.
14. Alfieri, A.; Srivastava, S.; Siow, R. C.; Modo, M.; Fraser, P. A.; Mann, G. E., Targeting the Nrf2-Keap1 antioxidant defence pathway for neurovascular protection in stroke. J Physiol 2011, 589 (17), 4125-36.
15. Zhao, J.; Redell, J. B.; Moore, A. N.; Dash, P. K., A novel strategy to activate cytoprotective genes in the injured brain. Biochemical and Biophysical Research Communications 2011, 407 (3), 501-506.
16. Hui, Q. Y.; Karlstetter, M.; Xu, Z. H.; Yang, J.; Zhou, L. L.; Eilken, H. M.; Terjung, C.; Cho, H.; Gong, J. S.; Lai, M. J.; Nassar, K.; Duh, E. J., Inhibition of the Keap1-Nrf2 protein-protein interaction protects retinal cells and ameliorates retinal ischemia-reperfusion injury. Free Radical Bio Med 2020, 146, 181-188.
17. Davies, D. A.; Adlimoghaddam, A.; Albensi, B. C., Role of Nrf2 in Synaptic Plasticity and Memory in Alzheimer's Disease. Cells 2021, 10 (8).
18. Sun, Y.; Huang, J. X.; Chen, Y. F.; Shang, H.; Zhang, W. N.; Yu, J. Q.; He, L.; Xing, C. G.; Zhuang, C. L., Direct inhibition of Keap1-Nrf2 Protein-Protein interaction as a potential therapeutic strategy for Alzheimer's disease. Bioorg Chem 2020, 103.
19. Johnson, J. A.; Johnson, D. A.; Kraft, A. D.; Calkins, M. J.; Jakel, R. J.; Vargas, M. R.; Chen, P. C., The Nrf2-ARE pathway: an indicator and modulator of oxidative stress in neurodegeneration. Ann N Y Acad Sci 2008, 1147, 61-9.
20. Davies, T. G.; Wixted, W. E.; Coyle, J. E.; Griffiths-Jones, C.; Hearn, K.; McMenamin, R.; Norton, D.; Rich, S. J.; Richardson, C.; Saxty, G.; Willems, H. M.; Woolford, A. J.; Cottom, J. E.; Kou, J. P.; Yonchuk, J. G.; Feldser, H. G.; Sanchez, Y.; Foley, J. P.; Bolognese, B. J.; Logan, G.; Podolin, P. L.; Yan, H.; Callahan, J. F.; Heightman, T. D.; Kerns, J. K., Monoacidic Inhibitors of the Kelch-like ECH-Associated Protein 1: Nuclear Factor Erythroid 2-Related Factor 2 (KEAP1: NRF2) Protein-Protein Interaction with High Cell Potency Identified by Fragment-Based Discovery. J Med Chem 2016.
21. Olagnier, D.; Farahani, E.; Thyrsted, J.; Blay-Cadanet, J.; Herengt, A.; Idorn, M.; Hait, A.; Hernaez, B.; Knudsen, A.; Iversen, M. B.; Schilling, M.; Jorgensen, S. E.; Thomsen, M.; Reinert, L. S.; Lappe, M.; Hoang, H. D.; Gilchrist, V. H.; Hansen, A. L.; Ottosen, R.; Nielsen, C. G.; Moller, C.; van der Horst, D.; Peri, S.; Balachandran, S.; Huang, J.; Jakobsen, M.; Svenningsen, E. B.; Poulsen, T. B.; Bartsch, L.; Thielke, A. L.; Luo, Y.; Alain, T.; Rehwinkel, J.; Alcami, A.; Hiscott, J.; Mogensen, T. H.; Paludan, S. R.; Holm, C. K., SARS-CoV2-mediated suppression of NRF2-signaling reveals potent antiviral and anti-inflammatory activity of 4-octyl-itaconate and dimethyl fumarate. Nat Commun 2020, 11 (1), 4938.
22. Yan, J.; Li, Y.; Ding, L.; Hou, R.; Xing, C.; Jiang, C.; Miao, Z.; Zhuang, C., Fragment-Based Discovery of Azocyclic Alkyl Naphthalenesulfonamides as Keap1-Nrf2 Inhibitors for Acute Lung Injury Treatment. J Med Chem 2023, 66 (12), 8267-8280.
23. Seedorf, K.; Weber, C.; Vinson, C.; Berger, S.; Vuillard, L. M.; Kiss, A.; Creusot, S.; Broux, O.; Geant, A.; Ilic, C.; Lemaitre, K.; Richard, J.; Hammoutene, A.; Mahieux, J.; Martiny, V.; Durand, D.; Melchiore, F.; Nyerges, M.; Paradis, V.; Provost, N.; Duvivier, V.; Delerive, P., Selective disruption of NRF2-KEAP1 interaction leads to NASH resolution and reduction of liver fibrosis in mice. JHEP Rep 2023, 5 (4), 100651.
24. Hammoutene, A.; Laouirem, S.; Albuquerque, M.; Colnot, N.; Brzustowski, A.; Valla, D.; Provost, N.; Delerive, P.; Paradis, V., A new NRF2 activator for the treatment of human metabolic dysfunction associated fatty liver disease. JHEP Reports 2023.
25. Hassanein, E. H. M.; Ibrahim, I. M.; Abd-alhameed, E. K.; Sharawi, Z. W.; Jaber, F. A.; Althagafy, H. S., Nrf2/HO-1 as a therapeutic target in renal fibrosis. Life Sciences 2023, 334, 122209.
26. Kaseda, S.; Sannomiya, Y.; Horizono, J.; Kuwazuru, J.; Suico, M. A.; Ogi, S.; Sasaki, R.; Sunamoto, H.; Fukiya, H.; Nishiyama, H.; Kamura, M.; Niinou, S.; Koyama, Y.; Nara, F.; Shuto, T.; Onuma, K.; Kai, H., Novel Keap1-Nrf2 Protein-Protein Interaction Inhibitor UBE-1099 Ameliorates Progressive Phenotype in Alport Syndrome Mouse Model. Kidney360 2022, 3 (4), 687-699.
27. Ogawa, T.; Ishitsuka, Y., The Role of KEAP1-NRF2 System in Atopic Dermatitis and Psoriasis. Antioxidants (Basel) 2022, 11 (7).
28. Manda, G.; Milanesi, E.; Genc, S.; Niculite, C. M.; Neagoe, I. V.; Tastan, B.; Dragnea, E. M.; Cuadrado, A., Pros and cons of NRF2 activation as adjunctive therapy in rheumatoid arthritis. Free Radic Biol Med 2022, 190, 179-201.
29. Lu, M. C.; Ji, J. A.; Jiang, Y. L.; Chen, Z. Y.; Yuan, Z. W.; You, Q. D.; Jiang, Z. Y., An inhibitor of the Keap1-Nrf2 protein-protein interaction protects NCM460 colonic cells and alleviates experimental colitis. Sci Rep-Uk 2016, 6.
30. Lu, M. C.; Zhang, X.; Wu, F.; Tan, S. J.; Zhao, J.; You, Q. D.; Jiang, Z. Y., Discovery of a Potent Kelch-Like ECH-Associated Protein 1-Nuclear Factor Erythroid 2-Related Factor 2 (Keap1-Nrf2) Protein-Protein Interaction Inhibitor with Natural Proline Structure as a Cytoprotective Agent against Acetaminophen-Induced Hepatotoxicity. J Med Chem 2019, 62 (14), 6796-6813.
31. Linker, R. A.; Lee, D. H.; Ryan, S.; van Dam, A. M.; Conrad, R.; Bista, P.; Zeng, W.; Hronowsky, X.; Buko, A.; Chollate, S.; Ellrichmann, G.; Bruck, W.; Dawson, K.; Goelz, S.; Wiese, S.; Scannevin, R. H.; Lukashev, M.; Gold, R., Fumaric acid esters exert neuroprotective effects in neuroinflammation via activation of the Nrf2 antioxidant pathway. Brain 2011, 134, 678-692.
32. Prosperini, L.; Pontecorvo, S., Dimethyl fumarate in the management of multiple sclerosis: appropriate patient selection and special considerations. Therapeutics and Clinical Risk Management 2016, 12, 339-350.
33. Blewett, M. M.; Xie, J. J.; Zaro, B. W.; Backus, K. M.; Altman, A.; Teijaro, J. R.; Cravatt, B. F., Chemical proteomic map of dimethyl fumarate-sensitive cysteines in primary human T cells. Science Signaling 2016, 9 (445).
34. Zhou, H.; Wang, Y.; You, Q.; Jiang, Z., Recent progress in the development of small molecule Nrf2 activators: a patent review (2017-present). Expert opinion on therapeutic patents 2020, 30 (3), 209-225.
35. Tran, K. T.; Pallesen, J. S.; Solbak, S. M. O.; Narayanan, D.; Baig, A.; Zang, J.; Aguayo-Orozco, A.; Carmona, R. M. C.; Garcia, A. D.; Bach, A., A Comparative Assessment Study of Known Small-Molecule Keap 1-Nrf2 Protein-Protein Interaction Inhibitors: Chemical Synthesis, Binding Properties, and Cellular Activity. J Med Chem 2019, 62 (17), 8028-8052.
36. Pallesen, J. S.; Narayanan, D.; Tran, K. T.; Solbak, S. M. O.; Marseglia, G.; Sorensen, L. M. E.; Hoj, L. J.; Munafo, F.; Carmona, R. M. C.; Garcia, A. D.; Desu, H. L.; Brambilla, R.; Johansen, T. N.; Popowicz, G. M.; Sattler, M.; Gajhede, M.; Bach, A., Deconstructing Noncovalent Kelch-like ECH-Associated Protein 1 (Keap1) Inhibitors into Fragments to Reconstruct New Potent Compounds. J Med Chem 2021, 64 (8), 4623-4661.
37. Narayanan, D.; Tran, K. T.; Pallesen, J. S.; Solbak, S. M. O.; Qin, Y.; Mukminova, E.; Luchini, M.; Vasilyeva, K. O.; Gonzalez Chichon, D.; Goutsiou, G.; Poulsen, C.; Haapanen, N.; Popowicz, G. M.; Sattler, M.; Olagnier, D.; Gajhede, M.; Bach, A., Development of Noncovalent Small-Molecule Keap1-Nrf2 Inhibitors by Fragment-Based Drug Discovery. J Med Chem 2022.
38. Heightman, T. D.; Callahan, J. F.; Chiarparin, E.; Coyle, J. E.; Griffiths-Jones, C.; Lakdawala, A. S.; McMenamin, R.; Mortenson, P. N.; Norton, D.; Peakman, T. M.; Rich, S. J.; Richardson, C.; Rumsey, W. L.; Sanchez, Y.; Saxty, G.; Willems, H. M. G.; Wolfe, L., 3rd; Woolford, A. J.; Wu, Z.; Yan, H.; Kerns, J. K.; Davies, T. G., Structure-Activity and Structure-Conformation Relationships of Aryl Propionic Acid Inhibitors of the Kelch-like ECH-Associated Protein 1/Nuclear Factor Erythroid 2-Related Factor 2 (KEAP1/NRF2) Protein-Protein Interaction. J Med Chem 2019, 62 (9), 4683-4702.
39. Erlanson, D. A.; Fesik, S. W.; Hubbard, R. E.; Jahnke, W.; Jhoti, H., Twenty years on: the impact of fragments on drug discovery. Nat Rev Drug Discov 2016, 15 (9), 605-19.
40. Kirsch, P.; Hartman, A. M.; Hirsch, A. K. H.; Empting, M., Concepts and Core Principles of Fragment-Based Drug Design. Molecules 2019, 24 (23).
41. Norton, D.; Bonnette, W. G.; Callahan, J. F.; Carr, M. G.; Griffiths-Jones, C. M.; Heightman, T. D.; Kerns, J. K.; Nie, H.; Rich, S. J.; Richardson, C.; Rumsey, W.; Sanchez, Y.; Verdonk, M. L.; Willems, H. M. G.; Wixted, W. E.; Wolfe, L., 3rd; Woolford, A. J.; Wu, Z.; Davies, T. G., Fragment-Guided Discovery of Pyrazole Carboxylic Acid Inhibitors of the Kelch-like ECH-Associated Protein 1: Nuclear Factor Erythroid 2 Related Factor 2 (KEAP1: NRF2) Protein-Protein Interaction. J Med Chem 2021, 64 (21), 15949-15972.
42. Rees, D. C.; Congreve, M.; Murray, C. W.; Carr, R., Fragment-based lead discovery. Nat Rev Drug Discov 2004, 3 (8), 660-72.
43. Nikolovska-Coleska, Z.; Wang, R.; Fang, X.; Pan, H.; Tomita, Y.; Li, P.; Roller, P. P.; Krajewski, K.; Saito, N. G.; Stuckey, J. A.; Wang, S., Development and optimization of a binding assay for the XIAP BIR3 domain using fluorescence polarization. Anal Biochem 2004, 332 (2), 261-273.

1. A compound of Formula (XI): 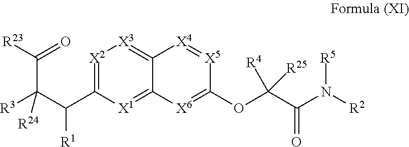
wherein
X¹ is CH or N;
X² is CH or N;
X³ is CH or N;
X⁴ is CH or N;
X⁵ is CH or N;
X⁶ is CH or N;
R¹ is phenyl, pyridinyl or H, wherein the phenyl or pyridinyl is optionally substituted with one or more, identical or different, substituents R⁶, wherein two of the substituents R⁶ optionally are linked together to form a ring;
R² is selected from the group consisting of C₅₋₇ cycloalkyl, C₅₋₇ cycloalkenyl, C₅₋₇ heterocycloalkyl, phenyl, C₅₋₈ bicycloalkyl, C₃₋₆ alkyl, and C₉₋₁₂ tricycloalkyl, wherein said C₅₋₇ cycloalkyl, C₅₋₇ cycloalkenyl, C₅₋₇ heterocycloalkyl, phenyl, C₅₋₈ bicycloalkyl, C₃₋₆ alkyl, and C₉₋₁₂ tricycloalkyl is optionally substituted with one or more, identical of different substituents R²⁸;
R⁵ is H or CH₃;
or R² is C₃₋₆ alkanediyl optionally substituted with R²⁸, R⁵ is a bond, and R² and R⁵ are linked together to form a ring;
R³ is H or CH₃;
R⁴ is H or CH₃;
R⁶ is selected from the group consisting of C₁₋₅ alkyl, halogen, —OH, —OC₁₋₅ alkyl and C₂₋₅ alkenyl,
wherein said C₁₋₅ alkyl, —OC₁₋₅ alkyl and C₂₋₅ alkenyl are optionally substituted with one or more substituents individually selected from C₁₋₃ alkyl and halogen;
wherein one or more methylene group of the C₁₋₅ alkyl, —OC₁₋₅ alkyl, or C₂₋₅ alkenyl is optionally replaced with O or N(R⁷); and/or
wherein one or more methine group of the C₂₋₅ alkenyl is optionally replaced with N;
R⁷ is H or C₁₋₃ alkyl;
R²³ is selected from the group consisting of —OH; 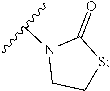 [SMILES: *N1CCSC1=O]
—OC₁₋₅ alkyl optionally substituted with one or more, identical or different, substituents R²⁶;—OC₂₋₅ alkenyl optionally substituted with one or more, identical or different, substituents R²⁶; —OC₂₋₅ alkynyl optionally substituted with one or more, identical or different, substituents R²⁶; —OC₃₋₆ cycloalkyl optionally substituted with one or more, identical or different, substituents R²⁶; —O-phenyl optionally substituted with one or more, identical or different, substituents R²⁷; and —O-benzyl optionally substituted with one or more, identical or different, substituents R²⁷; and —NH(CH₃);
R²⁴ is H or CH₃;
R²⁵ is H or CH₃;
R²⁶ is independently selected from the group consisting of H, deuterium, F, Cl and —OC₁₋₃ alkyl;
R²⁷ is independently selected from the group consisting of deuterium, methoxy, nitro, cyano, Cl, Br, I, and F; and
R²⁸ is individually selected from C₁₋₃ alkyl, —OC₁₋₃ alkyl, halogen, and deuterium, wherein said C₁₋₃ alkyl is optionally substituted with one or more halogens;
or a pharmaceutically acceptable salt, hydrate, polymorph, tautomer, or solvate thereof.

2. The compound according to claim 1, wherein:
X¹ is CH or N;
X² is CH or N;
X³ is CH or N;
X⁴ is CH or N;
X⁵ is CH or N;
X⁶ is CH or N;
R¹ is phenyl, pyridinyl or H, wherein the phenyl or pyridinyl is optionally substituted with one or more, identical or different, substituents R⁶, wherein two of the substituents R⁶ optionally are linked together to form a ring;
R² is selected from the group consisting of C₅₋₇ cycloalkyl, C₅₋₇ cycloalkenyl C₅₋₇ heterocycloalkyl and phenyl,
wherein said C₅₋₇ cycloalkyl, C₅₋₇ cycloalkenyl C₅₋₇ heterocycloalkyl and phenyl is optionally substituted with one or more substituents individually selected from C₁₋₃ alkyl, —OC₁₋₃ alkyl, halogen and deuterium,
wherein said C₁₋₃ alkyl is optionally substituted with one or more halogens;
R³ is H or CH₃;
R⁴ is H or CH₃;
R⁵ is H or CH₃;
R⁶ is selected from the group consisting of C₁₋₅ alkyl, halogen, —OH, —OC₁₋₅ alkyl and C₂₋₅ alkenyl,
wherein said C₁₋₅ alkyl, —OC₁₋₅ alkyl and C₂₋₅ alkenyl are optionally substituted with one or more substituents individually selected from C₁₋₃ alkyl and halogen;
wherein one or more methylene group of the C₁₋₅ alkyl, —OC₁₋₅ alkyl, or C₂₋₅ alkenyl is optionally replaced with O or N(R⁷); and/or
wherein one or more methine group of the C₂₋₅ alkenyl is optionally replaced with N;
R⁷ is H or C₁₋₃ alkyl;
R²³ is selected from the group consisting of —OH; 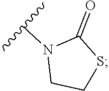 [SMILES: *N1CCSC1=O]
—OC₁₋₅ alkyl optionally substituted with one or more, identical or different, substituents R²⁶;—OC₂₋₅ alkenyl optionally substituted with one or more, identical or different, substituents R²⁶; —OC₂₋₅ alkynyl optionally substituted with one or more, identical or different, substituents R²⁶; —OC₃₋₆ cycloalkyl optionally substituted with one or more, identical or different, substituents R²⁶; —O-phenyl optionally substituted with one or more, identical or different, substituents R²⁷; and —O-benzyl optionally substituted with one or more, identical or different, substituents R²⁷; and —NH(CH₃); R²⁴ is H or CH₃;
R²⁵ is H or CH₃;
R²⁶ is independently selected from the group consisting of H, deuterium, F, Cl and —OC₁₋₃ alkyl; and
R²⁷ is independently selected from the group consisting of deuterium, methoxy, nitro, cyano, C₁, Br, I, and F;
or a pharmaceutically acceptable salt, hydrate, polymorph, tautomer, or solvate thereof.

3. The compound according to claim 1, wherein of Formula (XI):
X¹ is CH or N;
X² is CH or N;
X³ is CH or N;
X⁴ is CH or N;
X⁵ is CH or N;
X⁶ is CH or N;
R²³ is selected from the group consisting of —OH; 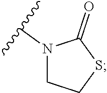 [SMILES: *N1CCSC1=O]
—OC₁₋₅ alkyl optionally substituted with one or more, identical or different, substituents R²⁶;—OC₂₋₅ alkenyl optionally substituted with one or more, identical or different, substituents R²⁶; —OC₂₋₅ alkynyl optionally substituted with one or more, identical or different, substituents R²⁶; —OC₃₋₆ cycloalkyl optionally substituted with one or more, identical or different, substituents R²⁶; —O-phenyl optionally substituted with one or more, identical or different, substituents R²⁷; and —O-benzyl optionally substituted with one or more, identical or different, substituents R²⁷; and —NH(CH₃);
R¹ is selected from the group consisting of Formula (III), Formula (V) and Formula (VI): 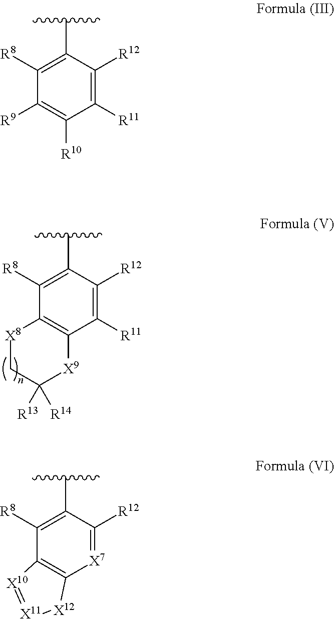
R² is selected from the group consisting of Formula (VIII), Formula (X), C₅₋₈ bicycloalkyl, C₃₋₆ alkyl, and C₉₋₁₂ tricycloalkyl, wherein said bicycloalkyl, C₃₋₆ alkyl, and C₉₋₁₂ tricycloalkyl is optionally substituted with one or more, identical of different substituents R²⁸: 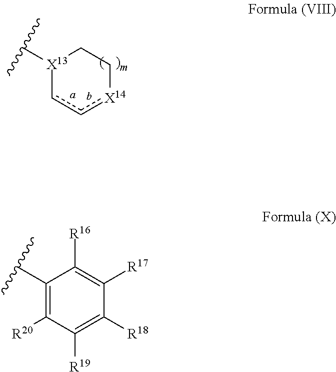
R³ is H or CH₃;
R²⁴ is H or CH₃;
R⁴ is H or CH₃;
R²⁵ is H or CH₃;
R⁵ is H or CH₃;
R⁸ is H or C₁₋₃ alkyl;
R⁹ is selected from the group consisting of H, C₁₋₃ alkyl, halogen, —OH and —OC₁₋₃ alkyl;
R¹⁰ is selected from the group consisting of H, halogen, —OH and —OC₁₋₃ alkyl;
R¹¹ is H;
R¹² is selected from the group consisting of H, C₁₋₃ alkyl and halogen;
R¹³ is selected from the group consisting of H, C₁₋₃ alkyl and halogen;
R¹⁴ is selected from the group consisting of H, C₁₋₃ alkyl and halogen;
R¹⁵ is H or C₁₋₅ alkyl;
R¹⁶ is H or C₁₋₃ alkyl;
R¹⁷ is H or C₁₋₃ alkyl;
R¹⁸ is H or C₁₋₃ alkyl;
R¹⁹ is H or C₁₋₃ alkyl; and
R²⁰ is H or C₁₋₃ alkyl,
R²¹ is selected from the group consisting of H, deuterium, halogen and C₁₋₃ alkyl, wherein said C₁₋₃ alkyl is optionally substituted with one or more halogens;
R²² is selected from the group consisting of H, deuterium, halogen and C₁₋₃ alkyl, wherein said C₁₋₃ alkyl is optionally substituted with one or more halogens;
R²⁶ is independently selected from the group consisting of H, deuterium, F, Cl and —OC₁₋₃ alkyl;
R²⁷ is independently selected from the group consisting of deuterium, methoxy, nitro, cyano, Cl, Br, I, and F; and
R²⁸ is individually selected from C₁₋₃ alkyl, —OC₁₋₃ alkyl, halogen, and deuterium, wherein said C₁₋₃ alkyl is optionally substituted with one or more halogens;
R²⁹ is H or C₁₋₃ alkyl;
X⁷ is C(R¹¹) or N;
X⁸ is O or CH₂;
X⁹ is O;
X¹⁰ is N or CH;
X¹¹ is N or CH;
X¹² is O or N(R¹⁵);
X¹³ is C(R²⁹) or N;
X¹⁴ is (1) C(H) and b is a double bond; (2) C(R²¹)(R²²) and b is a single bond; or (3) O and a is a single bond;
a is a double bond or a single bond;
b is a double bond or a single bond;
n is 0 or 1;
m is 0, 1 or 2;
with the proviso that no more than one of a and b is a double bond.

4. The compound according to claim 1, wherein the compound is of Formula (I): 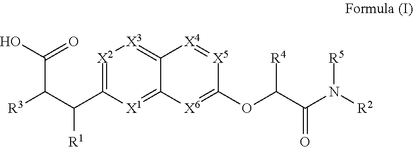
wherein
X¹ is CH or N;
X² is CH or N;
X³ is CH or N;
X⁴ is CH or N;
X⁵ is CH or N;
X⁶ is CH or N;
R¹ is phenyl or pyridinyl optionally substituted with one or more, identical or different, substituents R⁶, wherein two of the substituents R⁶ optionally are linked together to form a ring;
R² is selected from the group consisting of C₅₋₇ cycloalkyl, C₅₋₇ cycloalkenyl C₅₋₇ heterocycloalkyl and phenyl,
wherein said C₅₋₇ cycloalkyl, C₅₋₇ cycloalkenyl C₅₋₇ heterocycloalkyl and phenyl is optionally substituted with one or more substituents individually selected from C₁₋₃ alkyl, —OC₁₋₃ alkyl, halogen and deuterium,
wherein said C₁₋₃ alkyl is optionally substituted with one or more halogens;
R³ is H or CH₃;
R⁴ is H or CH₃;
R⁵ is H or CH₃;
R⁶ is selected from the group consisting of C₁₋₅ alkyl, halogen, —OH, —OC₁₋₅ alkyl and C₂₋₅ alkenyl,
wherein said C₁₋₅ alkyl, —OC₁₋₅ alkyl and C₂₋₅ alkenyl are optionally substituted with one or more substituents individually selected from C₁₋₃ alkyl and halogen;
wherein one or more methylene group of the C₁₋₅ alkyl, —OC₁₋₅ alkyl, or C₂₋₅ alkenyl is optionally replaced with O or N(R⁷); and/or
wherein one or more methine group of the C₂₋₅ alkenyl is optionally replaced with N; and
R⁷ is H or C₁₋₃ alkyl;
or a pharmaceutically acceptable salt, hydrate, polymorph, tautomer, or solvate thereof.

5. The compound according to any one of the preceding claims, wherein X¹, X², X³, X⁴, X⁵ and X⁶ are CH, and R³, R⁴ and R⁵ are H.

6. The compound according to any one of the preceding claims, wherein R¹ is of Formula (II): 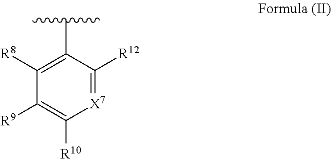
wherein
X⁷ is C(R¹¹) or N;
R⁷ is H or C₁₋₃ alkyl;
R⁸ is H or C₁₋₅ alkyl;
R⁹ is selected from the group consisting of H, C₁₋₅ alkyl, halogen, —OH, —OC₁₋₅ alkyl and C₂₋₅ alkenyl,
wherein said C₁₋₅ alkyl, —OC₁₋₅ alkyl and C₂₋₅ alkenyl are optionally substituted with one or more substituents individually selected from C₁₋₃ alkyl and halogen;
wherein one or more methylene group of the C₁₋₅ alkyl, —OC₁₋₅ alkyl, or C₂₋₅ alkenyl is optionally replaced with O or N(R⁷); and/or
wherein one or more methine group of the C₂₋₅ alkenyl is optionally replaced with N;
R¹⁰ is selected from the group consisting of H, C₁₋₅ alkyl, halogen, —OH, —OC₁₋₅ alkyl and C₂₋₅ alkenyl,
wherein said C₁₋₅ alkyl, —OC₁₋₅ alkyl and C₂₋₅ alkenyl are optionally substituted with one or more substituents individually selected from C₁₋₃ alkyl and halogen;
wherein one or more methylene group of the C₁₋₅ alkyl, —OC₁₋₅ alkyl, or C₂₋₅ alkenyl is optionally replaced with O or N(R⁷); and/or
wherein one or more methine group of the C₂₋₅ alkenyl is optionally replaced with N;
R¹¹ is H or —OC₁₋₅ alkyl;
R¹² is selected from the group consisting of H, C₁₋₅ alkyl, —OC₁₋₅ alkyl, and halogen; and
R⁹ and R¹⁰ are optionally linked together to form a ring.

7. The compound according to any one of the preceding claims, wherein R¹ is of Formula (III): 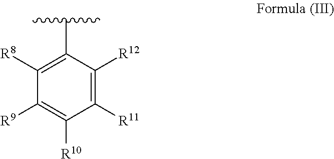
wherein
R⁸ is H or C₁₋₃ alkyl;
R⁹ is selected from the group consisting of H, C₁₋₃ alkyl, halogen, —OH and —OC₁₋₃ alkyl;
R¹⁰ is selected from the group consisting of H, halogen, —OH and —OC₁₋₃ alkyl;
R¹¹ is H; and
R¹² is selected from the group consisting of H, C₁₋₃ alkyl and halogen.

8. The compound according to any one of claims 1 to 5, wherein R¹ is of Formula (V): 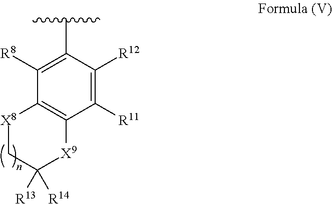
X⁸ is O or CH₂;
X⁹ is O;
R⁸ is H or C₁₋₃ alkyl;
R¹³ is selected from the group consisting of H, C₁₋₃ alkyl and halogen;
R¹⁴ is selected from the group consisting of H, C₁₋₃ alkyl and halogen;
R¹¹ is H;
R¹² is selected from the group consisting of H, C₁₋₅ alkyl, and halogen; and
n is 0 or 1.

9. The compound according to any one of claims 1 to 5, wherein R¹ is of Formula (VI): 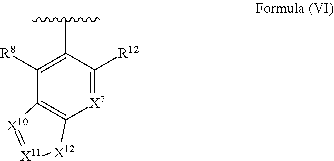
wherein
X⁷ is C(R¹¹) or N;
X¹⁰ is N or CH;
X¹¹ is N or CH;
X¹² is O or N(R¹⁵);
R⁸ is H or C₁₋₅ alkyl;
R¹¹ is H or C₁₋₅ alkyl;
R¹² is H or C₁₋₅ alkyl; and
R¹⁵ is H or C₁₋₅ alkyl.

10. The compound according to any one of claims 1 to 5, wherein R¹ is selected from the group consisting of:
phenyl,

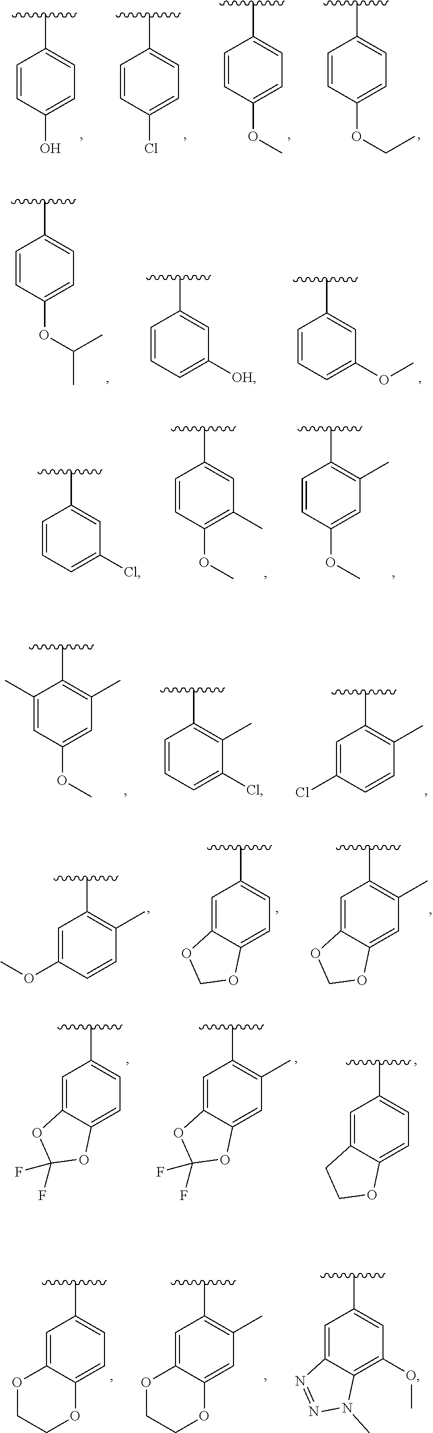

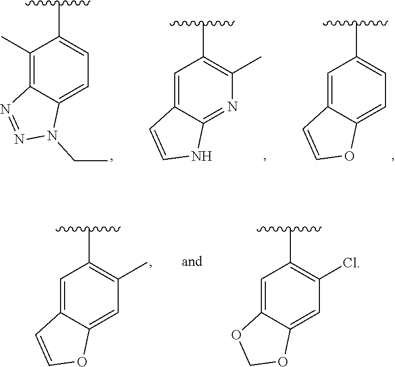 [SMILES: *c1ccc2c(nnn2CC)c1C ; *c1cc2cc[nH]c2nc1C ; *c1ccc2occc2c1 ; *c1cc2ccoc2cc1C ; *c1cc2c(cc1Cl)OCO2]

11. The compound according to any one of the preceding claims, wherein R² is of Formula (VIII): 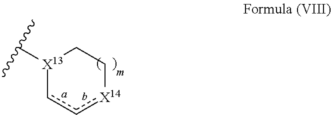
wherein
X¹³ is C(H) or N;
X¹⁴ is (1) C(H) and b is a double bond; (2) C(R²¹)(R²²) and b is a single bond; or (3) O and a is a single bond;
R²¹ is selected from the group consisting of H, deuterium, halogen and C₁₋₃ alkyl, wherein said C₁₋₃ alkyl is optionally substituted with one or more halogens;
R²² is selected from the group consisting of H, deuterium, halogen and C₁₋₃ alkyl, wherein said C₁₋₃ alkyl is optionally substituted with one or more halogens;
a is a double bond or a single bond;
b is a double bond or a single bond; and
m is 1 or 2,
with the proviso that no more than one of a and b is a double bond.

12. The compound according to any one of the preceding claims, wherein R² is of Formula (IX): 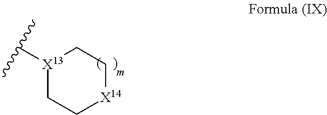
wherein
X¹³ is C(R²⁹) or N;
X¹⁴ is C(R²¹)(R²²) or O;
R²¹ is selected from the group consisting of H, deuterium, halogen and C₁₋₃ alkyl, wherein said C₁₋₃ alkyl is optionally substituted with one or more halogens;
R²² is selected from the group consisting of H, deuterium, halogen and C₁₋₃ alkyl, wherein said C₁₋₃ alkyl is optionally substituted with one or more halogens;
R²⁹ is H or C₁₋₃ alkyl; and
m is 0, 1 or 2.

13. The compound according to any one of claims 1 to 10, wherein R² is of Formula (X): 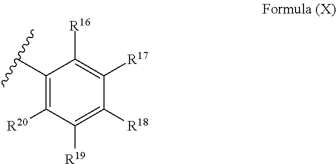
wherein
R¹⁶ is H or C₁₋₃ alkyl;
R¹⁷ is H or C₁₋₃ alkyl;
R¹⁸ is H or C₁₋₃ alkyl;
R¹⁹ is H or C₁₋₃ alkyl; and
R²⁰ is H or C₁₋₃ alkyl.

14. The compound according to any one of claims 1 to 10, wherein R² is selected from the group consisting of:

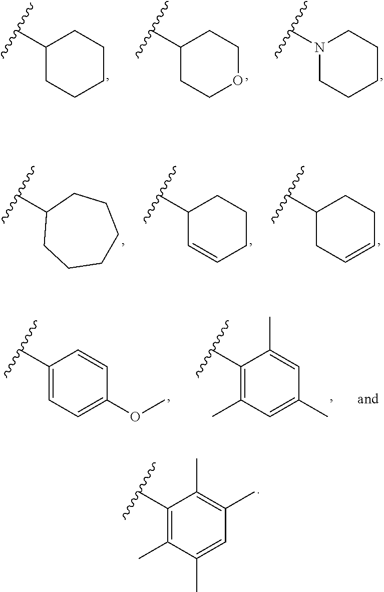

15. The compound according to any one of claims 1 to 10, wherein R² is C₅₋₈ bicycloalkyl, such as bicyclo[2.2.1]heptan-2-yl.

16. The compound according to any one of claims 1 to 10, wherein R² is C₉₋₁₂ tricycloalkyl, such as adamantyl.

17. The compound according to claim 1, wherein the compound is of Formula (I): 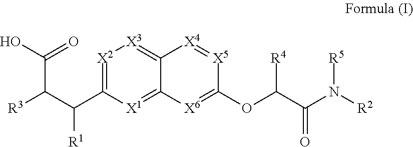
wherein
X¹ is CH;
X² is CH;
X³ is CH;
X⁴ is CH;
X⁵ is CH;
X⁶ is CH;
R¹ is selected from the group consisting of Formula (III), Formula (V) and Formula (VI): 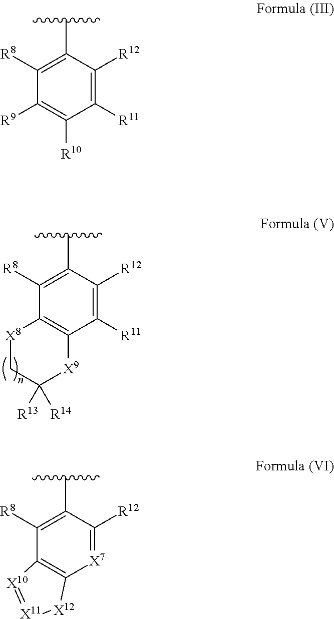
R² is of Formula (VIII) or Formula (X): 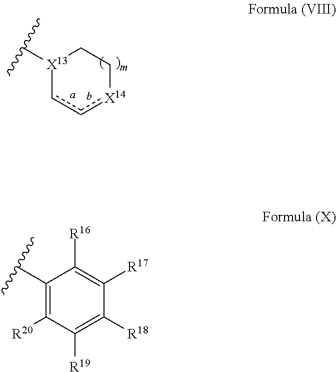
R³ is H or CH₃;
R⁴ is H or CH₃;
R⁵ is H or CH₃;
R⁸ is H or C₁₋₃ alkyl;
R⁹ is selected from the group consisting of H, C₁₋₃ alkyl, halogen, —OH and —OC₁₋₃ alkyl;
R¹⁰ is selected from the group consisting of H, halogen, —OH and —OC₁₋₃ alkyl;
R¹¹ is H;
R¹² is H or C₁₋₃ alkyl;
R¹³ is selected from the group consisting of H, C₁₋₃ alkyl and halogen;
R¹⁴ is selected from the group consisting of H, C₁₋₃ alkyl and halogen;
R¹⁵ is H or C₁₋₅ alkyl;
R¹⁶ is H or C₁₋₃ alkyl;
R¹⁷ is H or C₁₋₃ alkyl;
R¹⁸ is H or C₁₋₃ alkyl;
R¹⁹ is H or C₁₋₃ alkyl; and
R²⁰ is H or C₁₋₃ alkyl,
R²¹ is selected from the group consisting of H, deuterium, halogen and C₁₋₃ alkyl, wherein said C₁₋₃ alkyl is optionally substituted with one or more halogens;
R²² is selected from the group consisting of H, deuterium, halogen and C₁₋₃ alkyl, wherein said C₁₋₃ alkyl is optionally substituted with one or more halogens;
X⁷ is C(R¹¹) or N;
X⁸ is O or CH₂;
X⁹ is O;
X¹⁰ is N or CH;
X¹¹ is N or CH;
X¹² is O or N(R¹⁵);
X¹³ is C(H) or N;
X¹⁴ is (1) C(H) and b is a double bond; (2) C(R²¹)(R²²) and b is a single bond; or (3) O and a is a single bond;
a is a double bond or a single bond;
b is a double bond or a single bond;
n is 0 or 1;
m is 1 or 2;
with the proviso that no more than one of a and b is a double bond,
or a pharmaceutically acceptable salt, hydrate, polymorph, tautomer, or solvate thereof.

18. The compound according to claim 1, wherein the compound is selected from the group consisting of:
3-(7-(2-(Cyclohexylamino)-2-oxoethoxy)naphthalen-2-yl)propanoic acid;
3-(7-(2-Oxo-2-((tetrahydro-2H-pyran-4-yl)amino)ethoxy)naphthalen-2-yl)propanoic acid;
3-(7-(2-Morpholinoamino-2-oxoethoxy)naphthalen-2-yl)propanoic acid;
3-(7-(2-(Cycloheptylamino)-2-oxoethoxy)naphthalen-2-yl)propanoic acid;
3-(7-(2-((4-Methoxyphenyl)amino)-2-oxoethoxy)naphthalen-2-yl)propanoic acid;
3-(7-(2-(Mesitylamino)-2-oxoethoxy)naphthalen-2-yl)propanoic acid;
3-(7-(2-Oxo-2-((2,3,5,6-tetramethylphenyl)amino)ethoxy)naphthalen-2-yl)propanoic acid;
3-(7-(2-oxo-2-(phenylamino)ethoxy)naphthalen-2-yl)propanoic acid;
3-(7-(2-(Cycloheptylamino)-2-oxoethoxy)naphthalen-2-yl)-3-phenylpropanoic acid;
3-(7-(2-(Cycloheptylamino)-2-oxoethoxy)naphthalen-2-yl)-3-(4-hydroxyphenyl)propanoic acid;
3-(4-Chlorophenyl)-3-(7-(2-(cycloheptylamino)-2-oxoethoxy)naphthalen-2-yl)propanoic acid;
3-(7-(2-(Cycloheptylamino)-2-oxoethoxy)naphthalen-2-yl)-3-(4-methoxyphenyl)propanoic acid;
3-(7-(2-(Cycloheptylamino)-2-oxoethoxy)naphthalen-2-yl)-3-(4-ethoxyphenyl)propanoic acid;
3-(7-(2-(Cycloheptylamino)-2-oxoethoxy)naphthalen-2-yl)-3-(4-isopropoxyphenyl)propanoic acid;
3-(7-(2-(Cycloheptylamino)-2-oxoethoxy)naphthalen-2-yl)-3-(3-hydroxyphenyl)propanoic acid;
3-(7-(2-(Cycloheptylamino)-2-oxoethoxy)naphthalen-2-yl)-3-(3-methoxyphenyl)propanoic acid;
3-(3-Chlorophenyl)-3-(7-(2-(cycloheptylamino)-2-oxoethoxy)naphthalen-2-yl)propanoic acid;
3-(7-(2-(Cycloheptylamino)-2-oxoethoxy)naphthalen-2-yl)-3-(4-methoxy-3-methylphenyl)propanoic acid;
3-(7-(2-(Cycloheptylamino)-2-oxoethoxy)naphthalen-2-yl)-3-(4-methoxy-2-methylphenyl)propanoic acid;
3-(3-chloro-2-methylphenyl)-3-(7-(2-(cycloheptylamino)-2-oxoethoxy) naphthalen-2-yl)propanoic acid;
3-(benzo[d][1,3]dioxol-5-yl)-3-(7-(2-(cycloheptylamino)-2-oxoethoxy)naphthalen-2-yl)propanoic acid;
3-(7-(2-(cycloheptylamino)-2-oxoethoxy)naphthalen-2-yl)-3-(6-methylbenzo[d][1,3]dioxol-5-yl)propanoic acid;
3-(7-(2-(cycloheptylamino)-2-oxoethoxy)naphthalen-2-yl)-3-(7-methoxy-1-methyl-1H-benzo[d][1,2,3]triazol-5-yl)propanoic acid;
3-(7-(2-(cycloheptylamino)-2-oxoethoxy)naphthalen-2-yl)-3-(1-ethyl-4-methyl-1H-benzo[d][1,2,3]triazol-5-yl)propanoic acid;
3-(7-(2-(cycloheptylamino)-2-oxoethoxy)naphthalen-2-yl)-3-(2,3-dihydrobenzofuran-5-yl)propanoic acid;
3-(7-(2-(cycloheptylamino)-2-oxoethoxy)naphthalen-2-yl)-3-(2,2-difluorobenzo[d][1,3]dioxol-5-yl)propanoic acid;
3-(7-(2-(cycloheptylamino)-2-oxoethoxy)naphthalen-2-yl)-3-(2,3-dihydrobenzo[b][1,4]dioxin-6-yl)propanoic acid;
3-(Benzofuran-5-yl)-3-(7-(2-(cycloheptylamino)-2-oxoethoxy)naphthalen-2-yl)propanoic acid;
3-(7-(2-(Cycloheptylamino)-2-oxoethoxy)naphthalen-2-yl)-3-(7-methyl-2,3-dihydrobenzo[b][1,4]dioxin-6-yl)propanoic acid;
3-(5-chloro-2-methylphenyl)-3-(7-(2-(cycloheptylamino)-2-oxoethoxy) naphthalen-2-yl)propanoic acid;
3-(7-(2-(cycloheptylamino)-2-oxoethoxy)naphthalen-2-yl)-3-(6-methyl-2,3-dihydrobenzofuran-5-yl)propanoic acid;
3-(7-(2-(cycloheptylamino)-2-oxoethoxy)naphthalen-2-yl)-3-(6-methylbenzo[d][1,3]dioxol-5-yl)propanoic acid;
3-(6-chlorobenzo[d][1,3]dioxol-5-yl)-3-(7-(2-(cycloheptylamino)-2-oxoethoxy)naphthalen-2-yl)propanoic acid;
3-(7-(2-(cyclohexylamino)-2-oxoethoxy)naphthalen-2-yl)-3-(4-methoxyphenyl)propanoic acid;
3-(7-(2-(cyclohexylamino)-2-oxoethoxy)naphthalen-2-yl)-3-(4-methoxy-2-methylphenyl)propanoic acid;
3-(7-(2-(cyclohexylamino)-2-oxoethoxy)naphthalen-2-yl)-3-(4-methoxy-2,6-dimethylphenyl)propanoic acid;
3-(3-chlorophenyl)-3-(7-(2-(cyclohexylamino)-2-oxoethoxy)naphthalen-2-yl)propanoic acid;
3-(3-chloro-2-methylphenyl)-3-(7-(2-(cyclohexylamino)-2-oxoethoxy)naphthalen-2-yl)propanoic acid;
3-(benzo[d][1,3]dioxol-5-yl)-3-(7-(2-(cyclohexylamino)-2-oxoethoxy)naphthalen-2-yl)propanoic acid;
3-(7-(2-(cyclohexylamino)-2-oxoethoxy)naphthalen-2-yl)-3-(6-methylbenzo[d][1,3]dioxol-5-yl)propanoic acid;
3-(7-(2-(cyclohexylamino)-2-oxoethoxy)naphthalen-2-yl)-3-(1-ethyl-4-methyl-1H-benzo[d][1,2,3]triazol-5-yl)propanoic acid;
3-(7-(2-(cyclohexylamino)-2-oxoethoxy)naphthalen-2-yl)-3-(2,3-dihydrobenzofuran-5-yl)propanoic acid;
3-(7-(2-(cyclohexylamino)-2-oxoethoxy)naphthalen-2-yl)-3-(2,2-dimethylbenzo[d][1,3]dioxol-5-yl)propanoic acid;
3-(7-(2-(cyclohexylamino)-2-oxoethoxy)naphthalen-2-yl)-3-(2,2-difluorobenzo[d][1,3]dioxol-5-yl)propanoic acid;
3-(7-(2-(cyclohexylamino)-2-oxoethoxy)naphthalen-2-yl)-3-(2,3-dihydrobenzo[b][1,4]dioxin-6-yl)propanoic acid;
3-(7-(2-(cyclohexylamino)-2-oxoethoxy)naphthalen-2-yl)-3-(5-methoxy-2-methylphenyl)propanoic acid;
3-(7-(2-(cyclohexylamino)-2-oxoethoxy)naphthalen-2-yl)-3-(6-methyl-1H-pyrrolo[2,3-b]pyridin-5-yl)propanoic acid;
3-(Benzofuran-5-yl)-3-(7-(2-(cyclohexylamino)-2-oxoethoxy)naphthalen-2-yl)propanoic acid;
3-(5-Chloro-2-methylphenyl)-3-(7-(2-(cyclohexylamino)-2-oxoethoxy)naphthalen-2-yl)propanoic acid;
3-(7-(2-(cyclohexylamino)-2-oxoethoxy)naphthalen-2-yl)-3-(7-methyl-2,3-dihydrobenzo[b][1,4]dioxin-6-yl)propanoic acid;
3-(7-(2-(cycloheptylamino)-2-oxoethoxy)naphthalen-2-yl)-3-(2,2-difluoro-6-methylbenzo[d][1,3]dioxol-5-yl)propanoic acid;
3-(7-(2-(cyclohexylamino)-2-oxoethoxy)naphthalen-2-yl)-3-(6-methylbenzofuran-5-yl)propanoic acid;
3-(7-(2-(cyclohexylamino)-2-oxoethoxy)naphthalen-2-yl)-3-(6-methyl-2,3-dihydrobenzofuran-5-yl)propanoic acid;
3-(Benzo[d][1,3]dioxol-5-yl)-3-(7-(2-(cyclohex-2-en-1-ylamino)-2-oxoethoxy)naphthalen-2-yl)propanoic acid;
3-(7-(2-(Cyclohex-2-en-1-ylamino)-2-oxoethoxy)naphthalen-2-yl)-3-(6-methylbenzo[d][1,3]dioxol-5-yl)propanoic acid;
3-(3-Chloro-2-methylphenyl)-3-(7-(2-(cyclohex-2-en-1-ylamino)-2-oxoethoxy)naphthalen-2-yl)propanoic acid;
3-(7-(2-(cyclohex-2-en-1-ylamino)-2-oxoethoxy)naphthalen-2-yl)-3-(2,3-dihydrobenzo[b][1,4]dioxin-6-yl)propanoic acid;
3-(7-(2-(Cyclohex-2-en-1-ylamino)-2-oxoethoxy)naphthalen-2-yl)-3-(2,3-dihydrobenzofuran-5-yl)propanoic acid;
3-(7-(2-(Cyclohex-2-en-1-ylamino)-2-oxoethoxy)naphthalen-2-yl)-3-(2,2-difluorobenzo[d][1,3]dioxol-5-yl)propanoic acid;
3-(Benzofuran-5-yl)-3-(7-(2-(cyclohex-2-en-1-ylamino)-2-oxoethoxy)naphthalen-2-yl)propanoic acid;
3-(5-Chloro-2-methylphenyl)-3-(7-(2-(cyclohex-2-en-1-ylamino)-2-oxoethoxy)naphthalen-2-yl)propanoic acid;
3-(7-(2-(cyclohex-2-en-1-ylamino)-2-oxoethoxy)naphthalen-2-yl)-3-(7-methyl-2,3-dihydrobenzo[b][1,4]dioxin-6-yl)propanoic acid;
3-(3-chlorophenyl)-3-(7-(2-(cyclohex-2-en-1-ylamino)-2-oxoethoxy)naphthalen-2-yl)propanoic acid;
3-(7-(2-(cyclohex-2-en-1-ylamino)-2-oxoethoxy)naphthalen-2-yl)-3-(4-methoxy-2-methylphenyl)propanoic acid;
3-(7-(2-(cyclohex-2-en-1-ylamino)-2-oxoethoxy)naphthalen-2-yl)-3-(2,2-difluoro-6-methylbenzo[d][1,3]dioxol-5-yl)propanoic acid;
3-(7-(2-(cycloheptylamino)-2-oxoethoxy)naphthalen-2-yl)-3-(6-methylbenzo[d][1,3]dioxol-5-yl)propanoic acid;
3-(7-(2-(cyclohex-2-en-1-ylamino)-2-oxoethoxy)naphthalen-2-yl)-3-(6-methyl-2,3-dihydrobenzofuran-5-yl)propanoic acid;
3-(benzo[d][1,3]dioxol-5-yl)-3-(7-(2-(((1S,2S,4R)-bicyclo[2.2.1]heptan-2-yl)amino)-2-oxoethoxy)naphthalen-2-yl)propanoic acid;
3-(7-(2-(((1S,2S,4R)-bicyclo[2.2.1]heptan-2-yl)amino)-2-oxoethoxy)naphthalen-2-yl)-3-(6-chlorobenzo[d][1,3]dioxol-5-yl)propanoic acid;
3-(7-(2-(((1S,2S,4R)-bicyclo[2.2.1]heptan-2-yl)amino)-2-oxoethoxy)naphthalen-2-yl)-3-(6-methylbenzo[d][1,3]dioxol-5-yl)propanoic acid;
3-(7-(2-(((1S,2S,4R)-bicyclo[2.2.1]heptan-2-yl)amino)-2-oxoethoxy)naphthalen-2-yl)-3-(7-methyl-2,3-dihydrobenzo[b][1,4]dioxin-6-yl)propanoic acid;
3-(Benzo[d][1,3]dioxol-5-yl)-3-(7-(2-(cyclohex-3-en-1-ylamino)-2-oxoethoxy)naphthalen-2-yl)propanoic acid;
3-(benzo[d][1,3]dioxol-5-yl)-3-(7-(2-((4,4-difluorocyclohexyl)amino)-2-oxoethoxy)naphthalen-2-yl)propanoic acid;
3-(benzo[d][1,3]dioxol-5-yl)-3-(7-(2-(isobutylamino)-2-oxoethoxy)naphthalen-2-yl)propanoic acid;
3-(benzo[d][1,3]dioxol-5-yl)-3-(7-(2-(cyclopentylamino)-2-oxoethoxy)naphthalen-2-yl)propanoic acid;
3-(7-(2-(((1S,3s)-adamantan-1-yl)amino)-2-oxoethoxy)naphthalen-2-yl)-3-(benzo[d][1,3]dioxol-5-yl)propanoic acid;
3-(benzo[d][1,3]dioxol-5-yl)-3-(7-(2-((1-methylcyclohexyl)amino)-2-oxoethoxy)naphthalen-2-yl)propanoic acid;
3-(benzo[d][1,3]dioxol-5-yl)-3-(7-(2-((4,4-dimethylcyclohexyl)amino)-2-oxoethoxy)naphthalen-2-yl)propanoic acid;
3-(benzo[d][1,3]dioxol-5-yl)-3-(7-(2-(((1S,2R,4R)-bicyclo[2.2.1]heptan-2-yl)amino)-2-oxoethoxy)naphthalen-2-yl)propanoic acid;
3-(benzo[d][1,3]dioxol-5-yl)-3-(7-(2-oxo-2-((trans-4-(trifluoromethyl)cyclohexyl)amino)ethoxy)naphthalen-2-yl)propanoic acid;
3-(benzo[d][1,3]dioxol-5-yl)-3-(7-(2-oxo-2-(2-propylpiperidin-1-yl)ethoxy)naphthalen-2-yl)propanoic acid;
3-(benzo[d][1,3]dioxol-5-yl)-3-(7-(2-oxo-2-(phenylamino)ethoxy)naphthalen-2-yl)propanoic acid;
3-(Benzo[d][1,3]dioxol-5-yl)-3-(7-((1-(cycloheptylamino)-1-oxopropan-2-yl)oxy)naphthalen-2-yl)propanoic acid;
3-(benzo[d][1,3]dioxol-5-yl)-3-(7-(2-(cycloheptyl(methyl)amino)-2-oxoethoxy)naphthalen-2-yl)propanoic acid;
3-(benzo[d][1,3]dioxol-5-yl)-3-(7-(2-(cycloheptylamino)-2-oxoethoxy)naphthalen-2-yl)-2-methylpropanoic acid;
3-(benzo[d][1,3]dioxol-5-yl)-3-(7-(2-(cycloheptylamino)-2-oxoethoxy)naphthalen-2-yl)-2,2-dimethylpropanoic acid;
Methyl 3-(benzo[d][1,3]dioxol-5-yl)-3-(7-(2-(cycloheptylamino)-2-oxoethoxy)naphthalen-2-yl)propanoate;
Ethyl 3-(7-(2-(cyclohex-2-en-1-ylamino)-2-oxoethoxy)naphthalen-2-yl)-3-(6-methylbenzo[d][1,3]dioxol-5-yl)propanoate;
N-cycloheptyl-2-((7-(1-(6-methylbenzo[d][1,3]dioxol-5-yl)-3-oxo-3-(2-oxothiazolidin-3-yl)propyl)naphthalen-2-yl)oxy) acetamide;
N-cycloheptyl-2-((7-(1-7-methoxy-1-methyl-1H-benzo[d][1,2,3]triazol-5-yl)-3-oxo-3-(2-oxothiazolidin-3-yl)propyl)naphthalen-2-yl)oxy) acetamide;
3-(benzo[d][1,3]dioxol-5-yl)-3-(3-(2-(cycloheptylamino)-2-oxoethoxy) quinolin-6-yl)propanoic acid;
3-(benzo[d][1,3]dioxol-5-yl)-3-(2-(2-(cycloheptylamino)-2-oxoethoxy) quinolin-7-yl)propanoic acid; and
3-(benzo[d][1,3]dioxol-5-yl)-3-(6-(2-(cycloheptylamino)-2-oxoethoxy) quinolin-3-yl)propanoic acid,
or a pharmaceutically acceptable salt thereof.

19. A pharmaceutical composition comprising the compound according to any one of the preceding claims.

20. The compound according to any one of claims 1 to 18 for use as a medicament.

21. The compound according to any one of claims 1 to 18 for use in treating, ameliorating and/or preventing a disease selected from the group consisting of Central Nervous System (CNS) diseases; lung diseases; autoimmune disorders; cancer; metabolic and/or inflammatory liver conditions; chronic kidney disase (CKD), incl. Alport syndrome; skin diseases; retinal ischemia-reperfusion (I/R) injury; ulcerative colitis; and acetaminophen-induced hepatotoxicity.

22. The compound for use according to claim 21, wherein the disease is a CNS disease selected from the group consisting of ischemic stroke, traumatic brain injury, cerebral ischemia, intracerebral haemorrhage, Alzheimer's disease, Parkinson's disease, and multiple sclerosis.

23. The compound for use according to claim 21, wherein the disease is non-alcoholic steatohepatitis (NASH).

24. The compound for use according to claim 21, wherein the disease is CDK.
